(19) United States
(12) Patent Application Publication
(10) Pub. No.: US 2026/0248833 A1
(43) Pub. Date: Aug. 27, 2026

(54) PYRIMIDINE NUCLEOSIDE TREATMENTS

(71) Applicant: The Children`s Medical Center Corporation, Boston, MA (US)

(72) Inventors: Suneet Agarwal, Lexington, MA (US); William Mannherz, Cambridge, MA (US)

(21) Appl. No.: 18/875,933

(22) PCT Filed: Jun. 21, 2023
(86) PCT No.: PCT/US2023/025867

§ 371 Date: Dec. 17, 2024

Related U.S. Application Data
(60) Provisional application No. 63/354,226, filed on Jun. 21, 2022.
(60) Provisional application No. 63/394,588, filed on Aug. 2, 2022.

(51) Int. Cl. A61K 31/7072; A61K 31/4425; A61K 31/513; A61K 31/519; A61K 31/708; A61K 45/06; A61P 7/00; A61P 43/00

(52) U.S. Cl. CPC A61K 31/7072; A61K 31/4425; A61K 31/513; A61K 31/519; A61K 31/708; A61K 45/06; A61P 7/00; A61P 43/00

(57) ABSTRACT

The present disclosure provides pyrimidine nucleoside compounds, including torcitabine, thymidine, deoxyuridine, deoxycytidine and uridine, and methods to treat telomere biology disorders (TBDs) and aging-related diseases, including hematological disorder, liver disease, or hepatic fibrosis, comprising administering to a subject diagnosed with said telomere biology disorder a therapeutically effective amount of said compounds.

### CLAIM OF PRIORITY

This application claims priority to U.S. Provisional Patent Application Ser. No. 63/354,226, filed on Jun. 21, 2022, and to U.S. Provisional Patent Application Ser. No. 63/394,588, filed on Aug. 2, 2022, the entire contents of which are hereby incorporated by reference.

### FEDERALLY SPONSORED RESEARCH AND DEVELOPMENT

This invention was made with government support under grant number DK107716, awarded by National Institutes of Health (NIH). The government has certain rights in the invention.

### TECHNICAL FIELD

The present disclosure relates to using pyrimidine nucleosides (e.g., thymidine and/or deoxyruridine) to treat telomere biology disorders, such as dyskeratosis congenita, aplastic anemia, myelodysplastic syndrome, and interstitial lung diseases, and aging-related disorders.

### BACKGROUND

A telomere is a region of repetitive nucleotide sequences at each end of a chromosome, which protects the end of the chromosome from deterioration or from fusion with neighboring chromosomes. The length of a telomere is a key determinant of cellular self-renewal capacity. Short telomeres, due to genetic or acquired insults, cause a loss of cellular self-renewal and result in life-threatening diseases, such as dyskeratosis congenita, for which there are few if any effective medical therapies.

### SUMMARY

Controlled synthesis and degradation of DNA precursors is essential for faithful genome replication by DNA polymerases. Telomeres are unique DNA structures at chromosome termini which are elongated by the telomerase reverse transcriptase. Genetic determinants of telomere length are associated with severe diseases as well as longevity, but remain incompletely characterized. Described herein is CRISPR/Cas9 screening to identify pyrimidine nucleotide (e.g., deoxythymidine (dT) nucleotide) metabolism as a critical regulator of human telomere length.

Disrupting genes required for dT nucleotide production impaired telomere maintenance, whereas inhibiting SAMHD1, which depletes dT triphosphate (dTTP), lengthened telomeres. Remarkably, supplementation with pyrimidine nucleosides (e.g., dT or dU) alone or in combination with another nucleoside (e.g., dC) drove robust telomere elongation in a telomerase-dependent manner, and without interrupting cell cycle. In one non-limiting example, in cells derived from patients with fatal genetic telomere disorders, dT supplementation or inhibition of SAMHD1 promoted telomere restoration. Collectively, this disclosure demonstrates a critical role of pyrimidine nucleosides such as thymidine in regulating telomere length, which may be therapeutically actionable in patients with degenerative disorders.

In one general aspect, the present disclosure provides a method of treating a telomere biology disorder, the method comprising administering to a subject diagnosed with said telomere biology disorder a therapeutically effective amount of a compound of Formula (A):

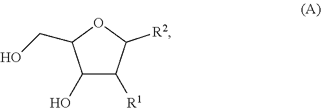

or a pharmaceutically acceptable salt thereof, wherein:
R¹ is selected from H and OH;
R² is selected from any one of the following moieties:

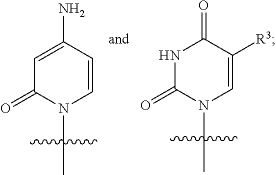

and
R³ is selected from H and CH₃.

In some embodiments, the compound has formula:

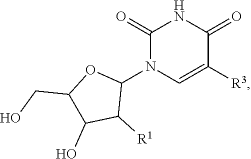

or a pharmaceutically acceptable salt thereof.

In some embodiments, R³ is H.

In some embodiments, R³ is CH₃.

In some embodiments, the compound has formula:

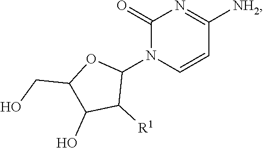

or a pharmaceutically acceptable salt thereof.

In some embodiments, R¹ is H.

In some embodiments, R¹ is OH.

In some embodiments, the compound of Formula (A) has Formula (I):

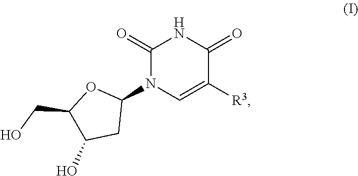

or a pharmaceutically acceptable salt thereof.

In some embodiments, the compound of Formula (I) is:

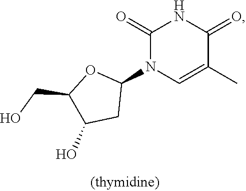 [SMILES: Cc1cn([C@H]2C[C@H](O)[C@@H](CO)O2)c(=O)[nH]c1=O]

or a pharmaceutically acceptable salt thereof.

In some embodiments, the compound of Formula (I) is:

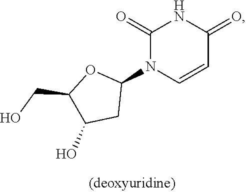 [SMILES: O=c1ccn([C@H]2C[C@H](O)[C@@H](CO)O2)c(=O)[nH]1]

or a pharmaceutically acceptable salt thereof.

In some embodiments, the compound of Formula (A) is:

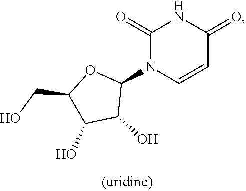 [SMILES: O=c1ccn([C@@H]2O[C@H](CO)[C@@H](O)[C@H]2O)c(=O)[nH]1]

or a pharmaceutically acceptable salt thereof.

In some embodiments, the compound of Formula (A) is:

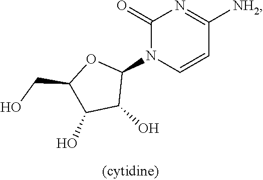 [SMILES: Nc1ccn([C@@H]2O[C@H](CO)[C@@H](O)[C@H]2O)c(=O)n1]

or a pharmaceutically acceptable salt thereof.

In some embodiments, the telomere biology disorder is selected from dyskeratosis congenita, aplastic anemia, myelodysplastic syndrome, pulmonary fibrosis, idiopathic pulmonary fibrosis, interstitial lung disease, hematological disorder, liver disease, hepatic fibrosis, Hoyeraal-Hreidarsson syndrome, Coats Plus syndrome, and Revesz syndrome.

In some embodiments, the telomere biology disorder is selected from dyskeratosis congenita, aplastic anemia, and myelodysplastic syndrome.

In some embodiments, the telomere biology disorder is dyskeratosis congenita.

In some embodiments, the telomere biology disorder is aplastic anemia.

In some embodiments, the telomere biology disorder is interstitial lung disease.

In some embodiments, the telomere biology disorder is myelodysplastic syndrome.

In some embodiments, the method comprises administering the compound to the subject orally.

In some embodiments, the method comprises administering the compound in a dosage form selected from a capsule, a tablet, and a sachet.

In some embodiments, the method comprises administering the compound to the subject intravascularly.

In some embodiments, the method comprises administering the compound in a dosage form comprising an injectable or infusible aqueous solution.

In some embodiments, the therapeutically effective amount of the compound is from about 50 mg/kg/day to about 500 mg/kg/day.

In some embodiments, the therapeutically effective amount of the compound is from about 130 mg/kg/day to about 400 mg/kg/day.

In some embodiments, the therapeutically effective amount is about 130 mg/kg/day, about 260 mg/kg/day, or about 400 mg/kg/day.

In some embodiments, the compound is administered once a day.

In some embodiments, the compound is administered twice a day.

In some embodiments, the compound is administered three times a day.

In some embodiments, the method comprises administering at least two compounds of Formula (A), or a pharmaceutically acceptable salt thereof.

In some embodiments, the method comprises co-administering:

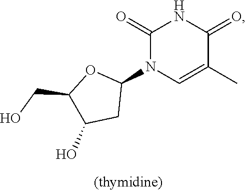 [SMILES: Cc1cn([C@H]2C[C@H](O)[C@@H](CO)O2)c(=O)[nH]c1=O]

or a pharmaceutically acceptable salt thereof, and

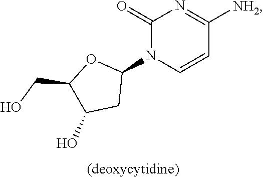 [SMILES: Nc1ccn([C@H]2C[C@H](O)[C@@H](CO)O2)c(=O)n1]

or a pharmaceutically acceptable salt thereof.

In some embodiments, the compound is administered in combination with an additional therapeutic agent useful in treating a telomere biology disorder

In some embodiments, the additional therapeutic agent is purine nucleoside, or a pharmaceutically acceptable salt thereof.

In some embodiments, the purine nucleoside is selected from adenosine (A), deoxyadenosine (dA), guanosine (G), and deoxyguanosine (dG), or a pharmaceutically acceptable salt thereof.

In some embodiments, the additional therapeutic agent is an inhibitor of dNTPase SAM domain and HD domain-containing protein 1 (SAMHD1), or a pharmaceutically acceptable salt thereof.

In some embodiments, the SAMHD1 inhibitor is selected from miRNA181a/b, an anti-SAMHD1 antibody, a T cell receptor, and a protein having at least 75% identity to VPX or VPR.

In some embodiments, the SAMHD1 inhibitor is selected from erythrotyrosine, sennoside A, evans blue, merbromin, phenylmercuric acetate, thiram, bronopol, cephalosporin C, pidolic acid, diphenhydramine, aurothiomalate, rose bengal, chlorambucil, pyrithione zinc, lomofungin, troglitazone, montelukast, pranlukast, L-thyroxine, ergotamine, amrinone, retinoic acid, ethacrynic acid, hexestrol, tolfenamic acid, bexarotene, sulindac, zolmitriptan, nifedipine, tetracycline, nisoldipine, medroxyprogesterone acetate, trifluoperazine, primaquine, adapalene, aprepitant, tolcapone, zafirlukast, delavirdine, topotecan, ceftazidime, zoledronic acid, anethole-trithione, and disulfiram, or a pharmaceutically acceptable salt thereof.

In some embodiments, the additional therapeutic agent is an inhibitor of thymidine phosphorylase (TYMP), or a pharmaceutically acceptable salt thereof.

In some embodiments, the TYMP inhibitor is selected from tipiracil, 6-aminothymine (6AT), amino-5-chlorouracil (6A5CU), 6-amino-5-bromouracil (6A5BU), 7-deazaxanthine (7DX), 7-(2-aminoethyl)-deazaxanthine, 6-(2-aminoethylamino)-5-chlorouracil (AEAC), 5-chloro-6-(1-imidazolylmethyl)uracil (CIMU), 6-methylenepyridinium, 6-(4-phenylbutylamino)uracil, 5-chloro-6-[1-(2-iminopyrrolidinyl)methyl]uracil hydrochloride (TPI), and N-(2,4-dioxo-1,2,3,4-tetrahydro-thieno[3,2-d]pyrimidin-7-yl)guanidine, or a pharmaceutically acceptable salt thereof.

In some embodiments, the present disclosure provides a method of treating a disorder associated with aging, the method comprising administering to a subject diagnosed with said disorder associated with aging a therapeutically effective amount of a compound of Formula (A):

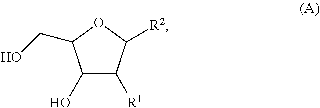

or a pharmaceutically acceptable salt thereof, wherein:
R¹ is selected from H and OH;
R² is selected from any one of the following moieties:

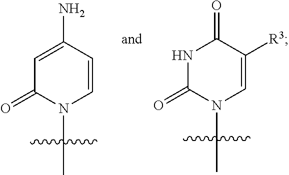

and
R³ is selected from H and CH₃.

In some embodiments, the compound has formula:

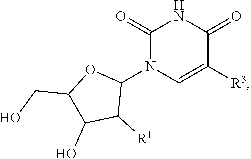

or a pharmaceutically acceptable salt thereof.

In some embodiments, R³ is H.

In some embodiments, R³ is CH₃.

In some embodiments, the compound has formula:

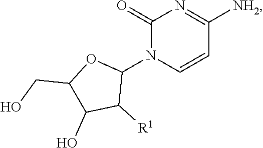

or a pharmaceutically acceptable salt thereof.

In some embodiments, R¹ is H.

In some embodiments, R¹ is OH.

In some embodiments, the compound of Formula (A) has Formula (I):

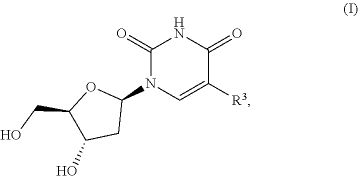

or a pharmaceutically acceptable salt thereof.

In some embodiments, the compound of Formula (I) is:

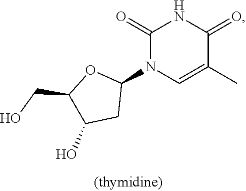 [SMILES: Cc1cn([C@H]2C[C@H](O)[C@@H](CO)O2)c(=O)[nH]c1=O]

or a pharmaceutically acceptable salt thereof.

In some embodiments, the compound of Formula (I) is:

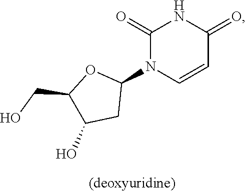 [SMILES: O=c1ccn([C@H]2C[C@H](O)[C@@H](CO)O2)c(=O)[nH]1]

or a pharmaceutically acceptable salt thereof.

In some embodiments, the compound of Formula (A) is:

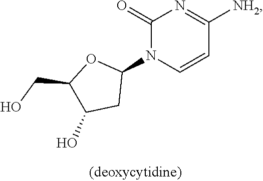 [SMILES: Nc1ccn([C@H]2C[C@H](O)[C@@H](CO)O2)c(=O)n1]

or a pharmaceutically acceptable salt thereof.

In some embodiments, the compound of Formula (A) is:

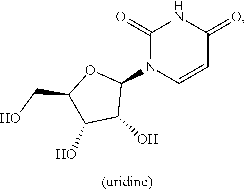 [SMILES: O=c1ccn([C@@H]2O[C@H](CO)[C@@H](O)[C@H]2O)c(=O)[nH]1]

or a pharmaceutically acceptable salt thereof.

In some embodiments, the compound of Formula (A) is:

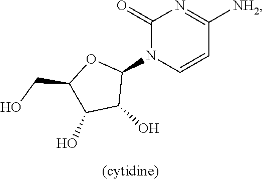 [SMILES: Nc1ccn([C@@H]2O[C@H](CO)[C@@H](O)[C@H]2O)c(=O)n1]

or a pharmaceutically acceptable salt thereof.

In some embodiments, the disorder associated with aging is selected from inflammatory disease, immune disease, adult disease, infectious disease, cardiovascular disease, dermatological disease, ophthalmic disease, neurological disease, wasting disorder, metabolic disorder, cancer, a pre-cancerous condition, and a disorder of the connective tissue

In some embodiments, the disorder associated with aging is selected from age-related anxiety, anemia, anorexia, arteriosclerosis, asthma, balance disorder, Bell's palsy, bone marrow failure, breathlessness, cachexia, chronic infection, cirrhosis, congestive heart failure, deafness, diabetes, emphysema, failure to thrive, flu, frailty, gastrointestinal ulcer, generalized anxiety disorder, gout, hair loss, hearing loss, hepatic insufficiency, high blood pressure, high fat, hip dislocation, hypercholesterolemia, hyperglycemia, hyperhomocysteinemia, hyperlipidemia, immunosenescence, impaired mobility, loss of appetite, loss of bone density, loss of sense of taste, metabolic syndrome, muscle loss, muscle wasting, muscular dystrophy, myocardial infarction, obesity, organ dysfunction, osteoporosis, peripheral artery disease, peripheral vascular disease, pneumonia secondary to impaired immune function, pulmonary disease, pulmonary emphysema, pulmonary fibrosis, reduced fitness, renal disease, renal insufficiency, scoliosis, spinal stenosis, syndrome X, tinnitus, urinary incontinence, vertebral fracture, weight loss, coronary artery disease, diabetes mellitus, type 2 diabetes, osteoarthritis, rheumatoid arthritis, sarcopenia, hypertension, atherosclerosis, ischemia, reperfusion injury, premature death, vascular insufficiency, interstitial lung disease, age-related decline in cognitive function, age-related decline in cardiopulmonary function, age-related decline in muscle strength, age-related decline in vision, and age-related decline in hearing.

In some embodiments, the dermatological disease is a senescence-associated dermatological disease selected from rough skin, formation of wrinkles, coloring or spots, abnormal coloration of skin, formation of sagging, easy skin damage, atrophy, diabetic ulcers, and other ulcers.

In some embodiments, the ophthalmic disease is senescence-associated ophthalmic disease selected from cataract, corneal abrasion, conjunctivitis, chalazion, glaucoma, macular degeneration, and age-related macular degeneration.

In some embodiments, the neurological disease is selected from Alzheimer's disease, hearing loss, dementia, chronic traumatic encephalopathy, brain atrophy, amyotrophic lateral sclerosis, Parkinson's disease, Gillian-Barre syndrome, peripheral neuropathy, Creutzfeldt-Jakob disease, frontotemporal dementia, spinal muscular atrophy, and Friedreich's ataxia, vascular dementia, mild cognitive impairment, severe cognitive impairment, memory loss, pontocerebellar hypoplasia, motor neuron disease, Machado-Joseph disease, spino-cerebellar ataxia, Multiple sclerosis, Huntington's disease, hearing impairment, balance impairment, ataxias, epilepsy, mood disorder, schizophrenia, bipolar disorder, depression, Pick's Disease, stroke, CNS hypoxia, cerebral senility, neural injury, and head trauma.

In some embodiments, the cancer is selected from bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

In some embodiments, the method includes administering the compound to the subject orally.

In some embodiments, the method includes administering the compound in a dosage form selected from a capsule, a tablet, and a sachet.

In some embodiments, the method includes administering the compound to the subject intravascularly.

In some embodiments, the method includes administering the compound in a dosage form comprising an injectable or infusible aqueous solution.

In some embodiments, the therapeutically effective amount of the compound is from about 50 mg/kg/day to about 500 mg/kg/day.

In some embodiments, the therapeutically effective amount of the compound is from about 130 mg/kg/day to about 400 mg/kg/day.

In some embodiments, the therapeutically effective amount is about 130 mg/kg/day, about 260 mg/kg/day, or about 400 mg/kg/day.

In some embodiments, the compound is administered one a day.

In some embodiments, the compound is administered twice a day.

In some embodiments, the compound is administered three times a day.

In some embodiments, the method includes administering at least two compounds of Formula (A), or a pharmaceutically acceptable salt thereof.

In some embodiments, the method includes co-administering:

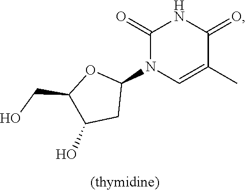 [SMILES: Cc1cn([C@H]2C[C@H](O)[C@@H](CO)O2)c(=O)[nH]c1=O]

or a pharmaceutically acceptable salt thereof, and

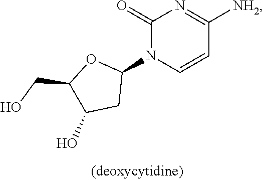 [SMILES: Nc1ccn([C@H]2C[C@H](O)[C@@H](CO)O2)c(=O)n1]

or a pharmaceutically acceptable salt thereof.

In some embodiments, the compound is administered in combination with an additional therapeutic agent useful in treating a telomere biology disorder

In some embodiments, the additional therapeutic agent is purine nucleoside, or a pharmaceutically acceptable salt thereof.

In some embodiments, the purine nucleoside is selected from adenosine (A), deoxyadenosine (dA), guanosine (G), and deoxyguanosine (dG), or a pharmaceutically acceptable salt thereof.

In some embodiments, the additional therapeutic agent is an inhibitor of dNTPase SAM domain and HD domain-containing protein 1 (SAMHD1), or a pharmaceutically acceptable salt thereof.

In some embodiments, the SAMHD1 inhibitor is selected from miRNA181a/b, an anti-SAMHD1 antibody, a T cell receptor, and a protein having at least 75% identity to VPX or VPR.

In some embodiments, the SAMHD1 inhibitor is selected from erythrotyrosine, sennoside A, evans blue, merbromin, phenylmercuric acetate, thiram, bronopol, cephalosporin C, pidolic acid, diphenhydramine, aurothiomalate, rose bengal, chlorambucil, pyrithione zinc, lomofungin, troglitazone, montelukast, pranlukast, L-thyroxine, ergotamine, amrinone, retinoic acid, ethacrynic acid, hexestrol, tolfenamic acid, bexarotene, sulindac, zolmitriptan, nifedipine, tetracycline, nisoldipine, medroxyprogesterone acetate, trifluoperazine, primaquine, adapalene, aprepitant, tolcapone, zafirlukast, delavirdine, topotecan, ceftazidime, zoledronic acid, anethole-trithione, and disulfiram, or a pharmaceutically acceptable salt thereof.

In some embodiments, the additional therapeutic agent is an inhibitor of thymidine phosphorylase (TYMP), or a pharmaceutically acceptable salt thereof.

In some embodiments, the TYMP inhibitor is selected from tipiracil, 6-aminothymine (6AT), amino-5-chlorouracil (6A5CU), 6-amino-5-bromouracil (6A5BU), 7-deazaxanthine (7DX), 7-(2-aminoethyl)-deazaxanthine, 6-(2-aminoethylamino)-5-chlorouracil (AEAC), 5-chloro-6-(1-imidazolylmethyl)uracil (CIMU), 6-methylenepyridinium, 6-(4-phenylbutylamino)uracil, 5-chloro-6-[1-(2-iminopyrrolidinyl)methyl]uracil hydrochloride (TPI), and N-(2,4-dioxo-1,2,3,4-tetrahydro-thieno[3,2-d]pyrimidin-7-yl)guanidine, or a pharmaceutically acceptable salt thereof.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1A Telomere length CRISPR/Cas9 screening reveals thymidine nucleotide metabolism genes contribute to telomere length control. Diagram of flow-FISH telomere content CRISPR/Cas9 screening strategy.

FIG. 1B Telomere length CRISPR/Cas9 screening reveals thymidine nucleotide metabolism genes contribute to telomere length control. Cas9 expressing K562 cells were transduces with a gRNA library targeting 53 nucleotide metabolism genes with 10 gRNAs per gene and 200 non targeting controls. Cells were cultured for 26 days prior to flow-FISH sorting of the 5% of cells with the highest and lowest telomere fluorescence. Screen was performed twice. gRNA enrichment was analyzed using the Mageck Robust Rank Algorithm (RRA). RRA gene enrichment score vs log 2 fold change gRNA abundance in high vs low 5th percentile TelC-A647 fluorescence populations. Black dots represent genes significantly enriched in long or short populations with a false discovery rate <0.05.

FIG. 1C Telomere length CRISPR/Cas9 screening reveals thymidine nucleotide metabolism genes contribute to telomere length control. Diagram of dT nucleotide metabolism. Cytidine and uridine can be converted to thymidine nucleotides, offering a biological rational for telomere length increases. The figure also shows genetic targets offering synergism (inhibition of TYMP and/or inhibition of SAMHD1). See e.g., Am J Hum Genet, 2020, 106, 3, 389-404; and Nature Genetics, 53, 1425-1433, 2021, which are incorporated herein by reference in their entirety.

FIG. 2A Thymidine treatment drives telomere lengthening in human cells in a telomerase dependent manner without disrupting cell cycle. Terminal restriction fragment (TRF) Southern blot probed for the telomere repeat of K562, 293T or TERC−/−293T cells treated with or without 100 μM each of dA, dC, dG and dT for 8 days.

FIG. 2B Thymidine treatment drives telomere lengthening in human cells in a telomerase dependent manner without disrupting cell cycle. TRF Southern blot of 293T cells treated with 100 μM of the indicated deoxynucleoside for 9 days. Untreated lanes are technical replicates.

FIG. 2C Thymidine treatment drives telomere lengthening in human cells in a telomerase dependent manner without disrupting cell cycle. TRF Southern blot of K562 cells treated with 100 μM of the indicated deoxynucleoside for 8 days.

FIG. 2D Thymidine treatment drives telomere lengthening in human cells in a telomerase dependent manner without disrupting cell cycle. Quantification of b,c, telomere length quantified using the WALTER telomere length analysis software. Students T statistic, ns=P>0.05, **=P<0.01, ***=P<0.001, ****=P<0.0001. Thymidine (dT), but not deoxyadenosine (dA) or deoxyguanosine (dG), rapidly and robustly increases telomere length in human cells. Figures show remarkable increase in telomere length in multiple cell lines by thousands of basepairs within 10 days, measured using gold standard Southern blot assay. 100 micromolar of each dN used.

FIG. 2E Thymidine treatment drives telomere lengthening in human cells in a telomerase dependent manner without disrupting cell cycle. TRF Southern blot of 293T or 293T TERC−/− cells treated with or without 100 μM of thymidine for 7 days.

FIG. 2F Thymidine treatment drives telomere lengthening in human cells in a telomerase dependent manner without disrupting cell cycle. TRF Southern blot of 293T cells treated with the indicated dose of thymidine for 10 days. Manufacturer 1=Sigma Aldrich, Manufacturer 2=Santa Cruz Biotechnology, Manufacturer 3=MP Biomedicals.

FIG. 2G Thymidine treatment drives telomere lengthening in human cells in a telomerase dependent manner without disrupting cell cycle. TRF Southern blot of K562 cells treated with the indicated dose of thymidine for 8 days. FIGS. 2F-H show that thymidine (dT) from multiple manufacturers lengthens telomeres in a dose dependent manner.

FIG. 2H Thymidine treatment drives telomere lengthening in human cells in a telomerase dependent manner without disrupting cell cycle. Quantification of f,g telomere length quantified using the WALTER telomere length analysis software.

FIG. 2I Thymidine treatment drives telomere lengthening in human cells in a telomerase dependent manner without disrupting cell cycle. 293T cells treated with or without 100 μM of thymidine for the indicated period of time. On day 15, at the time of cell passage, dT treated cells were split and continued to be cultured either with or without 100 μM dT for the indicated number of days. Day 33 untreated lanes are technical replicates.

FIG. 2J Thymidine treatment drives telomere lengthening in human cells in a telomerase dependent manner without disrupting cell cycle. Quantification of i using the WALTER telomere length analysis software. FIGS. 2I-J show that thymidine (dT) drives exposure dependent telomere lengthening. Time course of thymidine treatment in 293T cells, after 15 days, cells were either maintained in thymidine or had thymidine removed from the media. Removal of thymidine stopped telomere elongation, and cells which had been treated with thymidine had long telomere length persist for more than two weeks after dT withdrawal. 100 micromolar thymidine used.

FIG. 2K Thymidine treatment drives telomere lengthening in human cells in a telomerase dependent manner without disrupting cell cycle. Growth curves of 293T cells treated with the indicated dose of dT. At each passage, cells were quantified using hemocytometry and equal numbers of cells were plated.

FIG. 2L Thymidine treatment drives telomere lengthening in human cells in a telomerase dependent manner without disrupting cell cycle. TRF Southern Blot of 293T cells from k, after 34 days of culture in media containing the indicated dose of dT. 0 μM dT treated lanes are technical replicates.

FIG. 2M Thymidine treatment drives telomere lengthening in human cells in a telomerase dependent manner without disrupting cell cycle. Growth curves of K562 cells treated with the indicated dose of dT. At each passage, cells were quantified using hemocytometry and equal numbers of cells were plated. 0 μM dT treated lanes are technical replicates.

FIG. 2N Thymidine treatment drives telomere lengthening in human cells in a telomerase dependent manner without disrupting cell cycle. TRF Southern Blot of K562 cells from m, after 34 days of culture in media containing the indicated dose of dT. FIGS. 2L and 2N show that thymidine doses as low as 5 micromolar drive robust telomere length increases. Cells were cultured in low dose thymidine for 34 days prior to measurement.

FIG. 3A Thymidine nucleotide metabolism genes control thymidine mediated telomere lengthening. Diagram of dT salvage.

FIG. 3B Thymidine nucleotide metabolism genes control thymidine mediated telomere lengthening. TRF Southern blot of 293T cells which were CRISPR/Cas9 gene edited with sgRNA(s) targeting the indicated gene or the AAVS1 control locus, followed by treatment with the indicated dose of dT for 14 days.

FIG. 3C Thymidine nucleotide metabolism genes control thymidine mediated telomere lengthening. TRF Southern blot of K562 cells which were CRISPR/Cas9 gene edited with sgRNA(s) targeting the indicated gene or the AAVS1 control locus, followed by treatment with the indicated dose of dT for 10 days.

FIG. 3D Thymidine nucleotide metabolism genes control thymidine mediated telomere lengthening. Quantification of B and C displaying the difference in median telomere length between dT treated and untreated cells targeted with the same sgRNA(s). Telomere length quantified using the WALTER telomere length analysis software.

FIG. 3E Thymidine nucleotide metabolism genes control thymidine mediated telomere lengthening. Diagram of dT and dU salvage.

FIG. 3F Thymidine nucleotide metabolism genes control thymidine mediated telomere lengthening. TRF Southern blot of 293T cells which were CRISPR/−Cas9 gene edited with sgRNA(s) targeting the indicated gene or the AAVS1 control locus. All cells were maintained in 16 μM dT to support dTTP synthesis in TYMS null cells. Cells were treated with the indicated dose of additional dT or dU for 10 days.

FIG. 3G Thymidine nucleotide metabolism genes control thymidine mediated telomere lengthening. TRF Southern blot of K562 cells which were CRISPR/Cas9 gene edited with sgRNA(s) targeting the indicated gene or the AAVS1 control locus. All cells were maintained in 16 μM dT to support dTTP synthesis in TYMS null cells. Cells were treated with the indicated dose of additional dT or dU for 10 days.

FIG. 3H Thymidine nucleotide metabolism genes control thymidine mediated telomere lengthening. Quantification of median telomere length F and G using the WALTER telomere length analysis software. FIGS. 3E-H show that deoxyuridine supplementation also increases telomere length in human cells. Deoxyuridine (dU), a deoxynucleoside which can be converted into thymidine nucleotides in the cell, increases telomere length. Blots performed after 10 days of culture in the indicated compounds. Note that all cells were treated with a baseline of 16 μM dT for experimental purposes, and addition of dU increased telomere length in this context.

FIG. 3I Quantification of median telomere length in 3 g as in 3c. n=3 biological replicates (top bar graph). Quantification of median telomere length in 3 g and 3i (bottom bar graph). P values calculated using RM One-Way ANOVA with the Geisser-Greenhouse correction and Bonferroni's multiple comparison test, n=3 biological replicates for each cell line.

FIG. 4A SAMHD1 restricts human telomere length and limits thymidine mediated telomere elongation. Diagram of SAMHD1 dTTPase activity.

FIG. 4B SAMHD1 restricts human telomere length and limits thymidine mediated telomere elongation. TRF Southern blot of 293T or K562 cells cultured for 27 days following Cas9-sgRNA electroporation using sgRNAs targeting the AAVS1 control locus or SAMHD1.

FIG. 4C SAMHD1 restricts human telomere length and limits thymidine mediated telomere elongation. TRF Southern blot of K562 cells cultured for 33 days following infection with shRNA expression constructs targeting luciferase (control) or SAMHD1.

FIG. 4D SAMHD1 restricts human telomere length and limits thymidine mediated telomere elongation. TRF Southern blot of 293T or 293T TERC−/− cells cultured for 14 days following Cas9-gRNA electroporation using sgRNAs targeting the AAVS1 control locus or SAMHD1.

FIG. 4E SAMHD1 restricts human telomere length and limits thymidine mediated telomere elongation. TRF Southern blot of 293T cells transduced with shRNAs targeting the indicated gene (shRNA-SAMHD1-2 used) then supplemented with the indicated dose of dT for 8 days.

FIG. 4F SAMHD1 restricts human telomere length and limits thymidine mediated telomere elongation. TRF Southern blot of 293T cells transduced with the pCW57.1 dox inducible expression vector harboring either EGFP or SAMHD1 of the indicated genotype, maintained in 1 μg/ml doxycycline, and supplemented with the indicated dose of dT for 10 days.

FIG. 4G SAMHD1 restricts human telomere length and limits thymidine mediated telomere elongation. TRF Southern blot of K562 cells transduced with the pCW57.1 dox inducible expression vector harboring either EGFP or SAMHD1 of the indicated genotype, maintained in 1 μg/ml doxycycline, and supplemented with the indicated dose of dT for 10 days.

FIG. 4H SAMHD1 restricts human telomere length and limits thymidine mediated telomere elongation. Quantification of median telomere length in 4f and 4g using the WALTER telomere length analysis software.

FIG. 4I SAMHD1 restricts human telomere length and limits thymidine-mediated telomere elongation. Quantification of telomere length in 4c. P value calculated by RM One-Way ANOVA with the Geisser-Greenhouse correction and Dunnett's multiple comparison test, n=2 biological replicates.

FIG. 4J SAMHD1 restricts human telomere length and limits thymidine-mediated telomere elongation. Quantification of 4D. P value calculated using paired two-sided t test, n=3 biological replicates.

FIG. 4K SAMHD1 restricts human telomere length and limits thymidine-mediated telomere elongation. Quantification of 4E n=2 biological replicates.

FIG. 4L SAMHD1 restricts human telomere length and limits thymidine-mediated telomere elongation. Quantification and statistical analysis of 4F. n=3 biological replicates.

FIG. 4M SAMHD1 restricts human telomere length and limits thymidine-mediated telomere elongation. Quantification and statistical analysis of 4G. n=3 biological replicates.

FIG. 5A dT supplementation or SAMHD1 knockdown drives telomere lengthening in telomere biology disorder patient derived iPSCs. TRF Southern blot of wildtype iPSCs treated with or without 100 μM dT for three weeks.

FIG. 5B dT supplementation or SAMHD1 knockdown drives telomere lengthening in telomere biology disorder patient derived iPSCs. TRF Southern blot of iPSCs derived from a TBD patient harboring a heterozygous deletion in TERC were treated with or without 50 μM dT for three weeks.

FIG. 5C dT supplementation or SAMHD1 knockdown drives telomere lengthening in telomere biology disorder patient derived iPSCs. TRF Southern blot of iPSCs derived from a TBD patient harboring an A353V mutation in DKC1 were treated with or without 50 μM dT for three weeks.

FIG. 5D dT supplementation or SAMHD1 knockdown drives telomere lengthening in telomere biology disorder patient derived iPSCs. TRF Southern blot of iPSCs derived from a TBD patient harboring del37L mutation in DKC1 were treated with or without 50 μM dT for three weeks.

FIG. 5E dT supplementation or SAMHD1 knockdown drives telomere lengthening in telomere biology disorder patient derived iPSCs. TRF Southern blot of iPSCs derived from a TBD patient harboring a mutation in PARN were treated with or without 50 μM dT for three weeks.

FIG. 5F dT supplementation or SAMHD1 knockdown drives telomere lengthening in telomere biology disorder patient derived iPSCs. Quantification of median, 75th percentile and 25th percentile telomere length in 5b-e using the WALTER telomere length analysis software. FIGS. 5A-F show that thymidine treatment of stem cells (iPSCs) from patients with telomere biology disorders increases telomere length by hundreds of basepairs. Cells were treated with 100 micromolar (wildtype) or 50 micromolar (patient derived iPSCs) for three weeks prior to telomere length measurement. This experiment included cells from patients with mutations in three different genes commonly associated with telomere biology disorders.

FIG. 5G dT supplementation or SAMHD1 knockdown drives telomere lengthening in telomere biology disorder patient derived iPSCs. TRF Southern blot of wildtype iPSCs transduced with the indicated shRNA expression construct and cultured for 26 days.

FIG. 5H dT supplementation or SAMHD1 knockdown drives telomere lengthening in telomere biology disorder patient derived iPSCs. TRF Southern blot of iPSCs derived from TBD patients harboring mutations in the indicated genes were transduced with the indicated sh RNA expression construct and cultured for one month.

FIG. 5I dT supplementation or SAMHD1 knockdown drives telomere lengthening in telomere biology disorder patient derived iPSCs. Model depicting the intersection between thymidine nucleotide metabolism and telomere length.

FIG. 5J Growth curves of 293T cells treated with the indicated dose of dT. n=4 biological replicates, inset graph shows population doublings after 27 days, P value calculated using RM One-Way ANOVA with the Geisser-Greenhouse correction and Dunnett's multiple comparison test. Representative TRF of 293T cells from earlier experiment, after 34 days of culture in media containing the indicated dose of dT. Quantification of previous experiment. n=4 biological replicates. P values calculated as in previous experiment.

FIG. 5K Growth curves of K562 cells treated with the indicated dose of dT. n=4 biological replicates, quantification and statistical analysis as in previous experiment. Representative TRF of K562 cells from previous experiment, after 34 days of culture in media containing the indicated dose of dT. Quantification of previous experiment. n=4 biological replicates, P values calculated as in previous experiment. Data are presented as means; error bars indicate s.d.; ns, P>0.05.

FIG. 5L DAPI staining of 293T cells treated with the indicated of dose of dT for 7 or 8 days, respectively, measured by flow-cytometry, plotted as histograms of DAPI intensity, displaying representative samples from each treatment arm and the percentage of cells in different stages of the cell cycle (gated based on the lines drawn on the histogram, gates determined based on untreated samples. n=2 biological replicates for 293T cells treated with 200 μM dT and n=3 biological replicates for all other conditions. Data presented are means; error bars indicate standard deviation.

FIG. 5M DAPI staining of K562 cells treated with the indicated of dose of dT for 7 or 8 days, respectively, measured by flow-cytometry, plotted as histograms of DAPI intensity, displaying representative samples from each treatment arm and the percentage of cells in different stages of the cell cycle (gated based on the lines drawn on the histogram, gates determined based on untreated samples. n=2 biological replicates for 293T cells treated with 200 μM dT and n=3 biological replicates for all other conditions. Data presented are means; error bars indicate standard deviation.

FIG. 6A Telomere length CRISPR/Cas9 screening using flow-FISH. Histogram of GFP fluorescence from K562 cells expressing Cas9 measured by flow-cytometry.

FIG. 6B Telomere length CRISPR/Cas9 screening using flow-FISH. Histogram of GFP fluorescence from K562 cells which do not express Cas9 which were transduced with the pXPR-011 vector which expresses EGFP and a gRNA targeting EGFP measured by flow-cytometry.

FIG. 6C Telomere length CRISPR/Cas9 screening using flow-FISH. Histogram of GFP fluorescence from K562 cells expressing Cas9 which were transduced with the pXPR-011 vector which expresses EGFP and a gRNA targeting EGFP and cultured for two weeks, measured by flow-cytometry. Presence of GFP negative indicates cells have functional Cas9 nuclease activity.

FIG. 6D Telomere length CRISPR/Cas9 screening using flow-FISH. Representative gating strategy for flow-FISH telomere length screening. Data from Nucleotide metabolism library infected K562 cells, replicate 1. Cells are gated to enrich for single cells.

FIG. 6E Telomere length CRISPR/Cas9 screening using flow-FISH. Data from Nucleotide metabolism library infected K562 cells, replicate 1. Cells are gated to enrich for single cells.

FIG. 6F Telomere length CRISPR/Cas9 screening using flow-FISH. Data from Nucleotide metabolism library infected K562 cells, replicate 1. Cells are gated on low DAPI fluorescence to enrich for cells with 2n genome copy number and aid in identifying gRNAs which promote telomere elongation independent from changes in total DNA content.

FIG. 6G Telomere length CRISPR/Cas9 screening using flow-FISH. Data from Nucleotide metabolism library infected K562 cells, replicate 1. Cells are gated on high and low TelC-Alexa 647 probe fluorescence populations. Gates adjusted to maintain approximately 5% of cells throughout the duration of the sort.

FIG. 6H Telomere length CRISPR/Cas9 screening using flow-FISH. gRNA enrichment in high telomere fluorescence populations compared to unsorted populations from K562 cells expressing Cas9 which were cultured for 50 days followed by flow-FISH sorting of the 5% of cells with the highest and lowest telomere population. calculated using the Mageck RRA software. Known telomere length regulating genes indicated with orange dots, other genes indicated are involved in nucleotide metabolism.

FIG. 6I Telomere length CRISPR/Cas9 screening using flow-FISH. RNA enrichment in low telomere fluorescence populations compared to unsorted populations from K562 cells expressing Cas9 which were cultured for 50 days followed by flow-FISH sorting of the 5% of cells with the highest and lowest telomere population. calculated using the Mageck RRA software. Known telomere length regulating genes indicated with orange dots, other genes indicated are involved in nucleotide metabolism.

FIG. 6J Telomere length CRISPR/Cas9 screening using flow-FISH. KEGG pathway enrichment analysis using the hypergeometric test performed on the genes with gRNAs enriched in the sorted short telomere population, analysis performed using the MageckFlute software package. Plot includes top enriched KEGG terms, plotting-log 10 adjusted p value, dot size indicates number of genes identified in that pathway out of the short telomere enriched genes.

FIG. 7A Characterization of TERC-null 293T cells. Schematic of TERC genotype in TERC-null 293T cells including a deletion of the essential box H domain on one allele and an 821 bp deletion which encompasses the 5′ end of TERC including the box H domain, cell line generated using genome editing.

FIG. 7B Characterization of TERC-null 293T cells. Sanger sequencing of the TERC locus in the TERC-null 293T cell line demonstrating no allele harboring a complete box H domain is present.

FIG. 7C Characterization of TERC-null 293T cells. RT-qPCR of TERC expression relative to GAPDH in wildtype 293T and TERC-null 293T cells.

FIG. 7D Characterization of TERC-null 293T cells. TRF Southern blot of wildtype and TERC-null 293T cells. Telomere length gradually declines with passage until cells universally senesce (data not shown).

FIG. 7E Characterization of TERC-null 293T cells. Ethidium bromide stained agarose gel of PCR of 293T or 293T TERC-null genomic DNA using primers flanking the deletions indicated in 7a.

FIG. 7F Characterization of TERC-null 293T cells. Sanger sequencing of gel-purified PCR products from the (1) higher molecular weight bands in 7E, indicating that the non-deleted allele lacks the box H domain, and (2) the 4821 bp deleted band from 7E, with trace file showing the deletion junction in a genomic context.

FIG. 7G Characterization of TERC-null 293T cells. Telomerase activity measured via the TRAP assay, performed on 5-fold serial dilutions of lysates. HI indicates heat-inactivated lysate. IC indicates the internal control product.

FIG. 7H Characterization of TERC-null 293T cells. TRF of wild-type and TERC-null 293T cells. Days of culture were recorded beginning approximately two months after gene editing. Telomere length gradually declines with passage until cells universally senesce.

FIG. 7I Characterization of TERC-null 293T cells. Quantification of 7H, line fit using simple linear regression. Data presented in this figure are the results of single experiments unless otherwise indicated.

FIG. 8A Cell growth and telomere length effects of dT, hydroxyurea, and RO-3306. Growth curve of 293T cells treated with the indicated dose of dT. Cell counts quantified using a hemocytometery at the time of passage, equal cells plated at each passage.

FIG. 8B Cell growth and telomere length effects of dT, hydroxyurea, and RO-3306. TRF Southern blot of K562 cells treated with the indicated compound for 8 days.

FIG. 8C Cell growth and telomere length effects of dT, hydroxyurea, and RO-3306. TRF Southern blot of 293T cells treated with the indicated compound for 10 days.

FIG. 8D Cell growth and telomere length effects of dT, hydroxyurea, and RO-3306. TRF Southern blot of 293T cells treated with the indicated compound for 10 days.

FIG. 9 Deoxyuridine but not folic acid supplementation promotes telomere elongation. TRF Southern blot of 293T cells treated with the indicated compound for 10 days. dU=deoxyuridine.

FIG. 10A Cas9-gRNA electroporation gene editing of TK1, TK2 and TYMS. Genomic DNA from 293T or K562 cells manipulated with the indicated sgRNA(s) and dT treatment was PCR amplified using primers specific to the TK1 genomic locus and and separated by electrophoresis on an agarose gel demonstrating on-target genomic deletions (top), first lane is a molecular weight marker. PCR products were Sanger sequenced and editing efficiency was quantified using the Synthego ICE algorithm (bottom). Genomic DNA used here was the same sample as used to generate FIG. 3b,c.

FIG. 10B Cas9-gRNA electroporation gene editing of TK1, TK2 and TYMS. Genomic DNA from 293T or K562 cells manipulated with the indicated sgRNA(s) and dT treatment was PCR amplified using primers specific to the TK2 genomic locus and separated by electrophoresis on an agarose gel demonstrating on-target genomic deletions (top), first lane is a molecular weight marker. PCR products were Sanger sequenced and editing efficiency was quantified using the Synthego ICE algorithm (bottom). Genomic DNA used here was the same sample as used to generate FIG. 3b,c.

FIG. 10C Cas9-gRNA electroporation gene editing of TK1, TK2 and TYMS. Genomic DNA from 293T or K562 cells manipulated with the indicated sgRNA(s) and dT treatment was PCR amplified using primers specific to the TYMS genomic locus and run on an agarose gel demonstrating on-target genomic deletions (top), first lane is a molecular weight marker. PCR products were Sanger sequenced and editing efficiency was quantified using the Synthego ICE algorithm (bottom). Genomic DNA used here was the same sample as used to generate FIG. 3f,g.

FIG. 10D dT nucleotide metabolism perturbations and their effects on telomere length and polar metabolite homeostasis. Quantification of FIG. 3b. n=3 biological replicates for each cell line, P values calculated using paired two-sided t test.

FIG. 10E Manipulation of SAMHD1 levels by CRISPR/Cas9, shRNA, and lentiviral expression. TRF of indicated cell lines transduced with the indicated shRNA and cultured for 15 days, and quantification of the TRF using the WALTER webtool. The boxplot displays the 75th, 50th and 25th percentile molecular weight of the telomere signal distribution in the TRF blot.

FIG. 11 Liquid chromatography mass spectrometry (LCMS) quantifies nucleotide metabolite level changes from dT treatment. Polar metabolite profiling by liquid chromatography mass spectrometry of 293T cells treated with or without 100 μM dT for 24 hours, performed in biological triplicate, P value calculated by student's T test of average signal intensity in treatment vs control samples, nucleotide and nucleoside species detected in all samples displayed.

FIG. 12A Manipulation of SAMHD1 levels by CRISPR/Cas9, shRNA, and lentiviral expression. Immunoblot of 293T and K562 cells electroporated with Cas9 and the indicated gRNA(s) using primary antibodies targeting SAMHD1 and β-Actin, corresponding to cell lines evaluated in FIG. 4a.

FIG. 12B Manipulation of SAMHD1 levels by CRISPR/Cas9, shRNA, and lentiviral expression. Immunoblot of K562 cells transduced with vectors expressing the indicated shRNA generated using primary antibodies targeting SAMHD1 and β-Actin, corresponding to cell lines evaluated in FIG. 4b.

FIG. 12C Manipulation of SAMHD 1 levels by CRISPR/Cas9, shRNA, and lentiviral expression. qRT-PCR of SAMHD1 expression compared to β-Actin, performed in technical triplicate.

FIG. 12D Manipulation of SAMHD1 levels by CRISPR/Cas9, shRNA, and lentiviral expression. Immunoblot of 293T cells transduced with vectors expressing the indicated shRNA generated using primary antibodies targeting SAMHD1 and β-Actin.

FIG. 12E Manipulation of SAMHD1 levels by CRISPR/Cas9, shRNA, and lentiviral expression. TRF Southern blot of indicated cell lines transduced with the indicated shRNA and cultured for 15 days.

FIG. 12F Manipulation of SAMHD1 levels by CRISPR/Cas9, shRNA, and lentiviral expression. Immunoblot of 293T cells transduced with vectors to overexpress either EGFP or the indicated SAMHD1 variant, treated with the indicated dose of dT, generated using primary antibodies targeting SAMHD1 and β-Actin, corresponding to cell lines evaluated in FIG. 4f.

FIG. 13A Thymidine supplementation increases telomere length. TRF of patient derived iPSCs treated with or without 100 μM dT for 22 days.

FIG. 13B Thymidine supplementation increases telomere length. Polar metabolite profiling by LC-MS of 293T cells treated with 100 M dT or vehicle control, performed in biological triplicate, signals normalized for input material using standard procures, plotting mean of triplicates normalized to mean signal from vehicle treated controls vs P value calculated by student's t-test of treatment vs control replicates, displaying nucleotides detected in all samples.

FIG. 13C Thymidine supplementation increases telomere length. TRF of 293T cells edited with pool of three gRNAs targeting TK1 or TK2, or control AAVS1 gRNA, PCR product of TK1 and TK2 shows efficient editing by production of a deletion in the locus.

FIG. 14A SAMHD 1 restricts human telomere length. TRF Telomere Southern blots and western blots for SAMHD1 and ACTB for indicated cell lines 27 days following Cas9-9RNA electroporation using gRNAs against AAVS1 control locus or SAMHD1.

FIG. 14B SAMHD1 restricts human telomere length. Telomere Southern blots and western blots for SAMHD1 and ACTB for indicated cell lines 33 days post infection with shRNA expression constructs against luciferase control or SAMHD1.

FIG. 14C SAMHD1 restricts human telomere length. 293T cells or 293T TERC−/− cells 14 days post cas9-gRNA electroporation using gRNAs againstAAVS1 control locus or SAMHD1.

FIG. 15 is a schematic showing connection between supplementation of dT, increase of dTTP in the nucleotide pool, and telomere length.

FIG. 16 Efficient SAMDH1 Editing in Human HSPCs. HSPCs were edited by Cas9/gRNA electroporation with three gRNAs targeting SAMHD1 or an AAVS1 targeting control gRNA, two days later the genomic locus was PCR amplified and Sanger sequenced and quantified using Synthego ICE.

FIG. 17A Quantification of telomerase processivity in cells. Schematic of alternative template strategy to label telomerase synthesis events.

FIG. 17B Quantification of telomerase processivity in cells. Schematic of TrAEL seq strategy to sequence 3′ DNA ends including telomeres.

FIG. 17C Quantification of telomerase processivity in cells. Published TrAEL seq data set filtered for reads those containing G Rich=5×TTAGGG, C Rich=5×CCCTAA for human or a common yeast telomere sequence G Rich=TGGGTGTGGTG or C Rich-CACCACACCCA, other yeast telomere sequences gave similar results (Not shown). Biological replicates are displayed, if technical replicates were available they were averaged.

FIG. 18A Quantification of de novo synthesized and salvaged nucleotide pools used by telomerase. Schematic of the process.

FIG. 18B Quantification of de novo synthesized and salvaged nucleotide pools used by telomerase. TRF of 293T cells with a TBD causing TINF2 mutation, infected with TERC and TERT overexpression lentivectors, TRF quantified using the WALTER webtool.

FIG. 19 Thymidine treatment of HSPCs. TRF of HSPCs treated with the indicated dT dose for 7 days. Telomere length quantified using the WALTER webtool. Median, first and third quartile plotted.

FIG. 20 contains a bar graph showing effect of combining dT and dC on cellular growth in primary human CD34+ hematopoietic cells.

FIG. 21 contains a line plot showing evidence that dC and C reduce toxicity caused by dT (See DOI 10.1371/journal.pgen. 1002035).

FIG. 22 contains a bar graph showing evidence that dC and C reduce toxicity caused by dT (See 10.1101/2021.12.06.471399).

FIG. 23 shows that cytidine and uridine increase telomere length.

FIG. 24A-24U. Induction of replication stress is insufficient to explain telomere lengthening from dT treatment. a, Diagram of the effect of dT, 5FU, and hydroxyurea on dT nucleotide metabolism. RNR, ribonucleotide reductase. b, TR F Southern blot of 293T TERC-null cells transfected with the indicated expression vectors, cultured for 18 hours, then treated with the indicated dose of dT for 30 hours. Representative blot shown from two biological replicates. c, Immunoblot of cells from b using the indicated primary antibodies. UV-treated 293T cells used as a positive control. d, Cell cycle analysis of cells from b measured by DAPI staining and flow cytometry, displaying the percentage of cells in each gate. Data from four biological replicates shown for untreated cells and from two biological replicates shown for treated cells. Error bars indicate standard deviation, e-g, TRF Southern blot of 293T TERC-null cells transfected with the indicated expression vectors, cultured for 18 hours, then treated with the indicated doses of aphidicolin (e), 5FU (f), or hydroxyurea (g) for 30 hours. Representative blots from two biological replicates. h,i, Immunoblot of cells from e-g, as in c. j-l, Cell cycle analysis of cells in e-g by DAPI staining and flow cytometry, as in d. Data from four biological replicates shown for untreated cells and from two biological replicates shown for treated cells. Untreated samples are the same as in d. m, TRF Southern blot of 293T TERC-null cells transfected with the indicated expression vectors, cultured for 18 hours, then treated with the indicated doses of SFU and dT for 30 hours. Data in d, j, k and l are presented as means; error bars indicate s.d.; ns, P>0.05. Full-length western blots are provided as source data. n, TRF Southern blot of 293T cells treated with the indicated doses of aphidicolin and hydroxyurea for 10 days. o, TRF Southern blot of 293T TERC-null cells transfected with TERT in addition to the indicated vector, cultured for 18 hours, then treated with the indicated dose of dT for 30 hours. c, TRF Southern blot of 293T TERC-null cells transfected with TERT in addition to the indicated vector, cultured for 18 hours, then treated with the indicated dose of dT for two days. p, TRF Southern blot of 293T TERC-null cells transfected with the indicated expression vectors, cultured for 18 hours, then treated with the indicated dose of dT for five days. Q-u, Cell cycle analysis by DAPI staining and flow cytometry of 293T TERC-null cells transfected with TERC and TERT expression vectors, cultured for 18 hours, then treated with the indicated of dose of dT (r, aphidicolin(s), 5FU (t), or hydroxyurea (u, displayed as histograms of DAPI intensity of representative samples from each treatment arm, corresponding to cells in FIG. 24b-1. Gating based on untreated cells. TRFs presented in this figure show the results of single experiments.

FIG. 25A-25Z dT nucleotides enhance human telomerase activity independent of dTTP's role as a telomerase substrate. a, Representative TRAP assay of 293T cells treated as indicated for 3 days. b, Quantification of a. n=3 biological replicates, P value calculated using unpaired two-sided t test. c, Diagram of wildtype and ‘T-free’ telomerase. d, Direct telomerase assay using immunopurification of overexpressed, tagged TERT co-transfected with T-free TERC into 293T TERC-null cells. LC: loading control, 16-nt ³²P-end labeled oligo. Telomerase repeat products numbered. e, Direct telomerase assay using immunopurification of overexpressed, tagged TERT co-transfected with the indicated TERC vector or EGFP as control into 293T TERC-null cells. n=3 replicates. f, Quantification of e. P value calculated as in b. g, PCR to detect wildtype and T-free telomere junction (see Methods) performed on 293T TERC-null cells transfected with the indicated vectors, cultured for 18 hours, then treated with dT as indicated for three days. h, Sanger and nanopore sequencing of products from lane 4 in g. Sanger trace corresponding to cytosine omitted for clarity. i, Slot blot of DNA from 293T TERC-null cells overexpressing TERT as well as the indicated vector, cultured for 18 hours, then treated with the indicated dose of dT for 30 hours, performed in technical triplicates. Representative data from one of two biological replicates displayed. j, Quantification of GGAAAG signal in i. P values calculated using unpaired two-sided/tests, n=3 technical replicates. k, Slot blot of 293T TERC-null cells transfected as in i, and treated with the indicated dose of dT, 5FU (10 μM), or HU (500 μM) as indicated. Representative data from one of two biological replicates displayed. 1, Quantification of k as in j. n=3 technical replicates. Data in b, f, j and l are presented as means, error bars indicate s.d.; ns, P>0.05. Full-length gels are provided as source data. T-free telomerase is sensitive to dT nucleotide manipulations. m, Representative modified TRAP assay performed on super-telomerase extracts using the indicated dose of dTTP and physiologic levels of dATP, dCTP and dGTP (see Methods). m, Quantification of m. n=2 biological replicates. o GGAAAG TRAP assay performed on lysates from 293T TERC-null cells overexpressing T-free super-telomerase demonstrates linearity between cell input amount and telomerase signal. Five-fold serial dilutions performed. HI, heat inactivated. p, Quantification of lanes 1-3 o. q, Representative modified GGAAAG TRAP assay performed on super-telomerase extracts generated using the indicated TERC vector. Assay performed with the indicated dose of dTTP and physiologic levels of dATP, dCTP and dGTP (see Methods). HI, heat inactivated. r, Quantification of e using two-sided unpaired Student's t test; n=3 biological replicates. s, Representative modified GGAAAG TRAP assay performed on T-free super-telomerase extracts supplemented with the indicated dose of dTTP and physiologic levels of dATP and dGTP. t, Quantification of s as r, n=3 biological replicates.u, Diagram of GGAAAG TRAP product sequencing and analysis strategy. Note * indicates T's encoded by the partially complementary reverse primer, preventing analysis of base composition in that portion of the read. v, Quantification of base pair composition of representative GGAAAG TRAP products from g with 0 μM or 25 μM dTTP by nanopore sequencing (see Methods). Bits of information calculated using Shannon entropy and plotted using ggseqlogo. w, Quantification of base pair composition of GGAAAG TRAP products from s using nanopore sequencing (see Methods). P value calculated using two-sided Student's t test; n=3 biological replicates. x, Quantification of FIG. 7d, plotting the signal in the indicated telomerase product repeat relative to the signal of the corresponding repeat in the lane without dTTP added, normalized for loading (see Methods). y, TRF Southern blot of 293T TERC-null cells transfected with TERT in addition to the indicated vector, cultured for 18 hours, then treated with the indicated dose of dT for 30 hours, and probed with a GGTTAG complementary probe. Lanes 1~4 are the same blot shown in FIG. 24. z, Blot from w was stripped and re-probed with a probe complementary to the GGAAAG repeat.

FIG. 26A-26F. Slot blot of DNA from 293T TERC-null cells overexpressing EGFP and TERT showing linear relationship between DNA input and signal; rows are technical triplicates. b, Quantification of a. c, Slot blot of DNA from 293T TERC-null cells overexpressing T-free super-telomerase; rows are technical triplicates. d, Quantification of c. e, Slot blot of DNA from 293T-TERC null cells transfected with TERT and TERC, cultured for 18 hours, then treated with dT as indicated for 30 hours. Denatured DNA for each sample was split and loaded onto parallel blots, which were probed for the indicated target. Performed in technical triplicate. f, Quantification of s. P values calculated with one way.

FIG. 27A-27I. dT supplementation or SAMHD1 knockdown drives telomere lengthening in iPSCs from patients with telomere biology disorders. a, TRF of iPSCs derived from a healthy donor or from TBD patients harboring mutations in the indicated genes that were treated with or without 50 M dT for three weeks. Representative blot from three biological replicates. b, Quantification of a. P values calculated using paired two-sided t tests. Open circle indicates cells treated with 100 μM dT for three weeks. All other treated cells received 50 μM dT. All data points represent biologically independent samples. Sample size sizes for wild-type iPSC, untreated (n=3) or dT treated (n=3) and for TBD patient-derived iPSCs, untreated (n=12) or dT treated (n=12). c, TRF of wild-type iPSCs transduced with the indicated shRNA expression construct and cultured for 26 days. Representative blot from three biological replicates shown. d, TRF of iPSCs derived from TBD patients harboring mutations in the indicated genes that were transduced with the indicated shRNA expression construct and cultured for one month. e, Quantification of c and d, as in b. All data points represent biologically independent samples. Sample sizes for wild-type iPSCs, shLuciferase (n=3 samples) or shSAMHD1-2 (n=3 samples), and for TBD patient-derived iPSCs, shLuciferase (n=3 samples) or shSAMHD1-2 (n=3 samples). f, Model of the relationship between dTTP metabolism and telomere length. Data in b and e are presented as means, error bars indicate s.d.; ns, P>0.05. Effects of dT on iPSC cell cycle progression and replication stress signaling. g, Cell cycle analysis of wild-type iPSCs cultured in the indicated of dose of dT for 24 hours, measured by DAPI staining and flow cytometry, displayed as histograms of DAPI intensity. n=2 biological replicates; the mean of the replicates is presented. h, Representative histograms of DAPI signal for cells in g. Gates defined based on untreated cells. i, Immunoblot of cells treated as in g; all images of the same membrane blotted with the indicated primary antibodies. UV-treated cells used as a positive control. Blot shows the results from a single experiment. Full-length blots are provided as source data.

FIG. 28A-28E. Model of relationship between dT nucleotide metabolism and telomere synthesis. a-e, Schematics illustrate conditions of homeostasis (a), dT supplementation (b), dU supplementation (c), loss of SAMHD1 (d), and treatment with hydroxyurea or 5-fluorouracil (e).

FIG. 29A Overexpressing the Drosophila melanogaster deoxynucleoside kinase (dmDNK) increases telomere synthesis from deoxynucleoside treatment, including from dT. TERC−/− 293T cells stably expressing dmDNK or eGFP (control) were transfected with vectors to express TERT and TERC, then cultured in the indicated doses of nucleoside for 30 hrs, followed by analysis of telomere DNA content via blotting and detection with a complementary oligonucleotide probe.

FIG. 29B Overexpressing the Drosophila melanogaster deoxynucleoside kinase (dmDNK) increases telomere synthesis from deoxynucleoside treatment, including from dT. TERC−/− 293T cells TERC−/− 293T cells stably expressing dmDNK or eGFP (control) were transfected with vectors to express TERT and TERC, then cultured with 100 μM of each of the indicated nucleosides for 24 hrs, followed by Southern blot analysis.

FIG. 30A shows that the expression of TERT can increase telomere lengthening from dT, which was shown in primary fibroblasts. This data provides credible evidence that expression of TERT in addition to dT treatment is therapeutically useful.

FIG. 30B shows that the combination of dT+dC was better tolerated compared to dT alone in some cells, for example in primary human hematopoietic cells. This data supports the claim that combinations of dNs are therapeutically useful.

### DETAILED DESCRIPTION

Introduction. Telomere length has been associated with diseases and longevity, but the genetic determinants of human telomere length regulation remain incompletely defined. Here, genome-wide CRISPR/Cas9 screening and flow cytometry-FISH (flow-FISH) were used to identify novel telomere length control genes in human cells. Validating the screen, gRNAs were found targeting positive (e.g. TERT) and negative (e.g. POT1 and TRF1) regulators of telomere length enriched in sorted cells with short and long telomeres, respectively. Unbiased pathway analysis identified genes regulating nucleotide metabolism as highly associated with telomere length. Follow-up screening demonstrated that thymidine (dT) nucleotide metabolism is a critical regulator of human telomere length. Disrupting dT nucleotide synthesis genes impaired telomere maintenance, whereas inhibiting SAMHD1, which degrades dNTPs, lengthened telomeres. Remarkably, supplementation with dT drove rapid, telomerase-dependent telomere elongation, without interrupting the cell cycle. In targeted analyses, thymidine kinase 1 (TK1), which converts dT to dTMP in the cytosol, was required for telomere lengthening after dT supplementation, while disruption of the mitochondrial isoenzyme TK2 had no effect. Conversely, in cells with impaired de novo dT nucleotide synthesis from thymidylate synthase (TYMS) deletion, dT supplementation promoted robust telomere elongation. Manipulating levels of SAMHD1 impacted telomere elongation from dT treatment, suggesting dTTP abundance contributes to telomere length control. In induced pluripotent stem cells derived from patients with telomere biology disorders (TBDs), dT supplementation or inhibition of SAMHD1 promoted telomere restoration.

Collectively, this disclosure demonstrate a critical role of pyrimidine nucleotide (e.g., thymidine nucleotide) metabolism in regulating human telomere length, which may be therapeutically actionable in TBD patients.

### Compounds

In some embodiments, the present application provides a compound of Formula (I):

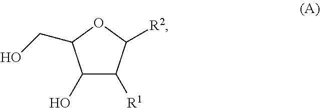

or a pharmaceutically acceptable salt thereof, wherein:
R¹ is selected from H and OH;
R² is selected from any one of the following moieties:

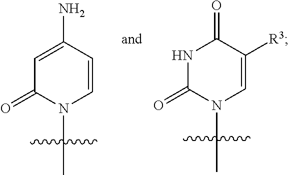

and
R³ is selected from H and CH₃.

In some embodiments, the compound has formula:

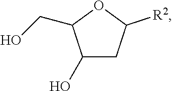

or a pharmaceutically acceptable salt thereof.

In some embodiments, the compound has formula:

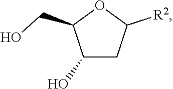

or a pharmaceutically acceptable salt thereof.

In some embodiments, the compound has formula:

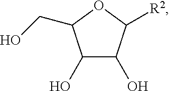

or a pharmaceutically acceptable salt thereof.

In some embodiments, the compound has formula:

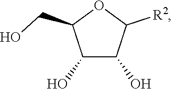

or a pharmaceutically acceptable salt thereof.

In some embodiments, the compound has formula:

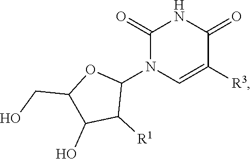

or a pharmaceutically acceptable salt thereof.

In some embodiments, R³ is H.

In some embodiments, R³ is CH₃.

In some embodiments, the compound has formula:

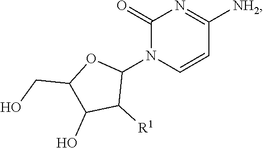

or a pharmaceutically acceptable salt thereof.

In some embodiments, R¹ is H.

In some embodiments, R¹ is OH.

In some embodiments, the present application provides a compound of Formula (I):

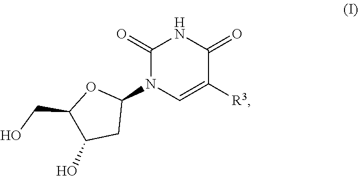

or a pharmaceutically acceptable salt thereof, wherein R¹ is selected from H and methyl. In some embodiments, R¹ is H. In some embodiments, R¹ is methyl.

In some embodiments, the present application provides a compound of formula:

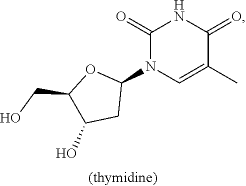 [SMILES: Cc1cn([C@H]2C[C@H](O)[C@@H](CO)O2)c(=O)[nH]c1=O]

or a pharmaceutically acceptable salt thereof. Generally, thymidine has a CAS Registry number 50-89-5, molecular formula C₁₀H₁₄N₂O₅, molecular weight of about 242.2 g/mol, and aqueous solubility of about 50 mg/ml. The compound may be referred to as 1-[(2R,4S,5R)-4-Hydroxy-5-(hydroxymethyl)oxolan-2-yl]-5-methylpyrimidine-2,4 (1H,3H)-dione, 5-methyldeoxyuridine, or “dT”, among other chemical and conventional names. In some embodiments, the thymidine is crystalline (e.g., any crystalline Form of thymidine, or a mixture thereof). In some embodiments, the thymidine is amorphous. In some embodiments, thymidine comprises a mixture of crystalline and amorphous forms.

In some embodiments, the present application provides a compound of formula:

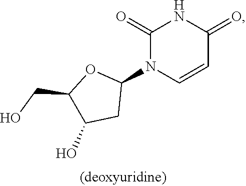 [SMILES: O=c1ccn([C@H]2C[C@H](O)[C@@H](CO)O2)c(=O)[nH]1]

or a pharmaceutically acceptable salt thereof. Generally, deoxyuridine has a CAS Registry number 951-78-0, molecular formula C₉H₁₂N₂O₅, molecular weight of 228.20 g/mol, and water solubility of about 5 mg/ml at pH of about 7. The compound may be referred to as 1-((2R,4S,5R)-4-Hydroxy-5-(hydroxymethyl)tetrahydrofuran-2-yl)pyrimidine-2,4 (1H,3H)-dione, uracil deoxyriboside, or “dU”, among other chemical and conventional names. In some embodiments, the deoxyuridine is crystalline (e.g., any crystalline Form of deoxyuridine, or a mixture thereof). In some embodiments, the deoxyuridine is amorphous. In some embodiments, deoxyuridine comprises a mixture of crystalline and amorphous forms.

In some embodiments, the present application provides a compound of formula:

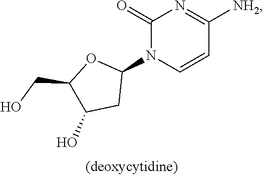 [SMILES: Nc1ccn([C@H]2C[C@H](O)[C@@H](CO)O2)c(=O)n1]

or a pharmaceutically acceptable salt thereof.

Generally, deoxycytidine has a CAS Registry number 951-77-9, molecular formula C₉H₁₃N₃O₄, molecular weight of 227.20 g/mol, and water solubility of about 870 mg/mL at pH of about 7. The compound may be referred to as 4-azmino-1-[(2R,4S,5R)-4-hydroxy-5-(hydroxymethyl)oxolan-2-yl]pyrimidin-2 (1H)-one, cytosine deoxyriboside, or “dC”, among other chemical and conventional names. In some embodiments, the deoxycytidine is crystalline (e.g., any crystalline Form of deoxycytidine, or a mixture thereof). In some embodiments, the deoxycytidine is amorphous. In some embodiments, deoxycytidine comprises a mixture of crystalline and amorphous forms.

In some embodiments, the present application provides a compound of formula:

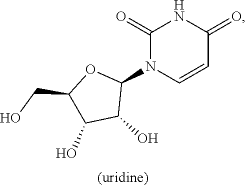 [SMILES: O=c1ccn([C@@H]2O[C@H](CO)[C@@H](O)[C@H]2O)c(=O)[nH]1]

or a pharmaceutically acceptable salt thereof.

Generally, uridine has a CAS Registry number 58-96-8, molecular formula C₉H₁₂N₂O₆, molecular weight of 244.20 g/mol, and water solubility of about 35 μg/ml at pH of about 7. The compound may be referred to as 1-[(2R,3R,4S,5R)-3,4-Dihydroxy-5-(hydroxymethyl)oxolan-2-yl]pyrimidine-2,4 (1H,3H)-dione, uracil riboside, or “U”, among other chemical and conventional names. In some embodiments, the uridine is crystalline (e.g., any crystalline Form of uridine, or a mixture thereof). In some embodiments, the uridine is amorphous. In some embodiments, uridine comprises a mixture of crystalline and amorphous forms.

In some embodiments, the present application provides a compound of formula:

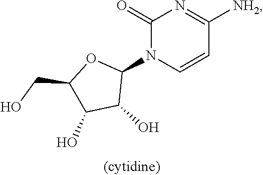 [SMILES: Nc1ccn([C@@H]2O[C@H](CO)[C@@H](O)[C@H]2O)c(=O)n1]

or a pharmaceutically acceptable salt thereof.

Generally, cytidine has a CAS Registry number 65-46-3, molecular formula C₉H₁₃N₃O₅, molecular weight of 243.20 g/mol, and water solubility of about 100 mg/mL at pH of about 7. The compound may be referred to as 4-amino-1-[(2R,3R,4S,5R)-3,4-dihydroxy-5-(hydroxymethyl)oxolan-2-yl]pyrimidin-2 (1H)-one, cytosine riboside, or “C”, among other chemical and conventional names. In some embodiments, the cytidine is crystalline (e.g., any crystalline Form of cytidine, or a mixture thereof). In some embodiments, the cytidine is amorphous. In some embodiments, cytidine comprises a mixture of crystalline and amorphous forms.

Without being bound by any particular theory or speculation, it is believed that deoxyuridine, cytidine, deoxycytidine, and uridine can be converted to thymidine nucleotides upon administration in vitro, in vivo, or ex vivo, thereby promoting and elongation. As such, in some embodiments, the compounds of Formula A (e.g., C, dC, U, and dU) are prodrugs of dTxP (e.g., dTMP, dTDP, and dTTP) which is are the substrates for telomere growth.

### Pharmaceutically Acceptable Salts

As used herein, the term “pharmaceutically acceptable salt” refers to a salt that is formed between an acid and a basic group of the compound of this disclosure, such as an amino functional group, or between a base and an acidic group of the compound, such as a carboxyl functional group. In some embodiments, the compound is a pharmaceutically acceptable acid addition salt. In some embodiments, acids commonly employed to form pharmaceutically acceptable salts of the therapeutic compounds described herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the therapeutic compounds described herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Methods of Use

The come embodiments, the present disclosure provides a method of treating a telomere biology disorder, the method comprising administering to a subject diagnosed with said telomere biology disorder a therapeutically effective amount of a compound of Formula (I) as described herein (e.g., thymidine or deoxyuridine), or a pharmaceutically acceptable salt thereof. Suitable examples of telomere biology disorders (e.g., dyskeratosis congenita, aplastic anemia, myelodysplastic syndrome, liver or lung fibrosis, or interstitial lung disease) are described below.

In some embodiments, prior to administering the compound to the subject, the method includes a step of diagnosing the subject as having or being at risk of developing the telomere biology disorder. In some embodiments, prior to administering the compound to the subject, the method includes a step of identifying the subject as diagnosed with the disorder. Suitable examples of diagnostic and identification methods are described below.

In some embodiments, the present disclosure provides a method of treating a disorder associated with aging, a pre-leukemic or pre-cancerous condition, a neurodevelopmental disorder, or an acquired or genetic disease or condition associated with alterations in RNA, the method comprising administering to a subject in need of treatment of said disorder (e.g., subject diagnosed with said disorder) a therapeutically effective amount of a compound of Formula (I) as described herein (e.g., thymidine or deoxyuridine), or a pharmaceutically acceptable salt thereof. Examples of these disorders, as well as the methods of diagnosing said disorders, are described below.

In some embodiments, the compound may be administered in any composition, formulation, or dosage form described more fully hereinbelow. The composition, formulation, or dosage form may include the compound, or a pharmaceutically acceptable salt thereof, and a suitable pharmaceutically acceptable excipient or carrier as described below. The suitable route of administering the compound may be determined by a treating physician on the basis of the subject's diagnosis, symptoms, and condition. Any of the administration routes described herein may be used. In some embodiments, the compound can be administered orally. For example, the compound can be administered in a tablet or a capsule comprising the compound, or a pharmaceutically acceptable salt thereof, and a suitable excipient or excipients. The compound may also be in a sachet in a powder form, for dissolving or suspending the compound in water or an aqueous solution suitable for drinking. The compound may also be administered intravascularly, in any suitable formulation or dosage form described hereinbelow for intravascular administration. In one example, the compound may be administered in an ampule comprising sterile injectable solution of the compound. In this example, the compound can be administered by an intravenous injection, such as by drawing the solution from the ampule by a syringe, followed by injecting the solution to the subject intravenously. In another example, the compound may be administered in an infusion bag. In this example, the compound may be dissolved in a suitable infusible aqueous solution as described below, such as saline or 5 wt. % dextrose solution, followed by infusing the solution comprising the determined dose of the compound to the subject using appropriate infusion rate (e.g., from about 50 ml/h to about 200 ml/h).

In some embodiments, the therapeutically effective amount of the compound, or a pharmaceutically acceptable salt thereof, as well as the administration regimen, may be determined by a treating physician. In some embodiments, the therapeutically effective amount of the compound is from about 50 mg/kg/day to about 500 mg/kg/day. In some embodiments, the therapeutically effective amount of the compound is from about 20 mg/kg/day to about 250 mg/kg/day, from about 30 mg/kg/day to about 200 mg/kg/day, or from about 50 mg/kg/day to about 200 mg/kg/day. In some embodiments, the therapeutically effective amount of the compound is from about 100 mg/kg/day to about 500 mg/kg/day, from about 120 mg/kg/day to about 480 mg/kg/day, or from about 130 mg/kg/day to about 400 mg/kg/day. In some embodiments, the therapeutically effective amount is about 130 mg/kg/day, about 260 mg/kg/day, or about 400 mg/kg/day. In some embodiments, the therapeutically effective amount is about 130 mg/kg/day. In some embodiments, the therapeutically effective amount is about 200 mg/kg/day. In some embodiments, the therapeutically effective amount is about 260 mg/kg/day. In some embodiments, the therapeutically effective amount is about 400 mg/kg/day. In some embodiments, the therapeutically effective amount of the compound is from about 1 g/m²/day to about 20 g/m²/day, from about 2 g/m²/day to about 15 g/m²/day, from about 3 g/m²/day to about 12 g/m²/day, or from about 10 g/m²/day. In some embodiments, the therapeutically effective amount of the compound is about 1 g/m²/day, about 3 g/m²/day, about 5 g/m²/day, about 8 g/m²/day, about 10 g/m²/day, or about 20 g/m²/day. The compound may be administered once a day or more than once a day. For example, the compound may be administered twice or three times a day. In some embodiments, on some days the compound may be administered once, and on other days the compound may be administered twice or thrice. The dosage amount and administration regimen may vary depending on the recommendation of the treating physician.

### Telomere Biology Disorders

In some embodiments, a telomere biology disorder (“TBD”) comprises telomere diseases or disorders associated with telomerase dysfunction. Without being bound by any theory, it is believed that the TBD is typically associated with changes in the length of telomeres. Suitable examples of symptoms, manifestations, and/or comorbidities of TBDs include aplastic anemia, myelodysplastic syndrome, acute myeloid leukemia, liver cirrhosis, hepatopulmonary syndrome, pulmonary fibrosis, interstitial lung disease, avascular necrosis, retinopathy, and gastrointestinal bleeding, or any combination of the foregoing. These symptoms, manifestations, and/or comorbidities can be used by a physician or diagnostician to aid in the diagnosis of the TBD (as described more fully below). Also, the compounds and methods of this disclosure are useful to reduce, prevent, and/or ameliorate any of these symptoms and conditions.

Among the telomere biology disorders is dyskeratosis congenita (DC), which is a rare, progressive bone marrow failure syndrome characterized by short telomeres. Patients can also display a triad of reticulated skin hyperpigmentation, nail dystrophy, and oral leukoplakia. Early mortality is often associated with bone marrow failure, infections, fatal pulmonary complications, or malignancy. Short-term treatment options for bone marrow failure in patients include anabolic steroids (e.g., oxymetholone), granulocyte macrophage colony-stimulating factor, granulocyte colony-stimulating factor, and erythropoietin. Other treatments include hematopoietic stem cell transplantation (SCT).

Idiopathic pulmonary fibrosis is a chronic and ultimately fatal disease characterized by a progressive decline in lung function. In some appropriate cases, the following agents are used to treat idiopathic pulmonary fibrosis: nintedanib, a tyrosine kinase inhibitor that targets multiple tyrosine kinases, including vascular endothelial growth factor, fibroblast growth factor, and PDGF receptors; and pirfenidone. Other treatments include lung transplantation. In some cases, lung transplantation for idiopathic pulmonary fibrosis (I-IPF) has been shown to confer a survival benefit over medical therapy.

In some embodiments, the present disclosure provides a method of treating a telomere biology disorder selected from dyskeratosis congenita, aplastic anemia, myelodysplastic syndrome, pulmonary fibrosis, idiopathic pulmonary fibrosis, interstitial lung disease, hematological disorder, liver disease, hepatic fibrosis, Hoyeraal-Hreidarsson syndrome, Coats Plus syndrome, and Revesz syndrome.

In some embodiments, the present disclosure provides a method of treating a telomere biology disorder selected from dyskeratosis congenita, aplastic anemia, and myelodysplastic syndrome.

In some embodiments, the method of this disclosure includes administering the compound of Formula (I) as described herein (e.g., thymidine or deoxyuridine), or a pharmaceutically acceptable salt thereof, administered in combination with an additional therapeutic agent useful in treating a telomere biology disorder. For example, the method may include administering to the subject a PAPD5 inhibitor, or a pharmaceutically acceptable salt thereof. In another example, the method includes administering to the subject an inhibitor of dNTPase SAM domain and HD domain-containing protein 1 (SAMHD1), or a pharmaceutically acceptable salt thereof. The SAMHD 1 inhibitor may be a biomolecule (having a molecular weight of 200 daltons or more produced by living organisms or cells) or a small-molecule drug (typically about 2000 daltons or less). In some embodiments, the SAMHD1 inhibitor is a protein or a nucleic acid, such as siRNAs, shRNAs, or gRNA.

Aging (Disorders where Short Telomeres are Implicated)

In some embodiments, a telomere biology disorder (“TBD”) comprises a disorder associated with aging. Without being bound by any theory or speculation, it is believed that telomeres shorten over the human life span. In large population based studies, short or shortening telomeres are associated with numerous diseases. Thus, telomeres have an important role in the aging process, and can contribute to various diseases. The role of telomeres as a contributory and interactive factor in aging, disease risks, and protection is described, e.g., in Blackburn, Elizabeth H., Elissa S. Epel, and Jue Lin. “Human telomere biology: A contributory and interactive factor in aging, disease risks, and protection,” Science 350.6265 (2015): 1193-1198, which is incorporated by reference in its entirety.

Telomere attrition is also a major driver of the senescence associated response. In proliferating human cells, progressive telomere erosion ultimately exposes an uncapped free double-stranded chromosome end, triggering a permanent DNA damage response (DDR). The permanent DNA damage response has a profound impact on cell functions. For example, the damage sensor ataxia telangiectasia mutated (ATM) is recruited to uncapped telomeres, leading to the stabilization of tumor suppressor protein 53 (p53) and upregulation of the p53 transcriptional target p21. In turn, p21 prevents cyclin-dependent kinase 2 (CDK2)-mediated inactivation of RB, subsequently preventing entry into the S phase of the cell cycle. Cellular senescence contributes to various age-related diseases, e.g., glaucoma, cataracts, diabetic pancreas, type 2 diabetes mellitus, atherosclerosis, osteoarthritis, inflammation, atherosclerosis, diabetic fat, cancer, pulmonary fibrosis, and liver fibrosis, etc. The permanent DNA damage response and age-related diseases are described, e.g., in Childs, Bennett G., et al. “Cellular senescence in aging and age-related disease: from mechanisms to therapy.” Nature medicine 21.12 (2015): 1424, which is incorporated herein by reference in its entirety.

As used herein, the term “aging” refers to degeneration of organs and tissues over time, in part due to inadequate replicative capacity in stem cells that regenerate tissues over time. In some embodiments, “aging” may refer to cellular senescence. Aging may be due to natural disease processes that occur over time, or those that are driven by cell intrinsic or extrinsic pressures that accelerate cellular replication and repair. Such pressures include natural chemical, mechanical, and radiation exposure; biological agents such as bacteria, viruses, fungus, and toxins; autoimmunity, medications, chemotherapy, therapeutic radiation, cellular therapy. As the telomere is an important factor in aging and disease development, the methods described herein can be used for treating, mitigating, or minimizing the risk of, a disorder associated with aging (and/or one or more symptoms of a disorder associated with aging) in a subject. The methods include the step of identifying a subject as having, or being at risk of a disorder associated with aging; and administering a pharmaceutical composition comprising a compound of this disclosure to the subject.

As used herein, the term “disorders associated with aging” or “age-related diseases” refers to disorders that are associated with the aging process. Exemplary disorders include, e.g., macular degeneration, diabetes mellitus (e.g., type 2 diabetes), osteoarthritis, rheumatoid arthritis, sarcopenia, cardiovascular diseases such as hypertension, atherosclerosis, coronary artery disease, ischemia/reperfusion injury, cancer, premature death, vascular insufficiency, interstitial lung disease, as well as age-related decline in cognitive function, cardiopulmonary function, muscle strength, vision, and hearing. In some embodiments, the present disclosure provides a method of treating a neurodevelopmental disorder. In some embodiments, the neurodevelopmental disorder is pontocerebellar hypoplasia.

The disorder associated with aging can also be a degenerative disorder, e.g., a neurodegenerative disorder. Degenerative disorders that can be treated or diagnosed using the methods described herein include those of various organ systems, such as those affecting brain, heart, lung, liver, muscles, bones, blood, gastrointestinal and genito-urinary tracts. In some embodiments, degenerative disorders are those that have shortened telomeres, decreased levels of TERC, and/or decreased levels of telomerase relative to normal tissues. In some embodiments, the degenerative disorder is a neurodegenerative disorder. Exemplary neurodegenerative disorders include Motor Neuron Disease, Creutzfeldt-Jakob disease, Machado-Joseph disease, Spino-cerebellar ataxia, Multiple sclerosis (MS), Parkinson's disease, Alzheimer's disease, Huntington's disease, hearing and balance impairments, ataxias, epilepsy, mood disorders such as schizophrenia, bipolar disorder, and depression, dementia, Pick's Disease, stroke, CNS hypoxia, cerebral senility, and neural injury such as head trauma. Recent studies have shown the association between shorter telomeres and Alzheimer's disease. The relationship between telomere length shortening and Alzheimer's disease is described, e.g., in Zhan, Yiqiang, et al. “Telomere length shortening and Alzheimer disease-a Mendelian Randomization Study,” JAMA neurology 72, 10 (2015), 1202-1203, which is incorporated by reference in its entirety. In some embodiments, the neurodegenerative disorder is dementia, e.g., Alzheimer's disease.

It has also been determined that there an inverse association between leucocyte telomere length and risk of coronary heart disease. This relationship is described, e.g., in Haycock, Philip C., et al. “Leucocyte telomere length and risk of cardiovascular disease: systematic review and meta-analysis.” (2014), g4227, and Codd et al. “Identification of seven loci affecting mean telomere length and their association with disease.” Nature genetics, 45, 4 (2013), 422-427, each of which is incorporated by reference in its entirety. Thus, there is strong evidence for a causal role of telomere-length variation in cardiovascular disease (CVD), or coronary artery disease (CAD). In some embodiments, the disorder is a cardiovascular disease (CVD), and/or coronary artery disease (CAD), and the present disclosure provides methods of treating, mitigating, or minimizing the risk of, these disorders. In some cases, the disorder is an atherosclerotic cardiovascular disease.

Furthermore, a meta-analysis of 5759 cases and 6518 controls indicated that shortened telomere length was significantly associated with type 2 diabetes mellitus risk. The relationship between telomere length and type 2 diabetes mellitus is described, e.g., in Zhao, Jinzhao, et al. “Association between telomere length and type 2 diabetes mellitus: a meta-analysis.” PLOS One 8.11 (2013): e79993, which is incorporated by reference in its entirety. In some embodiments, the disorder is a metabolic disorder, e.g., type 2 diabetes mellitus.

Additional suitable examples of aging-related disorders (where insufficient telomere length is implicated in the pathology of the disorder) include inflammatory disease, immune disease, infectious disease, cardiovascular disease, dermatological disease, ophthalmic disease, neurological disease, wasting disorder, metabolic disorder, and a disorder of the connective tissue. In some embodiments, any of the aforementioned and foregoing disorders are not necessarily associated with aging but are simply known for being associated with short telomeres. In one example, a person diagnosed with any of the inflammatory diseases, immune diseases, infectious diseases, wasting disorders, metabolic disorders, disorders of the connective tissue, or any other disorders described herein, is a young person with no signs of aging. Examples of those disorders include age-related anxiety, anemia, anorexia, arteriosclerosis, asthma, balance disorder, Bell's palsy, bone marrow failure, breathlessness, cachexia, chronic infection, cirrhosis, congestive heart failure, deafness, diabetes, emphysema, failure to thrive, flu, frailty, gastrointestinal ulcer, generalized anxiety disorder, gout, hair loss, hearing loss, hepatic insufficiency, high blood pressure, high fat, hip dislocation, hypercholesterolemia, hyperglycemia, hyperhomocysteinemia, hyperlipidemia, immunosenescence, impaired mobility, loss of appetite, loss of bone density, loss of sense of taste, metabolic syndrome, muscle loss, muscle wasting, muscular dystrophy, myocardial infarction, obesity, organ dysfunction, osteoporosis, peripheral artery disease, peripheral vascular disease, pneumonia secondary to impaired immune function, pulmonary disease, pulmonary emphysema, pulmonary fibrosis, reduced fitness, renal disease, renal insufficiency, scoliosis, spinal stenosis, syndrome X, tinnitus, urinary incontinence, vertebral fracture, weight loss, coronary artery disease, diabetes mellitus, type 2 diabetes, osteoarthritis, rheumatoid arthritis, sarcopenia, hypertension, atherosclerosis, ischemia, reperfusion injury, premature death, vascular insufficiency, interstitial lung disease, age-related decline in cognitive function, age-related decline in cardiopulmonary function, age-related decline in muscle strength, age-related decline in vision, and age-related decline in hearing.

Additional suitable examples of aging-related disorders (where insufficient telomere length is implicated in the pathology of the disorder) include skin diseases and senescence-associated dermatological diseases and disorders. Suitable examples of these disorders include rough skin, formation of wrinkles, coloring or spots, abnormal coloration of skin, formation of sagging, easy skin damage, atrophy, diabetic ulcers, and other ulcers.

Additional suitable examples of aging-related disorders (where insufficient telomere length is implicated in the pathology of the disorder) include senescence-associated ophthalmic diseases and disorders. Suitable examples of these disorders include cataract, corneal abnormalities, scleral abnormalities including pinguecula and pterygium, lacrimal duct abnormalities, conjunctival abnormalities, chalazion, glaucoma, macular degeneration, age-related macular degeneration, vascular retinopathy, and other retinopathy.

Additional suitable examples of aging-related disorders (where insufficient telomere length is implicated in the pathology of the disorder) include neurodevelopmental, neurological, psychiatric, and neurodegenerative disorders. Suitable examples include Alzheimer's disease, hearing loss, dementia, chronic traumatic encephalopathy, brain atrophy, amyotrophic lateral sclerosis, Parkinson's disease, Gillian-Barre syndrome, peripheral neuropathy, Creutzfeldt-Jakob disease, frontotemporal dementia, spinal muscular atrophy, and Friedreich's ataxia, vascular dementia, mild cognitive impairment, severe cognitive impairment, memory loss, pontocerebellar hypoplasia, motor neuron disease, Machado-Joseph disease, spino-cerebellar ataxia, Multiple sclerosis, Huntington's disease, hearing impairment, balance impairment, ataxias, epilepsy, mood disorder, schizophrenia, bipolar disorder, depression, Pick's Disease, stroke, CNS hypoxia, cerebral senility, neural injury, and head trauma.

Additions examples of age-related disorders, as well as the symptoms associated with those disorders and methods to diagnose them are described in WO2012050162, EP3988112, WO2019069070, and US20220143218, which are incorporated herein by reference in their entirety. In some embodiments, the age-related illness is simply old age.

In addition, various types of cancer (e.g., those described herein) may be considered age-related illnesses, particularly where the cancerous cells contain short telomeres. In some embodiments, the age-related illness treatable or preventable by the compounds and methods of this disclosure is simply the altered form and function typically associated with old chronological age in humans.

Further, in relation to ageing, the compounds and methods of this disclosure may also be used as a cosmetic aid, to prevent, delay, or ameliorate age-related deterioration in appearance of skin, hair, bone structure, posture, eye clarity, or other cosmetic traits that decline with aging. For example, the compounds may be used to help maintain skin elasticity, thickness, smoothness, and appearance, since the loss of these characteristics is associated with telomere shortening. In cases of physical trauma such as a bone fracture or a tissue crush or cut injury or burn, the compounds may be used to increase the lengths of telomeres in cells which participate in healing the trauma, to increase their replicative capacity. In cases of chronic physical stress, which causes telomere shortening, treatment with the compounds of this disclosure may lengthen telomeres in affected cells increasing their replicative capacity and ability to repair tissue damage. Since telomere shortening accumulates over generations, for example in humans with haploinsufficiency of telomerase components such as hTR or hTERT (See Armanios (2009) Annu. Rev. Genomics Hum. Genet. 10, 45-61, which is incorporated herein by reference), the treatments of the instant disclosure may be applied to germ line cells such as eggs, sperm, or their precursors, or to fertilized eggs or embryos, for example during in vitro fertilization procedures. The compounds may also be useful for aiding other treatments of various diseases or conditions, for example, transdifferentiation of cells in vivo. The treatment methods and compounds of this disclosure may also be useful in advance of or during surgery or chemotherapy, or radiotherapy, to increase the ability of cells to replicate to repair damage resulting from these procedures.

### Cancer

The compounds and compositions of this may be used for treating pre-leukemic conditions, pre-cancerous conditions, dysplasia and/or cancers. Pre-leukemic conditions include, e.g., myelodysplastic syndrome, and smoldering leukemia. Dysplasia refers to an abnormality of development or an epithelial anomaly of growth and differentiation, including e.g., hip dysplasia, fibrous dysplasia, and renal dysplasia, Myelodysplastic syndromes, and dysplasia of blood-forming cells.

A precancerous condition or premalignant condition is a state of disordered morphology of cells that is associated with an increased risk of cancer. If left untreated, these conditions may lead to cancer. Such conditions are can be dysplasia or benign neoplasia.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells.

Many cancer cells have abnormal telomeres. Thus, compounds and treatments described herein can also be used to treat cancers. Cancers that can be treated or diagnosed using the methods described herein include malignancies of the various organ systems, such as affecting skin, head, neck, oropharyngeal and nasal mucosa, brain, lung, breast, thyroid, lymphoid, gastrointestinal, liver, genito-urinary tract, and blood vessel constituents such as angiosarcoma, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In some embodiments, the methods described herein are used for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Without being bound by any particular theory or speculation, it is believed that various types of cancer may be prevented or delayed by treatment with compounds and methods of this disclosure. Indeed chromosome-chromosome fusions and gene mutations caused by critically short telomeres are believed to be a cause of cancer. The compounds of this disclosure may also advantageously be selectively lengthened in healthy cells in an individual, while not lengthening telomeres in cancer cells, which may allow the instant compounds, compositions, and methods to be used, for example, to lengthen telomeres of the immune system to increase its ability to fight a cancer. Further, immune system cells may be harvested from an individual for treatment using the invention ex vivo followed by reintroduction into the individual.

In some embodiments, suitable examples of cancers treatable or preventable by the compounds and methods of this disclosure include bladder cancer, blood vessel cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. In some embodiments, the cancer is selected from sarcoma, angiosarcoma, fibrosarcoma, rhabdomyosarcoma, liposarcoma, myxoma, rhabdomyoma, fibroma, lipoma, teratoma, lung cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), malignant giant cell tumor, chordoma, osteochrondroma (osteocartilaginous exostoses), benign chondroma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, lymphoma, leukemia, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, adrenal gland cancer, and neuroblastoma.

### Diagnosing a Subject in Need of Treatment

The present methods of this disclosure may include a step of diagnosing a subject in need of treatment (e.g., as having any one of telomere diseases described herein). For example, the method may include a step of identifying a subject having a disease or disorder as described herein. In some embodiments, the method includes a step of identifying a subject in need of treatment. In some embodiments, the method includes a step of identifying a subject diagnosed with a disorder described herein (e.g., by a treating physician, a specialist (e.g., oncologist or radiologist), or a diagnostician).

As an example, the subject may be diagnosed by a physician on the basis of one or more symptoms associated with telomere disease (e.g., aplastic anemia, pulmonary fibrosis, hepatic cirrhosis). The subject may also be diagnosed as having or being at risk of developing a disease described herein (e.g., a telomere disease). As one aspect of the diagnosis, an absence of an alternative cause of disease in combination with telomere length testing and genetic sequencing (as described more fully below) can be used to aid in diagnosis of a telomere biology disorder, or an aging disorder or a related disorder associated with short telomeres. Diagnosis can be based on physical examination, blood testing, pulmonary function testing or any other diagnostic testing (e.g., analysis of a specimen for a subject or an imaging technique) appropriate for a particular disease or disorder.

In some embodiments, the subject may be diagnosed because the level of telomere length or activity of telomerase in the subject is comparable to the level of telomere length or activity of telomerase in a subject having a telomere disease. For example, telomere length testing for the diagnostic purposes can be performed using the clinically validated flow-FISH assay which is ordered and interpreted by the physician (See, e.g., Nature Protocols, 1, 2365-2376, 2006, which is incorporated herein by reference in its entirety). Without being bound by any particular theory or speculation, mean telomere length in lymphocytes less than the first percentile for a given age as determined by flow-FISH has been shown to be sensitive in the detection of TBDs in children. At older ages telomere length testing is less sensitive and specific. In some embodiments, telomere length less than 10th percentile is indicative of a diagnosis of a telomere-related disorder. Telomere length >50th percentile can aid in excluding the disease (See, e.g., Blood, 2007, 110, 5, 1439-47; Haematologica, 2012, 97, 3, 353; PNAS, 2018, 115, 10, E2358-E2365, which are incorporated herein by reference in their entirety).

In some embodiments, the subject may be diagnosed because the level or activity of TERC and/or PARN in the subject is comparable to the level or activity of TERC and/or PARN in a subject having a telomere disease. In some embodiments, if the level or activity of TERC and/or PARN in a subject is comparable to the level or activity of TERC and/or PARN in a control subject who does not have a telomere disease, then the subject can be diagnosed as not having telomere disease or not being at risk of developing a telomere disease.

In some embodiments, the subject is determined to have or being at risk of developing disorder described herein (e.g., a telomere biology disease) if there is a mutation that impairs telomere biology. Without being bound by a theory, a telomere disorder can be diagnosed on the basis of genetic testing. Numerous peer-reviewed publications provide credible evidence that mutations in the telomere-associated genes were shown to cause disease associated with short telomeres as discussed herein. Examples of those genes include DKC1 (See, e.g., Nat Genet, 1998, 19, 1, 32-8, which is incorporated herein by reference in its entirety), TERC (See, e.g., Nature, 2001, 27, 413, 6854, 432-5, which is incorporated herein by reference in its entirety), TERT (See, e.g., N Engl J Med, 2005, 7, 352, 14, 1413-24, which is incorporated herein by reference in its entirety), NOP10 (See, e.g., Hum Mol Genet, 2007, 16, 13, 1619-29, which is incorporated herein by reference in its entirety), NHP2 (See, e.g., PNAS, 2008, 105, 23, 8073-8, which is incorporated herein by reference in its entirety), TINF2 (See, e.g., Am J Hum Genet, 2008, 82, 2, 501-9, which is incorporated herein by reference in its entirety), WRAP53/TCAB1 (See, e.g., Genes Dev, 2011, 25, 1, 11-6, which is incorporated herein by reference in its entirety), CTC1 (See, e.g., Pediatr Blood Cancer, 2012, 59, 2, 311-4, which is incorporated herein by reference in its entirety), RTEL1 (See, e.g., Hum Genet, 2013, 132, 4, 473-80, which is incorporated herein by reference in its entirety), ACD (See, e.g., Genes Dev, 2014, 28, 19, 2090-102, which is incorporated herein by reference in its entirety), PARN (See, e.g., J Clin Invest, 2015, 125, 5, 2151-60, which is incorporated herein by reference in its entirety), NAF1 (See, e.g., Sci Transl Med, 2016, 8, 351, 351ra107, which is incorporated herein by reference in its entirety), STN1 (See, e.g., J Exp Med, 213, 8, 1429-40, which is incorporated herein by reference in its entirety), ZCCHC8 (See, e.g., Genes Dev, 2019, 33, 19-20, 1381-1396, which is incorporated herein by reference in its entirety), and RPA1 (See, e.g., Blood, 2022, 139, 7, 1039-1051, which is incorporated herein by reference in its entirety). In some embodiments, detection (by any suitable scientific technique) of known or predicted pathogenic variants in the foregoing genes can aid the diagnosis of TBDs. These mutations can be detected using targeted panels or genome/exome sequencing. A skilled diagnostician would know how to interpret the results of these tests. For example, mutations commonly found in the public may be excluded, while mutations previously associated with telomere disease may be diagnostic.

In some embodiments, the method of this disclosure comprises identifying a subject having a mutation in any factor that regulates telomere biology, such as PARN, NOP10, NHP2, NAF1, TCAB1/WRAP53, ZCCHC8, TERC, TERT, TINF2, ACD/TPP1, STN1, CTC1, POT1, or RPA1. In some embodiments, the method of this disclosure comprises identifying a subject having a mutation in any factor regulating nucleotide metabolism, and as a consequence, telomere biology. In some embodiments, the method of this disclosure comprises a step of identifying a subject having a mutation in a factor that regulates telomere biology. In some embodiments, the factor that regulates telomere biology is PARN, NOP10, NHP2, NAF1, TCAB1/WRAP53, ZCCHC8, TERC, TERT, TINF2, ACD/TPP1, STN1, CTC1, POT1, or RPA1. In some embodiments, the method of this disclosure comprises identifying a subject having a mutation in DKC1. The mutation can be a missense mutation, deletion or truncation mutation, omission of single or groups of nucleotides encoding one or several amino acids, non-coding mutation such as promoter, enhancer, or splicing mutation, or other mutations. The mutation can be a deletion containing part of the gene or the entire gene (e.g., part of PARN gene or the entire PARN gene, or part of DKC1 gene or the entire DKC1 gene). In one example, mutation can be a mutation at position 7 and/or 87 of PARN, e.g., the amino acid residue at position 7 is not asparagine, and/or the amino acid residue at position 87 of PARN is not serine. For example, the mutation can be a missense variant c. 19A>C, resulting in a substitution of a highly conserved amino acid p.Asn7His. In some cases, the mutation is a missense mutation c.260C>T, encoding the substitution of a highly conserved amino acid, p.Ser87Leu. In some embodiments, the subject is determined to have or be at risk of developing a telomere disease if there is a mutation in any factor that regulates TERC, including NOP10, NHP2, NAF1, GAR1, TCAB1/WRAP53, ZCCHC8, or in TERC itself. In some embodiments, the method of this disclosure comprises a step of identifying a subject having a mutation in a factor that regulates TERC. In some embodiments, the factor is NOP10, NHP2, NAF1, GAR1, TCAB1/WRAP53, ZCCHC8, or TERC.

In some embodiments, in relation to telomere biology disorders, if a subject has no overt signs or symptoms of a telomere disease, but the level or activity of PARN, NOP10, NHP2, NAF1, TCAB1/WRAP53, ZCCHC8, TERC, TERT, TINF2, ACD/TPP1, STN1, CTC1, POT1, or RPA1 may be associated with the presence of a telomere disease, then the subject has an increased risk of developing a telomere biology disease. In some embodiments, once it has been determined that a person has telomere disease, or has an increased risk of developing telomere disease, then a treatment, e.g., thymidine or deoxyuridine, or a combination thereof, can be administered as described herein.

For diagnostic purposes, suitable reference values can be determined using methods known in the art, e.g., using standard clinical trial methodology and statistical analysis. The reference values can have any relevant form. In some cases, the reference comprises a predetermined value for a meaningful level of telomere length, e.g., a control reference level that represents a normal level of telomere length, e.g., a level in an unaffected subject or a subject who is not at risk of developing a disease described herein, and/or a disease reference that represents a level of the proteins associated with conditions associated with telomere disease, e.g., a level in a subject having telomere disease (e.g., pulmonary fibrosis, hepatic cirrhosis or aplastic anemia). In some cases, the reference comprises a predetermined value for a meaningful level of TERC RNA or PARN protein. In another embodiment, the reference comprises a predetermined value for a meaningful level of thymidine, nucleosides, or nucleotides in the plasma, serum, other bodily fluid, or in cells. In another embodiment, the reference comprises a predetermined value for a meaningful level of telomerase activity, e.g., a control reference level that represents a normal level of telomerase activity, e.g., a level in an unaffected subject or a subject who is not at risk of developing a disease described herein, and/or a disease reference that represents a level of the proteins associated with conditions associated with telomere disease, e.g., a level in a subject having telomere disease (e.g., pulmonary fibrosis, hepatic cirrhosis or aplastic anemia).

In some embodiments, the predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein. In some embodiments, it may be desirable that the control subject is deficient in telomere length (e.g., dyskeratosis congenita), and in other embodiments, it may be desirable that a control subject has cancer. In some embodiments, it may be desirable that the control subject is deficient in PARN gene or TERC gene. In some cases, it may be desirable that the control subject has high telomerase activity, and in other cases it may be desirable that a control subject does not have substantial telomerase activity.

In some embodiments, the level of telomere length in a subject being less than or equal to a reference level of telomere length is indicative of a clinical status (e.g., indicative of a disorder as described herein, e.g., telomere disease). In some embodiments, the activity of telomere length in a subject being greater than or equal to the reference activity level of telomere length is indicative of the absence of disease.

The predetermined value can depend upon the particular population of subjects (e.g., human subjects or animal models) selected. For example, an apparently healthy population will have a different ‘normal’ range of levels of telomere length than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art. In characterizing likelihood, or risk, numerous predetermined values can be established.

In some embodiments, the methods described in this disclosure involves identifying a subject as having, being at risk of developing, or suspected of having a disorder associated with telomerase dysfunction. The methods include determining the level of telomere length in a cell from the subject; comparing the level of telomere length to the reference level of telomere length; and identifying the subject as having, being at risk of developing, or suspected of having a disorder associated with telomerase dysfunction if the level of telomere length is significantly different from the reference level of telomere length. In some embodiments, the reference level of telomere length is determined by cells obtained from subjects without disorders associated with telomerase dysfunction.

The methods of obtaining a cell from a subject can include obtaining cells from a subject, and transforming these cells to induced pluripotent stem (iPS) cells, and these iPS cells can be used to determine the level or activity of relevant gene or protein, e.g., PARN, NOP10, NHP2, NAF1, TCAB1/WRAP53, ZCCHC8, TERC, TERT, TINF2, ACD/TPP1, STN1, CTC1, POT1, or RPA1. These cells can be, e.g., primary human cells or tumor cells. Pluripotent stem cells (e.g. iPS) cells can be generated from somatic cells by methods known in the art (e.g., somatic cells may be genetically reprogrammed to an embryonic stem cell-like state by being forced to express genes and factors important for maintaining the defining properties of embryonic stem cells). In some embodiments, the methods of diagnosing a subject include analyzing blood sample of the subject, or a sample of hair, urine, saliva, or feces of the subject (e.g., a subject may be diagnosed without any cell culture surgically obtained from the subject). In some embodiments, the method of diagnosing a identifying a subject in need to treatment includes identifying a relevant biomarker in a cell or tissue obtained from the subject (e.g., a blood sample as described above).

The subject may be one having a mutation at PARN, e.g., a deletion containing part of PARN gene or the entire PARN gene. For example, the mutation may be one wherein the amino acid residue at position 7 of PARN is not asparagine or serine. For example, the subject can have a missense variant c. 19A>C, resulting in a substitution of a highly conserved amino acid p.Asn7His. The subject can have a missense mutation c.260C>T, encoding the substitution of a highly conserved amino acid, p.Ser87Leu.

In some embodiments, any of the methods of this disclosure includes a step of monitoring treatment of any of the diseases described herein. For example, upon administration of the pyrimidine nucleoside to a subject, the method many include a step of determining blood thymidine levels. Furthermore, the monitoring step may include determining increase in telomere length (as compared to telomere length before the treatment) as measured by flow-FISH on peripheral blood. The monitoring step may also include observation of an improvement in symptoms of the disease being treated. Those improvements may include increased blood counts, improved lung function, improved liver function, skin and nail changes, mucosal changes, blood vessel changes, and improved bone health and strength.

### Additional Aspects

In some embodiments, the present disclosure provides a method of promoting telomere length in a cell, the method comprising contacting the cell with a compound of this disclosure (e.g., thymidine), or a pharmaceutically acceptable salt thereof. In some embodiments, the method can be carried out in vitro, in vivo, or ex vivo.

In some embodiments, the present disclosure provides a method of restoring telomere length in a cell, the method comprising contacting the cell with a compound of this disclosure (e.g., thymidine), or a pharmaceutically acceptable salt thereof. In some embodiments, the method can be carried out in vitro, in vivo, or ex vivo.

In some embodiments, the present disclosure provides a method of increasing amount of dTMP, dTDP, and/or dTTP in a cell, the method comprising contacting the cell with a compound of this disclosure (e.g., thymidine), or a pharmaceutically acceptable salt thereof. In some embodiments, the method can be carried out in vitro, in vivo, or ex vivo. In some embodiments, the contacting results in about 4-fold increase in the level of dTMP, dTDP, and/or dTTP in the cell compared to cellular level of dTMP, dTDP, and/or dTTP prior to said contacting. In some embodiments, the contacting results in no change in cellular levels of dATP and/or dGTP.

In some embodiments of any of the foregoing methods, contacting the cell with the compound of Formula (A) (e.g., thymidine) results in increase in telomere basepairs in the cell. In some embodiments, the increase is a 2-fold, a 4-fold, a 10-fold, a 1,000-fold, a 2,000-fold, a 10,000-fold, or a 50,000-fold increase. In some embodiment, the increase is about 10%, about 200%, about 50%, about 100%, about 150%, or about 200% increase. In some embodiments, said contacting does not impact cellular growth and/or does not disrupt the cell cycle. In some embodiments, the contacting results in cellular concentration of the compound of Formula (A) (e.g., thymidine) of about 0.5 μM, about 1 μM, about 1.5 μM, about 2 μM, about 3 μM, or about 5 μM. In some embodiments, the contacting results in a concentration of the compound of Formula (A) (e.g., thymidine) in the nucleus of a cell of about 0.5 μM, about 1 μM, about 1.5 μM, about 2 μM, about 3 μM, or about 5 M. In some embodiments, these concentrations do not lead to any disruption of cellular cycle, replication, and/or cellular growth.

In some embodiments, this disclosure provides a method of treating or preventing a disorder associated with a short telomere length. Suitable examples of such disorders include coronary artery disease, coronary heart disease, abdominal aortic aneurysm, celiac disease, and interstitial lung disease. This disorders, as well as the involvement of shortened telomeres in their pathology, are described, for example, in Codd et al, Nature Genetics volume 53, pages 1425-1433 (2021) and Haycock et al, JAMA Oncol, 2017 May 1, 3 (5), 636-651, which are incorporated herein by reference in their entirety. The compounds and methods of this disclosure may also benefit subjects at risk of age-related diseases or conditions, or who are already suffering from such diseases, and may also benefit subjects who have experienced, are experiencing, or are at risk of experiencing physical trauma or chronic physical stress such as hard exercise or manual labor, or psychological trauma or chronic psychological stress, since all of these conditions cause telomere shortening; physical stress or trauma requires cell division in order to repair the resultant damage, thus shortening telomeres, and these conditions may also cause oxidative stress, which also shortens telomeres.

For all the disease states described herein, where telomere length is implicated in the pathology, the need for cell replication (and the cell replication is often the cellular response to address the problem caused by short telomeres) results in even more rapid telomere shortening than normal, which in turn exhausts the replicative capacity of cells, leading to tissue dysfunction, exacerbated or additional symptoms, disability, and often death. In some embodiments, no disease state is yet manifested but the subject is at risk (is identified or about to be identified as to be at risk) for a condition or disease involving short telomeres, or where the cells obtained from the subject contain shortened telomeres compared to healthy control.

In some embodiments, the methods and compounds of this disclosure are useful for treating cells in vitro for various applications, including autologous or heterologous cell therapy, bioengineering, tissue engineering, growth of artificial organs, generation of induced pluripotent stem (iPS) cells, and cellular differentiation, dedifferentiation, or transdifferentiation. In these applications, cells may be required to divide many times, which may lead to loss of telomere length, which may be counteracted by the compounds of this disclosure before, during, or after the application.

In some embodiments, suitable examples of cells that may be contacted with a compound of this disclosure include cells from any tissue or cell type that may suffer the effects of shortened telomeres or that may in any way benefit from lengthening of the cell's telomeres. Examples of cells may include somatic cells or germ cells, as well as stem cells and other progenitor cells and/or undifferentiated cells. Examples of cells may include tumor cells and non-tumor cells.

Examples of cells that may be contacted (e.g., in vitro, in vivo, or ex vivo) with a compound of this disclosure include cells that are derived primarily from endoderm, cells that are derived primarily from ectoderm, and cells that are derived primarily from mesoderm. Examples of cells derived primarily from the endoderm include, for example, exocrine secretory epithelial cells and hormone-secreting cells. Examples of cells derived primarily from the ectoderm include, for example, cells of the integumentary system (e.g., keratinizing epithelial cells and wet stratified barrier epithelial cells) and the nervous system (e.g., sensory transducer cells, autonomic neuron cells, sense organ and peripheral neuron supporting cells, central nervous system neurons and glial cells, and lens cells). Examples of cells derived primarily from the mesoderm include, for example, metabolism and storage cells, barrier-function cells (e.g., cells of the lung, gut, exocrine glands, and urogenital tract), extracellular matrix cells, contractile cells, blood and immune system cells, germ cells, nurse cells, and interstitial cells. Accordingly, in some embodiments, the cell that may be contacted with the compound of this disclosure is a somatic cell of endodermal, mesodermal, or ectodermal lineage. In some embodiments the cell is a germ line cell or an embryonic cell.

Suitable examples of cells that may be treated and/or contacted according to the instant methods include, e.g., salivary gland mucous cells, salivary gland serous cells, von Ebner's gland cells in tongue, mammary gland cells, lacrimal gland cells, ceruminous gland cells in ear, eccrine sweat gland dark cells, eccrine sweat gland clear cells, apocrine sweat gland cells, gland of Moll cells in eyelid, sebaceous gland cells, Bowman's gland cells in nose, Brunner's gland cells in duodenum, seminal vesicle cells, prostate gland cells, bulbourethral gland cells, Bartholin's gland cells, gland of Littre cells, uterus endometrium cells, isolated goblet cells of the respiratory and digestive tracts, stomach lining mucous cells, gastric gland zymogenic cells, gastric gland oxyntic cells, pancreatic acinar cells, paneth cells of the small intestine, type II pneumocytes of the lung, clara cells of the lung, anterior pituitary cells (e.g., somatotropes, lactotropes, thyrotropes, gonadotropes, and corticotropes), intermediate pituitary cells (e.g., those secreting melanocyte-stimulating hormone), magnocellular neurosecretory cells (e.g., those secreting oxytocin or vasopressin), gut and respiratory tract cells, (e.g., those secreting serotonin, endorphin, somatostatin, gastrin, secretin, cholecystokinin, insulin, glucagon, or bombesin), thyroid gland cells (e.g., thyroid epithelial cells and parafollicular cells), parathyroid gland cells (e.g., parathyroid chief cells and oxyphil cells), adrenal gland cells (e.g., chromaffin cells and cells secreting steroid hormones such as mineralcorticoids and glucocorticoids), Leydig cells of testes, theca interna cells of the ovarian follicle, corpus luteum cells of the ruptured ovarian follicle, granulosa lutein cells, theca lutein cells, juxtaglomerular cells, macula densa cells of the kidney, peripolar cells of the kidney, mesangial cells of the kidney, epidermal keratinocytes, epidermal basal cells, keratinocytes of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of the stratified squamous epithelium of the cornea, tongue, oral cavity, esophagus, anal canal, distal urethra, and vagina, basal cells of the epithelia of the cornea, tongue, oral cavity, esophagus, anal canal, distal urethra, and vagina, urinary epithelium cells (e.g., lining the urinary bladder and urinary ducts), auditory inner hair cells of the organ of Corti, auditory outer hair cells of the organ of Corti, basal cells of the olfactory epithelium, cold-sensitive primary sensory neurons, heat-sensitive primary sensory neurons, Merkel cells of the epidermis, olfactory receptor neurons, pain-sensitive primary sensory neurons, photoreceptor cells of the retina in the eye (e.g., photoreceptor rod cells, photoreceptor blue-sensitive cone cells, photoreceptor green-sensitive cone cells, and photoreceptor red-sensitive cone cells), proprioceptive primary sensory neurons, touch-sensitive primary sensory neurons, type I and II carotid body cells, type I and II hair cells of the vestibular apparatus of the ear, type I taste bud cells, cholinergic neural cells, adrenergic neural cells, peptidergic neural cells, inner and outer pillar cells of the organ of Corti, inner and outer phalangeal cells of the organ of Corti, border cells of the organ of Corti, Hensen cells of the organ of Corti, vestibular apparatus supporting cells, taste bud supporting cells, olfactory epithelium supporting cells, Schwann cells, satellite glial cells, enteric glial cells, astrocytes, neuron cells, oligodendrocytes, spindle neurons, anterior lens epithelial cells, crystallin-containing lens fiber cells, hepatocytes, adipocytes (e.g., white fat cells and brown fat cells), liver lipocytes, kidney parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells (e.g., principal cells and intercalated cells), duct cells (of seminal vesicle, prostate gland, etc.), intestinal brush border cells (with microvilli), exocrine gland striated duct cells, gall bladder epithelial cells, ductulus efferens nonciliated cells, epididymal principal cells, epididymal basal cells, ameloblast epithelial cells, planum semilunatum epithelial cells of the vestibular apparatus of the ear, organ of Corti interdental epithelial cells, loose connective tissue fibroblasts, corneal fibroblasts (corneal keratocytes), tendon fibroblasts, bone marrow reticular tissue fibroblasts, other nonepithelial fibroblasts, pericytes, nucleus pulposus cells of the intervertebral disc, cementoblasts/cementocytes, odontoblasts/odontocytes, hyaline cartilage chondrocytes, fibrocartilage chondrocytes, elastic cartilage chondrocytes, osteoblasts/osteocytes, osteoprogenitor cells, hyalocytes of the vitreous body of the eye, stellate cells of the perilymphatic space of the ear, hepatic stellate cells (Ito cells), pancreatic stelle cells, skeletal muscle cells (e.g., red skeletal muscle cells (slow) and white skeletal muscle cells (fast)), intermediate skeletal muscle cells, nuclear bag cells of the muscle spindle, nuclear chain cells of the muscle spindle, satellite cells, heart muscle cells (e.g., ordinary heart muscle cells, nodal heart muscle cells, and Purkinje fiber cells, smooth muscle cells (various types), myoepithelial cells of the iris, myoepithelial cells of the exocrine glands, erythrocytes, megakaryocytes, monocytes, connective tissue macrophages (various types), epidermal Langerhans cells, osteoclasts, dendritic cells, microglial cells, neutrophil granulocytes, eosinophil granulocytes, basophil granulocytes, hybridoma cells, mast cells, helper T cells, suppressor T cells, cytotoxic T cells, natural killer T cells, B cells, natural killer cells, reticulocytes, stem cells and committed progenitors for the blood and immune system (various types), oogonia/oocytes, spermatids, spermatocytes, spermatogonium cells, spermatozoa, nurse cells, ovarian follicle cells, sertoli cells, thymus epithelial cells, and interstitial kidney cells.

In some embodiments, the cells treated with a compound of this disclosure are stem or progenitor cells, since these cells give rise to other cells of the body. For example, the cells are the cells in which telomeres shorten more quickly than in other cell types, for example endothelial cells, fibroblasts, keratinocytes, cells of the blood forming (e.g., hematopoietic) system such as neutrophils, red blood cells or platelets and their progenitors, cells of the immune system such as lymphocytes and their progenitors, cells of the blood vessels, intestines, liver, mucosal membrane cells, e.g., in the esophagus and colon, and cells of the gums and dental pulp.

### Cell Expansion

The present disclosure provides methods of expanding a cell population by culturing one or more cells in the presence of compounds as disclosed herein (e.g., compounds of Formula A). In some embodiments, cell expansion can involve contacting the cells with an effective amount of compound of the present disclosure (e.g., dT, dU, dC, or a combination thereof). The compounds can increase or maintain the length of the telomere.

Cellular therapies often rely on robust cellular division (either ex vivo or upon reintroduction to the body) for their therapeutic effect. Telomere length is associated with cellular replicative capacity: telomere attrition, which occurs during each cell cycle, can lead to critically short telomere length, triggering senescence and halting cell division. Thus, elongating telomeres in cellular products ex vivo may enhance cellular replicative capacity and thereby therapeutic efficacy. For example, there have been reports of telomere elongation increasing CAR-T cell efficacy in mouse models. (Paper: Cell Discov. 2015, 1, 15040). Many cells commonly expanded for therapeutic purposes ex vivo (e.g., stem cells, T cells), express telomerase, which is required for dT mediated telomere lengthening. Therefore, treatment of cells ex vivo with dT or combinations of nucleosides thus may promote telomere elongation and enhance the efficacy of cellular therapies The present disclosure provides methods of promoting cell expansion, and methods of inhibiting, slowing, or preventing cell aging.

In some embodiments, the cell is a stem cell. Stem cells can include, but are not limited to, for example, pluripotent stem cells, embryonic stem cells, hematopoietic stem cells, lymphoid stem cells, bone marrow stem cells, peripheral blood mobilized stem cells, adipose derived stem cells, mesenchymal stem cells, umbilical cord blood stem cells, placentally derived stem cells, exfoliated tooth derived stem cells, hair follicle stem cells, or neural stem cells. In some embodiments, the cell is a peripheral blood mononuclear (PBMC) cell.

The cells can be derived from the subject with a disease or condition associated with any disorder described herein, e.g., cancer, a telomere or telomerase dysfunction, a disorder associated with aging, a pre-leukemic or pre-cancerous condition, cancer, and a neurodevelopment disorder. The cells can be isolated and derived, for example, from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, or mesentery tissue.

The cells can be isolated from any mammalian organism, e.g., human, mouse, rats, dogs, or cats, by any means know to one of ordinary skill in the art. One skilled in the art can isolate embryonic or adult tissues and obtain various cells (e.g., stem cells).

The expanded cell population can be further enriched by using appropriate cell markers. For example, stem cells can be enriched by using specific stem cell markers, e.g., FLK-1, AC133, CD34, c-kit, CXCR-4, Oct-4, Rex-1, CD9, CD13, CD29, CD34, CD44, CD166, CD90, CD105, SH-3, SH-4, TRA-1-60, TRA-1-81, SSEA-4, and Sox-2. One skilled in the art can enrich a specific cell population by using antibodies known in the art against any of these cell markers. In some embodiments, expanded stem cells can be purified based on desired stem cell markers by fluorescence activated cell sorting (FACS), or magnet activated cell sorting (MACS).

The cells (e.g., stem cells) can be cultured and expanded in suitable growth media. Commonly used growth media include, but are not limited to, Iscove's modified Dulbecco's Media (IMDM) medium, McCoy's 5A medium, Dulbecco's Modified Eagle medium (DMEM), KnockOut™ Dulbecco's Modified Eagle medium (KO-DMEM), Dulbecco's Modified Eagle Medium/Nutrient Mixture F-12 (DMEM/F12), Roswell Park Memorial Institute (RPMI) medium, minimum essential medium alpha medium (α-MEM), F-12K nutrient mixture medium (Kaighn's modification, F-12K), X-vivo™ 20 medium, Stemline™ medium, StemSpan™ CC100 medium, StemSpan™ H2000 medium, MCDB 131 Medium, Basal Media Eagle (BME), Glasgow Minimum Essential medium (GMEM), Modified Eagle Medium (MEM), Opti-MEM I Reduced Serum medium, Waymouth's MB 752/1 Medium, Williams' Medium E, NCTC-109 Medium, neuroplasma medium, BGJb Medium, Brinster's BMOC-3 Medium, Connaught Medical Research Laboratories (CMRL) Medium, CO₂-Independent Medium, and Leibovitz's L-15 medium.

The compounds of the present disclosure (e.g., compounds of Formula A) can be used to expand various cell population, e.g., by adding the compound in cell culture media in a tube or plate. The concentration of the compound can be determined by, but limited to, the time of cell expansion. For example, the cells can be in culture with high concentration of the compound for a short period of time, e.g., at least or about 1 day, 2 days, 3 days, 4 days, or 5 days. In some embodiments, the cells can be cultured with a low concentration of the compound for a long period of time, e.g., at least or about 3 days, 4 days, 5 days, 6 days, 1 week, 2 weeks, 3 weeks, or 4 weeks.

In some embodiments, growth factors are also added to the growth medium to expand cells. Examples of suitable growth factors include, but are not limited to, thrombopoietin, stem cell factor, IL-1, IL-3, IL-7, flt-3 ligand, G-CSF, GM-CSF, Epo, FGF-1, FGF-2, FGF-4, FGF-20, IGF, EGF, NGF, LIF, PDGF, bone morphogenic proteins, activin-A, VEGF, forskolin, and glucocorticords. Further, one skilled in the art, using methods known in the art, can add a feeder layer to the culture medium. A feeder layer can include cells such as, placental tissue or cells thereof.

The methods described herein can also be used to produce and expand Chimeric Antigen Receptor (CAR) T-Cells. CAR-T cell therapies involve genetic modification of patient's autologous T-cells to express a CAR specific for a tumor antigen, following by ex vivo cell expansion and re-infusion back to the patient. PBMCs can be collected from a patient and cultured in the presence of the compounds as described herein (e.g., compounds of Formula A), with appropriate media (e.g., complete media containing 30 U/mL interleukin-2 and anti-CD3/CD28 beads). The cells can be expanded for about 3 to 14 days (e.g., about 3 to 7 days). Subsets of T cells can be sorted by FACS. Gating strategies for cell sorting can exclude other blood cells, including granulocytes, monocytes, natural killer cells, dendritic cells, and B cells. Primary T cells are then transduced by incubating cells with the CAR-expressing lentiviral vector in the culture media. In some embodiments, the culture media can be supplemented with the compounds as described herein. The transduced cells are then cultured for at least a few days (e.g., 3 days) before being used in CAR-T cell therapies.

In some embodiments, the present disclosure provides a method of expanding a cell, the method comprising culturing the cell in the presence of an effective amount of a compound as described herein (e.g., a compound of Formula A), or a pharmaceutically acceptable salt thereof.

In some embodiments, the cell is selected from the group consisting of: stem cell, pluripotent stem cell, hematopoietic stem cell, and embryonic stem cell. In some embodiments, the cell is a pluripotent stem cell. In some embodiments, the cell is a hematopoietic stem cell. In some embodiments, the cell is an embryonic stem cell. In some embodiments, the cell is collected from a subject with a disease or condition selected from the group consisting of a telomere biology disorder, a disorder associated with telomere or telomerase dysfunction, a disorder associated with aging, a disorder associated with short telomeres, a pre-leukemic or pre-cancerous condition, cancer, and a neurodevelopment disorder. In some embodiments, the method further comprises culturing the cell with a feeder layer in a medium. In some embodiments, the cell has at least one stem cell marker selected from the group consisting of FLK-1, AC133, CD34, c-kit, CXCR-4, Oct-4, Rex-1, CD9, CD13, CD29, CD34, CD44, CD166, CD90, CD105, SH-3, SH-4, TRA-1-60, TRA-1-81, SSEA-4, and Sox-2. In some embodiments, the stem cell marker is CD34. In some embodiments, the method further comprising enriching stem cells by isolating CD34+ cells. In some embodiments, the subject is a mammal. In some embodiments, the subject is a human. In some embodiments, the method comprises culturing the cell in a medium selected from the group consisting of Iscove's modified Dulbecco's Media (IMDM) medium, Dulbecco's Modified Eagle Medium (DMEM), Roswell Park Memorial Institute (RPMI) medium, minimum essential medium alpha medium (α-MEM), Basal Media Eagle (BME) medium, Glasgow Minimum Essential Medium (GMEM), Modified Eagle Medium (MEM), Opti-MEM I Reduced Serum medium, neuroplasma medium, CO₂-independent medium, and Leibovitz's L-15 medium. In some embodiments, the cell is a Chimeric Antigen Receptor (CAR) T-Cell. In some embodiments, the cell is a lymphocyte. In some embodiments, the cell is a T cell, an engineered T cell, or a natural killer cell (NK).

### Combination Therapies

In some embodiments, any of the compounds of this disclosure can be co-administered with a second therapeutic, or a pharmaceutically acceptable salt thereof, to treat any of the disorders described herein. The compound of the present disclosure may be administered to the patient simultaneously with the additional therapeutic agent (in the same pharmaceutical composition or dosage form or in different compositions or dosage forms) or consecutively (the additional therapeutic agent may be administered in a separate pharmaceutical composition or dosage form before or after administration of the compound of the present disclosure).

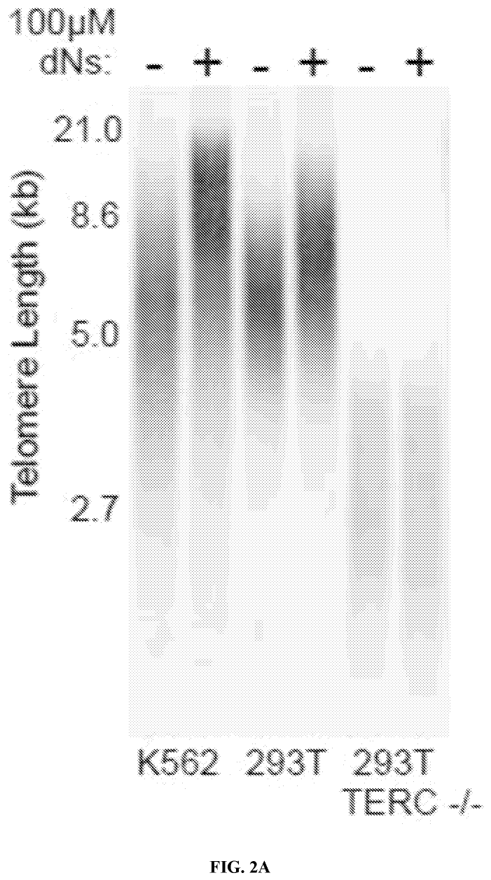

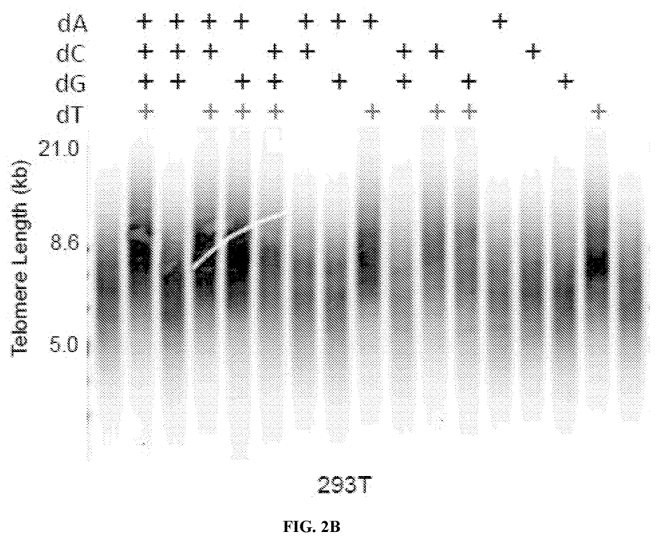

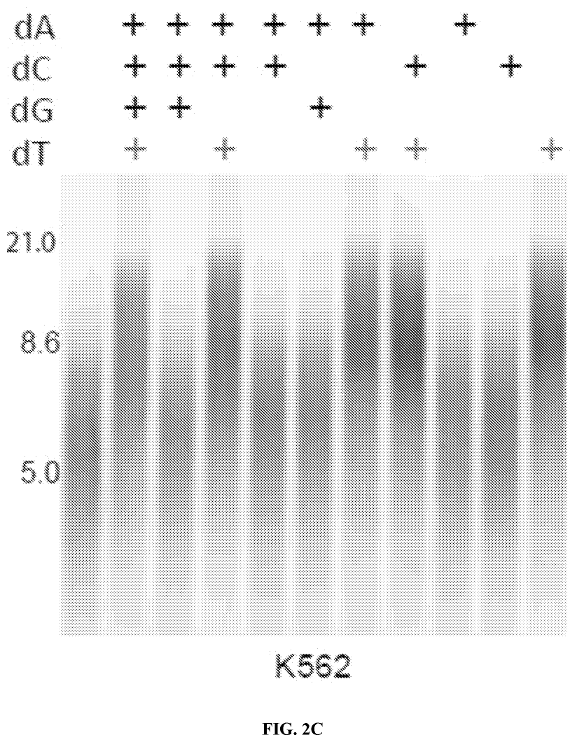

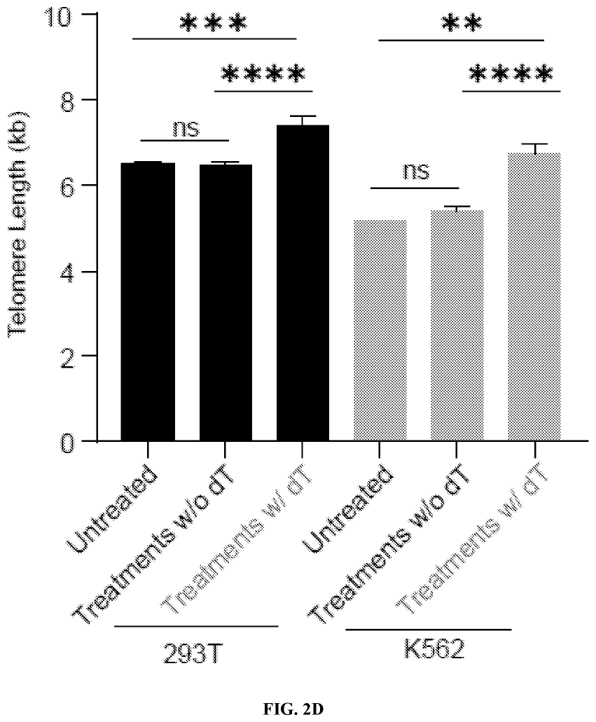

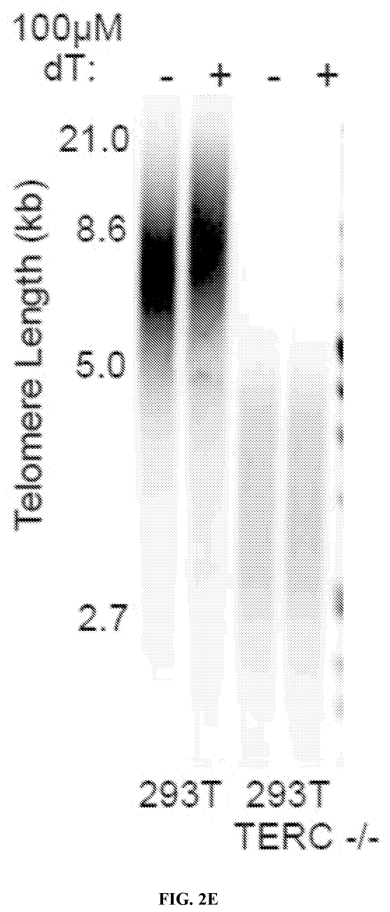

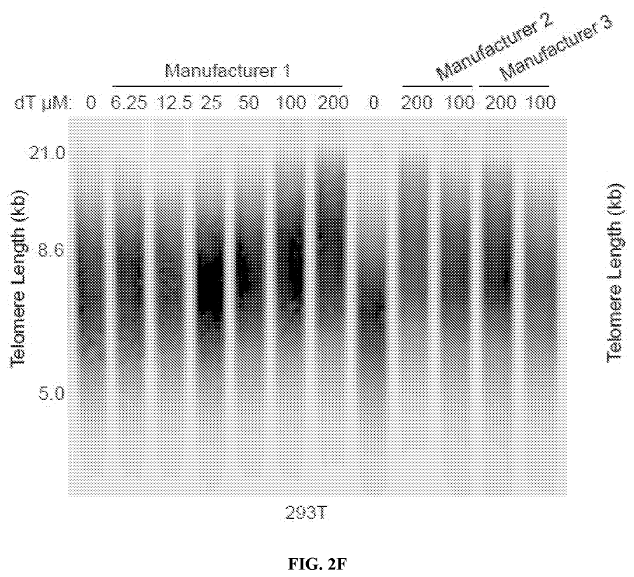

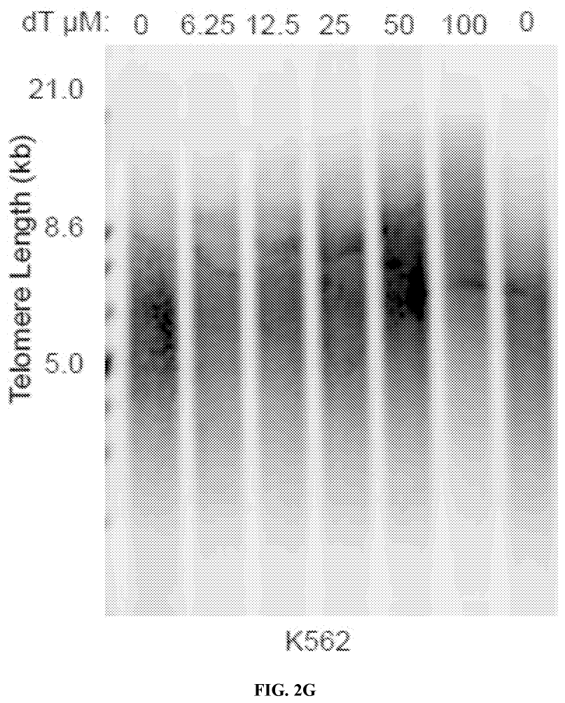

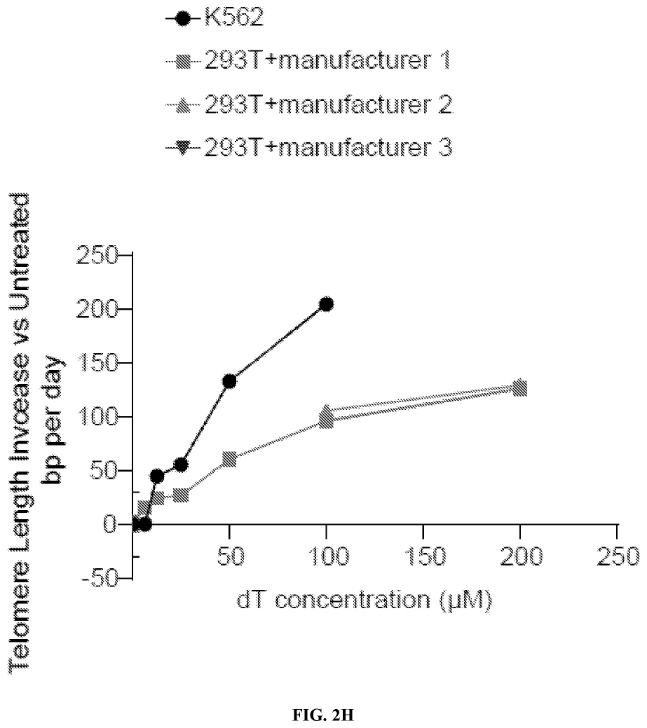

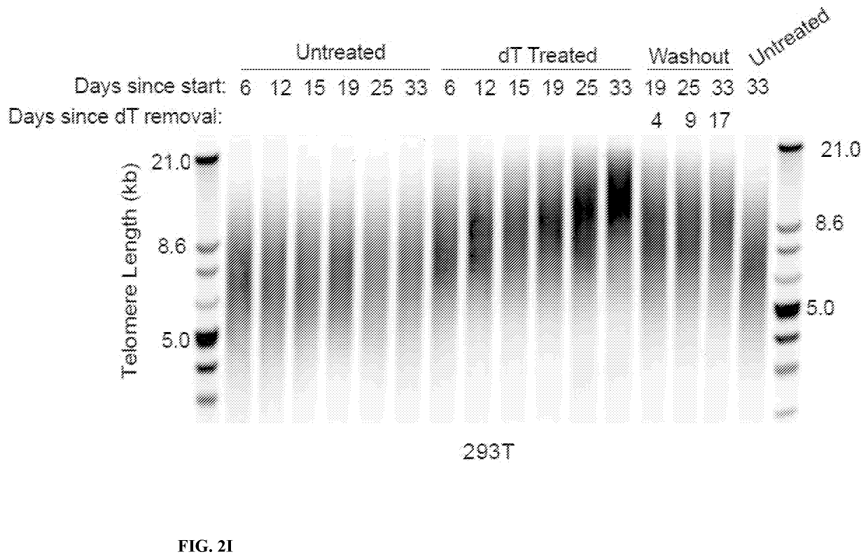

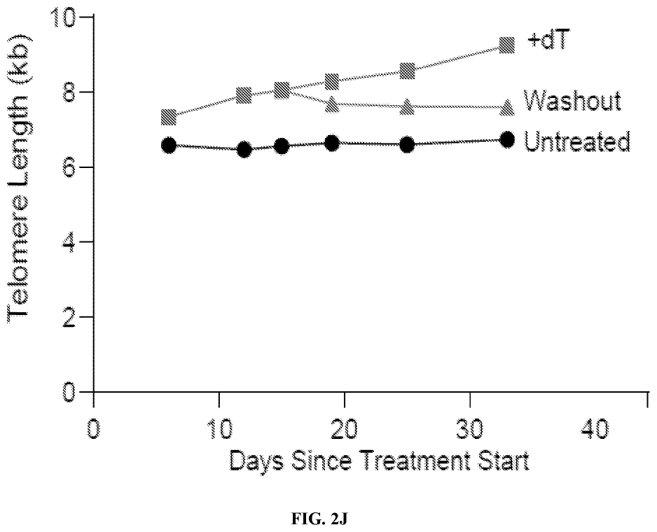

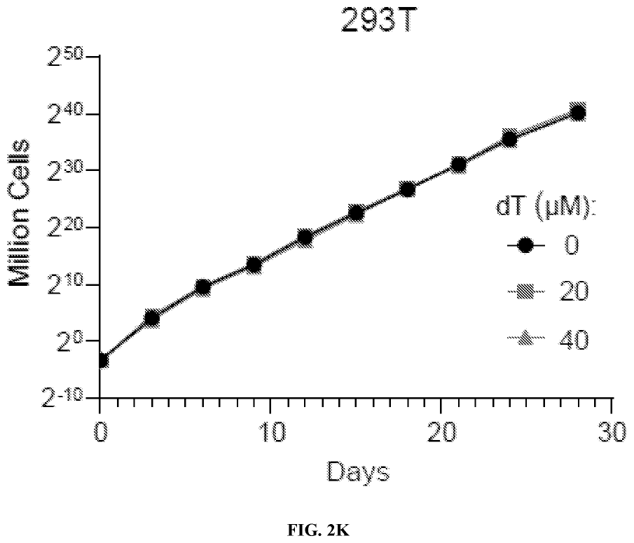

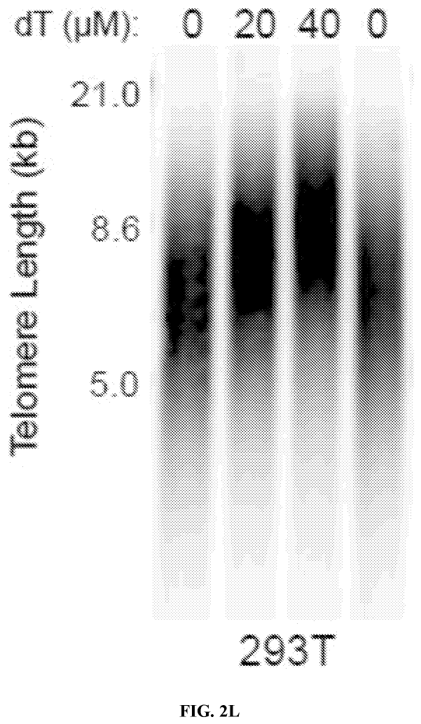

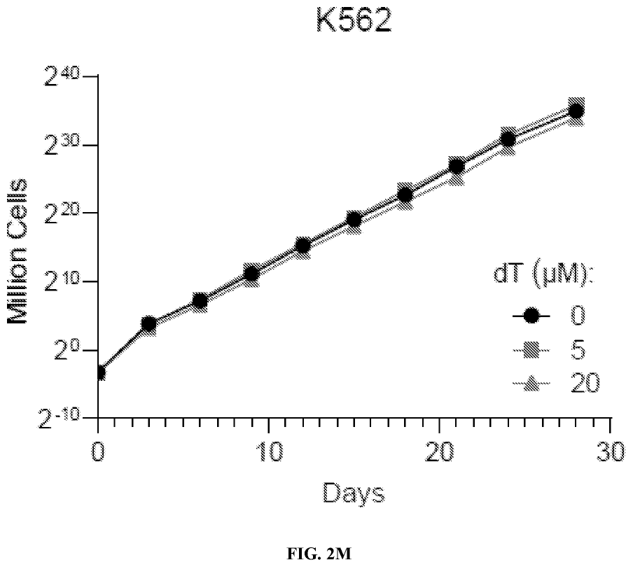

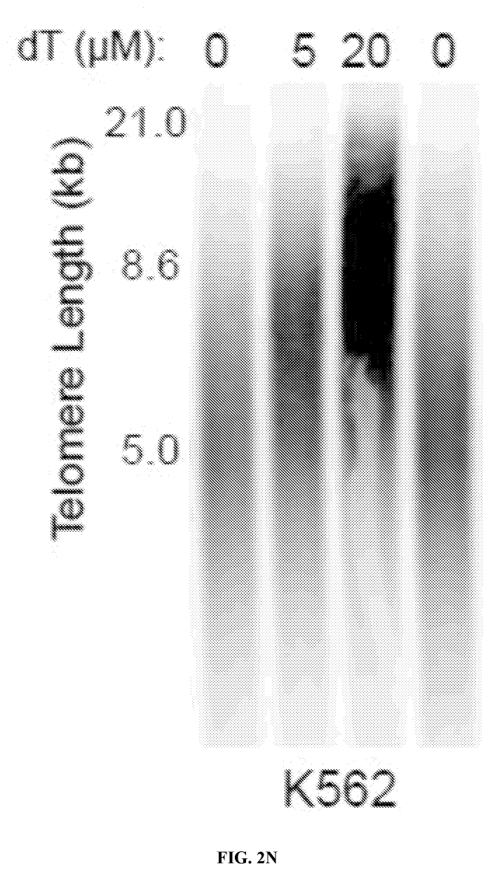

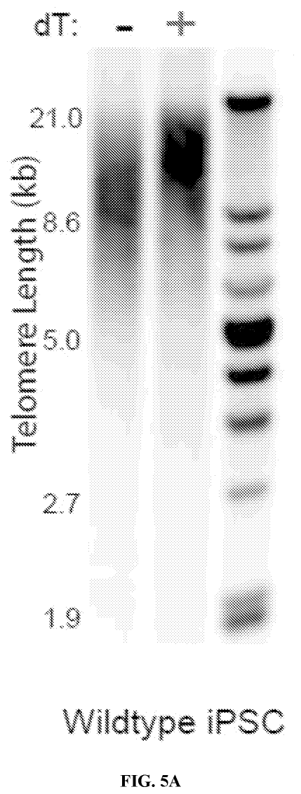

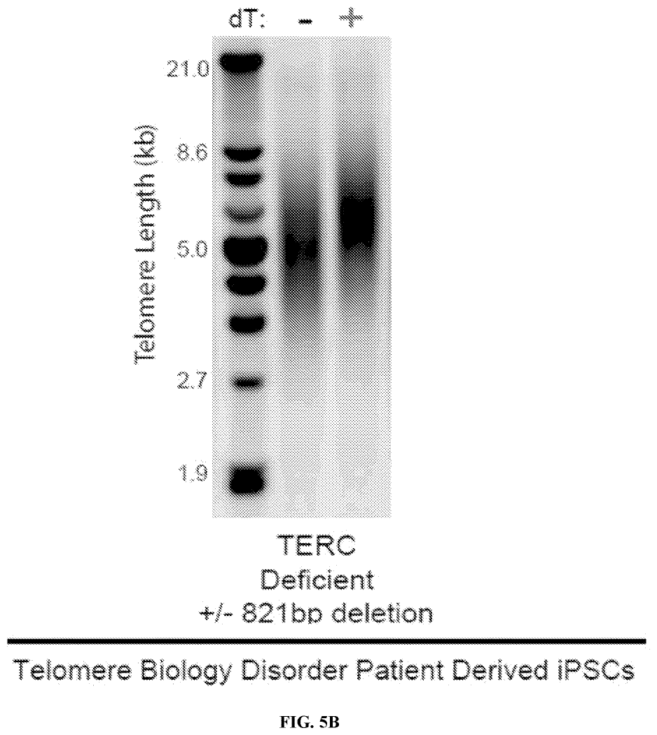

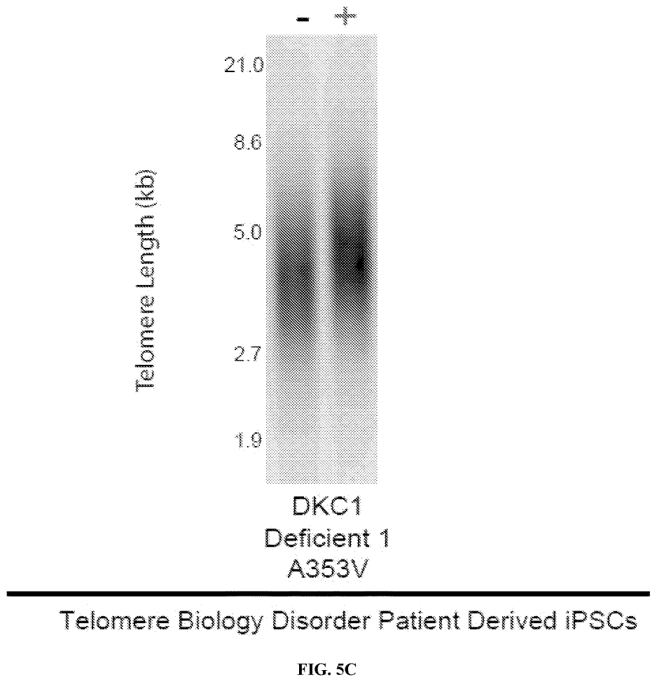

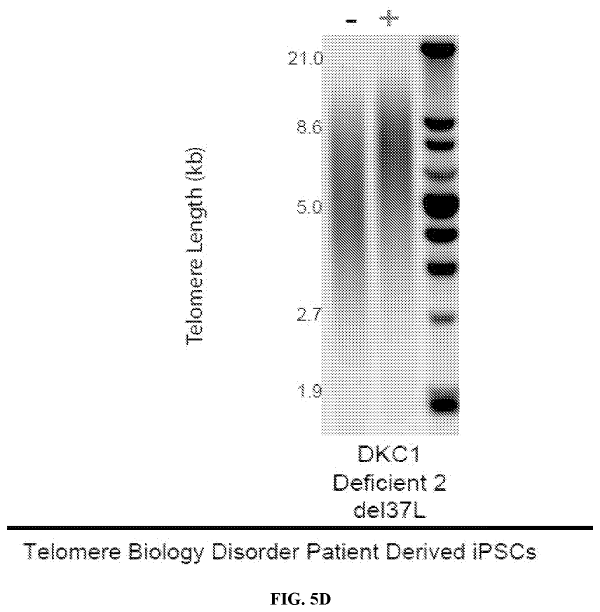

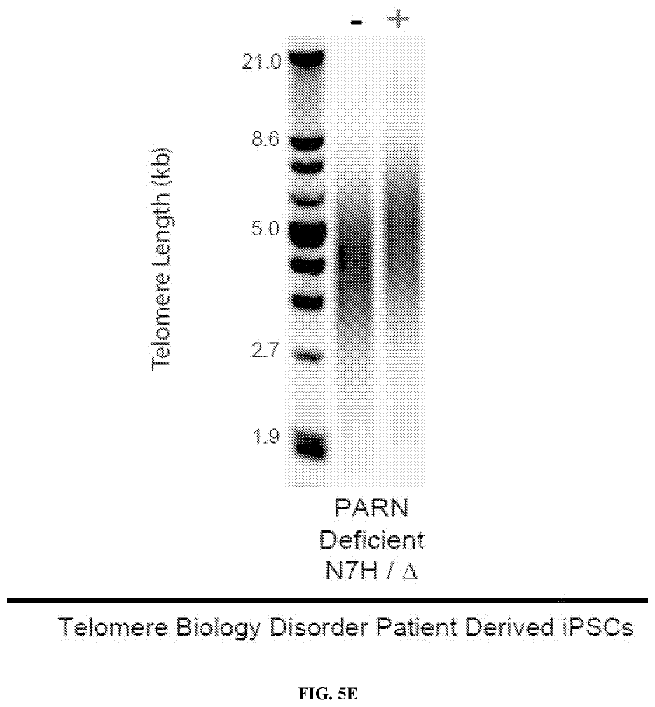

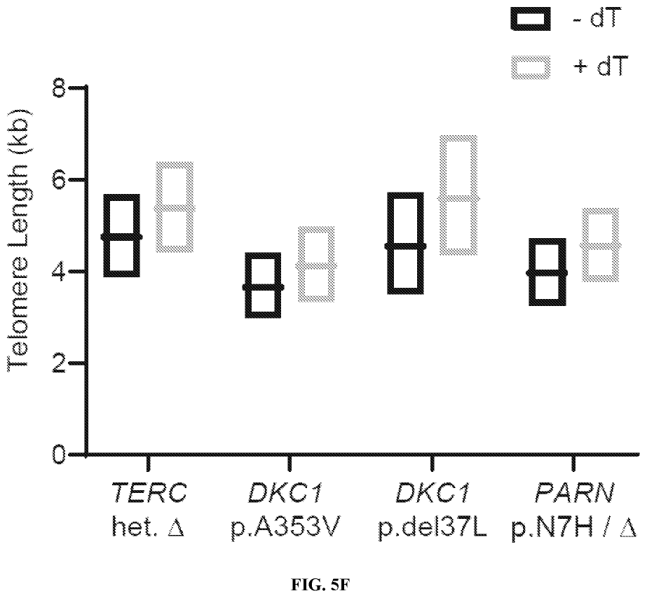

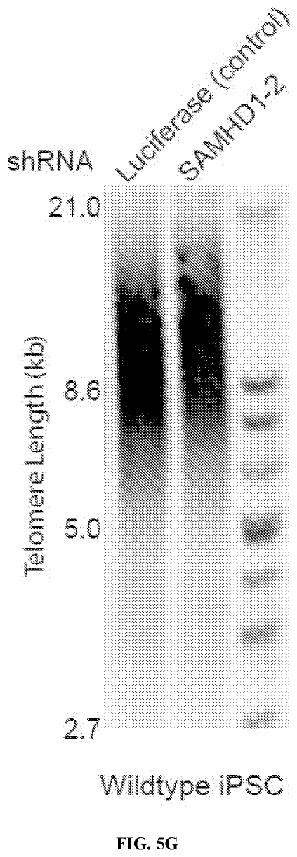

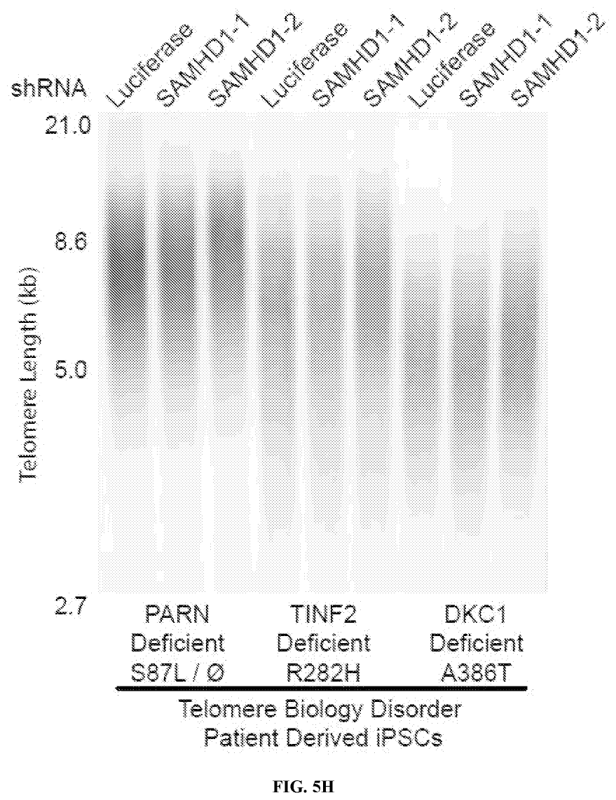

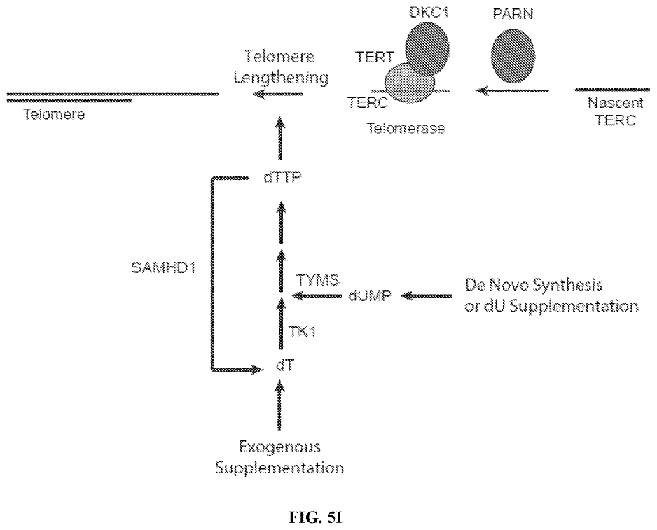

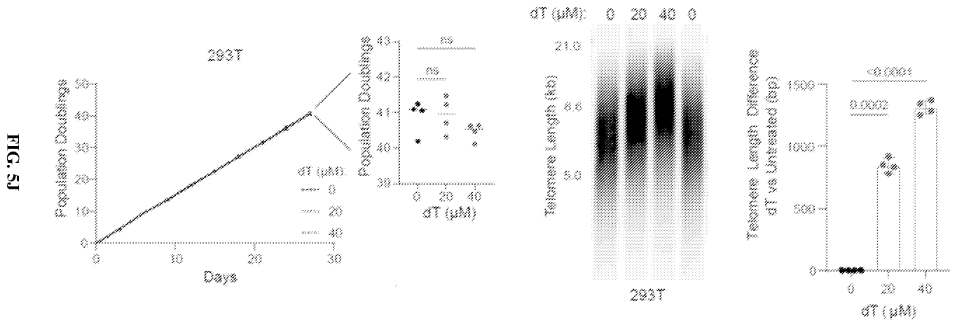

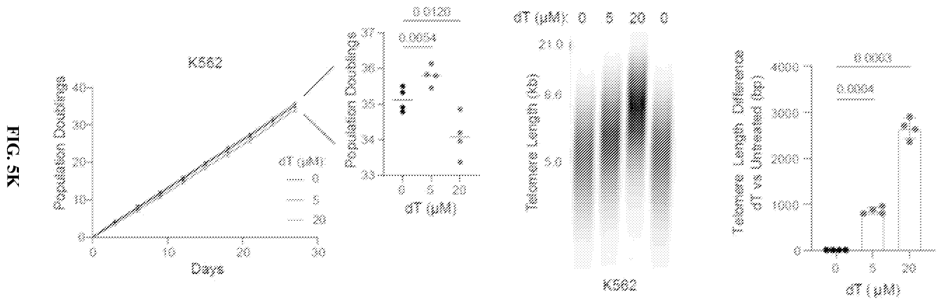

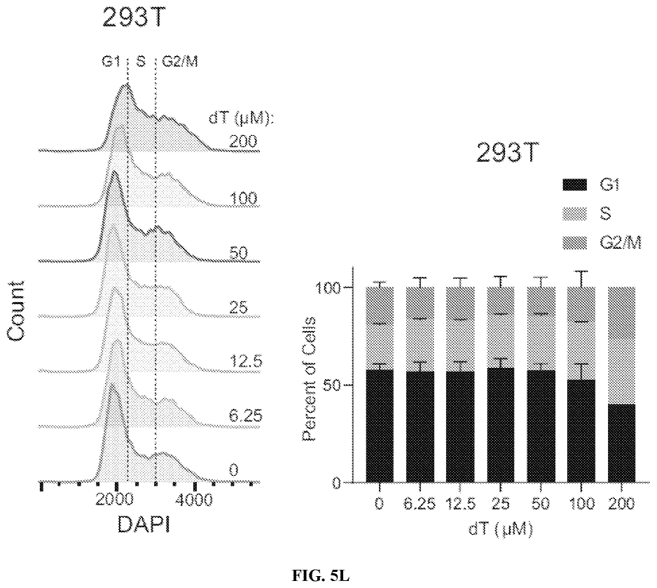

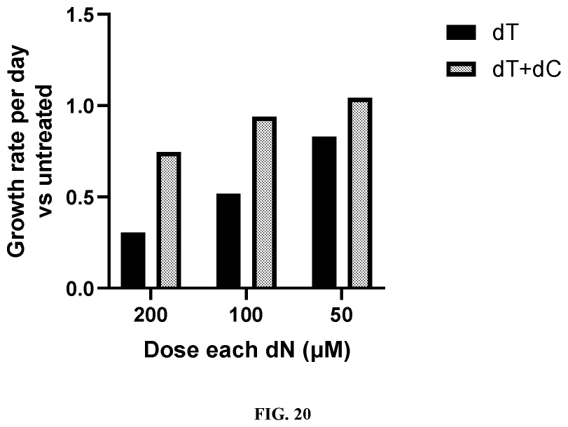

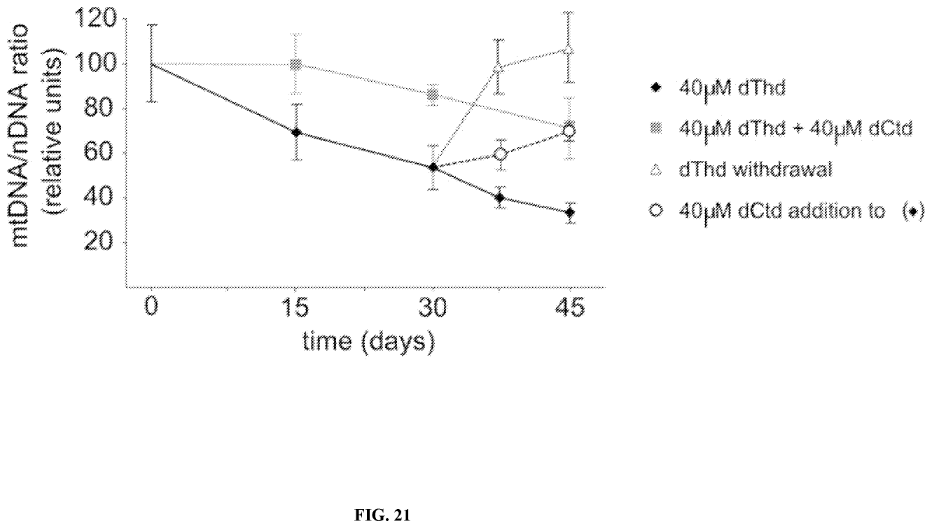

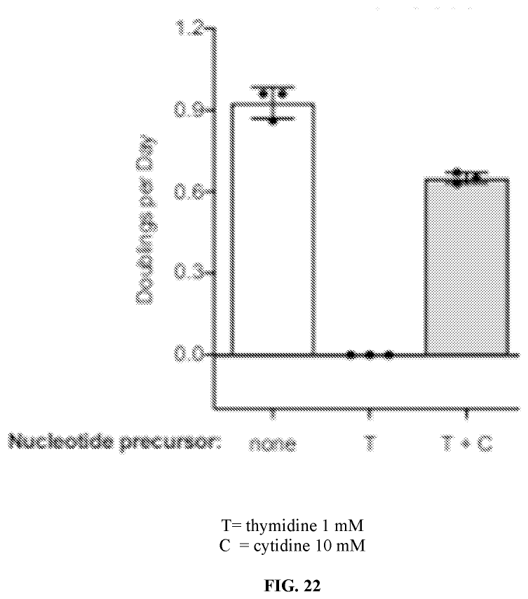

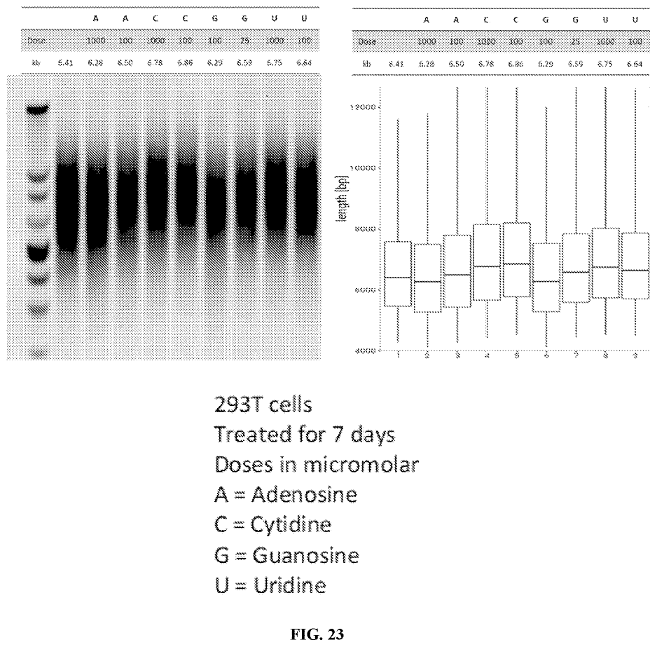

In some embodiments, the present disclosure provides co-administering at least two compounds of Formula (A), or a pharmaceutically acceptable salt thereof. For example, any of the methods of this disclosure may include co-administering dT, or a pharmaceutically acceptable salt thereof, and dC, or a pharmaceutically acceptable salt thereof. While this combination was used previously for treatment of mitochondrial TK2 deficiency (NCT03639701), nothing suggest that the combination could have been useful for treatment of telomere-related conditions as described herein. In some embodiments, co-administering dC with dT therapy advantageously reduces toxicity of dT and improves cell growth compared to dT alone (See DOI 10.1101/2021.12.06.471399, FIG. 2f). In relation to mitochondria-related disorders, combination of dT with dC improves mitochondria copy number compared to dT alone (See PLOS Genet, 2011 Mar. 7 (3), e1002035, FIG. 5). Experimental data of this disclosure shows telomere elongation from dT in combination with dC (See FIGS. 2a-d and Example 1 herein). The combination of dT and dC advantageously improved cellular growth compared to dT alone (FIG. 20).

Without being bound by any particular theory, it is believed that at a significant concentration, dT has been shown to inhibit cell growth. The proposed mechanism for growth inhibition is from increased dTTP levels allosterically inhibiting ribonucleotide reductase from producing dC nucleotide. This reduces dCTP levels and prevents genome replication. Experimental evidence indicates that co-administration of deoxycytidine (dC) (and cytidine, C), alleviates growth inhibition from dT treatment. dT can promote telomere lengthening in the presence of dC (as well as dA and dG), thus the combination of dT with dC (or C, or another nucleoside), enables telomere elongation with fewer side effects on cell growth than dT alone.

In some embodiments, any of the methods of this disclosure may include co-administering dT, or a pharmaceutically acceptable salt thereof, and dU, or a pharmaceutically acceptable salt thereof.

In some embodiments, any of the methods of this disclosure may include co-administering dT, or a pharmaceutically acceptable salt thereof, and U, or a pharmaceutically acceptable salt thereof.

In some embodiments, any of the methods of this disclosure may include co-administering dT, or a pharmaceutically acceptable salt thereof, and C, or a pharmaceutically acceptable salt thereof.

In some embodiments, any of the methods of this disclosure may include co-administering dU, or a pharmaceutically acceptable salt thereof, and dC, or a pharmaceutically acceptable salt thereof.

In some embodiments, any of the methods of this disclosure may include co-administering dU, or a pharmaceutically acceptable salt thereof, and C, or a pharmaceutically acceptable salt thereof.

In some embodiments, any of the methods of this disclosure may include co-administering dU, or a pharmaceutically acceptable salt thereof, and U, or a pharmaceutically acceptable salt thereof.

In some embodiments, any of the methods of this disclosure may include co-administering dC, or a pharmaceutically acceptable salt thereof, and C, or a pharmaceutically acceptable salt thereof.

In some embodiments, any of the methods of this disclosure may include co-administering dC, or a pharmaceutically acceptable salt thereof, and U, or a pharmaceutically acceptable salt thereof.

In some embodiments, any of the methods of this disclosure may include co-administering U, or a pharmaceutically acceptable salt thereof, and C, or a pharmaceutically acceptable salt thereof.

In some embodiments, the methods of this disclosure include co-administering a compound of Formula (A), or a pharmaceutically acceptable salt thereof, and a purine nucleoside, or a pharmaceutically acceptable salt thereof. In some embodiments, the purine nucleoside is selected from adenosine (A), deoxyadenosine (dA), guanosine (G), and deoxyguanosine (dG), or a pharmaceutically acceptable salt thereof.

In some embodiments, the second therapeutic agent is a PAPD5 inhibitor, or a pharmaceutically acceptable salt thereof. In some embodiments, the PAPD5 inhibitor is a biomolecule (having a molecular weight of 200 daltons or more produced by living organisms or cells) or a small-molecule drug (typically about 2000 daltons or less). In some embodiments, the PAPD5 inhibitor is a protein (e.g., antibody) or a nucleic acid (e.g., siRNAs, shRNAs, or gRNA). In some embodiments, PAPD5 inhibitor is a small-molecule. In some embodiments, PAPD5 inhibitor is of the dihydroquinolizinone class of molecules. In some embodiments, PAPD5 inhibitor is the dihydroquinolizinone RG7834, (6S)-6-Isopropyl-10-methoxy-9-(3-methoxypropoxy)-2-oxo-6,7-dihydrobenzo[a]quinolizine-3-carboxylic acid, with CAS number 2072057-17-9 (S-isomer). In some embodiments the PAPD5 inhibitor is a quinoline derivative. Examples of PAPD5 inhibitors are disclosed in US20210177827, US20210330678, 63/273,871, and WO2020219729, which are incorporated herein by reference.

In some embodiments, the second therapeutic agent is an inhibitor of dNTPase SAM domain and HD domain-containing protein 1 (SAMHD1), or a pharmaceutically acceptable salt thereof. The SAMHD1 inhibitor may be a biomolecule (having a molecular weight of 200 daltons or more produced by living organisms or cells) or a small-molecule drug (typically about 2000 daltons or less). In some embodiments, the SAMHD1 inhibitor is a protein or a nucleic acid, such as siRNAs, shRNAs, or gRNA. In some embodiments, the SAMHD1 inhibitor is an antibody (e.g., monoclonal or polyclonal antibody).

The experimental data presented in this disclosure shows that the SAMHD1 protein degrades dNTPs to dNs, and inhibition of SAMHD1 increases telomere lengthening from dT treatment (see FIG. 5e). Without being bound by any particular theory, it is believed that SAMHD1 is the target of the lentiviral accessory protein family VPX and VPR. These peptides which are encoded by lentiviruses (in the same family as HIV) co-opt the cellular protein degradation machinery to deplete SAMHD1. The proposed reasoning for this is that inhibition of SAMHD 1 increases the dNTPs available for viral replication via reverse transcription. These peptides are typically delivered within a lentiviral particle (or virus like particles) or encoded in a lentiviral vector.

In some embodiments, the SAMHD1 inhibitor is selected from a nucleic acid miRNA181a/b, an anti-SAMHD1 antibody, a T cell receptor, VPX, VPR, VPX encoded by HIV2, SIVSM, SIVrcm, SIVmac, or SIVMAC, and VPR encoded by SIVmus or SIV deb. In some embodiments, the SAMHD1 inhibitor is a peptide having amino acid sequence having at least 75%, 80%, 85%, 90%, or 95% identity to VPX. In some embodiments, the SAMHD1 inhibitor is a peptide having amino acid sequence having at least 75%, 80%, 85%, 90%, or 95% identity to VPR. The peptide can be delivered in any suitable form described herein, for example, the peptide may be delivered via a lentivirus. In some embodiments, VPX is fused to VPR to promote packaging into lentiviral virions. For example, the VPX or VPR protein can be included in lentiviral particle or included on the lentiviral genome and expressed in the target cell. In some embodiments, VPX can be fused with a cell-penetrating peptide (e.g., TAT or CPP44) to promote SAMHD1 degradation in the target cell without having to incorporate VPX into a viral particle. An example of such a fused VPX peptide is described in Nair et al. (See DOI 10.1136/jitc-2021-ITOC8.8). Other suitable examples of SAMHD1 inhibitors are disclosed, for example, in US20180313843 and CN104583231, which are incorporated herein by reference in their entirety. In some embodiments, the SAMHD1 inhibitor is a small-molecule. Suitable examples of SAMHD1 inhibitors include those described, for example, in Mauney et al. (Biochemistry, 201, 27, 57, 47, 6624-6636) and Seamon et al., Journal of Biomolecular Screening, 2015, 20, 6, 801-809, which are incorporated herein by reference in their entirety.

Suitable examples of SAMHD1 inhibitors include Erythrotyrosine, Sennoside A, Evans Blue, Merbromin, Phenylmercuric Acetate, Thiram, Bronopol, Cephalosporin C, Pidolic Acid, Diphenhydramine, Aurothiomalate, Rose Bengal, Chlorambucil, Pyrithione Zinc, Lomofungin, Troglitazone, Montelukast, Pranlukast, L-thyroxine, Ergotamine, Amrinone, Retinoic Acid, Ethacrynic Acid, Hexestrol, Tolfenamic acid, Bexarotene, Sulindac, Zolmitriptan, Nifedipine, Tetracycline, Nisoldipine, Medroxyprogesterone acetate, Trifluoperazine, Primaquine, Adapalene, Aprepitant, Tolcapone, Zafirlukast, Delavirdine, Topotecan, Ceftazidime, Zoledronic acid, Anethole-trithione, and Disulfiram, or a pharmaceutically acceptable salt thereof.

In some embodiments, the second therapeutic agent is an inhibitor of thymidine phosphorylase (TYMP). Without being bound by any particular theory or speculation, it is believed that TYMP degrades dT to thymine. Inherited mutations in TYMP cause elevated blood levels of dT, providing credible evidence that inhibition of TYMP enhances dT treatment. TYMP is known to metabolize and degrade chemotherapeutic trifluridine. The TYMP inhibitor tipiracil is approved for the treatment of colorectal cancer in combination with trifluridine, with the rationale that inhibition of TYMP tipiracil will increase the efficacy of trifluridine. In some embodiments, the second therapeutic is tipiracil, or a pharmaceutically acceptable salt thereof. In some embodiments, the second therapeutic is a TYMP inhibitor selected from 6-aminothymine (6AT), amino-5-chlorouracil (6A5CU), 6-amino-5-bromouracil (6A5BU), 7-deazaxanthine (7DX), 7-(2-aminoethyl)-deazaxanthine, 6-(2-aminoethylamino)-5-chlorouracil (AEAC), 5-chloro-6-(1-imidazolylmethyl)uracil (CIMU), 6-methylenepyridinium, 6-(4-phenylbutylamino)uracil, 5-chloro-6-[1-(2-iminopyrrolidinyl)methyl]uracil hydrochloride (TPI), and N-(2,4-dioxo-1,2,3,4-tetrahydro-thieno[3,2-d]pyrimidin-7-yl)guanidine, or a pharmaceutically acceptable salt thereof. Additional examples of TYMP inhibitors include those described in Perez et al., Mini-Reviews in Medicinal Chemistry, 2005, 5, 1113-1123, and WO2020232263A1, which are incorporated herein by reference in their entirety.

In some embodiments (e.g., in case of genetic TBDs described herein), the compound of Formula (A), or a pharmaceutically acceptable salt thereof, can be co-administered with nucleic acids, such as the normal, functional versions of the coding sequences of the genes that are affected by such genetic TBDs, for example TERT, PARN, RTEL1, DKC1, TINF2, NOP10, NHP2, TERC, or other genes. Any of the genes described herein as affected by the TBD can be used for co-administering to the patient with a TBD. Additional examples are described in Armanios, 2009, Ann. Rev. Genomics Hum. Genet., 10, 45-61, which is incorporated herein by reference in its entirety.

### Compositions, Formulations, and Routes of Administration

The present application also provides pharmaceutical compositions comprising an effective amount of a compound of the present disclosure (e.g., thymidine), or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

### Dosages and Regimens

In the pharmaceutical compositions of the present application, a compound of the present disclosure (e.g., thymidine) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound (e.g., thymidine) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) (e.g., thymidine) is about 0.1 mg/kg, about 0.5 mg/kg, about 1 mg/kg, about 2 mg/kg, or about 5 mg/kg. The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Definitions

As used herein, the term “about” means “approximately” (e.g., plus or minus approximately 10% of the indicated value)

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the telomerase with a compound of the invention includes the administration of a compound of the present invention to an individual or patient, such as a human, having telomerase, as well as, for example, introducing a compound of the invention into a sample containing a cellular or purified preparation containing the telomerase.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### Example 1

### Materials and Methods

Cell culture: K562 cells (ATCC) were grown in RPMI1 1640 (Gibco) media supplemented with 10% fetal bovine serum. 293T cells (ATCC) were grown in DMEM (Gibco) supplemented with 10% fetal bovine serum, and were subcultured using 0.05% trypsin (Gibco). iPSCs were derived, characterized and cultured as previously described. iPSCs were grown in Essential 8 Media (Life Technologies) supplemented with the ROCK inhibitor Y271632 (Stem Cell Technologies) at 10 μM on plates coated with hES qualitied Matrigel matrix (Corning) and were subcultured using Accutase (Stem Cell Technologies).

Lentiviral transduction of shRNA and overexpression constructs was performed in media supplemented with protamine sulfate (Sigma Aldrich) at 10 μg/ml for 16 hours. Puromycin (Sigma Aldrich) selection of transduced cells occurred at 2 μg/ml for 3-5 days, blasticidin (InvivoGen) selection of transduced cells occurred at 10 μg/ml for 5-10 days. SAMHD1 overexpression experiments occurred in media containing 1 μg/ml doxycycline (Sigma Aldrich) to induce transgene expression.

Quantification of cell growth was performed using hemocytometry with trypan blue staining to identify dead cells. For growth curves, 50k cells were plated per 12 well or 100k cells were plated per 6 well and cells were passaged when confluent, counted using a hemocytometer, and replated at equal numbers in fresh media.

Lentivirus Production: 293T cells were transfected with psPAX2 and pMD2.G as well as the appropriate transfer vector using calcium phosphate as previously described. Virus containing media was harvested 48 and 72 hours after transfection. shRNA and overexpression construct virus containing media was filtered with 0.45 μM filters (VWR) and stored at −80 C. For gRNA libraries, virus containing media was 0.45 μM filtered and concentrated by ultracentrifugation followed by resuspension in DMEM and storage at −80 C.

Cas9 Expressing Cell line generation and validation: K562 cells (ATCC) were transduced with lentivirus containing the Lenti-Cas9-2A-Blast construct followed by selection in 10 μg/ml blasticidin for 10 days. After selection, cells were transduced with the PXPR011 vector and cultured for two weeks followed by flow cytometry to quantify the percentage of GFP positive cells.

gRNA library design and production: for the secondary screening nucleotide metabolism library, genes were selected for inclusion based on annotated involvement nucleotide salvage and deoxyribonucleotide metabolism by Gene Ontology. 10 gRNAs per gene were designed using the Broad GPP sgRNA design tool. 200 non-targeting gRNAs sequences were selected from the Brunello gRNA library. A pool of ssDNA encoding the gRNAs flanked by BsmBI recognition sites and overhang sequences for PCR amplification (as described previously) was synthesized by Twist Bioscience. The library was PCR amplified using Q5 High Fidelity Taq Polymerase (NEB) and cloned into lentiGuide-Puro (Invitrogen) using golden gate cloning with BsmBI-v2 (NEB) and T4 DNA Ligase (NEB) in T4 DNA Ligase buffer followed by transformation into chemically competent Stb13 cells (Invitrogen) which were prepared using the Zymo Mix and Go! E. coli Transformation Kit. Sufficient transformation reactions were performed to attain >40 colonies per gRNA. Library representation was established by PCR amplification using Titanium Taq Polymerase (Takara Bio) followed by next generation sequencing and library quantification using the Mageck RRA software. Plasmid library Gini Indexes were <0.07. Gene specific and non-targeting gRNA libraries were cloned separately and pooled in appropriate quantities to maximize gRNA representation prior to lentivirus production.

Flow-FISH telomere length CRISPR Screening: Cas9 expressing K562 cells were plated with 3M cells per well in a twelve well dish with sufficient wells for >3000× fold library coverage. Cells were spinfected at 931G for 2 hrs at 30° C. in 1 ml of media per well media containing 10 μg/ml protamine sulfate (Sigma Aldrich). After the spin, 3 ml of fresh media was added and cells were incubated overnight. Virus was removed on day 1. Puromycin selection at 2 μg/ml was performed from days 2-6. Sufficient virus was used to ensure between 20% and 50% of cells were infected. After puromycin selection, cells were cultured at >1000× library coverage for the amount of time indicated in the figure legend. Flow-FISH was performed using an Alexa-647 conjugated telC-PNA probe (PNA bio) as described. Cells were counterstained with DAPI and gated on DAPI low cells and then based on Alexa 647 fluorescence as described in the figure legend. DNA from sorted cells was purified by phenol chloroform extraction using standard procedures. Next, gRNA target site was PCR amplified using Titanium Taq Polymerase using primers containing illumina P5 and P7 binding sites. PCR products were purified using a 1:1 ratio of sample purification SPR1 magnetic beads (Beckman Coulter) and sequenced on an illumina Next seq using 75 bp High Output chemistry.

RNA isolation and RT-qPCR: RNA was isolated from cells using the Trizol Reagent (Invitrogen). RNA was DNAse treated with the TURBO DNA-free™ Kit (Invitrogen). cDNA was synthesized using the SuperScript™ III Reverse Transcriptase (Invitrogen). qPCR was performed using SsoAdvanced Universal SYBR Green Supermix (Biorad) in biological and technical triplicate. TERC expression relative to GAPDH was quantified using delta-delta-Ct methodology.

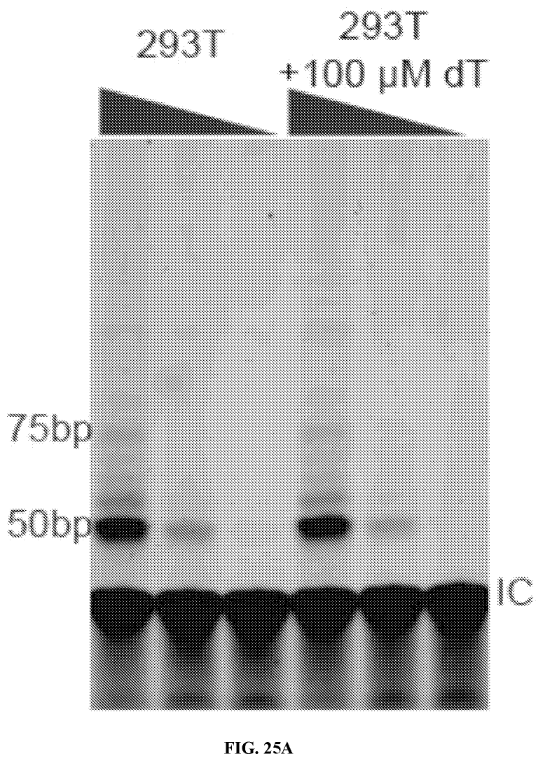

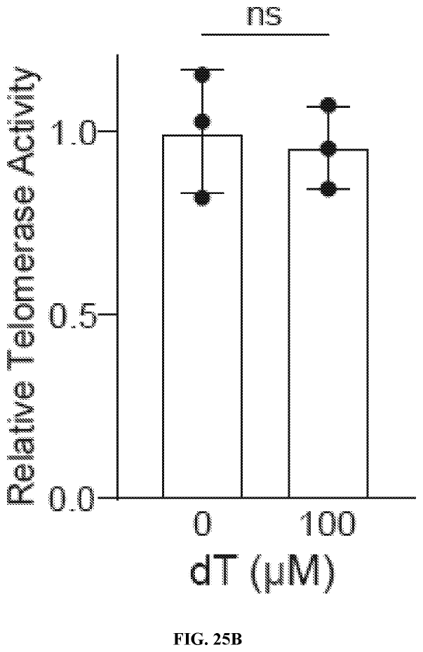

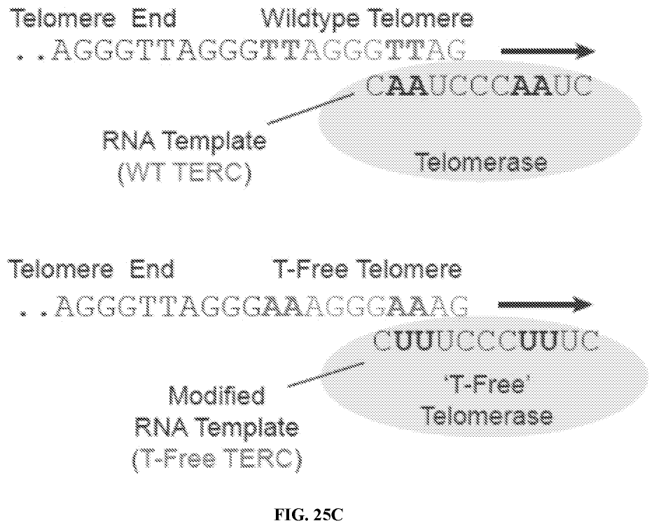

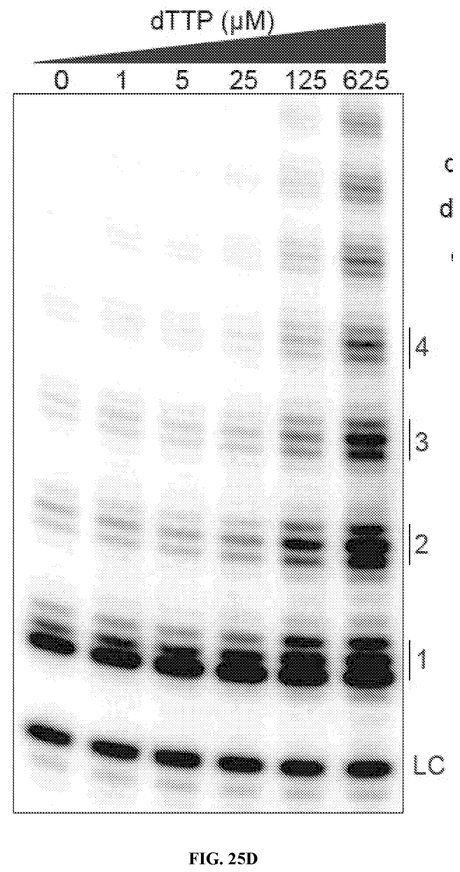

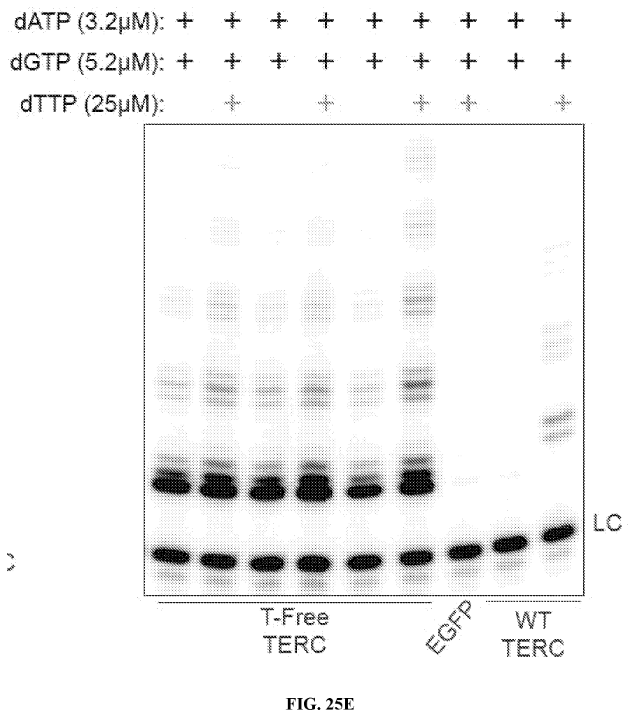

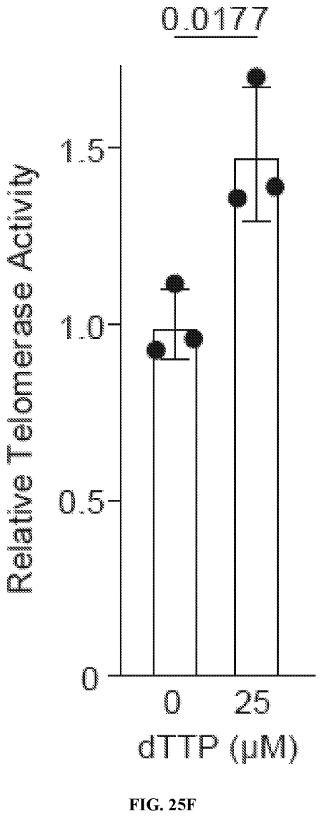

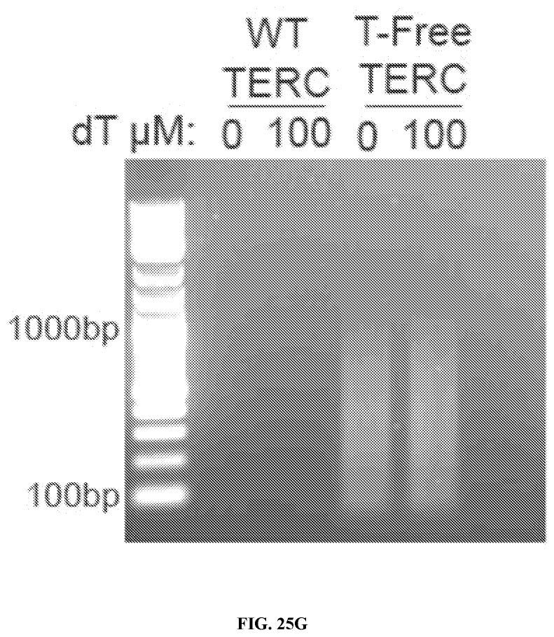

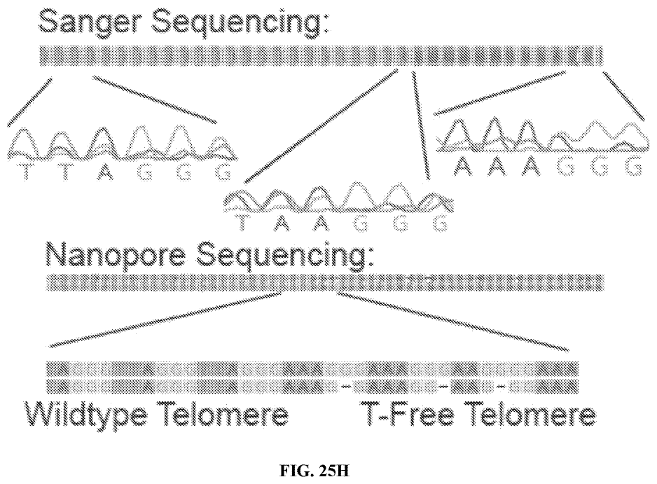

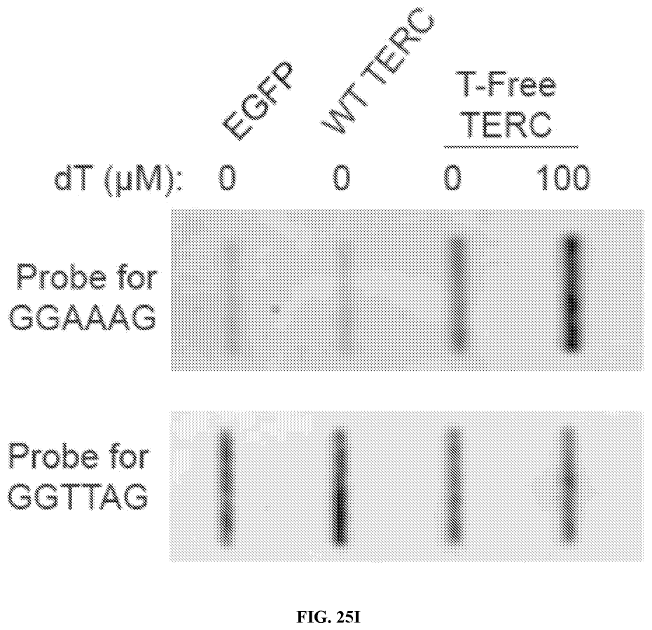

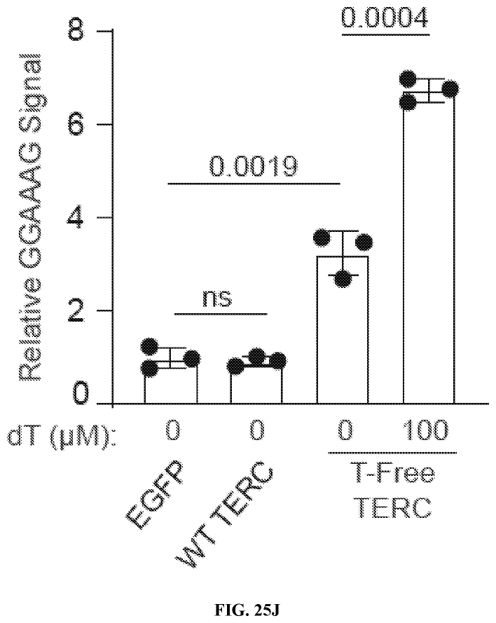

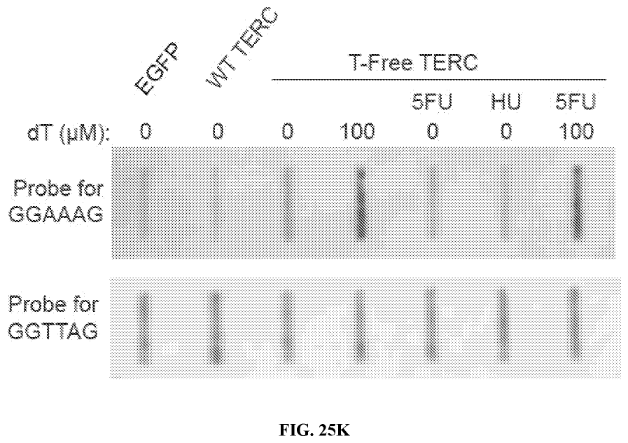

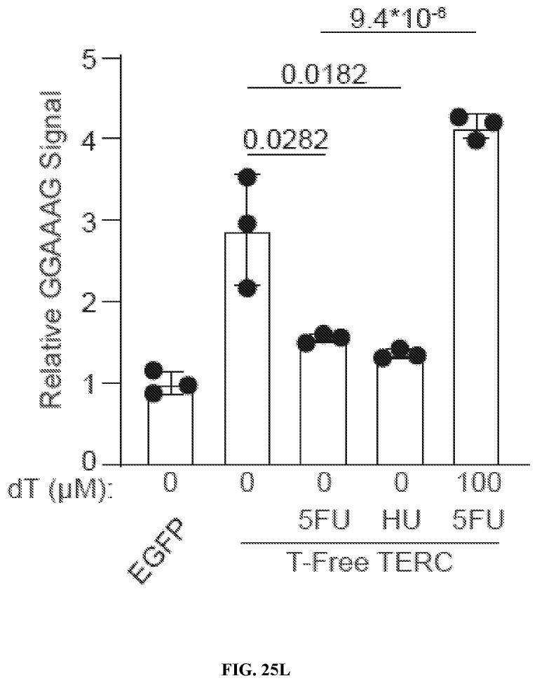

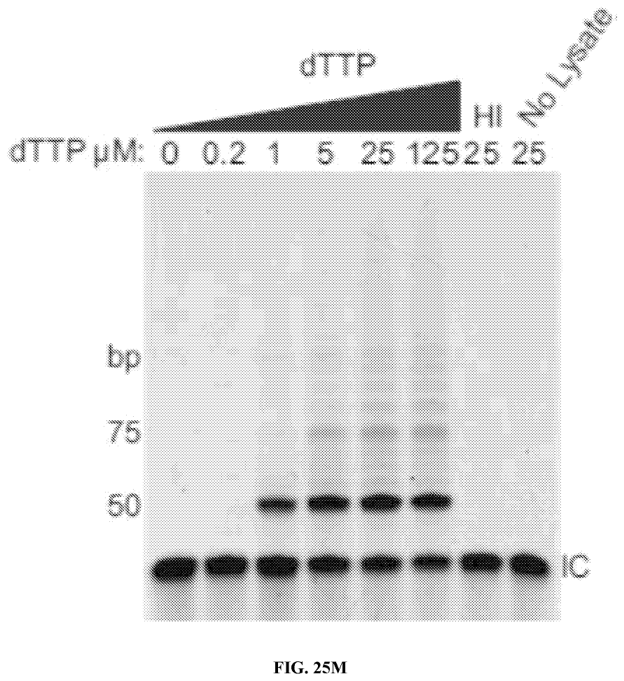

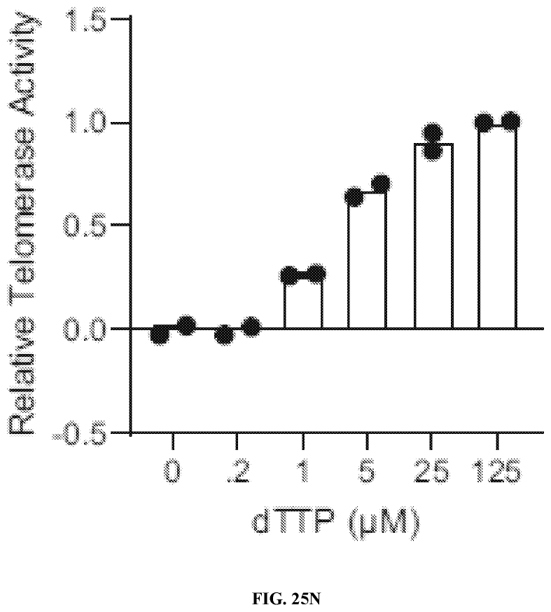

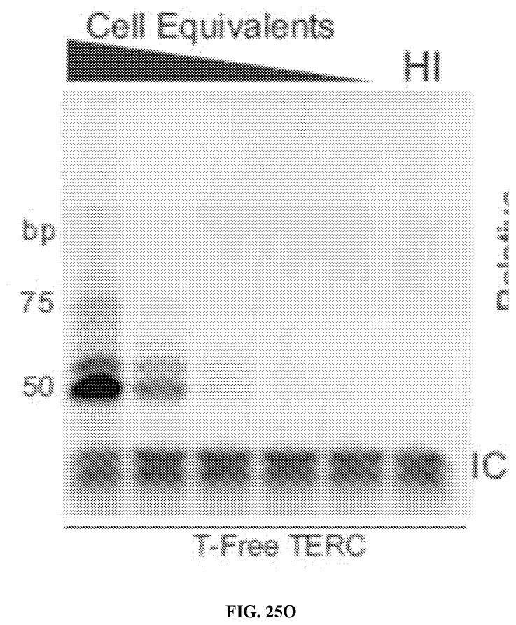

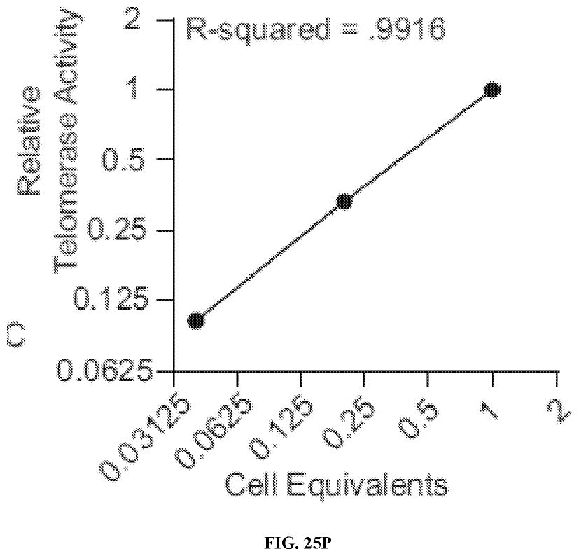

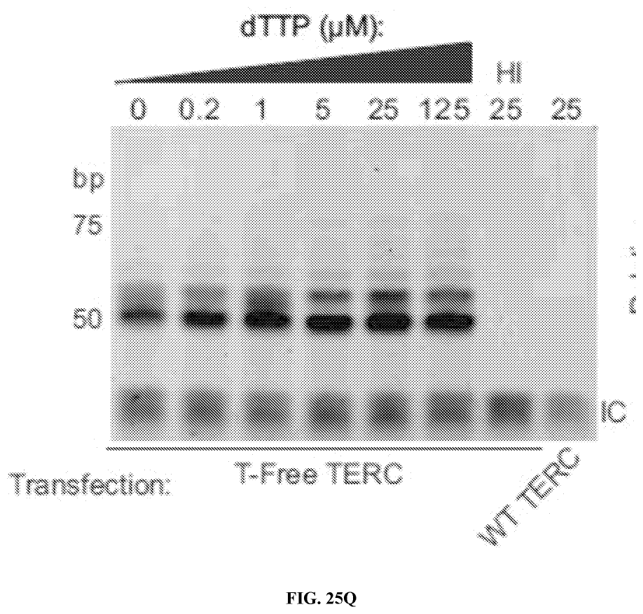

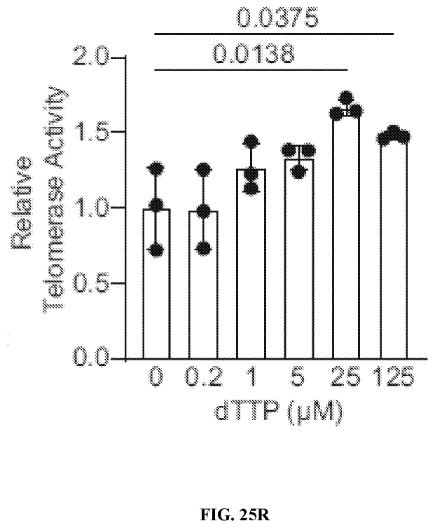

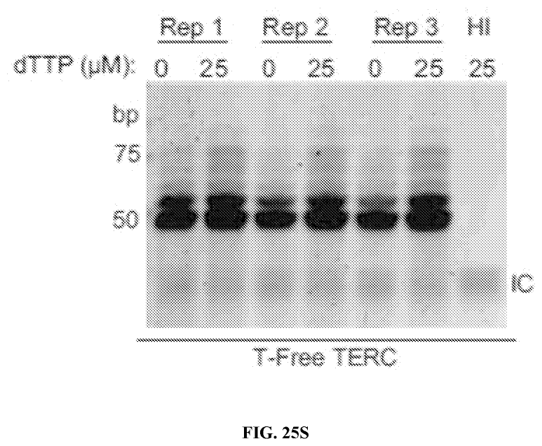

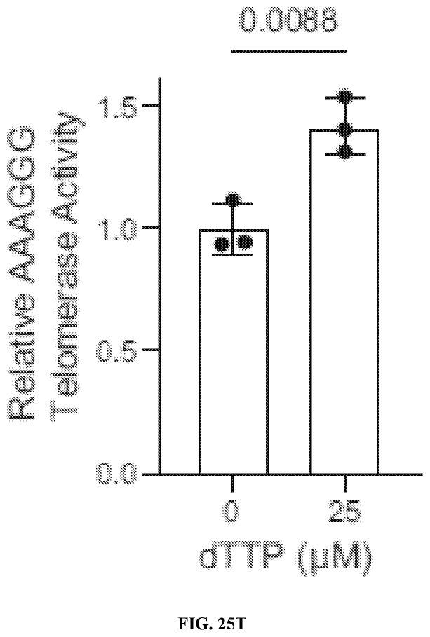

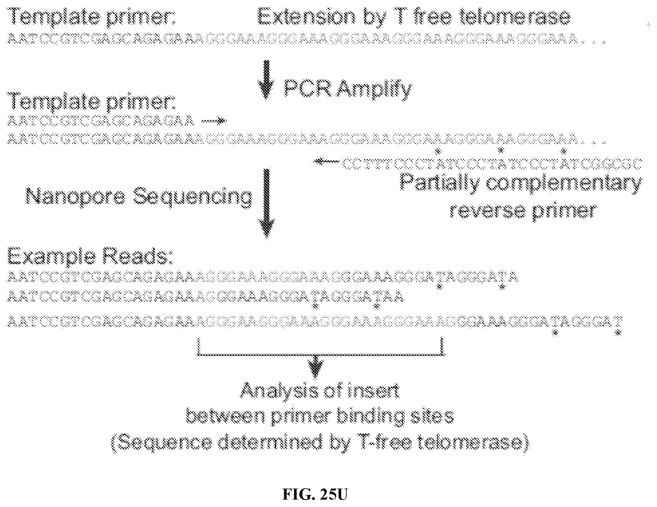

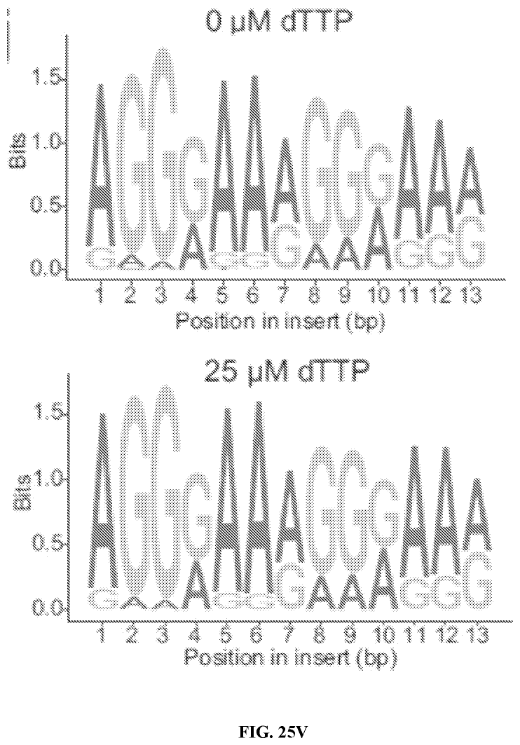

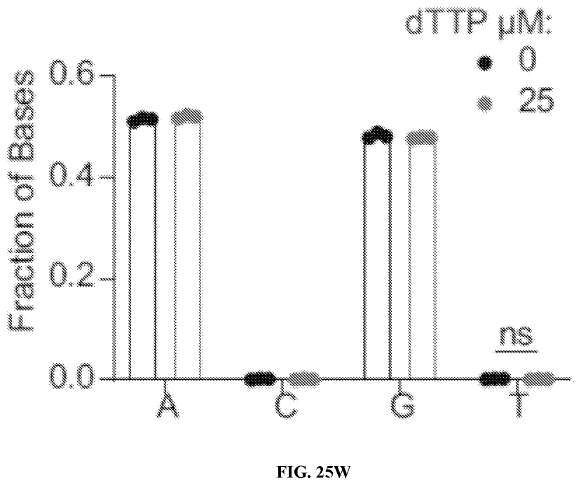

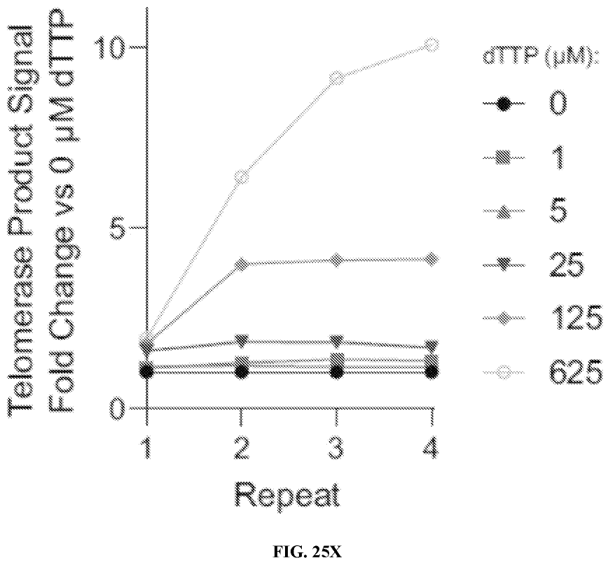

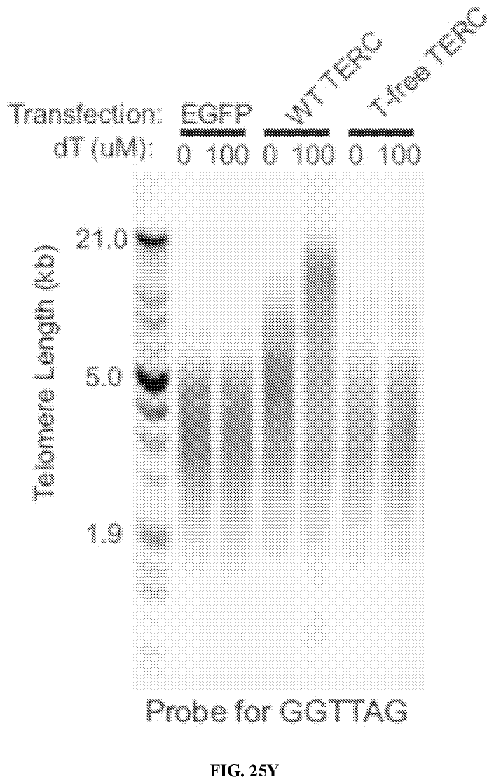

TRAP assay: cell extracts for TRAP assays were made using TRAPeze™1×CHAPS Lysis Buffer (Roche) supplemented with RNASEin (Promega). Standard TRAP reactions (in FIG. 25a) were performed as described, with cell input normalized across samples using the Bio-Rad DC assay. TRAP assays were modified to enable unique dNTP concentrations in telomerase reactions from PCR reactions (FIG. 25) as follows: reactions were assembled with 1×TRAP buffer (20 mM Tris-HCl, pH 8.3; 1.5 mM MgCl₂; 63 mM KCl; 0.05% Tween 20; 1 mM EGTA), TS, ACX, TNST, and NT primers as described (see Primers/Oligos below), dNTPs were added at physiologic concentrations (dATP 24 μM, dGTP 5.2 μM, dCTP 29 μM, dTTP 37 μM) unless otherwise mentioned in the figure legend, followed by telomerase extracts. TRAP assays were modified to detect GGAAAG repeats using the following primers: TS GGAAAG, TNST GGAAAG, ACX GGAAAG, and NT (see Primers/Oligos below). After addition of telomerase extracts, reactions were incubated for 30 minutes at 30° C. followed by heat inactivation at 95° C. for 5 minutes. Reactions were purified in order to remove dNTPs using the Oligo Clean and Concentrator Kit (Zymo) and eluted in 15 μl of water. 10 μl of elutate was then used in TRAP PCR reactions along with 1×TRAP buffer, 0.4 U Titanium Taq Polymerase, and 50 μM of each dNTP, and PCR was performed as described. PCR products were resolved on 10% polyacrylamide gels (Bio-Rad), stained with Gel Red (Biotium), and imaged on a Bio-Rad Chemi-Doc Touch Imager. Images were quantified for a given lane using ImageJ by measuring the signal of the telomerase repeat sized products divided by the signal of the internal control band for the lane, normalized to the same ratio for the untreated sample(s) in a given experiment. Statistical analysis performed using Graphpad Prism V9.1.0.

Terminal Restriction Fragment Length Analysis: genomic DNA was isolated from cells using the Invitrogen Purelink Genomic DNA mini kit (Invitrogen). 1-3 μg of gDNA was digested with RsaI and HinfI for 2-3 hrs at 37° C. and loaded onto a 0.6% agarose gel followed by Southern blotting using the Telotaggg Telomere Length Assay Kit (Roche). Quantification was performed using the WALTER webtool.

Targeted CRISPR/Cas9 Gene Editing: 37 μmol of Alt-R® S.p. Cas9 Nuclease V3 (IDT) and 50 total pmol of chemically modified sgRNA(s) (synthego) were complexed at room temperate for 20 minutes. Cells were harvested, washed in PBS, then 200-400 thousand cells were combined with cas9/gRNA complexes in 20 μM Buffer R (Thermo Fisher) with Alt-R® Cas9 Electroporation Enhancer (IDT) followed by electroporation using the Neon Transfection System (Thermo Fisher) and the Neon Transfection 10 μl kit. K562 cells were electroporated with three pulses at 1150 V for 10 ms. 293T cells were electroporated with one pulse of 1200 V for 30 ms.

CRISPR/Cas9 Editing Efficiency Determination: genomic DNA was isolated from cells using the Purelink Genomic DNA mini kit (Invitrogen) followed by PCR amplification with Q5® High-Fidelity DNA Polymerase (NEB) with the high GC enhancer, followed by either running on a 2% agarose gel or Sanger sequencing and editing efficiency calculation using the Synthego ICE webtool.

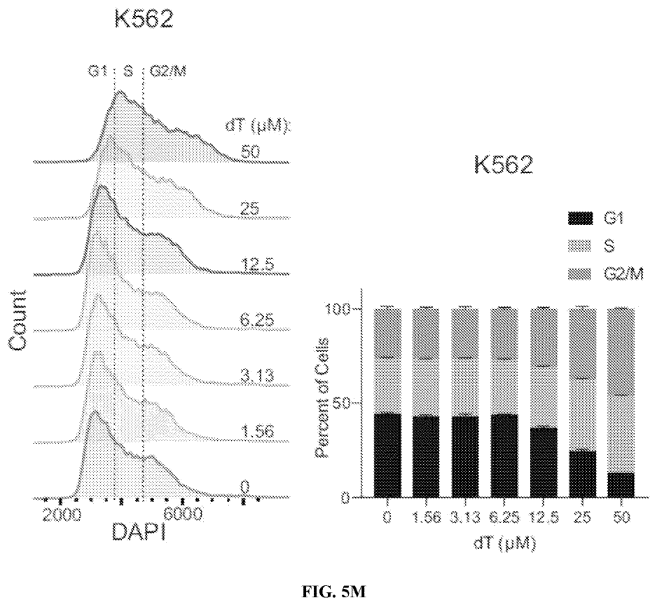

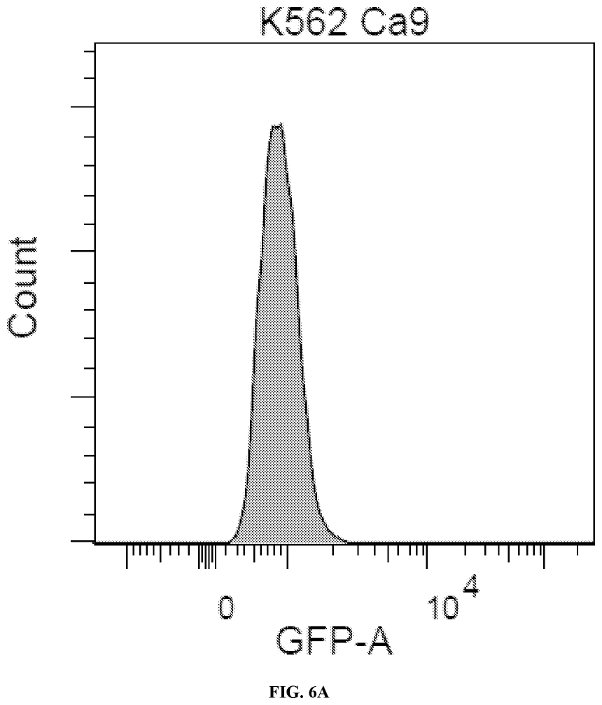

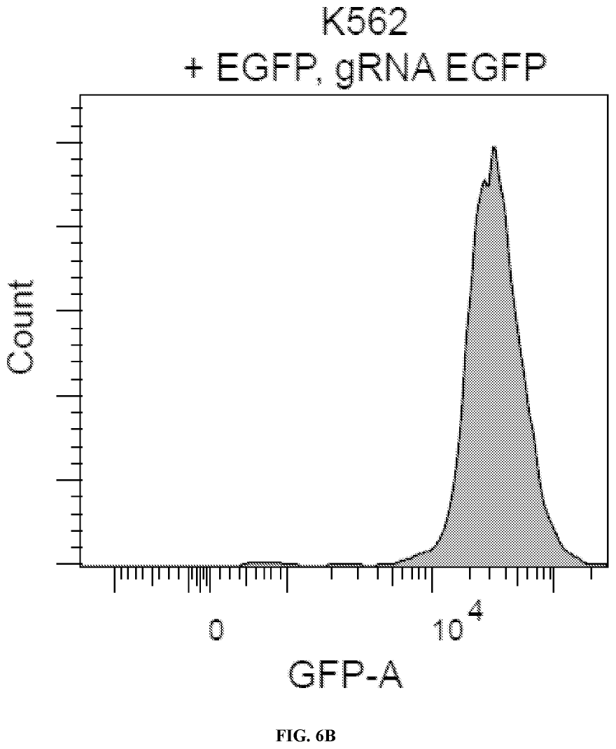

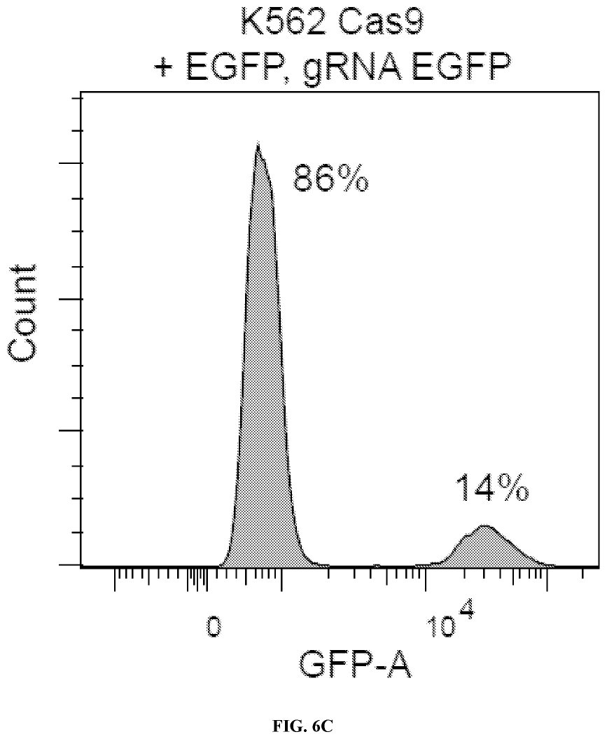

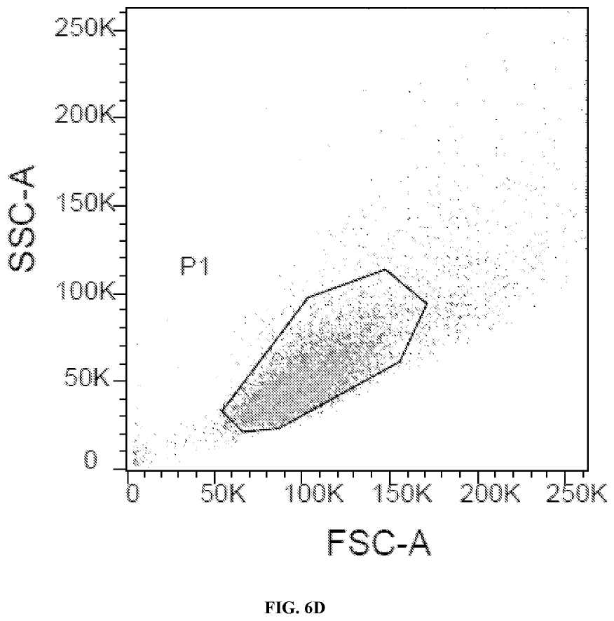

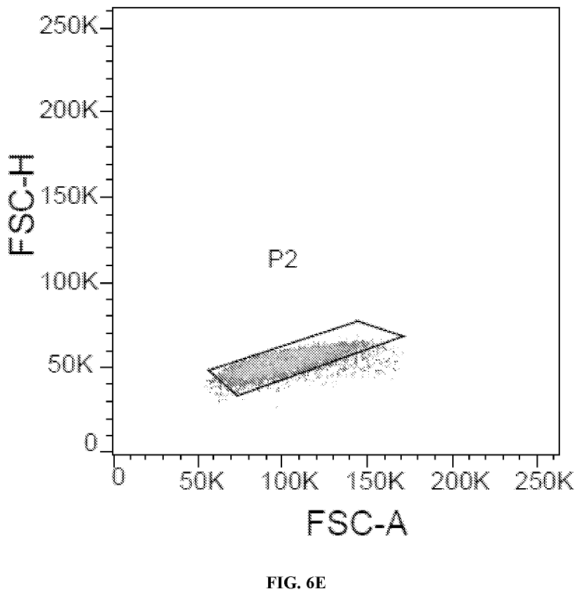

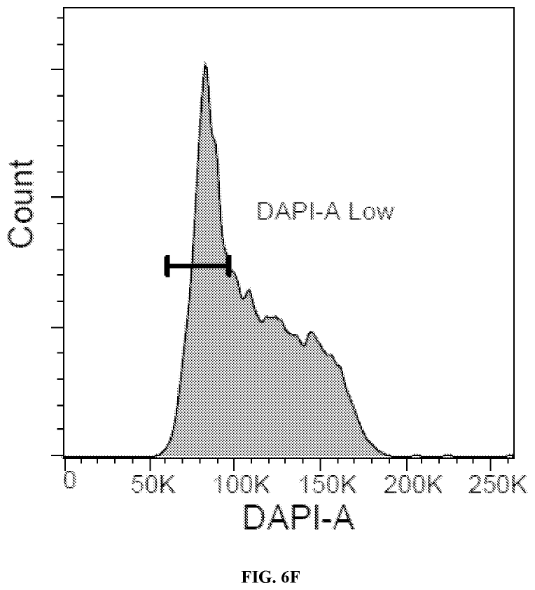

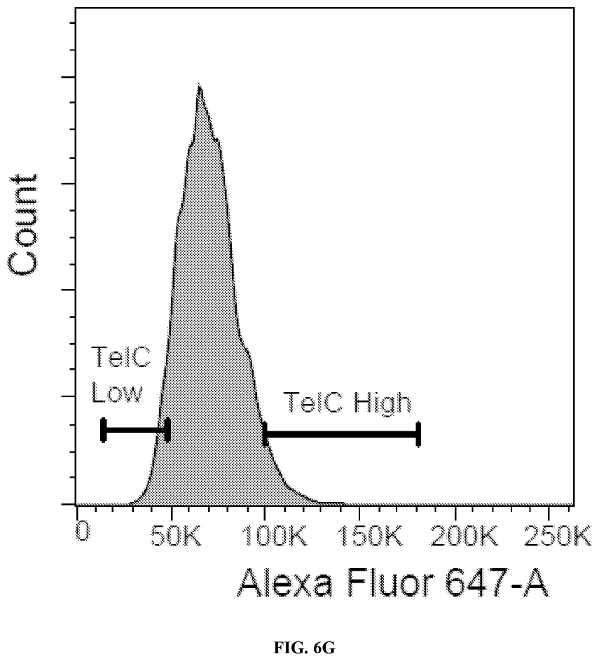

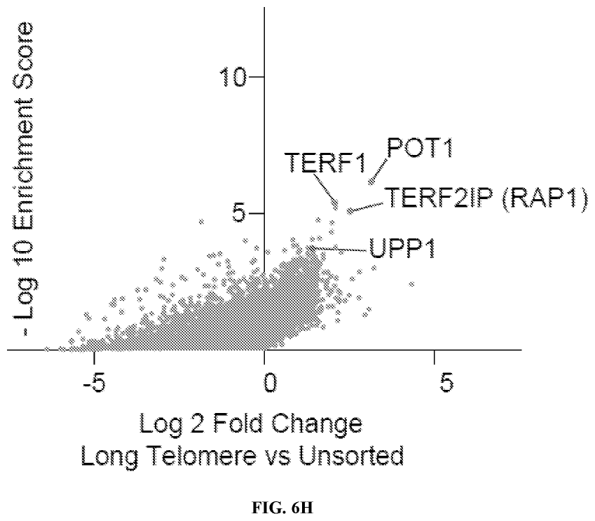

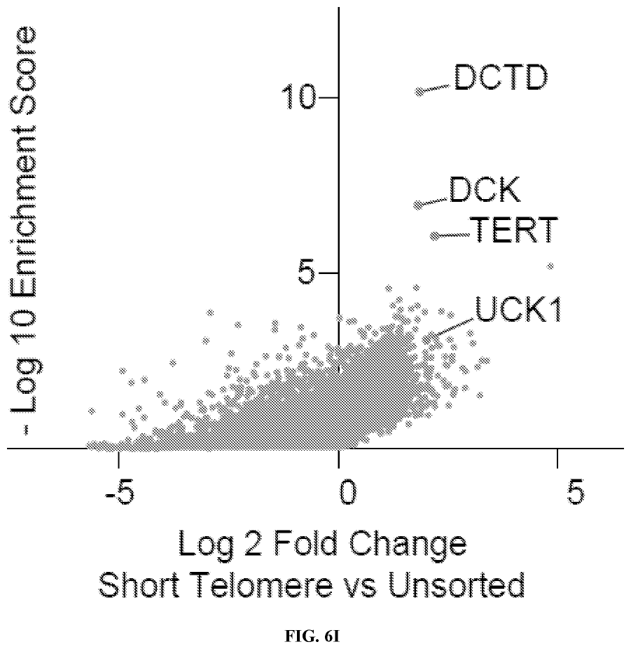

Immunoblotting: cells were lysed in 1μ Laemmli sample buffer (Bio-Rad) and run on a 10% SDS-PAGE gel (Bio-Rad) followed by transfer to PVDF membrane (Bio-Rad) using standard procedures. Human SAMHD1 was detected using primary antibody from either Abcam ab67820 (Extended Data FIG. 6a,c) or Origene OTI3F5 (Extended Data FIG. 6b) at 1:500 dilution and horseradish peroxidase conjugated goat-anti-mouse IgG H&L ab205719 at 1:1000 dilution. Anti-beta-actin antibody directly conjugated to horseradish peroxidase (Santa Cruz Biotechnology C4 sc-47778 HRP) at 1:1000 was used to quantify relative loading. Imaging was performed using a Bio-Rad Chemi Doc Imager.

Cell cycle analysis by DNA content staining and flow cytometry: Cells were prepared either for flow-FISH staining as described (for experiments in FIG. 5) or by ethanol fixation (for data in FIG. 6, 27), followed by rehydration and counterstaining using DAPI (BD Biosciences) and analysis on an LSR-II or Fortessa analyzer (BD Biosciences) using BD FACSDiva 8.0.2. Cells were gated on FSC/SSC as described in FIG. 6d using FlowJo V10.7.1.

Liquid Chromatography-Mass Spectrometry: extraction of and quantification of polar metabolites was performed as described using HPLC grade methanol (Fisher Scientific). If a given species was detected in multiple ion modes, the mode with the highest average signal in the untreated cells was used for analysis.

Nanopore sequencing: for sequencing of telomere end PCR products (FIG. 25h,i), amplicons were purified using the Qiagen PCR Purification kit and sequenced using the Plasmidsaurus amplicon sequencing service. Data from Plasmidsaurus nanopore sequencing and Sanger sequencing were aligned and displayed using Geneious Prime. T-free TRAP products were prepared for nanopore sequencing using the Ligation Sequencing Kit with the Native Barcoding Expansion (Oxford Nanopore Technologies SQK-LSK109, EXP-NBD104) according to the manufacturer's instructions, except that a ratio of 4:1 of SPR1 Purification Beads (Beckman Coulter) was used for all purification steps. Samples were pooled after barcode ligation, adapters were ligated per the manufacturer's instructions, then products were loaded onto a Flongle flow cell (FLO-FLG001). Reads were first analyzed using the high accuracy mode (MinKNOW/Guppy) and reads which had passed quality standards (Q score >9) from the high accuracy base calling were re-base called using super-high accuracy algorithm (MinKNOW/Guppy). Then, super-high accuracy base-called reads that had Q score >10 were used for further analysis. FASTQ files were analyzed in MATLAB as follows. Reads containing a match to the TS GGAAAG forward primer and the TAGGGAT portion of the reverse primer reverse complement were extracted and the base pairs between the primer binding sites were analyzed for their base pair composition using Version 1.0 of a custom MATLAB script which has been posted to public repository (see FIG. 25). Software versions used: Geneious Prime 2019.2.3, MinKNOW 22.05.5, Bream 7.1.3, Configuration 5.1.5, Guppy 6.1.5, MinKNOW Core 5.1.0, MATLAB R²⁰²¹a. Base pair frequency analysis and plotting in FIG. 25 was performed using ggseqlogo with R version 4.1.2.

Telomerase immunopurification: 293T TERC-null cells were transfected as described above with 3×HA-TERT and TERC/EGFP in 10-cm diameter dishes with reagents scaled up proportional to cell growth area. Two days after transfection, cells were harvested, washed once in PBS, and lysed in TRAPeze™1×CHAPS Lysis Buffer (Roche) supplemented with RNasin Plus (Promega) and HALT protease inhibitor cocktail (Thermo Fisher), followed by incubation on ice for 30 minutes and precipitation of insoluble material by centrifugation. Immunoprecipitation was performed as described previously with minor modifications. Briefly, 75 μl of anti-HA magnetic beads (Sigma-Aldrich, SAE0197, mouse IgG1 monoclonal antibody, Clone HA-7, 50% beads by volume) were added to clarified lysates followed by 2 hours incubation at 4° C. while rotating. Prior to addition, beads were prepared by washing four times in 50 mM Tris-HCl pH 8.0, 1 mM MgCl₂, 1 mM Spermidine, and 5 mM β-mercaptoethanol. After immunoprecipitation, telomerase bound beads were washed four times with 1 ml of telomerase buffer with 30% glycerol (50 mM Tris-HCl pH 8.0, 50 mM KCl, 1 mM MgCl₂, 1 mM Spermidine, 5 mM β-mercaptoethanol, 30% glycerol). After washing, a 50% bead slurry was made with telomerase buffer with 30% glycerol, then beads were aliquoted, snap frozen, and stored at −80° C.

Direct telomerase assay: reactions were performed as described previously with minor modifications. 20 μl reactions were assembled containing 6 μl of immunopurified telomerase, 5.2 μM dGTP, 3 μM dATP, 0.166 μM [α-P³²] dATP (3,000 Ci/mmol, Perkin Elmer), 1 μM PAGE purified 3× (TTAGGG) primer (IDT), 50 mM Tris-HCl pH 8.0, 50 mM KCl, 1 mM MgCl₂, 1 mM Spermidine, an 5 mM β-mercaptoethanol. dTTP was added as indicated in the figure legend. Reactions were then incubated at 30° C. for 1 hour, then 100 μl of stop buffer (3.6 M ammonium acetate, 10 mg/ml glycogen) was added as well as ³²P end-labeled PAGE purified TTAGGGTTAGGGTTAG primer (IDT) followed by the addition of 500 μl ethanol. Products were then incubated at −80° C. for 45 minutes, pelleted, washed with 1 ml 70% ethanol, dried, and resuspended in 10 μl water. Purified products were combined 1:1 with electrophoresis buffer (0.1×TBE, 50 mM EDTA, 0.1% Bromophenol blue, 0.1% Xylene Cyanol, 93% formamide), denatured at 95° C. for 5 minutes, then centrifuged at 18,000 g for 5 minutes to precipitate insoluble material. 9 μl of samples were then loaded onto 10% acrylamide/7 M urea gels. After electrophoresis, gels were then dried using the Bio-Rad Gel Air Drying System and imaged using phosphorimaging on an Amersham Typhoon 5 Biomolecular Imager (GE Healthcare). End labeling of the loading control primer was performed using T4 PNK (NEB) and γ-³²P-dATP 6,000 Ci/mmol (Perkin Elmer). Images were quantified using ImageJ as follows. For total telomerase activity measurement (FIG. 25f), telomerase product signal in a lane was quantified and normalized to the loading control band signal within the same lane. This ratio was then normalized to the average product to loading control ratio for the 0 μM dTTP lanes. For the analysis of telomerase repeat intensity (FIG. 25x), the signal of each repeat (defined as the three consecutive intense bands in the laddering pattern, as indicated in FIG. 25d), was quantified and first normalized to the loading control band signal for that lane. Then the ratio of a given repeat to loading control was divided by the corresponding ratio for the 0 μM dTTP lane, yielding a measure of relative repeat intensity, which was plotted for repeats 1-4. Statistical analysis was performed using Graphpad Prism V9.1.0.

shRNA construct cloning: olligonucleotides encoding shRNAs targeting SAMHD1 or luciferase control were annealed and cloned into the pLKO.1-puro vector using the Quick Ligation™ Kit (NEB).

SAMHD 1 expression construct cloning: primers with flanking Attb sites were used to amplify SAMHD1 sequence derived from cDNA prepared as described above or EGFP from the pXPR-011 vector using q5 High Fidelity DNA Polymerase (NEB). Amplicons were cloned into the pCW5a7.1 vector in a single reaction using Gateway™ LR Clonase™ II Enzyme mix (Invitrogen), Gateway™ BP Clonase™ II Enzyme mix, (Invitrogen) and the Gateway™ pDONR™221 Vector (Invitrogen). Point mutations were introduced using the Q5 site directed mutagenesis kit (NEB) and verified using Sanger sequencing.

Plasmids: lenti-Cas9-2A-Blast was a gift from Jason Moffat (Addgene plasmid #73310). pXPR_011 was a gift from John Doench & David Root (Addgene plasmid #59702). Human Brunello CRISPR knockout pooled library in lentiGuide-Puro was a gift from David Root and John Doench (Addgene #73178). lentiGuide-Puro was a gift from Feng Zhang (Addgene plasmid #52963). pLKO.1-TRC cloning vector was a gift from David Root (Addgene plasmid #10878). pCW57.1 was a gift from David Root (Addgene plasmid #41393). psPAX2 was a gift from Didier Trono (Addgene plasmid #12260). pMD2.G was a gift from Didier Trono (Addgene plasmid #12259).

Slot blotting: slot blotting was performed as described with minor modifications. Briefly, DNA concentration was normalized to 20 ng/μl using a nanodrop spectrophotometer. 3.3 μl of DNA was added to 16.5 μl of denaturation solution (0.5 M NaOH, 1.5 M NaCl) and heated to 55° C. for 30 minutes. 495 μl of neutralization solution (0.5 M Tris-HCl, 1.5 M NaCl) was added to denatured DNA and 156 μl of this solution was added in triplicate to different slots on a Bio-Dot SF Apparatus (Bio-Rad) and blotted onto Hybond N+ Membrane (Cytivia). Membrane preparation and washing was performed as described. For samples with paired GGTTAG and GGAAAG slot blots, the above recipe was doubled, and 156 μl was loaded onto parallel membranes in triplicate on each membrane. After blotting, membranes were hybridized and detected as described above for TRF Southern blots using the probe indicated in the figure. Blots were analyzed using ImageJ. Statistical analysis was performed using Graphpad prism V9.1.0.

### Nucleoside and Other Small Molecule Suppliers:

| Compound | Supplier | Purity | Note |
|---|---|---|---|
| Thymidine | Sigma Aldrich | ≥99% | Default source used, |
| | | | referred to as |
| | | | manufacturer 1 in |
| | | | FIG. 2F |
| Thymidine | Santa Cruz | >98% | Referred to as |
| | Biotechnology | | manufacturer 2 in |
| | | | FIG. 2F |
| Thymidine | MP Biochemicals | >99.9% | Referred to as |
| | | | manufacturer 3 in |
| | | | FIG. 2F |
| Deoxyuridine | Sigma Aldrich | >98.5% | Thymidine free |
| | | | according to |
| | | | manufacturer |
| Deoxyadenosine | Sigma Aldrich | ≥99% | |
| Deoxyguanosine | Sigma Aldrich | ≥99% | |
| Deoxycytidine | Thermo Scientific | ≥99% | |
| Folic Acid | Sigma Aldrich | >97% | |
| Hydroxyurea | Thermo Scientific | ≥97.5% | |
| | (Acros Organics) | | |
| Ro-3306 | Selleck | >98% | |
| | Chemicals | | |

### Primers Used:

| Name | Sequence (5′ to 3′) |
|---|---|
| shSAMHD1-1 oligo 1 | CCGGCCCTGAAGAAGATATTTGCTTCTCG |
| | AGAAGCAAATATCTTCTTCAGGGTTTTTG |
| shSAMHD1-1 oligo 2 | AATTCAAAAACCCTGAAGAAGATATTTG |
| | CTTCTCGAGAAGCAAATATCTTCTTCAG |
| | GG |
| shSAMHD1-1 oligo 1 | CCGGGCCATCATCTTGGAATCCAAACTC |
| | GAGTTTGGATTCCAAGATGATGGCTTTT |
| | TG |
| shSAMHD1-1 oligo 2 | AATTCAAAAAGCCATCATCTTGGAATCC |
| | AAACTCGAGTTTGGATTCCAAGATGATG |
| | GC |
| shLuciferase oligo 1 | CCGGCGCTGAGTACTTCGAAATGTCCTC |
| | GAGGACATTTCGAAGTACTCAGCGTTTT |
| | TG |
| shLuciferase oligo 2 | AATTCAAAAACGCTGAGTACTTCGAAAT |
| | GTCCTCGAGGACATTTCGAAGTACTCAG |
| | CG |
| SAMHD1 K312A | TGATGTGGACgcaTGGGATTATTTTG |
| SDM F | |
| SAMHD1 K312A | ATGCCATTTCTTTTATTAGATAC |
| SDM R | |
| SAMHD1 H215A | GCCATTTTCTgccATGTTTGATGGAC |
| SDM F | |
| SAMHD1 H215A | CCATGACCGAGATCATGAC |
| SDM R | |
| P5 Lenti Guide Puro | ATGATACGGCGACCACCGAGATCTACAC |
| Seq | TCTTTCCCTACACGACGCTCTTCCGATCT |
| | [s]TTGTGGAAAGGACGAAACACCG |
| P7 Lenti Guide Puro | CAAGCAGAAGACGGCATACGAGATNNN |
| Seq | NNNNNGTGACTGGAGTTCAGACGTGTGC |
| | TCTTCCGATCTTCTACTATTCTTTCCCCT |
| | GCACTGT |
| TERC qPCR Fw | GGGAGGGGTGGTGGCCATTTTT |
| TERC qPCR Rv | CACCGCGAAGAGTTGGGCTCTG |
| GAPDH qPCR Fw | CAGTCTTCTGGGTGGCAGTGA |
| GAPDH qPCR Rv | CGTGGAAGGACTCATGACCA |
| AAVS1 sgRNA | CUCCCUCCCAGGAUCCUCUC |
| TK1 sgRNA 1 | AAGGCACAGGACACACUGUU |
| TK1 sgRNA 2 | GCAGCUUCUGCACACAUGAC |
| TK1 sgRNA 3 | ACUGAGCAAUCUGGAAGCGA |
| TK2 sgRNA 1 | CCAAGACGCGCGUUACCGGG |
| TK2 sgRNA 2 | GCCGGGAAGUCGCGGGAGCC |
| TK2 sgRNA 3 | CUCGCCCGGCUAUGCUGCUG |
| TYMS sgRNA 1 | CGCCCGCCGCGCCAUGCCUG |
| TYMS sgRNA 2 | CGAGCCGCGUCCGCCGCACG |
| TYMS sgRNA 3 | CGUCAGGAAGGACGACCGCA |
| SAMHD1 sgRNA 1 | AAAGCCACCGCGCCUGAGGA |
| SAMHD1 sgRNA 2 | UCUGCGGAAGGGGUGUUUGA |
| SAMHD1 sgRNA 3 | CUUGGAGGGCUGCUCGGAAU |
| TK1 FW | AAAGCCCAAAAGCATCCCCT |
| TK1 RV | TCTGCCCTGCTCTTTTCTGG |
| TK2 FW | ATCCCAGAACCAAAGCCGAG |
| TK2 RV | AAATGCGACCGGGTCTCTTT |
| TYMS FW | TGCTGCTGGCTTAGAGAAGG |
| TYMS RV | GTAAGGCGAGGAGGACGATG |
| SAMHD1 Gateway | GGGGACAAGTTTGTACAAAAAAGCAGGC |
| Cloning F | TTAGCCGCCACCATGCAGCGAGCCGATT |
| | CCG |
| SAMHD1 Gateway | GGGGACCACTTTGTACAAGAAAGCTGGG |
| Cloning R | TATCACATTGGGTCATCTTTAAAAAGCTG |
| EGFP Gateway | GGGGACAAGTTTGTACAAAAAAGCAGGC |
| Cloning F | TTAGCCGCCACCATGGTGAGCAAGGGCG |
| | AGG |
| EGFP Gateway | GGGGACCACTTTGTACAAGAAAGCTGGG |
| Cloning R | TATTAGGACTTGTACAGCTCGTC |

gRNAs in Library (all were Enriched in Long Telomere Population)

| sgRNA number | sgRNA | Gene Target |
|---|---|---|
| sg_0381 | GGTGTAAAGAGTTGCGAGTG | SAMHD1 |
| sg_0382 | CTTCGATACATCAAACAGCT | SAMHD1 |
| sg_0383 | GGATGTCTAGTTCACGCACT | SAMHD1 |
| sg 0384 | TTACCTTCATTGTATCAACG | SAMHD1 |
| sg_0385 | AAACGAGACTCATCAAGACA | SAMHD1 |
| sg_0386 | TCCATCCCGACTACAAGACA | SAMHD1 |
| sg_0387 | TCTTCGACAGGTGATTCAAG | SAMHD1 |
| sg_0388 | TTCACCTCCGGGCGAGCAAG | SAMHD1 |
| sg_0389 | GATGACTACATAGAGATTAC | SAMHD1 |
| sg_0390 | TAAAAGAAATGGCATTGATG | SAMHD1 |

Telomeres are repetitive DNA elements which flank linear chromosomes and promote genomic stability. Telomere length decreases as cells divide because DNA polymerases are unable to fully replicate linear chromosomes. When critically short, telomeres initiate cellular senescence, arresting cell division. This shortening is counteracted by the telomerase reverse transcriptase, which uses an RNA template, TERC, to elongate telomeres by synthesizing new TTAGGG repeats. In Mendelian randomization studies, long telomeres are associated with increased lifespan, while inherited defects in genes regulating telomere maintenance are associated with a spectrum of lethal diseases including idiopathic pulmonary fibrosis, liver cirrhosis, and bone marrow failure.

### CRISPR Screening Links Thymidine Flux and Telomere Length

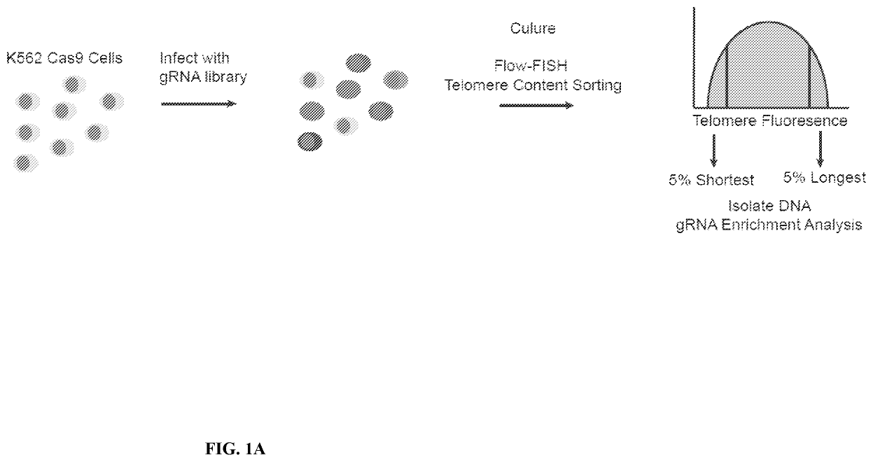

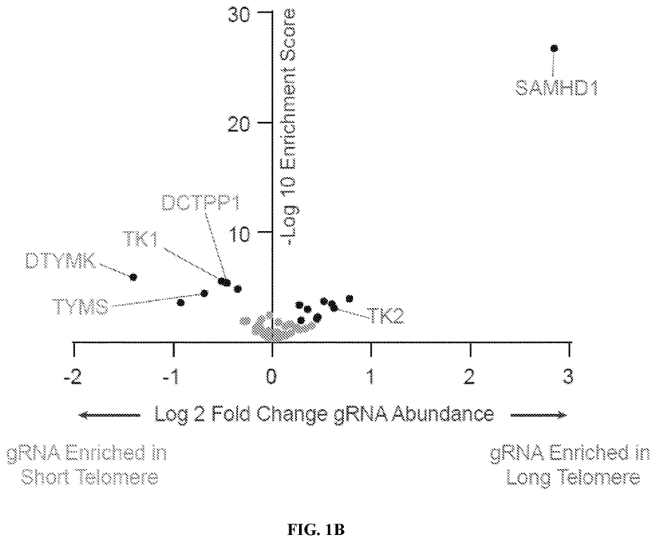

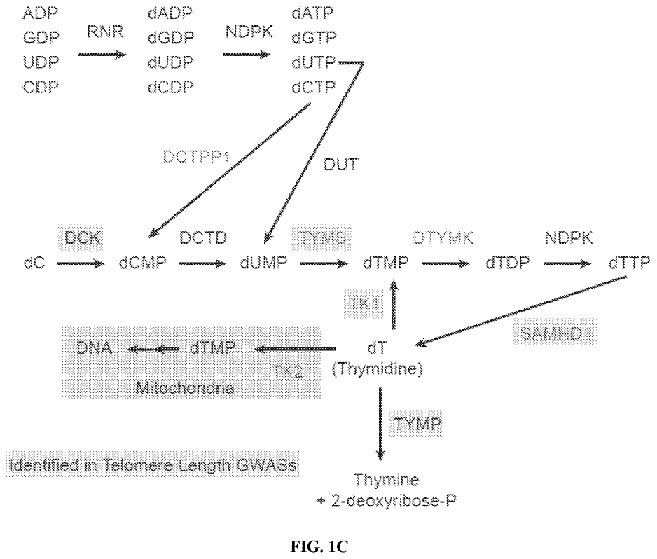

Fluorescence in situ-hybridization using a peptide nucleic acid (PNA) telomere repeat probe coupled with flow cytometry (flow-FISH) is a clinically validated telomere length measurement assay performed on intact single cells. Flow-FISH was used as a phenotypic readout for CRISPR/Cas9 screening in human cells to identify novel genes regulating telomere length (FIG. 1a). K562 cells expressing spCas9 (FIG. 6a-c) were transduced with the Brunello genome-wide gRNA library and cultured for 50 days, followed by isolation of intact cells harboring the longest and shortest 5th percentiles of telomere length using flow-FISH (FIG. 6d-g). gDNA was isolated from the sorted populations, subjected to deep sequencing, and gRNA representation was compared in these populations to that of the unsorted population using the MAGeCK Robust Rank Algorithm (RRA) (FIG. 6 h,i). gRNAs targeting telomerase reverse transcriptase (TERT) were found to be enriched in the short telomere population while gRNAs targeting several components of the shelterin telomere chromatin protein complex that negatively regulate telomerase length were enriched in the long telomere population, including POT1, TERF1, and TERF2IP (RAP1). These data validate the CRISPR screening strategy to identify tolerable genetic loss-of-function associated with human telomere length.

Unbiased pathway analysis of screening hits using the MAGeCKFlute analysis suite revealed an enrichment of pyrimidine nucleotide metabolism genes in the sorted short telomere population (FIG. 6j), including the top two genes with the highest enrichment scores. Recent genome-wide association studies have also implicated nucleotide metabolism genes as associated with telomere length control. Based on the screening results and these human genetic data (FIG. 1b), a second round of flow-FISH based screening using a custom gRNA library targeting 53 nucleotide metabolism genes was performed (ten gRNAs per gene). spCas9-expressing K562 cells were infected with lentiviruses encoding the nucleotide metabolism gRNA library and cultured cells for 26 days followed by flow-FISH sorting. Comparing the gRNAs enriched in the sorted long and short telomere populations, several genes were identified as implicated specifically in dT nucleotide metabolism (FIG. 1a). In cells with short telomeres, there was an enrichment of gRNAs that disrupted genes predicted to promote dT nucleotide synthesis, including DTYMK, TYMS, TK1, and DCTPP1. Conversely, gRNAs targeting genes predicted to reduce nuclear dT nucleotide levels, including the dNTPase SAMHD1, were enriched in cells with long telomeres. Taken together, these results point to dT nucleotide levels as a novel regulator of telomere length in human cells.

### Thymidine Treatment Increases Telomere Length in Human Cells

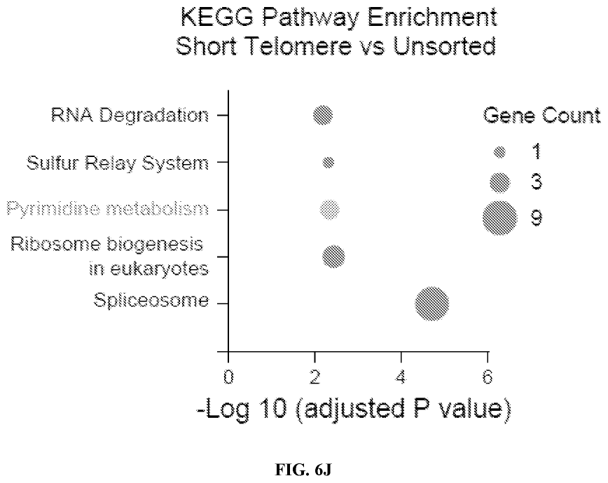

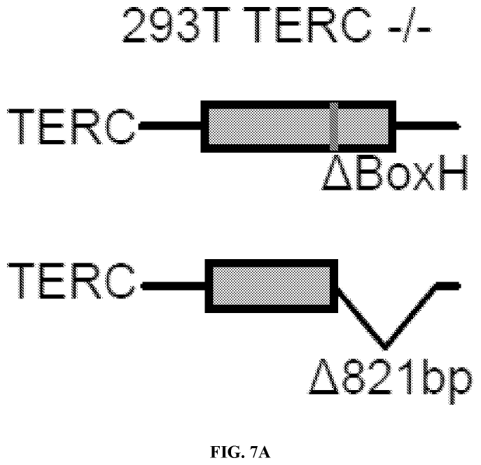

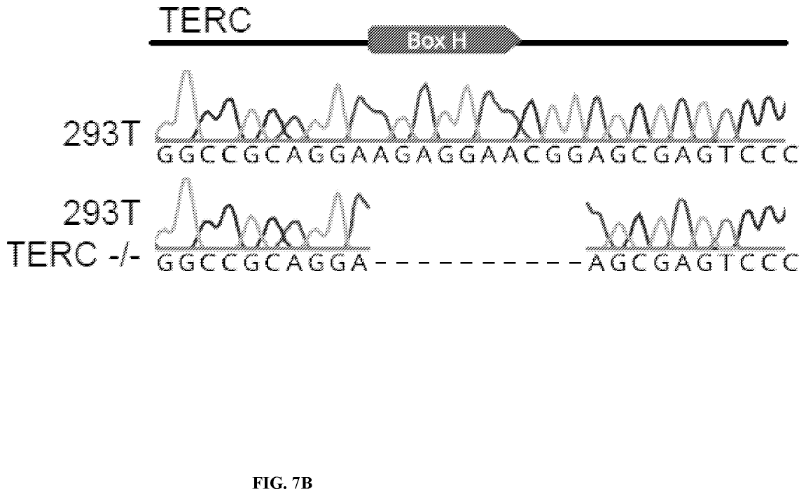

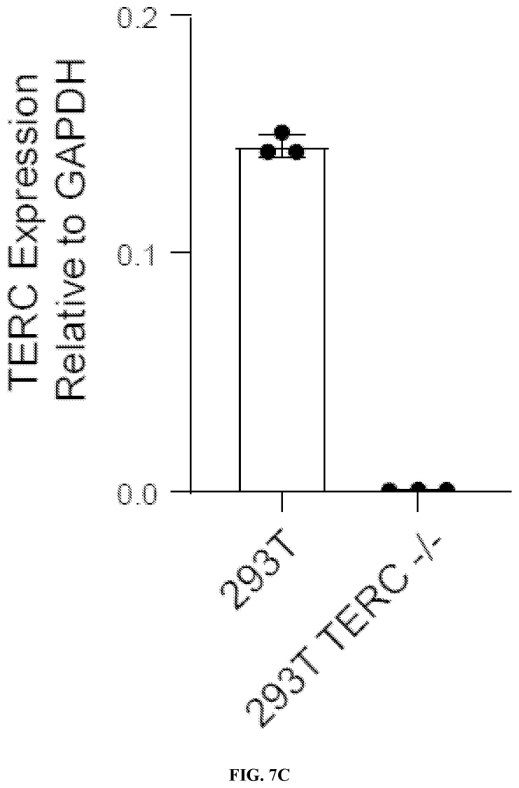

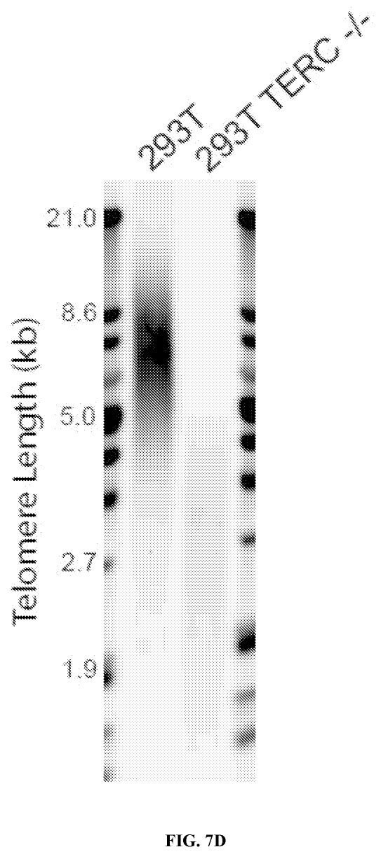

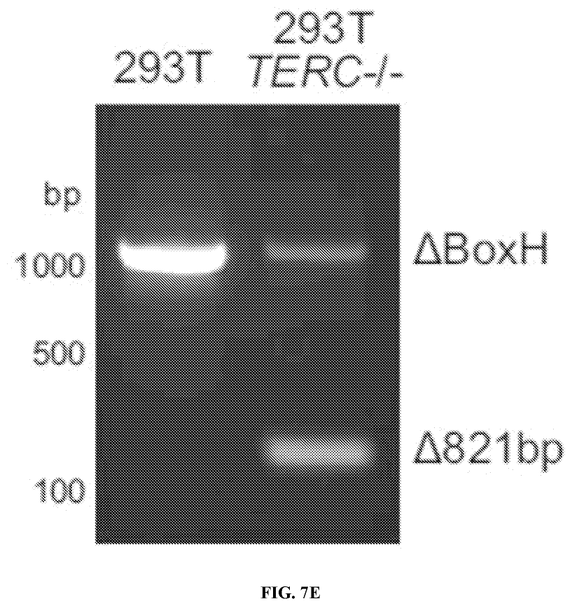

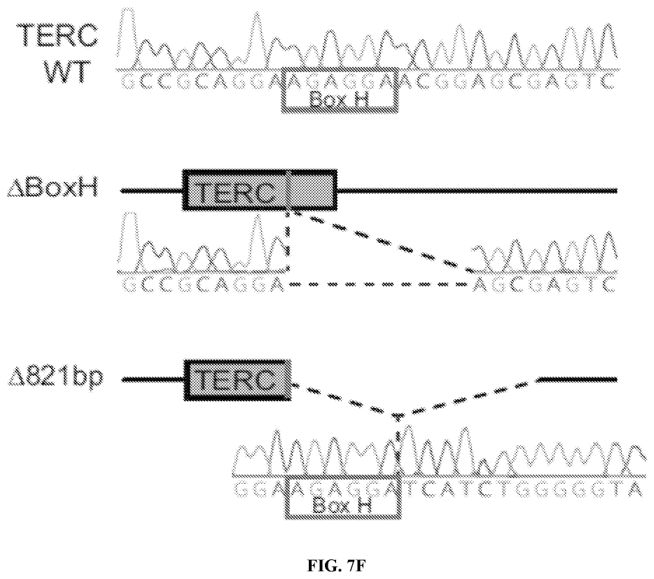

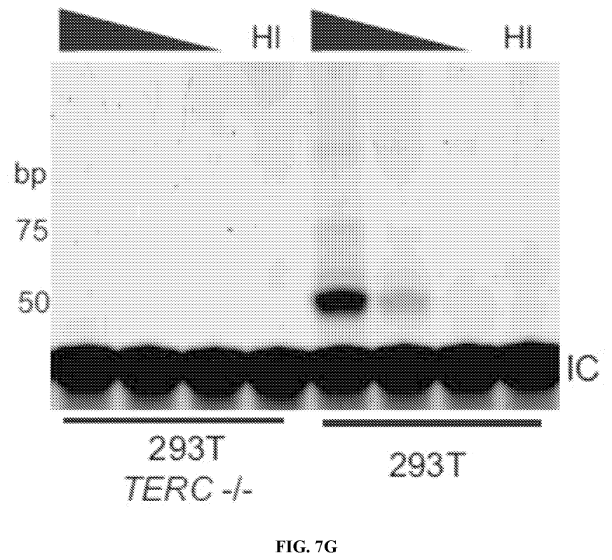

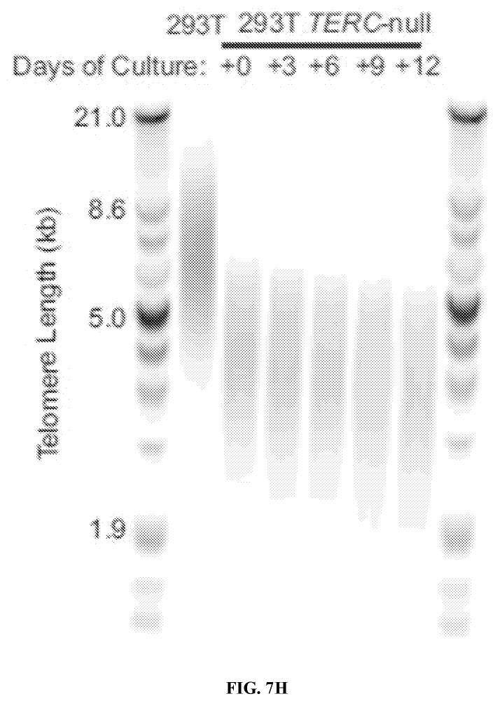

Based on these data, it was tested whether manipulation of nucleotides could alter telomere length in human cells. Deoxynucleosides (dNs) including deoxyadenosine (dA), dT, deoxycytidine (dC), and deoxyguanosine (dG), can increase dN nucleotide levels via salvage pathway kinases. Supplementing K562 or 293T cell culture media with these four canonical dNs drove rapid telomere lengthening within 8 days of treatment (FIG. 2a). When the experiment was repeated in telomerase negative TERC-null 293T cells (FIG. 7a-d), no increase was found in telomere length, indicating that telomerase is required for dN-mediated telomere lengthening. To identify which dNs promote telomere elongation, 293T (FIGS. 2b) and K562 cells (FIG. 2c) were treated with dNs individually or in combinations. It was found that combinations that included dT promoted telomere lengthening, as did dT alone, while no combination lacking dT could elongate telomeres (FIG. 2b-d). Again, dT treatment of telomerase-negative TERC-null 293T cells failed to drive detectable telomere elongation, indicating that dT-mediated telomere elongation occurs in a telomerase-dependent mechanism (FIG. 2e). Collectively, these findings demonstrate that dT supplementation alone can drive rapid and robust telomere lengthening in human cells and offer an explanation for the genetic data implicating nucleotide metabolism in human telomere length regulation: driving increased thymidine metabolism promotes telomere elongation.

It was next evaluated how treatment with escalating doses of dT impacted telomere length. Telomere elongation after dT treatment showed a continuous dose response up to 200 M in both 293T and K562 cells (FIG. 2f-h). Doses at or above 50 μM led to robust telomere elongation in both cell lines in less than 10 days. K562 cells were more sensitive to dT treatment in terms of telomere elongation compared to 293T cells (increase of 204 base pairs (bp) per day vs 96 bp per day at 100 μM; and 132 bp per day vs 60 bp per day at 50 M). It was confirmed that dT from different manufacturers, each of >98% purity, resulted in telomere elongation similarly, arguing against contaminating elements from one source confounding the interpretation (FIG. 2f). To study how telomere length changes from treatment or withdrawal of dT, a time-course of dT treatment was performed in 293T cells and it was found that dT supplementation gradually and continually increased telomere length, over the course of 33 days (FIG. 2i-j). Withdrawal of dT after 15 days of treatment aborted telomere elongation, indicating that this effect was dependent on exposure to dT as opposed to invoking autonomous mechanisms of telomere lengthening (FIG. 2i-j). These data indicate that dT supplementation promotes gradual and exposure-dependent telomere elongation in human cells.

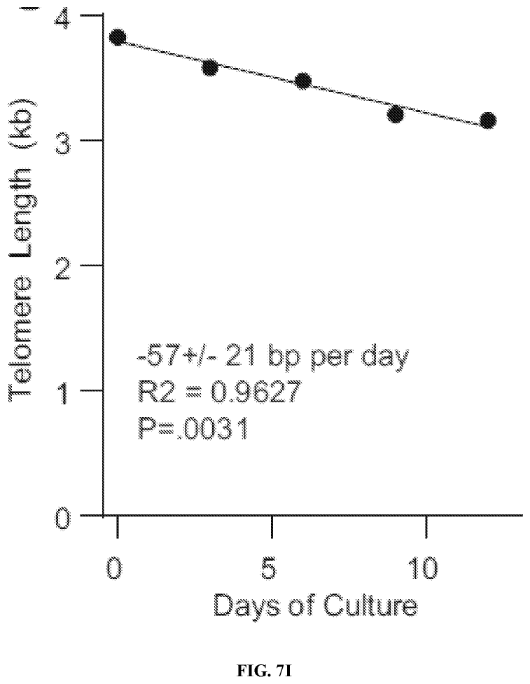

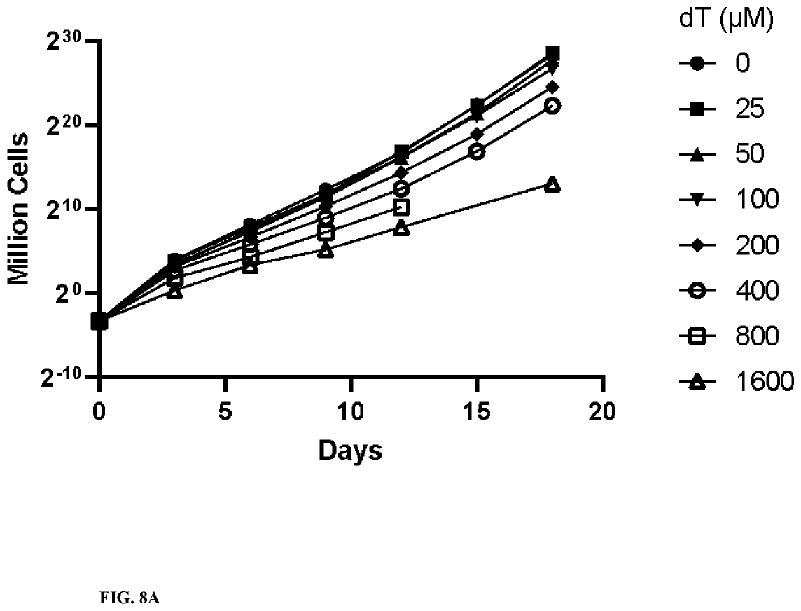

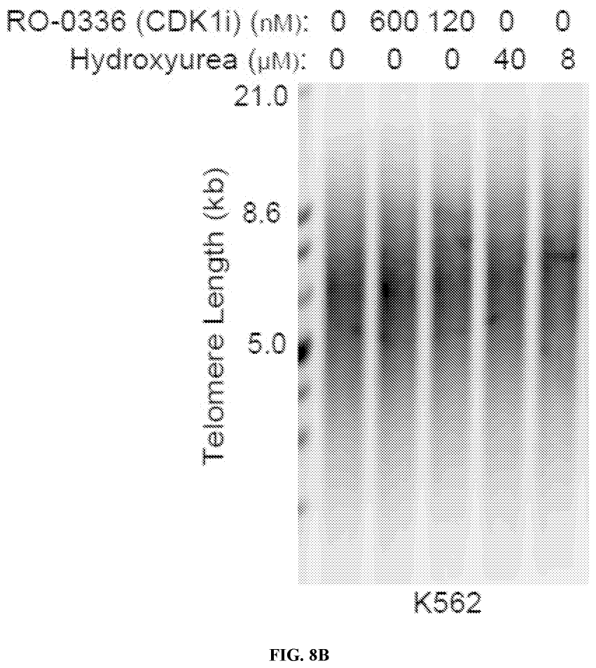

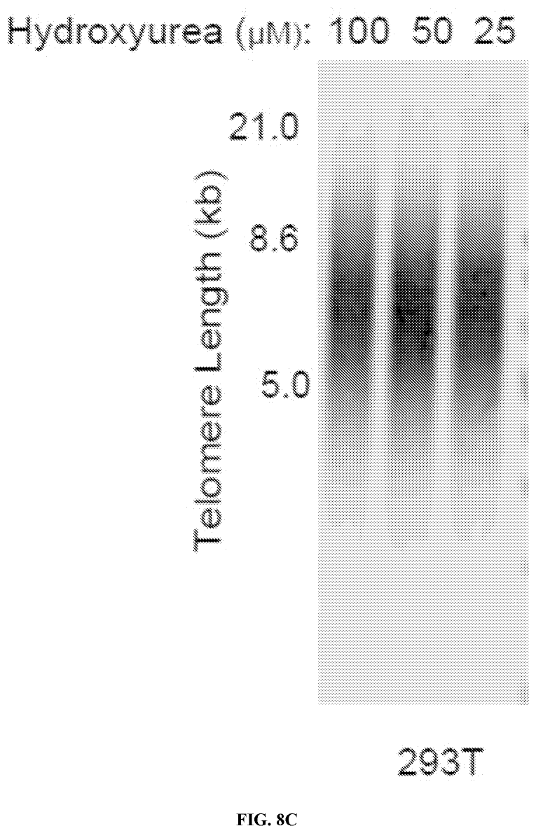

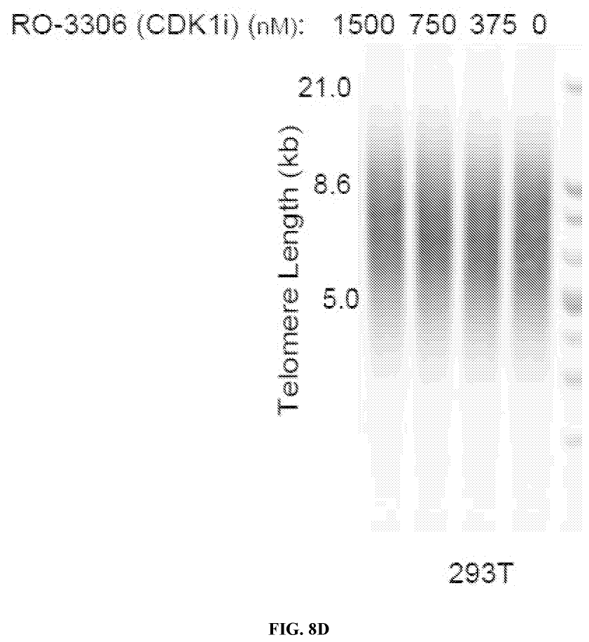

dT is commonly used to inhibit cell cycle progression, arresting cells in S phase at doses in the millimolar range. It was determined whether the effects of dT supplementation on telomere lengthening could be dissociated from its effects on the cell cycle. When 293T cells were treated with increasing doses of dT, inhibition of growth was detected beginning at 50 μM (FIG. 8a). Doses were chosen at which there was no discernable effect on cellular growth to evaluate the impact on telomere length. Whereas supplementation of 293T with 20 or 40 μM dT showed minimal telomere length changes on TRF analysis after 10 days (FIG. 2f), treatment for 34 days revealed robust telomere lengthening, but with undetectable changes in cellular growth curves (FIG. 2k,l). Similar results were seen in K562 cells treated with as low as 5 μM dT for 34 days (FIG. 2 m,n). To further determine whether cell cycle inhibition could cause the telomere length increases seen with dT treatment, cells were treated with the ribonucleotide reductase inhibitor hydroxyurea that arrests cells in S phase or the CDK1 inhibitor RO-3306, which synchronizes cells in G1 phase. No changes were found in telomere length after culturing K562 cells for 8 days or 293T cells for 10 days with hydroxyurea or RO-3306 at doses up to those maximally tolerated without causing cytotoxicity (FIG. 8b-d). Collectively, these data indicate that telomere lengthening after dT treatment is achieved at doses that do not impact cellular growth, and by mechanisms that are independent of effects on the cell cycle.

### TK1 is Required for Telomere Lengthening by Thymidine

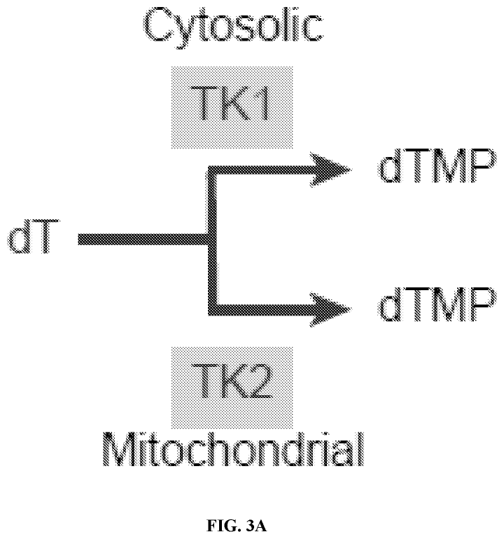

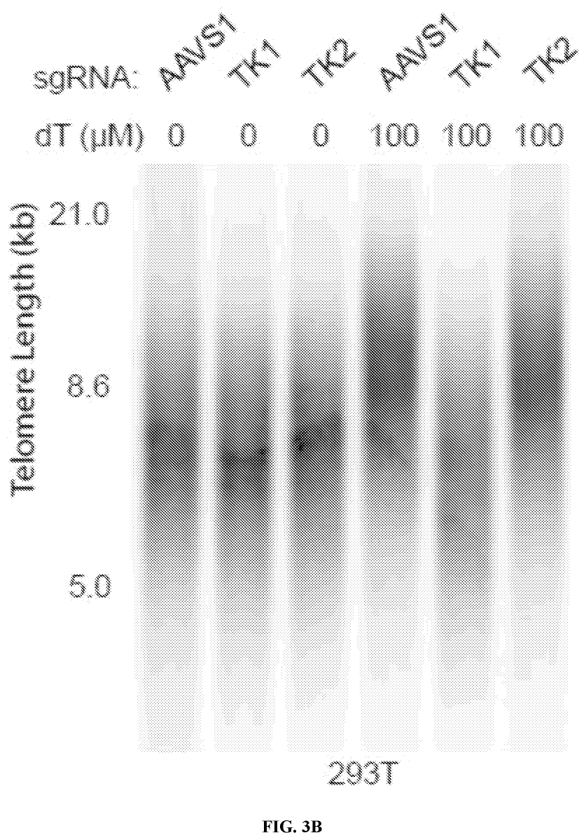

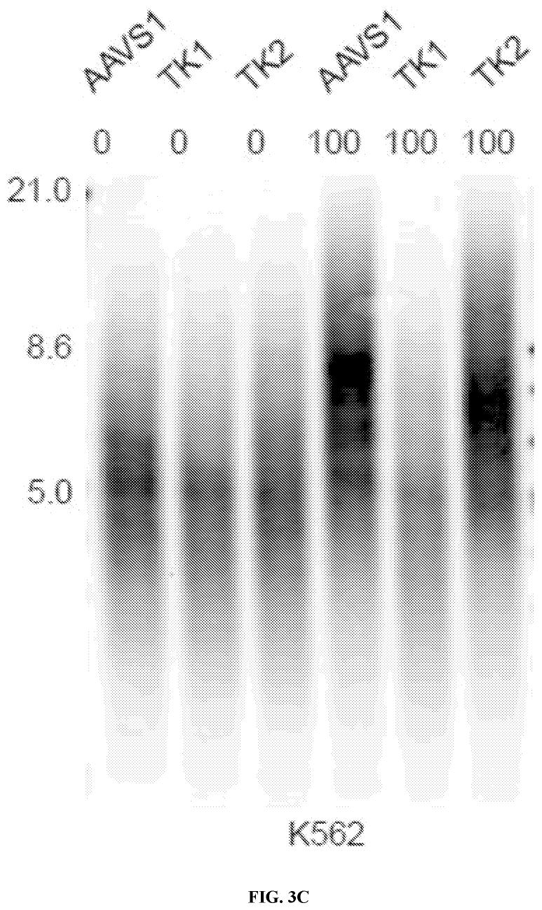

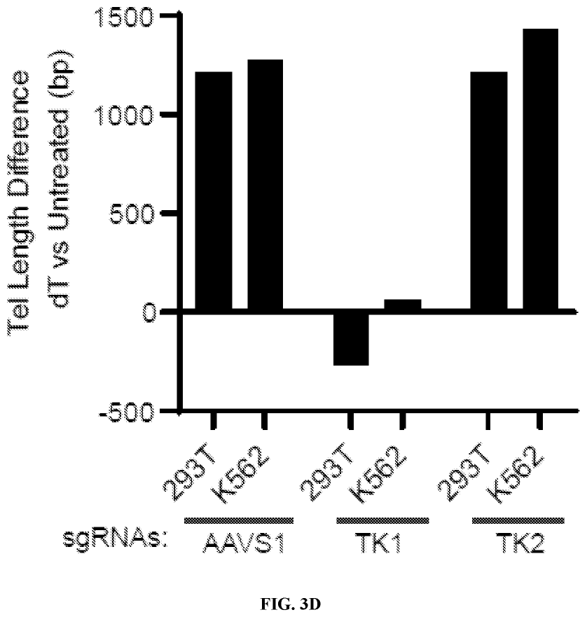

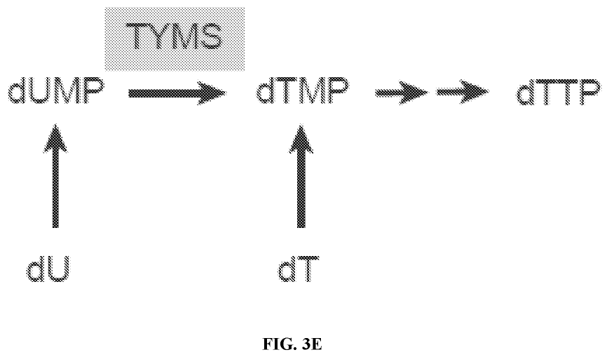

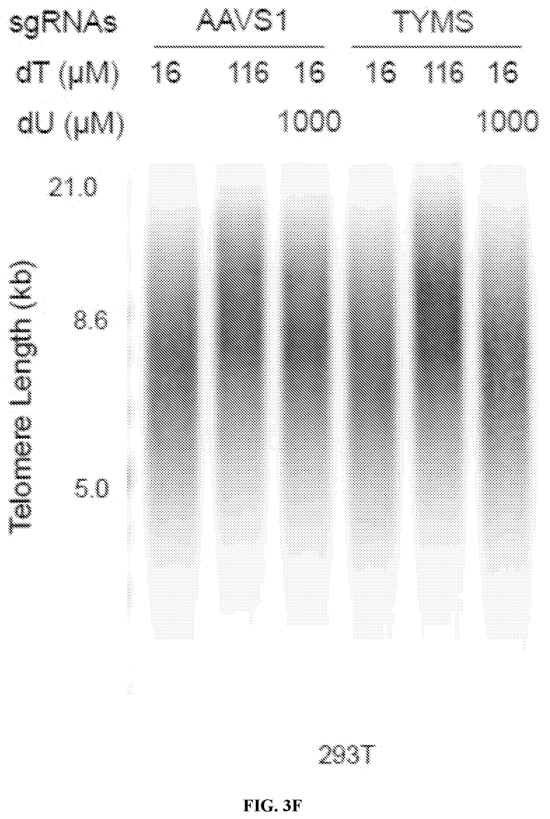

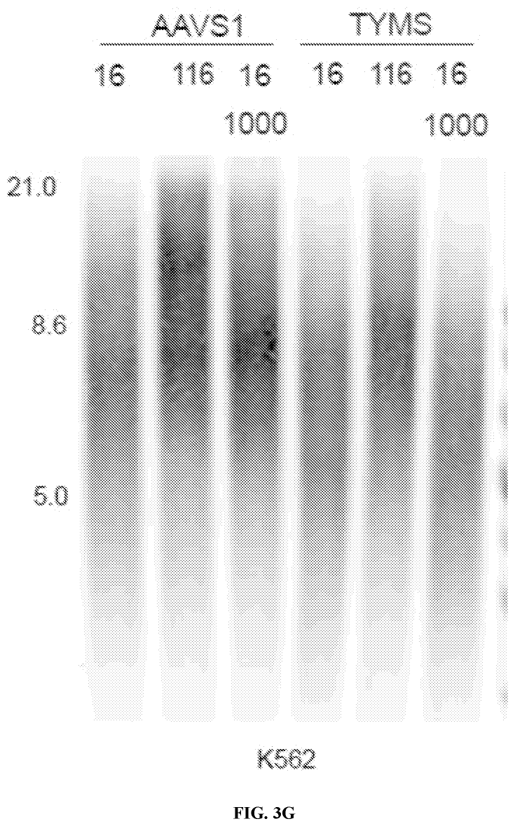

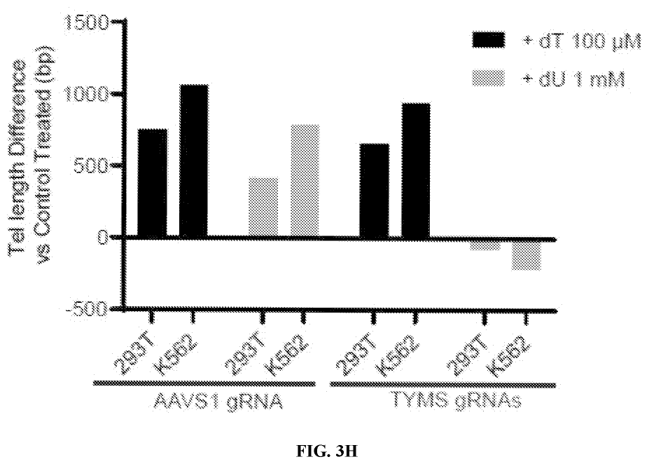

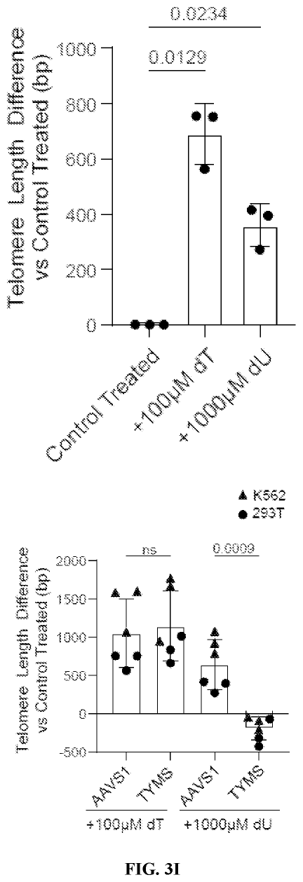

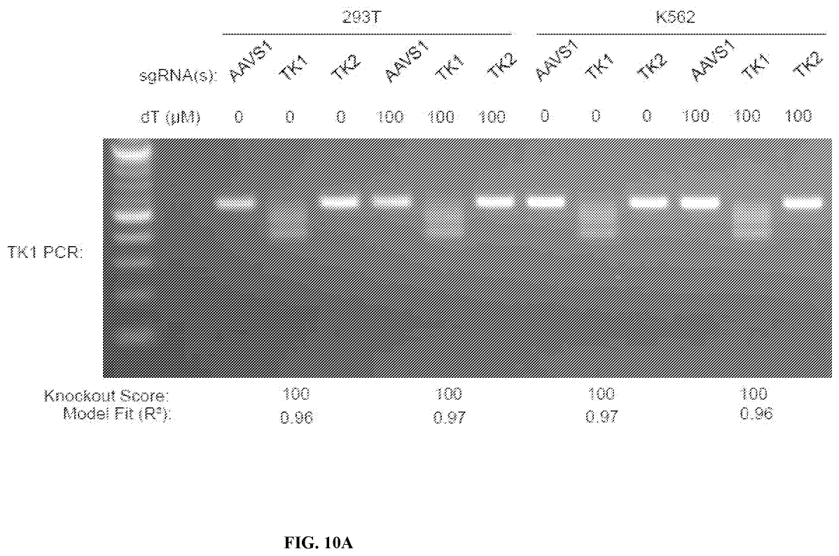

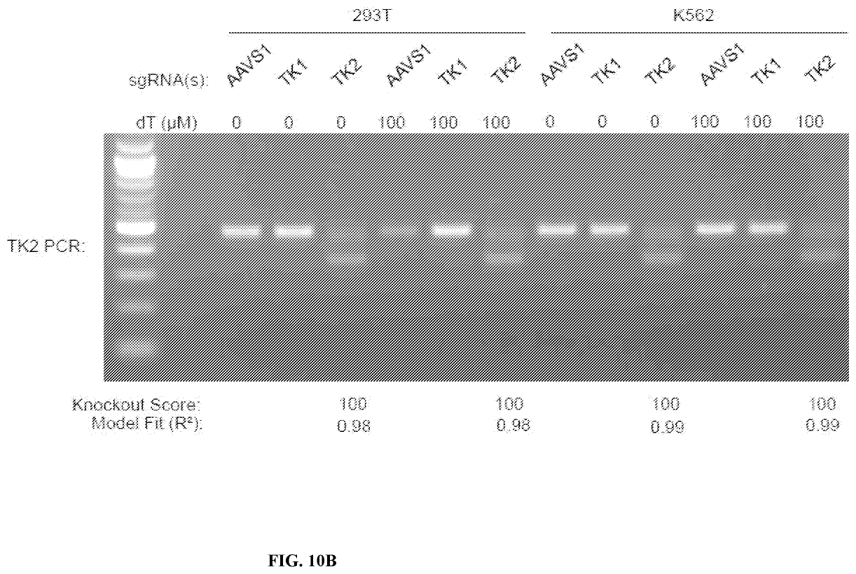

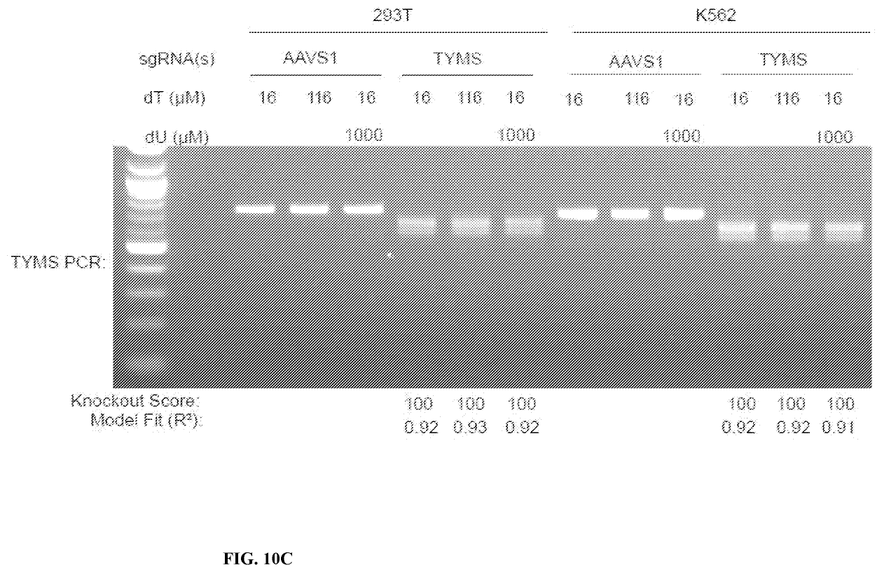

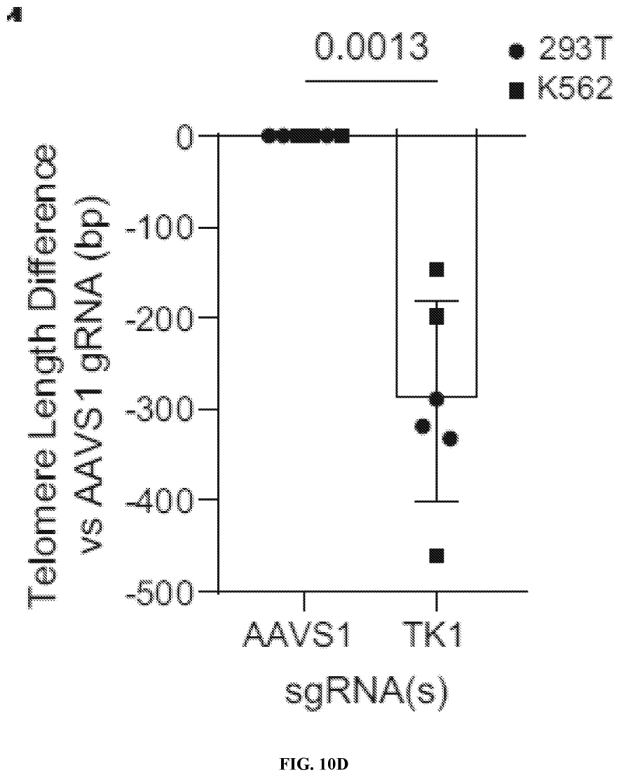

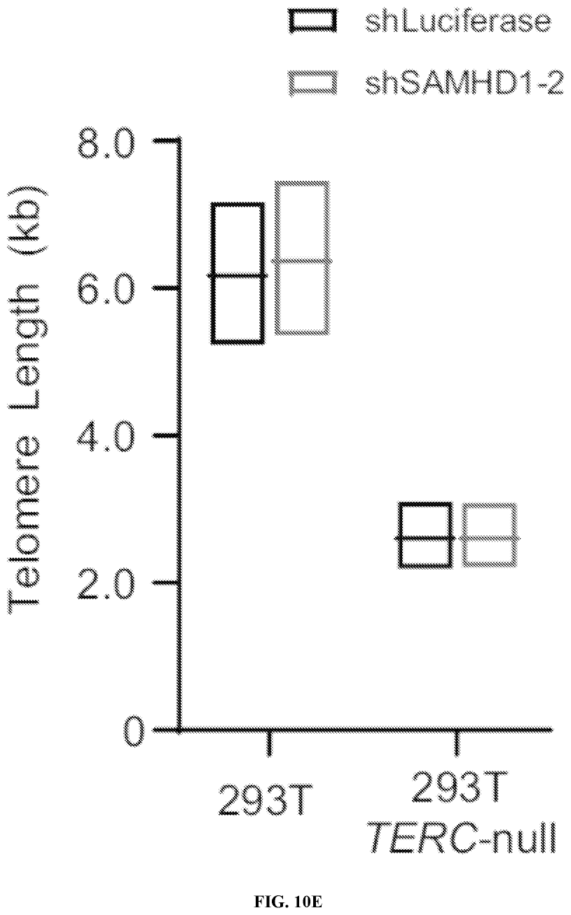

dT salvage occurs through distinct pathways to generate dTTP for mitochondrial versus nuclear genome synthesis (FIG. 3a). Thymidine kinase 1 (TK1) acts in the cytosol to generate dTMP available for nuclear genome replication, while thymidine kinase 2 (TK2) acts in the mitochondria to generate dT nucleotides for mitochondrial genome synthesis. As telomeres reside in the nuclear genome, it was hypothesized that dT mediated telomere lengthening requires functional TK1 but not TK2. To test this hypothesis, 293T or K562 cells were electroporated with spCas9 and pools of gRNAs targeting either TK1 or TK2, or with a gRNA targeting the AAVS1 control locus. Efficient gene editing was confirmed by PCR amplifying and sequencing the gRNA target site which revealed that the pool of three gRNAs yielded ~100% gene disruption in all cases in either TK1 or TK2 (FIG. 10 a,b). Consistent with this hypothesis, it was found that TK1-edited cells showed a complete abrogation of the telomere lengthening that is observed upon dT treatment (FIG. 3b-d), whereas TK2-edited cells showed no difference in telomere length increases compared to control AAVS1-targeted cells (FIG. 3b-d). Without dT treatment, a modest telomere length decrease was found from TK1 knockout, as anticipated from the screening data (FIG. 3b-c). These data indicate that cytosolic phosphorylation of dT to generate dTMP by TK1 is required for telomere lengthening after dT treatment.

### TYMS is Required for Telomere Lengthening by Deoxyuridine

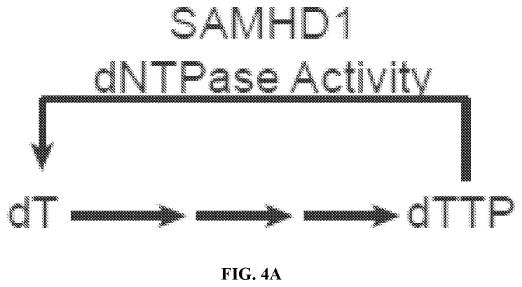

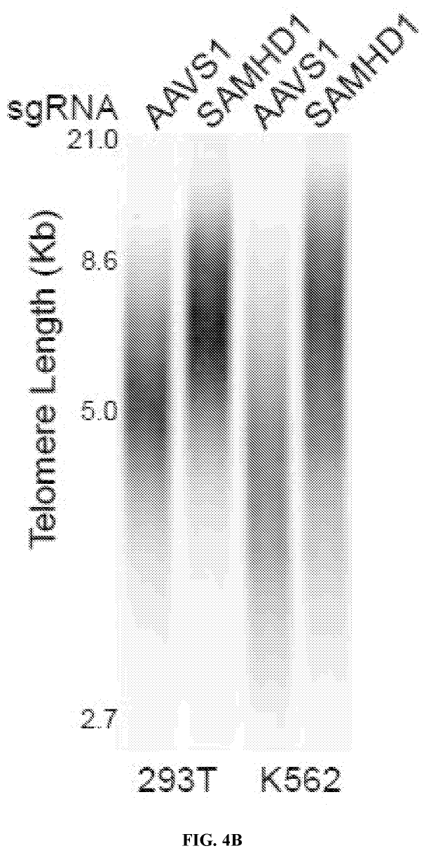

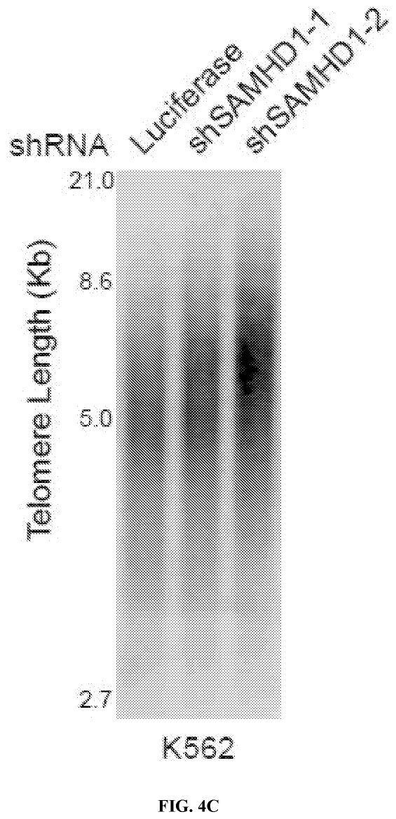

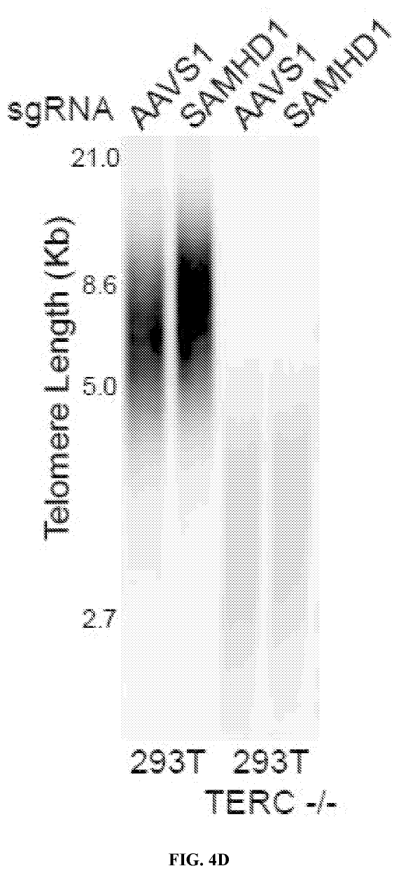

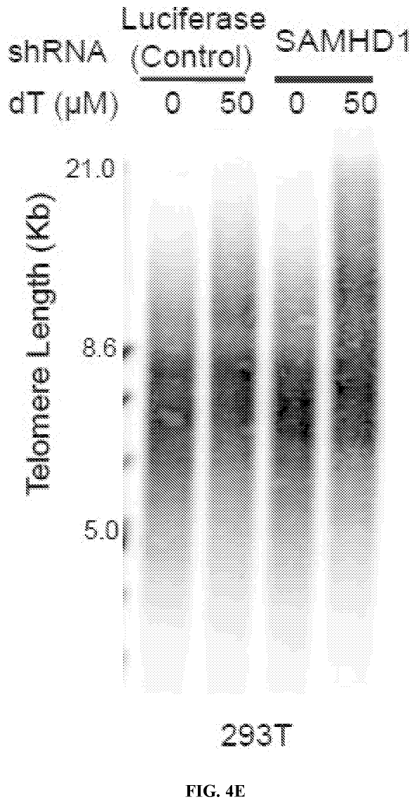

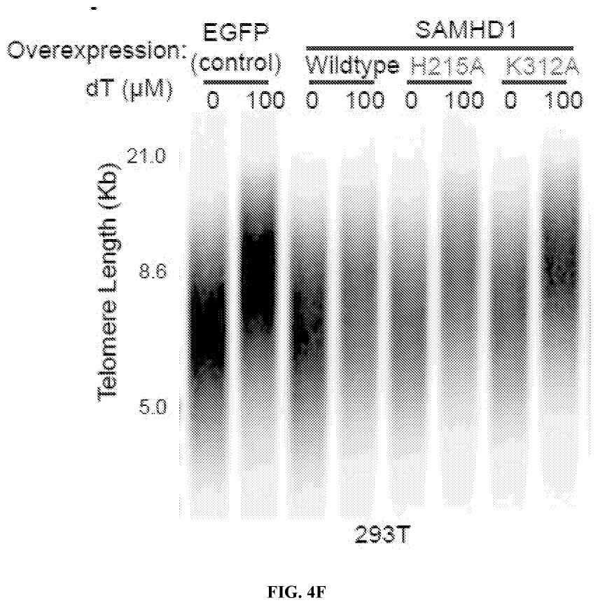

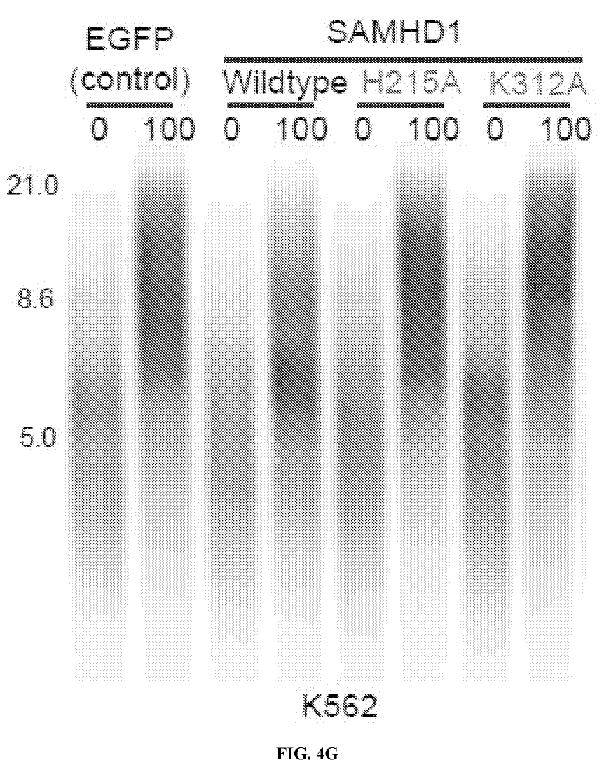

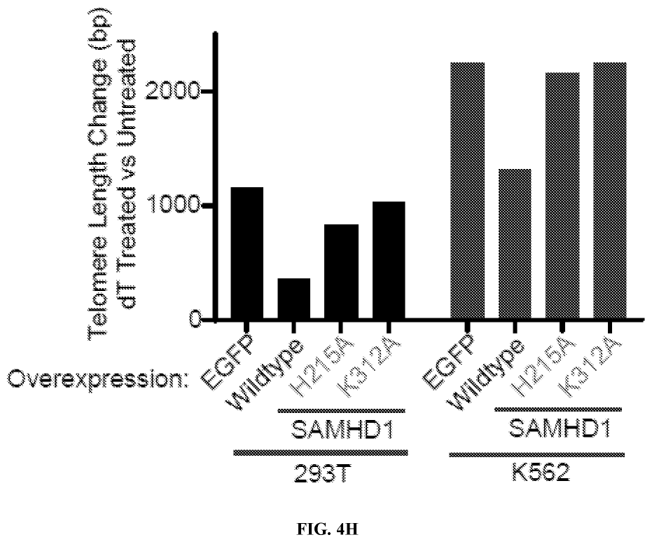

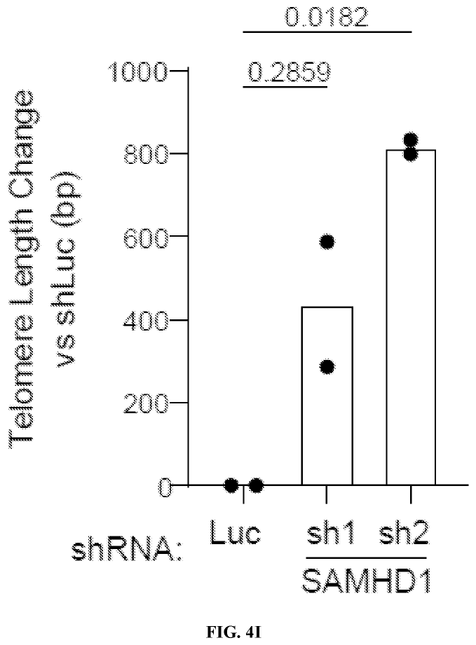

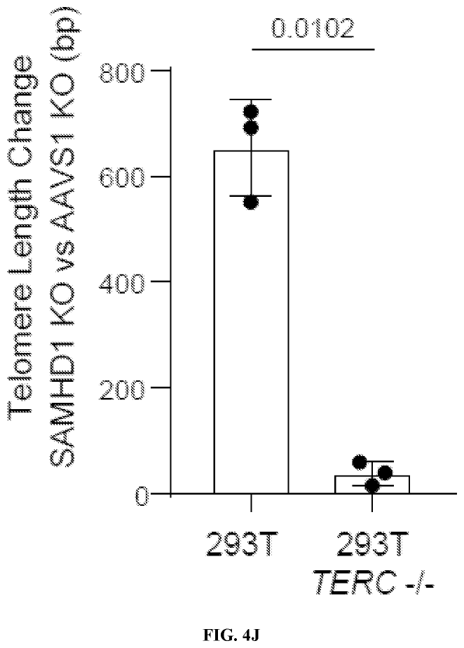

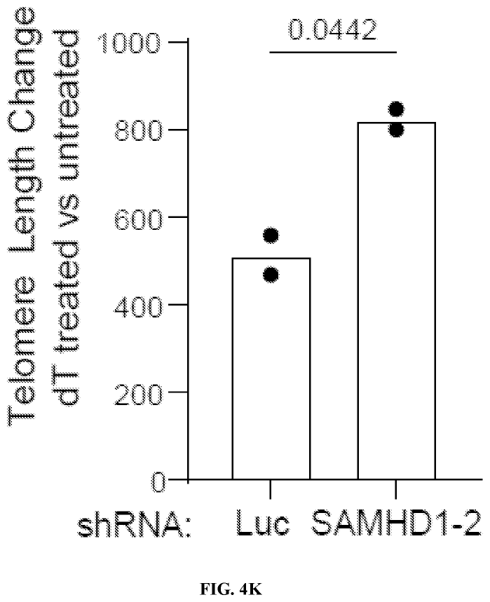

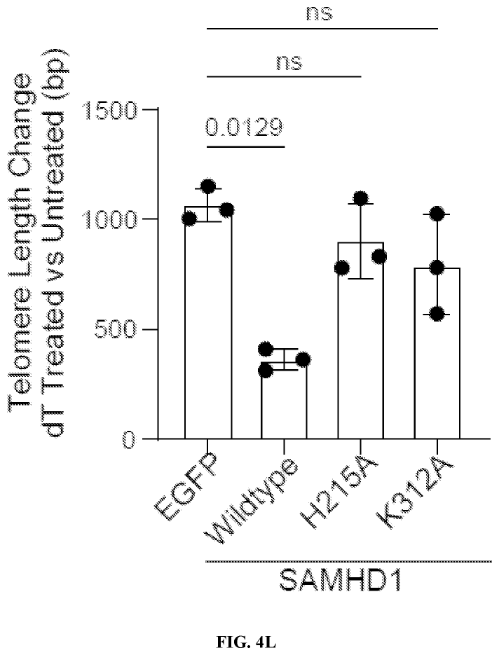

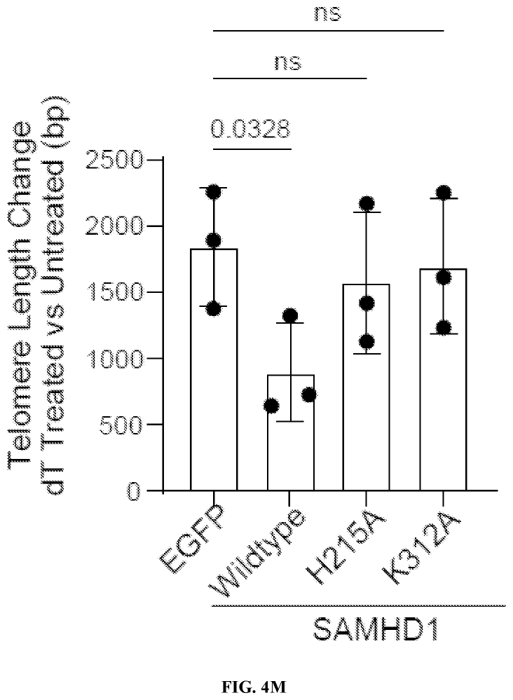

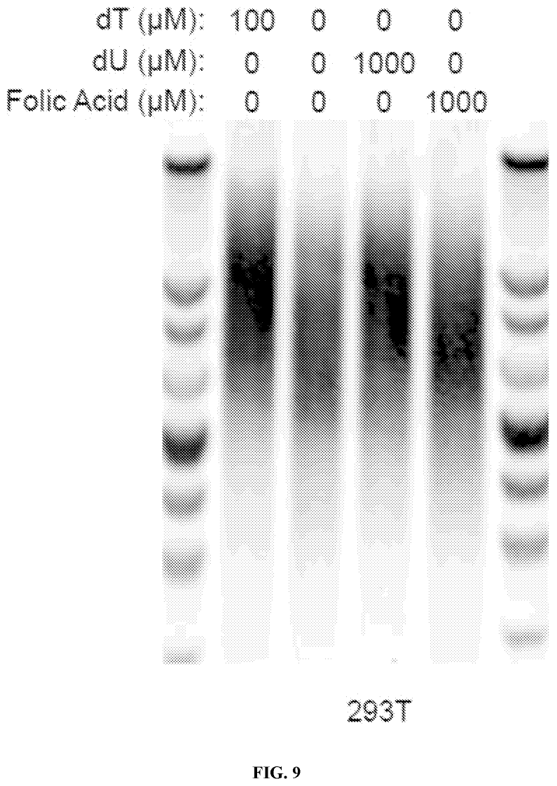

In addition to phosphorylation of dT by TK1 through the salvage pathway, dTMP can also be generated by the conversion of deoxyuridine monophosphate (dUMP) to dTMP by thymidylate synthase (TYMS) using one-carbon transfer from 5,10-methylene-tetrahydrofolate. This conversion is required for de novo synthesis of dTMP in settings without available dT. Deoxyuridine (dU) nucleoside can be taken up by cells and is phosphorylated by TK1 to form dUMP, and therefore the impact of dU supplementation on telomere length was tested. It was found that treatment of 293T cells with dU was tolerated, and could also elongate telomeres to a degree similar to dT, albeit at 10× the concentration (1 mM) (FIG. 9). However, folate did not appear to be limiting, as its supplementation in 293T cells up to 1 mM did not yield telomere elongation (FIG. 4a). To test whether dU increases telomere length via conversion of dUMP to dTMP by TYMS (FIG. 4e), TYMS was targeted in 293T and K562 cells for disruption using CRISPR/Cas9 and confirmed efficient gene knockout by PCR and Sanger sequencing (FIG. 10c). TYMS-deficient cells required salvaged dT for survival (data not shown), thus both TYMS edited and control AAVS1 edited cells were treated with a baseline level of 16 μM of dT. The cells were then treated with either 100 μM of additional dT (116 UM total) or with 1 mM of dU for 10 days and evaluated telomere length. TYMS-knockout cells were found responsive to dT to promote telomere lengthening, while dU was no longer able to promote telomere lengthening in TYMS-deficient cells (FIG. 4f-g). Taken together, these results demonstrate that dU lengthens telomeres via conversion into dTMP, and that promoting dT nucleotide salvage by treatment with dT can lengthen telomeres in cells with deficient de novo dT nucleotide synthesis machinery.

Telomerase reverse transcriptase synthesizes hexanucleotide repeats using cellular nucleotides, and is known to be sensitive to the levels of dNTPs including dTTP in vitro. In various experimental systems, telomerase reverse transcriptase has also been shown to incorporate and be inhibited by non-canonical nucleotides. Having established that conversion into dTMP is required for dT mediated telomere lengthening (FIG. 3B-D), the effect of dT supplementation on the levels of cellular nucleotides was measured as follows: liquid chromatography-mass spectrometry was performed on 293T cells treated with 100 μM of dT for 24 hours, and showing that dT treatment increases levels of dTMP, dTDP and dTTP (FIG. 11). These data are consistent with the genetic data that dT exerts its effects on telomere length by its specific conversion into dT nucleotides within the cell.

### SAMHD1 Restricts Human Telomere Length

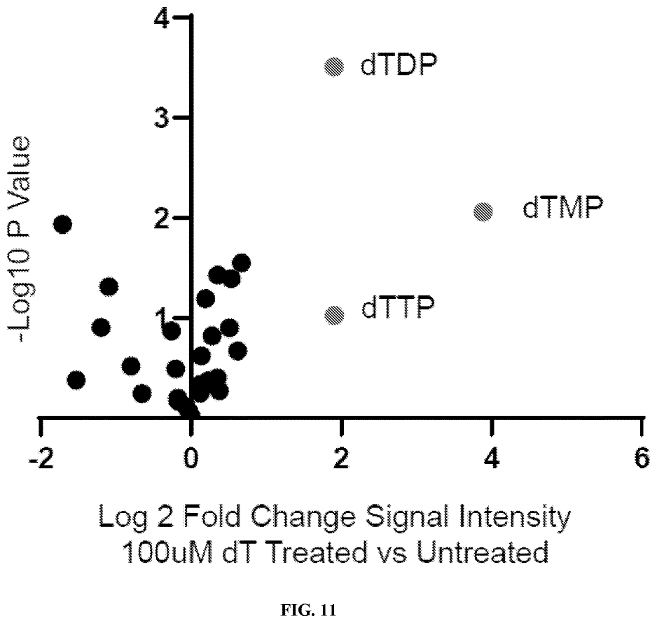

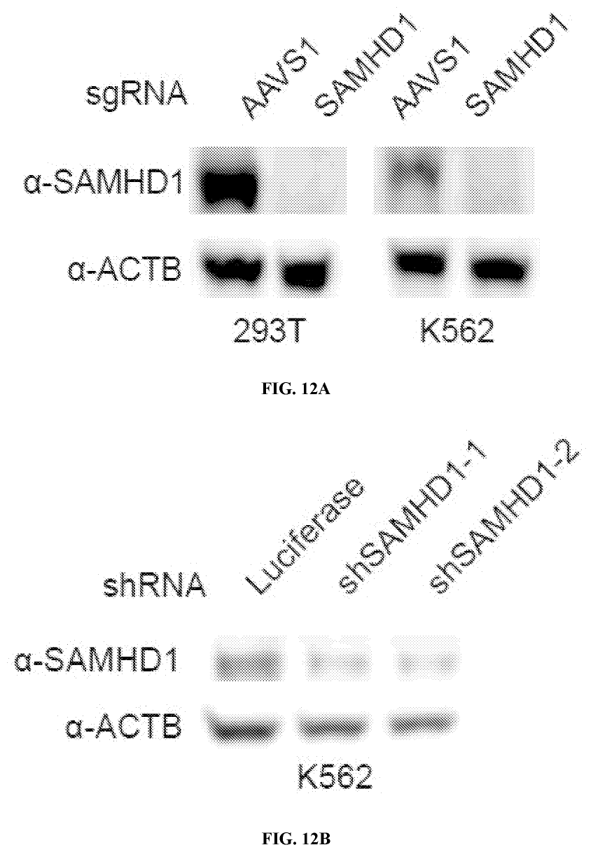

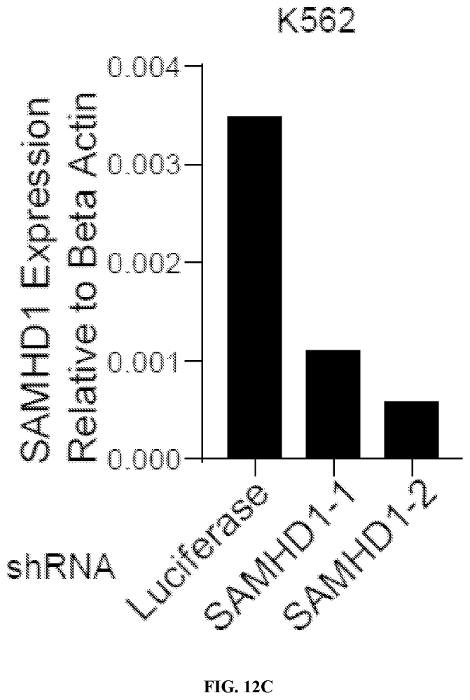

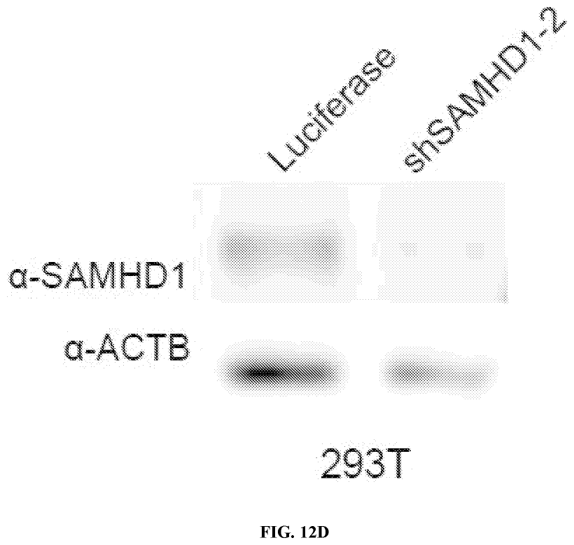

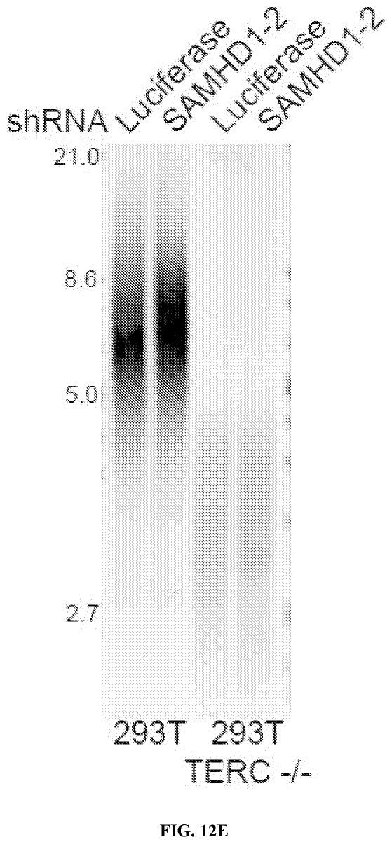

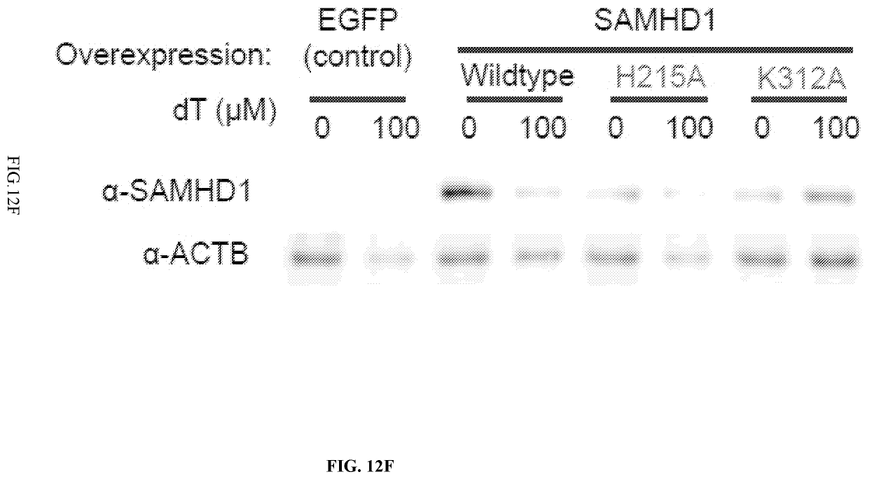

Cellular NTPases play a major role in controlling dNTP levels. The NTPase encoded by the SAMHD1 gene has been implicated in controlling dNTP levels in cells via its capacity to degrade all four canonical dNTPs into dNs, a function which has been suggested to restrict the proliferation of retroviruses by limiting dNTP pools available for reverse transcription. One recent study demonstrated SAMHD1 is enriched at telomeric chromatin, and suggests it may be involved in telomere stability. However, the prior literature do not implicate SAMHD1 in the control of telomere length or telomerase reverse transcriptase activity. In the flow-FISH CRISPR/Cas9 screen for telomere length regulators, SAMHD1 gRNAs scored most strongly as drivers of robust telomere elongation (FIG. 1b). It was therefore directly evaluated how disruption of SAMHD1 could alter telomere length homeostasis in human cells lines, and its relationship to the finding that dT nucleotides elongate telomeres. The screening results were first verified and it was found that deletion of SAMHD1 by CRISPR/Cas9 gene editing (FIG. 4b, FIG. 12a) was tolerated and promoted robust telomere lengthening by thousands of nucleotides in human cell lines. This observation was further validated by inhibiting SAMHD1 using shRNA-mediated RNA interference, which again resulted in telomere lengthening that correlated with the degree of knockdown (FIG. 4c, FIG. 12 b-e). However, no change in telomere length upon SAMHD1 knockout was detected in TERC-null 293T cells (FIG. 4d), indicating that, as it was observed with dT supplementation, telomere lengthening with SAMHD1 loss-of-function occurs by a telomerase-dependent mechanism. Similar results were observed with shRNA knockdown of SAMHD1 in TERC-deficient 293T cells (FIG. 4e). Because SAMHD1 is known to convert dTTP to dT (FIG. 4a), it was determined whether SAMHD1 restricts the degree of telomere lengthening observed after dT treatment. Five days after transduction with a SAMHD1 targeting shRNA, cells were treated with or without 50 μM dT for eight days. It was found that the combined effect of SAMHD1 knockdown and dT treatment produced rapid telomere lengthening (~800 bp increase) that exceeded the sum of telomere lengthening from dT treatment (~470 bp) or SAMHD1 knockdown (~75 bp) individually, showing synergy in telomere elongation from these two perturbations. (FIG. 4e). SAMHD1 has several proposed functions beyond its dNTPase activity including contributing to homology directed repair and replication fork progression. To investigate whether SAMHD1 dNTPase activity is responsible for restricting dT-mediated telomere lengthening, two different dNTPase-deficient versions of SAMHD1 were expressed in cells. It was found that overexpression of wild-type SAMHD1 led to reduced telomere lengthening following dT treatment, compared to that observed in control cells overexpressing EGFP. In contrast, overexpression of the dNTPase-deficient SAMHD 1H215A or SAMHD1K312A variants could not restrict telomere lengthening following dT supplementation (FIG. 4f-h, FIG. 12f). Collectively, these data indicate that SAMHD1 dNTPase activity regulates telomere length homeostasis in human cells, and point to dTTP as the active downstream metabolite responsible for telomere elongation after dT supplementation.

Telomere biology disorders (TBDs) encompass a broad span of genetic disorders of children and adults, and are caused by mutations in at least 15 genes regulating telomere maintenance. To determine that manipulation of dT nucleotide metabolism is therapeutically applicable, it was determined whether treatment with dT or disruption of SAMHD1 could promote telomere lengthening in induced pluripotent stem cells (iPSCs) derived from patients with TBDs. It was first confirmed that dT supplementation was capable of modulating telomere length in iPSCs from a healthy donor (FIG. 5a), which demonstrated elongation of ~2000 bp after treatment with 100 μM dT after three weeks. Next, a panel of iPSCs was tested from TBD patients with varying genetic defects including hypomorphic mutations in in TERC, the telomerase RNA template; DKC1, a component of the telomerase holoenzyme; and PARN which promotes TERC maturation (FIG. 5b-f). Treatment with 50 μM dT for three weeks produced telomere elongation in all cases, including an increase of 640 bp in TERC deficient iPSCs, an increase of 990 bp and 460 bp in DKC1 deficient iPSCs from two different patients, and an increase of 570 bp in PARN deficient iPSCs. Next, the capacity of stable SAMHD1 knockdown to alter telomere length was evaluated in both wildtype iPSCs (FIG. 5g) and patient iPSCs harboring TBD causing mutations, and telomere elongation was found from shSAMHD1-2 across the genotypes tested including DKC1, PARN, and TINF2, a telomere shelterin component (FIG. 5h). Collectively these data demonstrate that manipulation of nucleotide metabolism can restore telomere lengthening in stem cells from patients harboring TBD-causing genetic defects.

Impaired telomere length maintenance is associated with reduced lifespan and fatal degenerative diseases. Using phenotypic CRISPR/Cas9 screening in intact cells, dT nucleotide metabolism was identified as a critical pathway controlling human telomere length homeostasis. DNA precursor levels are tightly controlled though a balance of de novo synthesis, salvage, and catabolismthe results herein demonstrate that telomere length is highly sensitive to changes in dT nucleotide levels: loss of dT nucleotide synthesis or salvage genes led to reduced telomere length while loss of the dNTP degrading gene SAMHD1 led to long telomeres, in a telomerase-dependent manner. While dTTP is a canonical substrate of human telomerase alongside dATP and dGTP, this striking impact of dT nucleotide metabolism on telomere length in human cells is unexpected given prior studies using reconstituted telomerase and yeast indicating dGTP is rate limiting for telomerase activity. Results presented herein, in line with emerging human population genetic data, demonstrate the critical importance of dT nucleotide metabolism in human telomere length control, and highlights the value of examining human telomere length homeostasis in cells.

While defects of nucleotide synthesis are associated with diseases including mitochondrial deficiency and cancer, manipulation of nucleotide metabolism is widely used in life-saving therapies including those for cancer, autoimmune, and infectious diseases. Remarkably, it was found that supplementation with dT promotes rapid telomere lengthening at low micromolar doses, in a telomerase-dependent manner, and without disrupting the cell cycle. This effect was observed across various cell lines as well as in iPSCs derived from patients with TBDs caused by diverse, hypomorphic genetic defects. Based on these findings, along with promising clinical trials currently underway using oral dT supplementation to treat a mitochondrial disorder, it is hypothesized that there may be a therapeutic window to modulate telomere length via manipulating dT metabolism in patients with a range of proliferative and degenerative disorders.

Results in this disclosure establish dT nucleotide metabolism as a critical pathway for the maintenance of telomere length. Whereas dNTP metabolism is commonly considered in relation to DNA replication and repair by DNA polymerases, this disclosure uncovers a strong sensitivity of telomerase reverse transcriptase activity to dT nucleotide homeostasis. Evolutionary pressures on dNTP metabolism have likely faced a tradeoff between telomere length maintenance, nuclear and mitochondrial genomic integrity, and other forces including the restriction of retroviral infections. Telomere length homeostasis offers a new lens to examine the regulation and evolution of DNA precursor metabolism in humans.

### Experiment 1

Thymidine elongates telomeres without inhibiting cell growth. dT is commonly used to inhibit cell cycle progression, arresting cells in S phase at doses in the millimolar range. It was determined whether dT-mediated telomere lengthening could be dissociated from cell cycle effects of dT. When 293T cells were treated with increasing doses of dT, growth was minimally impacted at doses below 100 μM (FIG. 8a). Doses were therefore chosen with no discernable effects on growth and evaluated their impact on telomere length. Supplementation of 293T cells with 20 μM or 40 μM dT for 34 days drove robust telomere lengthening with undetectable impact on cellular growth (FIG. 5). Similar results were seen in K562 cells treated with low doses of dT for 34 days (FIG. 5J, K). DNA content staining and flow cytometry similarly showed no detectable changes in cell cycle distribution at dT doses below 100 μM in 293T cells and below 12.5 μM in K562 cells (FIG. 5L, M). These data indicate that the slowing of cell growth is not required for telomere lengthening from thymidine treatment.

Thymidine Elongates Telomeres without Replication Stress

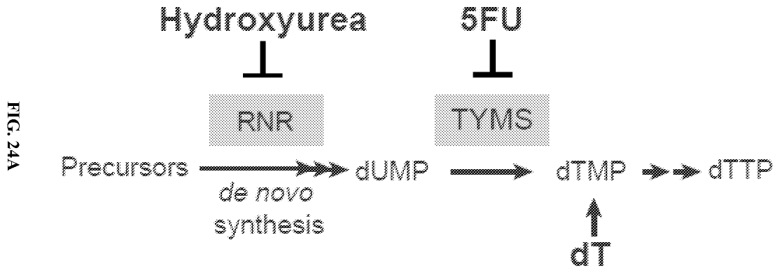

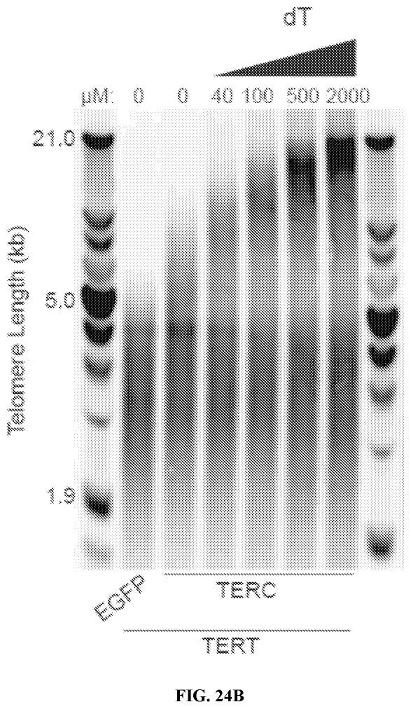

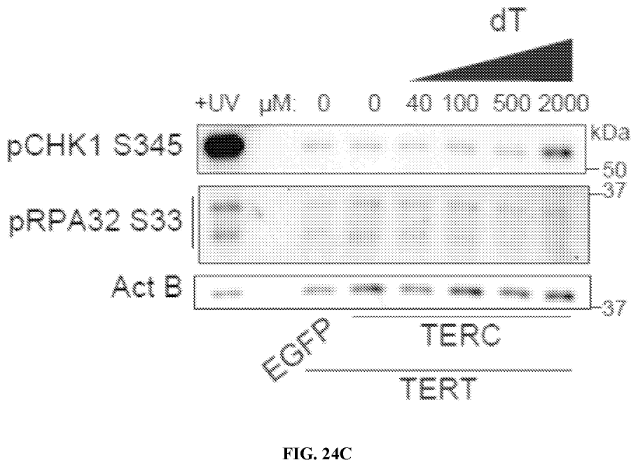

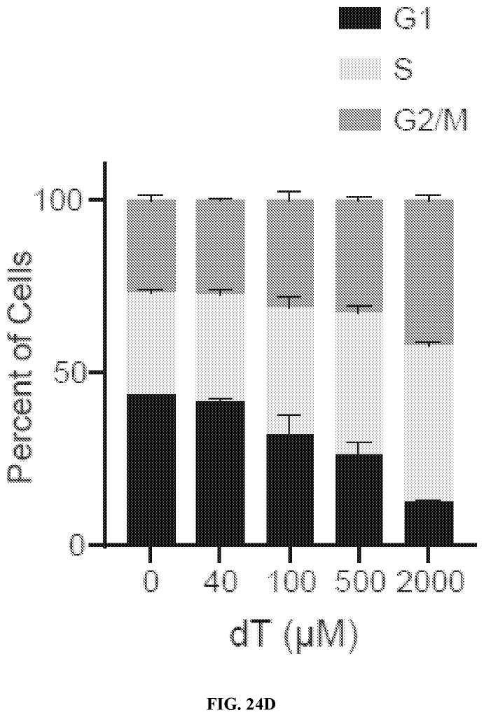

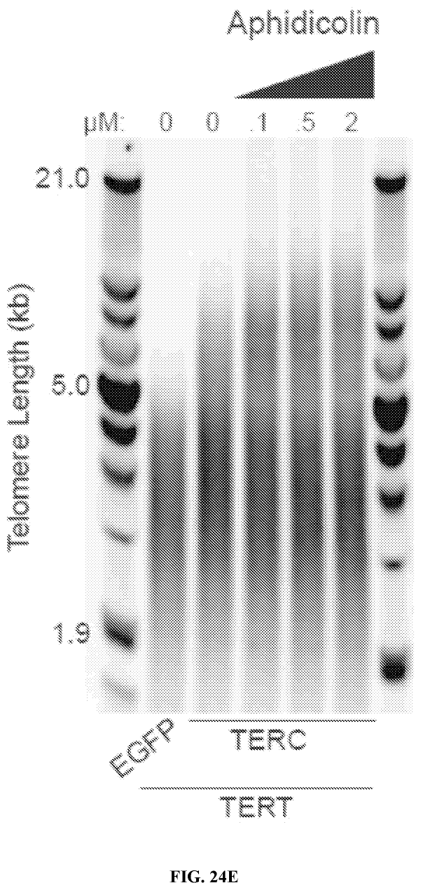

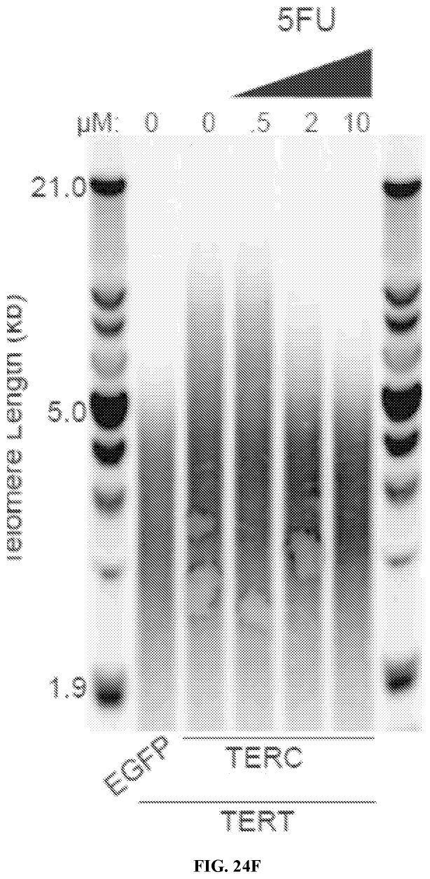

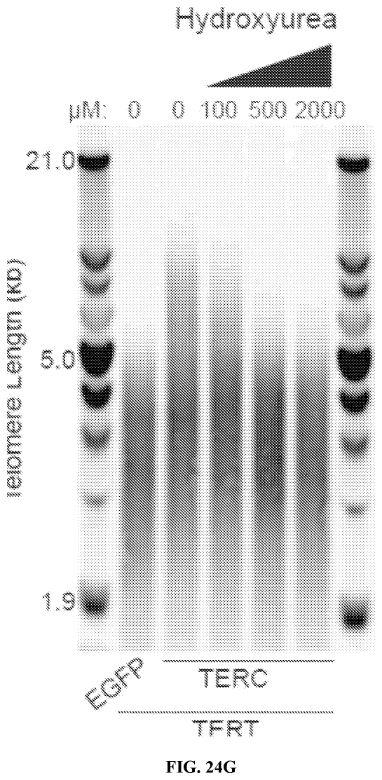

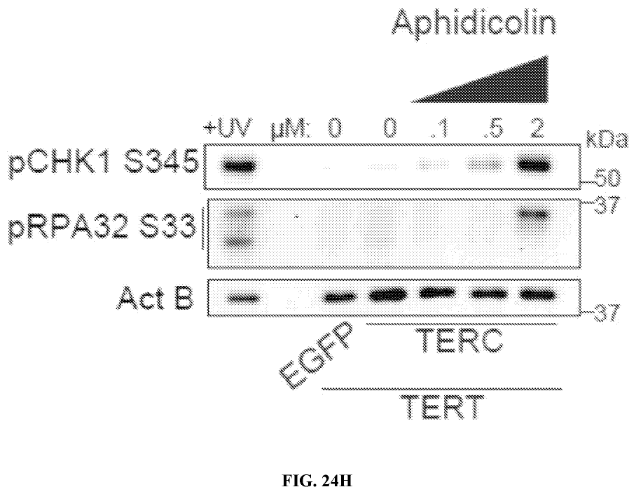

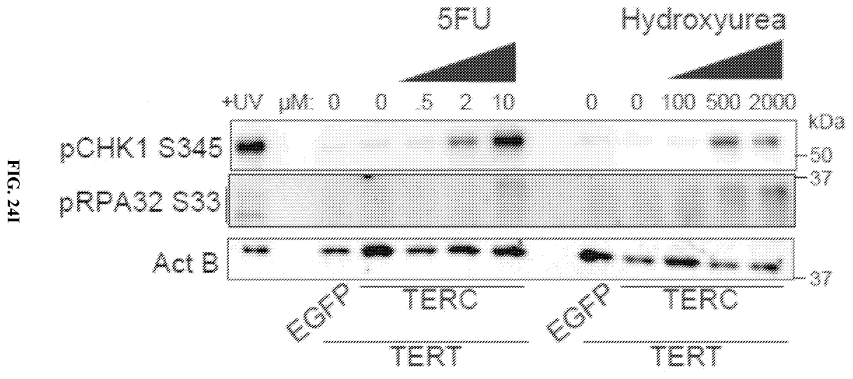

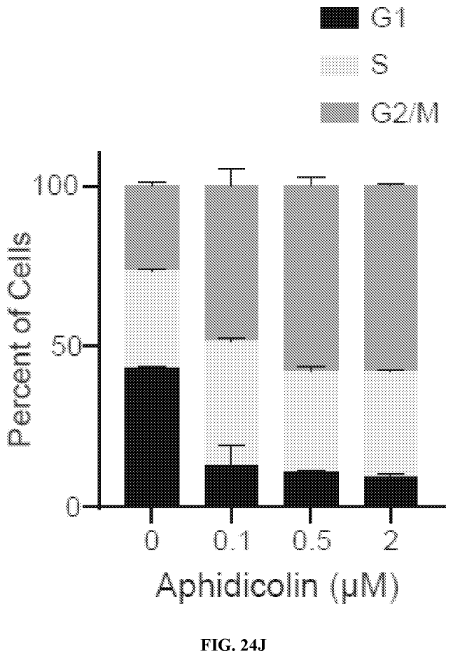

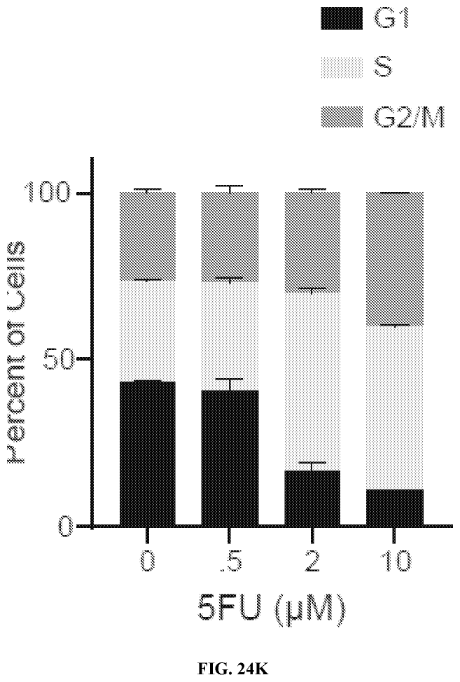

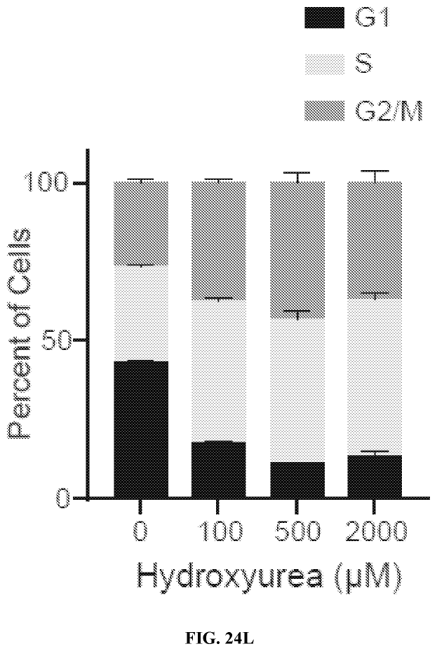

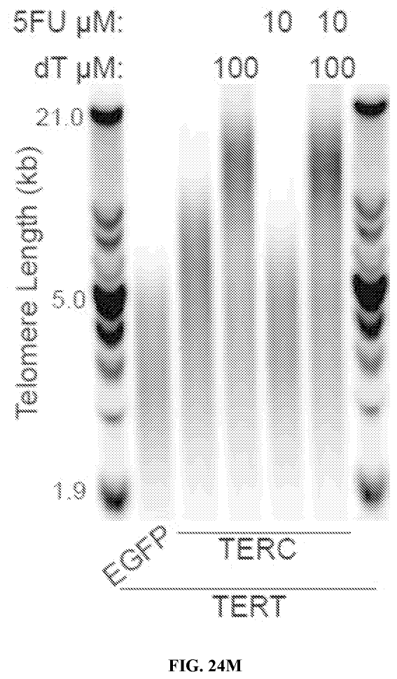

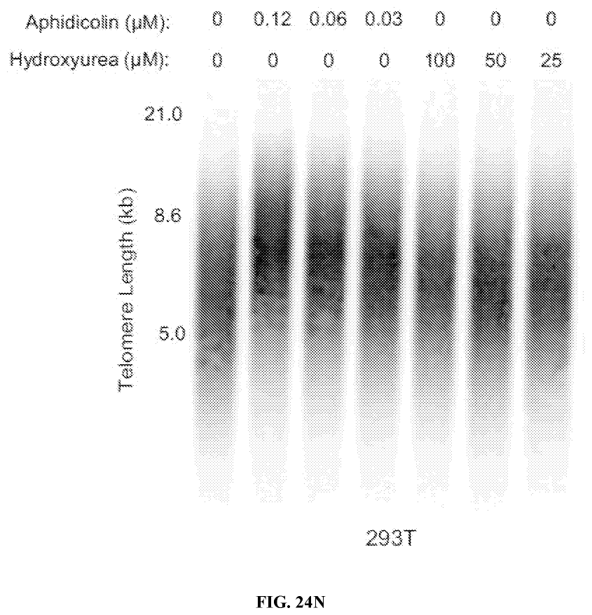

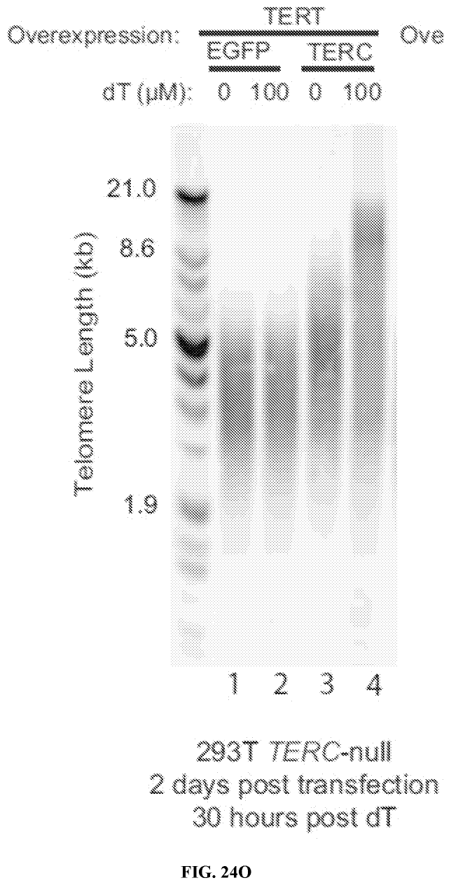

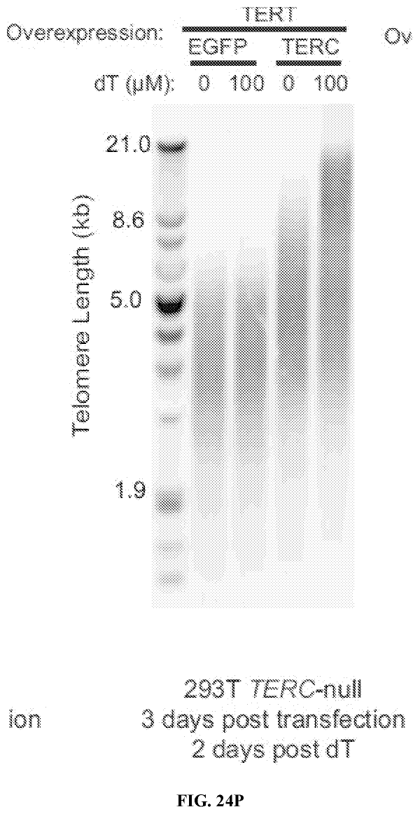

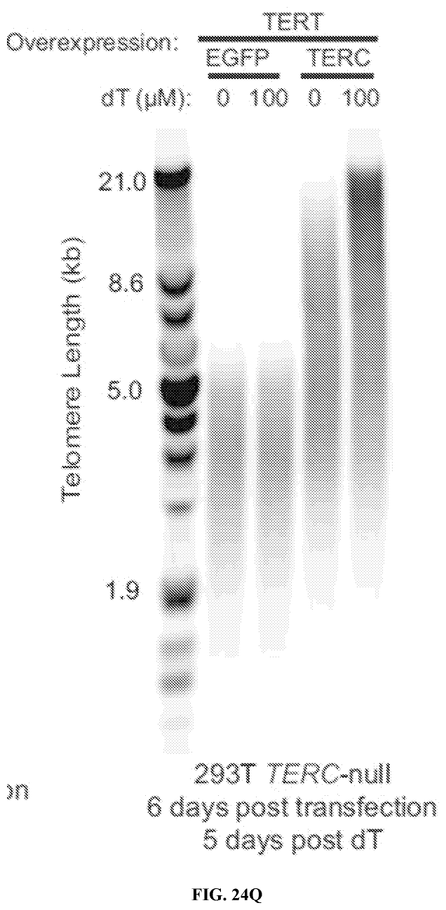

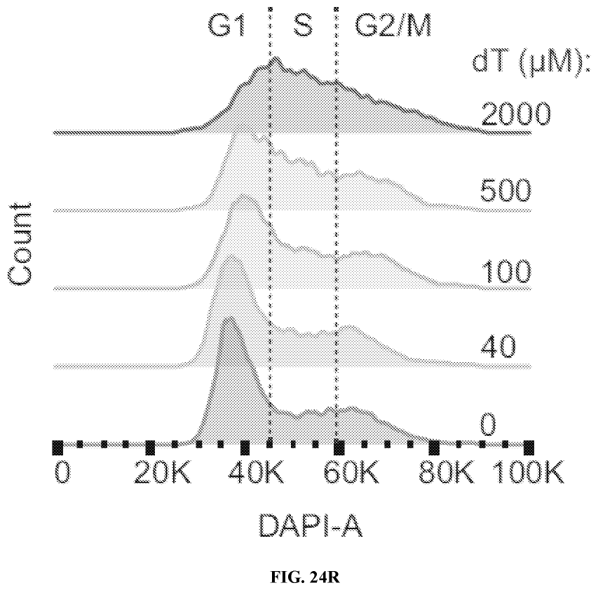

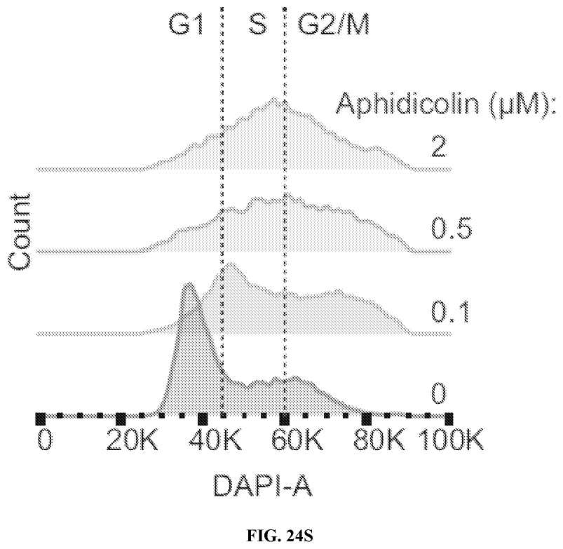

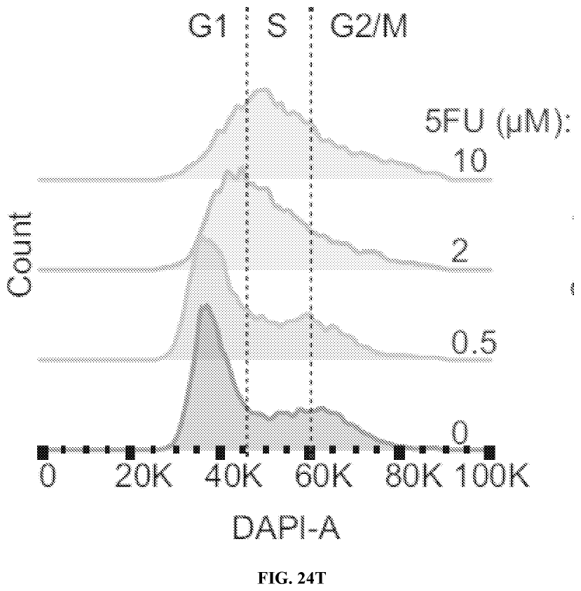

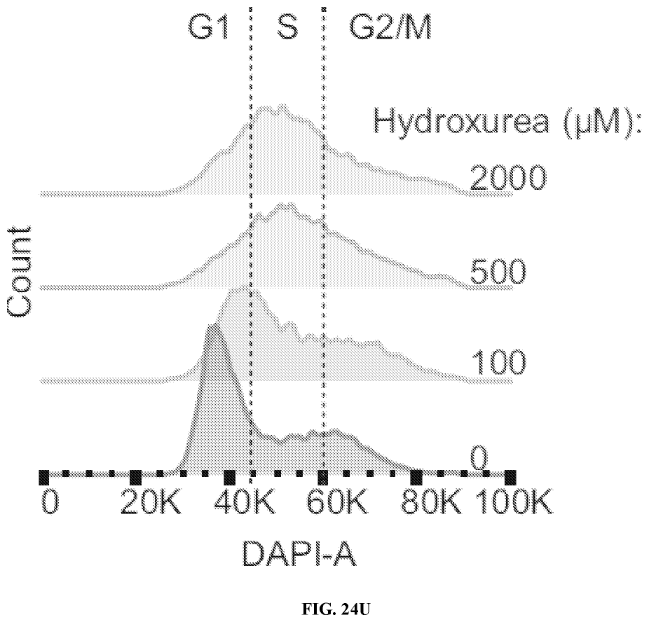

dT supplementation increases levels of dTTP and can reduce dCTP levels, leading to replication stress at high doses. Because replication stress signaling has been implicated in telomerase biology we asked whether replication stress induction explained the telomere length increases seen with dT treatment. We first tested whether telomere elongation is a universal response to replication stress-inducing compounds. When we treated 293T cells with aphidicolin, which inhibits DNA polymerases, at doses maximally-tolerated for cell growth, we found telomere elongation after 10 days (see FIG. 24), as previously observed. However, treatment with hydroxyurea, which blocks deoxyribonucleotide production by ribonucleotide reductase (RNR; FIG. 24a), up to doses permissible for cell growth, did not change telomere length after 10 days (see FIG. 24). This suggested to us that replication stress induction may not cause telomere elongation in all cases. However, cytotoxicity limited our ability to apply higher doses of replication stress-inducing compounds for time periods long enough to study telomere length changes. To study the relationship between replication stress, dT, and telomere length more acutely, we transfected TERC-null 293T cells with expression vectors encoding TERT and TERC, generating ‘super-telomerase’ cells, which showed telomere elongation within two days (see FIG. 24). Remarkably, we found that treatment of super-telomerase cells with dT for only 30 hours robustly increased telomere repeat synthesis in a dose-responsive and telomerase-dependent manner (FIG. 24b). Of note, this effect could be observed with only 40 μM dT, far below doses that induce replication stress signaling as measured by immunoblotting for pCHK1-S345 and pRPA32-S33 (FIG. 24c), and below those that significantly impacted cell cycle progression (FIG. 24d). Using this system, we next asked how high-dose treatment with replication stress-inducing agents such as aphidicolin, hydroxyurea, and the TYMS inhibitor 5FU influence telomere synthesis compared to dT. We found that treatment of super-telomerase cells with aphidicolin for 30 hours drove detectable increases in telomere length, albeit to a lesser extent than dT (FIG. 24e). However, rather than elongating telomeres like dT or aphidicolin, treatment with hydroxyurea or 5FU ablated telomere elongation by telomerase (FIG. 24f,g). Despite the markedly different effects on telomere biology seen at the highest doses of dT, aphidicolin, 5FU, or hydroxyurea, all induced similar levels of replication stress signaling (FIG. 24h,i) and had similar impacts on cell cycle progression (FIG. 24j-l). Collectively, these data demonstrate that replication stress signaling induction does not universally promote telomere synthesis and does not explain the telomere lengthening in cells treated with dT at low doses.

Substrate-Independent Enhancement of Telomerase by dTTP

The timeframe and magnitude of the effects of dT and 5FU on telomeres in super-telomerase cells strongly suggested to us that thymidine nucleotides might directly impact telomerase activity. Given that high dose 5FU completely inhibited telomere elongation in super-telomerase cells (FIG. 6g) and acts by inhibiting TYMS to limit de novo dTTP production, we asked whether dT supplementation could rescue telomere synthesis following 5FU treatment. Indeed, we found that dT treatment restored telomere elongation despite maximal doses of 5FU (FIG. 6m). These data show that 5FU inhibits telomerase activity by limiting de novo thymidine nucleotide production and provide further evidence that thymidine nucleotide synthesis is required for telomerase activity.

The effects of thymidine nucleotides on telomerase activity could be from increasing the quantity of telomerase holoenzymes per cell, or alternatively by enhancing telomerase function. To test the former possibility, we measured the abundance of active telomerase enzymes in cells using the telomeric repeat amplification protocol (TRAP) assay and found no difference in telomerase activity in lysates from 293T cells treated with or without 100 μM dT (FIG. 7a,b). These data indicate that dT treatment does not change the overall quantity of telomerase holoenzymes in cells. Next, to test the effects of dTTP on telomerase function in vitro, we performed the TRAP assay with increasing levels of exogenous dTTP and found enhanced telomerase activity (Extended Data FIG. 7a,b). However, because dTTP is a direct substrate for GGTTAG repeat synthesis by telomerase, these experiments could not distinguish substrate-dependent versus substrate-independent effects of dTTP on telomerase activity. To study this further, we engineered a modified TERC expression vector with the template region encoding “T-free” repeats (GGAAAG) rather than wild-type (GGTTAG) repeats (FIG. 7c), allowing us to interrogate potential substrate-independent effects of dTTP on telomerase activity. When we tested T-free super-telomerase cell extracts in a modified TRAP assay (Extended Data FIG. 7c,d), we found increased T-free telomerase activity with increasing dTTP levels (Extended Data FIG. 7e-h). Sequencing T-free telomerase TRAP products demonstrated that thymidine was not represented in the extended products (Extended Data FIG. 7i-k). These results confirmed that T-free telomerase does not use dTTP as a substrate, and surprisingly suggested a substrate-independent effect of dTTP on telomerase activity. To determine this without the potential confounder of PCR amplification in the TRAP assay, we directly tested the effects of dTTP on immunopurified T-free telomerase, as measured by incorporation of α-³²P-dATP on a telomere-repeat oligonucleotide substrate. Remarkably, dTTP enhanced T-free telomerase activity in a dose-dependent manner (FIG. 7d), with levels approximately 50% higher at a concentration of 25 μM (FIG. 7e,f), and with a greater effect on longer telomerase products suggestive of increased telomerase processivity (Extended Data FIG. 7l). Taken together, these results demonstrate that dTTP is capable of increasing telomerase activity in a manner independent of its role as a substrate, potentially through an allosteric mechanism.

### Substrate-Independent Control of Telomerase in Cells by dT

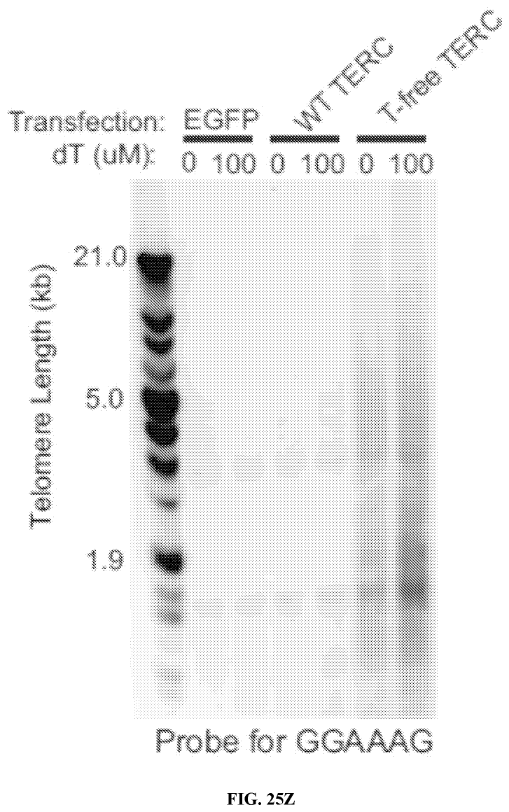

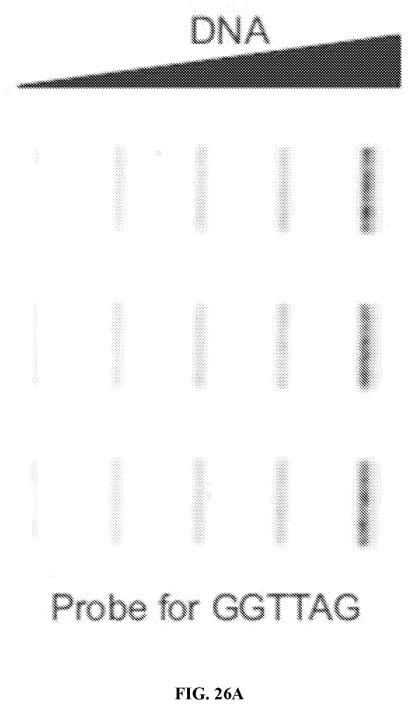

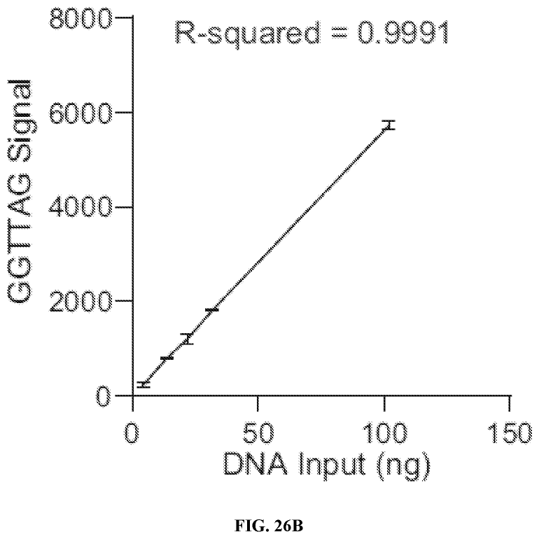

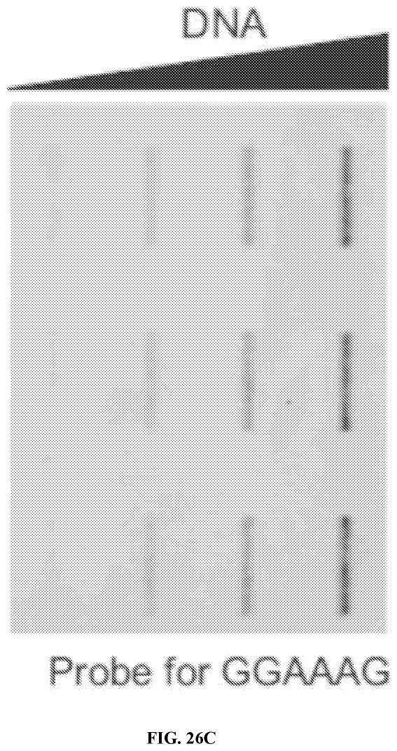

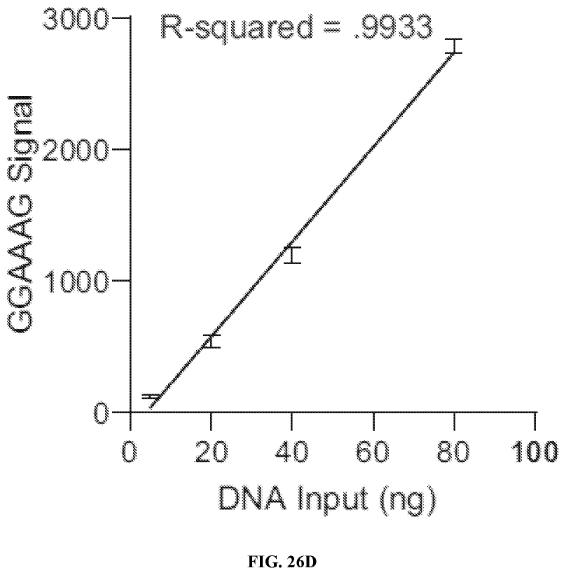

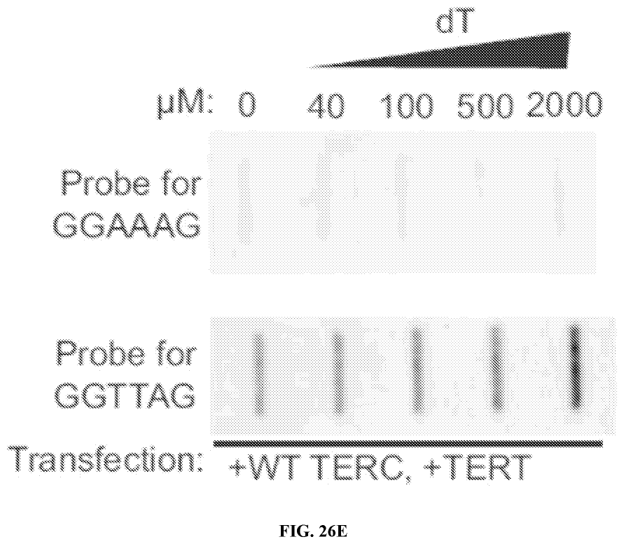

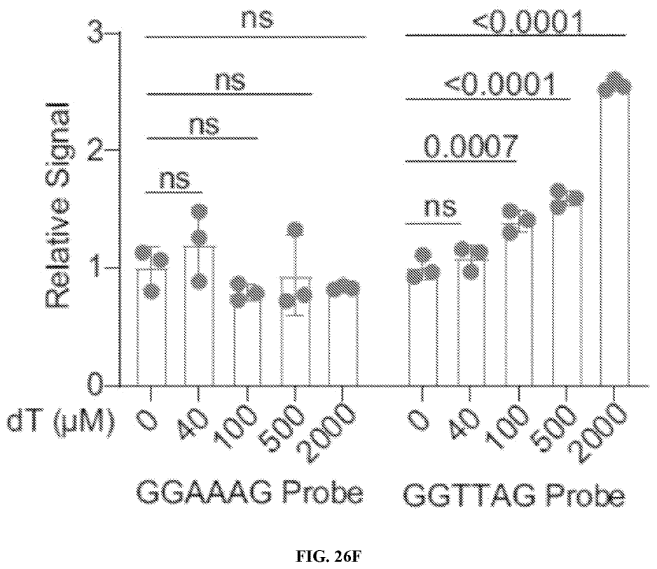

After observing this substrate-independent effect of dTTP on telomerase activity in vitro, we next asked whether dT treatment could enhance T-free super-telomerase activity in cells. When we transfected TERC-null 293T cells with TERT and T-free TERC vectors, we detected altered repeat sequences on native telomere ends by PCR (FIG. 25g,h). Southern analysis showed increases in telomerase-dependent GGAAAG signals in response to dT. However, these signals appeared over a range of molecular weights rather than just elongation of pre-existing telomere ends as seen with the native GGTTAG template (FIG. 25y,z). This might be explained by instability of an unprotected G-rich GGAAAG repeat extension, which likely cannot be bound by POT1, cannot undergo C-strand fill-in due to the requirement for pol-alpha/primase to begin the RNA primer with a purine (templated by “C” or “T”, now absent from T-free G-rich strand), and cannot efficiently complement the native telomere sequence to form a T-loop, thus potentially subject to nucleolytic cleavage. Nevertheless, to quantify T-free telomerase repeat synthesis in cells, we used slot blotting to measure GGAAAG repeat content in cellular DNA (FIG. 26a-d) and found a specific increase in GGAAAG repeats in cells overexpressing T-free super-telomerase (FIG. 25i,j). When we treated cells overexpressing wild-type versus T-free super-telomerase with 100 μM dT, we found an increase in GGAAAG repeat content exclusively in cells expressing T-free telomerase (FIG. 25i,j and FIG. 26e, f). Furthermore, we found that treatment with 5FU or hydroxyurea reduced levels of GGAAAG repeats in T-free super-telomerase expressing cells, and that treatment with dT could rescue the inhibition of GGAAAG repeat synthesis following 5FU treatment (FIG. 25k,l), patterns identical to those found with the native template (FIG. 24m). These findings support a model wherein dTTP increases telomerase activity in human cells by a mechanism independent of its role as a telomerase substrate.

Thymidine Manipulation Elongates Telomeres in Patient iPSCs

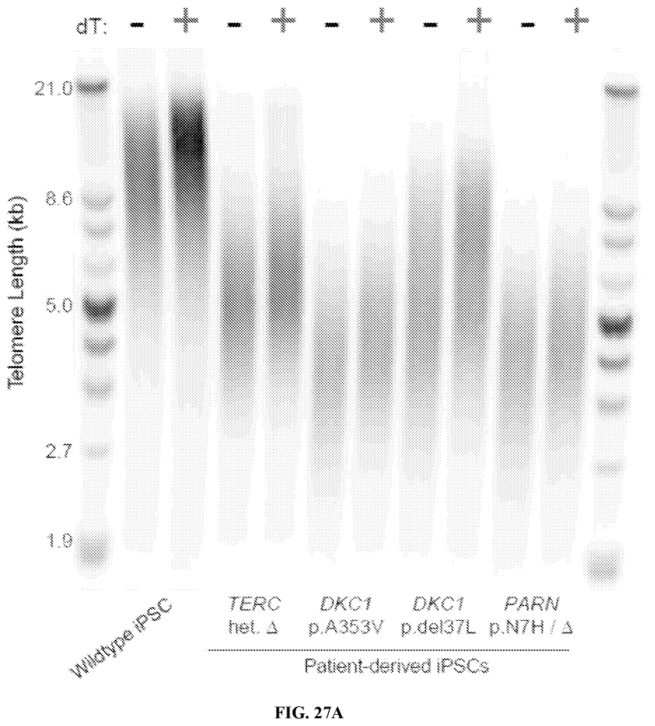

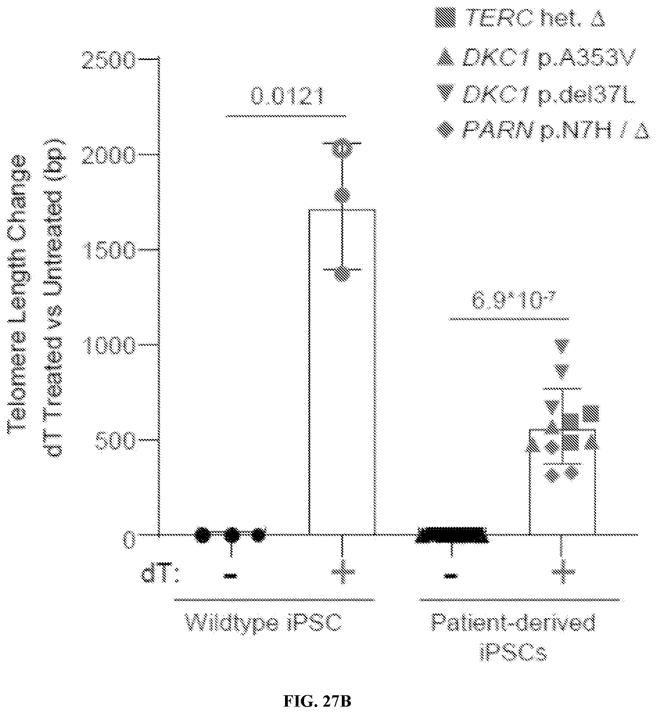

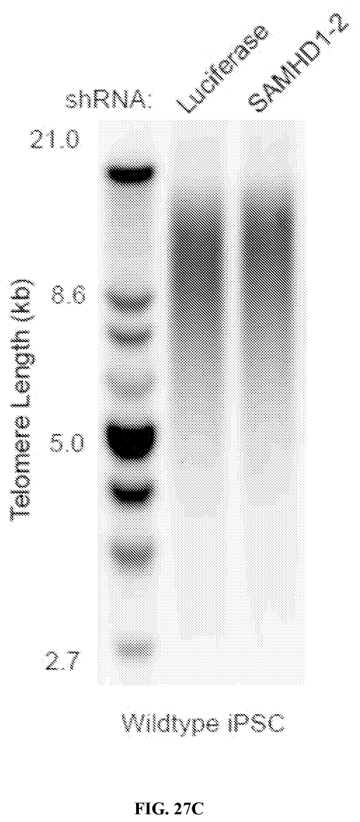

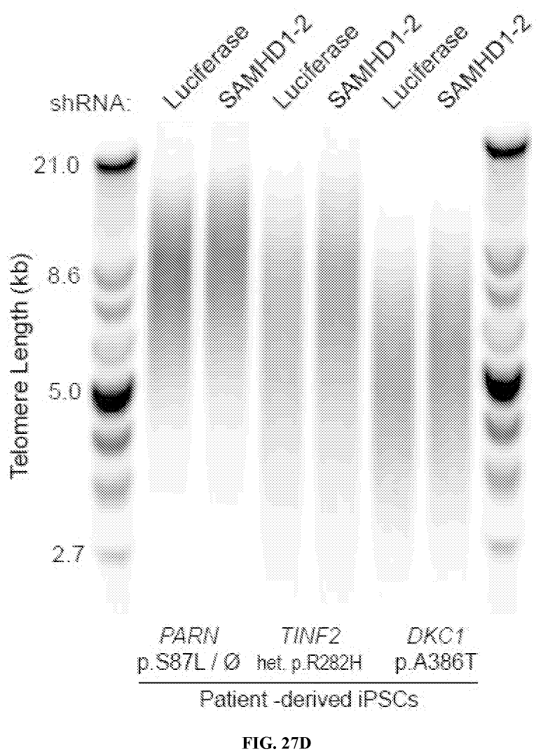

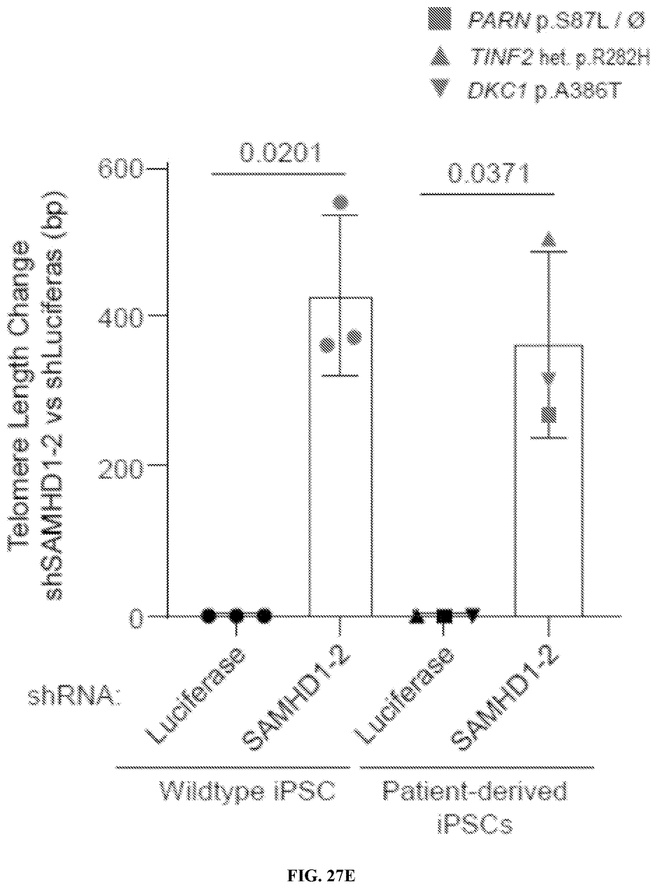

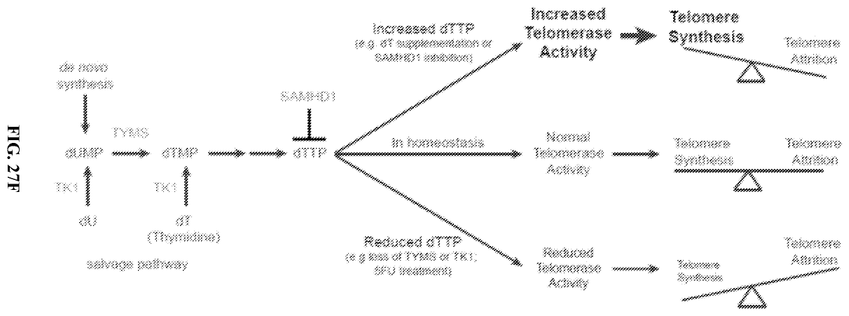

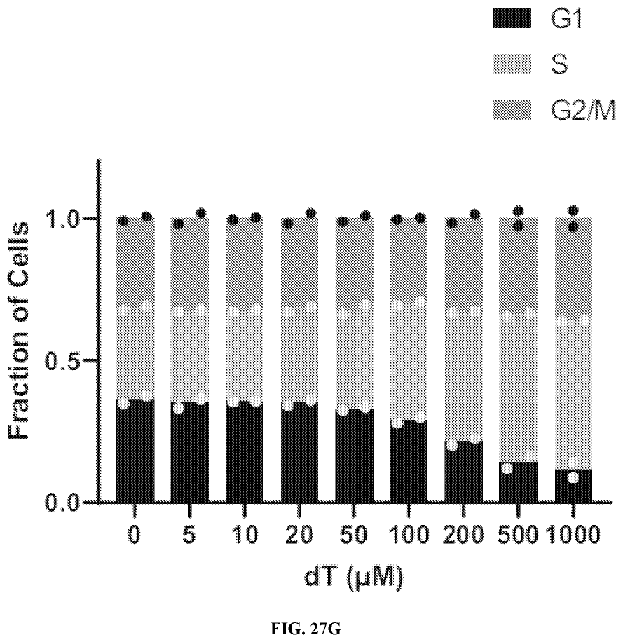

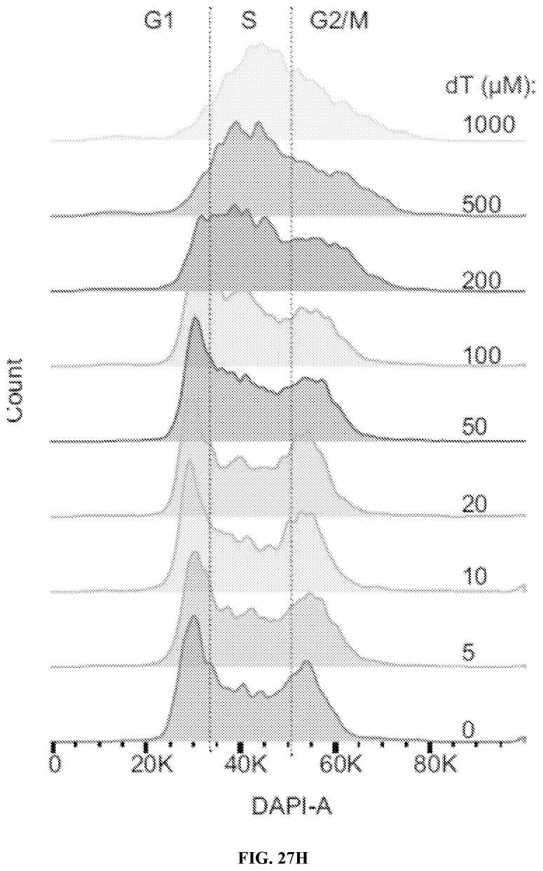

Telomere biology disorders (TBDs) are caused by mutations in at least 18 genes regulating telomere maintenance. We asked whether treatment with dT or disruption of SAMHD1 could promote telomere lengthening in induced pluripotent stem cells (iPSCs) derived from patients with TBDs. We first confirmed that dT supplementation for three weeks significantly elongated telomeres in iPSCs from a healthy donor (FIG. 27a,b). We next tested a panel of iPSCs from TBD patients with hypomorphic genetic defects, including mutations in TERC, DKC1 (encoding a component of the telomerase holoenzyme), or PARN, which promotes TERC maturation. We found that treatment with 50 μM dT for three weeks produced telomere elongation in all cases (FIG. 27a,b). When we examined the impact of dT treatment on cell cycle progression in iPSCs, we found doses≤50 μM had minimal effects (FIG. 8g-i). Assessment of pCHK1-S345 and pRPA32-S33 by immunoblotting revealed no indication of replication stress signaling in iPSCs upon treatment with dT (FIG. 27i), in line with prior literature. We next evaluated the capacity of stable SAMHD 1 knockdown to alter telomere length in both wild-type iPSCs and patient iPSCs harboring TBD-causing mutations and found telomere elongation across the genotypes tested, including DKC1, PARN, and TINF2, which encodes a telomere shelterin component (FIG. 27c-e). Collectively, these data demonstrate that manipulation of thymidine nucleotide metabolism can restore telomere lengthening in stem cells harboring TBD-causing genetic defects.

### Experiment 2

Telomeres are repetitive DNA-protein structures which prevent chromosome ends from being recognized as DNA breaks, promoting genomic stability. Hematopoietic stem cells (HSCs) express telomerase to maintain telomere length, which gets shorter with age in blood cells. Inherited defects in telomere related genes can cause abnormally short telomeres and have been associated with predisposition to a spectrum of hematopoietic defects. These include the severe childhood bone marrow failure syndrome dyskeratosis congenita (DC), isolated aplastic anemia, and myelodysplastic syndrome. DC is one of a broader spectrum of telomere biology disorders (TBDs) characterized by degenerative diseases throughout the body including liver cirrhosis and pulmonary fibrosis. Bone marrow transplant can be used to treat hematopoietic defects, but this is not feasible for all patients, and does not treat the underlying cause of this systemic disease, leaving transplanted patients at risk for secondary organ failure. Despite our growing appreciation for the prevalence of these disorders, there are currently no effective systemic therapies to treat TBDs, and accordingly the thousands of patients diagnosed with these disorders each year face poor prognoses. There is substantial human genetic evidence that interventions to increase telomere length would be beneficial to these patients. For example, blood cells from TBD patients have been found to harbor somatic mutations predicted to increase telomere length through multiple mechanisms including activating mutations in the promoter of the telomerase reverse transcriptase gene (TERT), encoding the catalytic subunit of telomerase which synthesizes new telomere repeats. This and other human genetic data indicate that cells with restored telomere maintenance mechanisms have a growth advantage, and suggests that a therapy to lengthen telomeres could improve hematopoietic output and tissue regenerative capacity in TBD patients. Therefore, new systemic therapies to increase telomere length and treat the underlying cause of TBDs are needed. A genome-wide screen in human blood cells was recently performed and identified deoxyribonucleotide (dNTP) metabolism genes as powerful regulators of telomere length, findings independently corroborated by human population-level genetic studies. This example shows data that loss of the dNTPase SAMHD1 and/or supplementation with thymidine, a precursor to dTTP, can rapidly increase telomere length in human cells. Based on the strength of this evidence, there is a critical need to understand the control of telomere homeostasis by nucleotide metabolism in hematopoiesis. This knowledge establishes a link between telomere biology and dNTP metabolism and leads to advancement of human health. The overall objectives for this example are to uncover the mechanisms by which SAMHD1 and thymidine metabolism control telomere length, and to show the extent to which manipulating SAMHD1 and/or thymidine metabolism promotes telomere lengthening in hematopoietic models of TBDs. The experiment studies how SAMHD1 and thymidine metabolism control hematopoietic cell telomere length by regulating telomere repeat synthesis by telomerase.

The role of SAMHD1 in hematopoietic telomere length homeostasis. Based on the data herein, the loss of SAMHD1 dNTPase activity at the telomere increases dNTPs available for telomere synthesis by telomerase, promoting telomere elongation. Interrogating SAMHD1 separation of function mutants shows impact on telomere length regulation. Gene editing of primary human hematopoietic stem and progenitor cells is expected to show SAMHD1 loss of function impacts telomere length and telomerase function in hematopoietic models of TBDs.

Mechanisms by which thymidine metabolism contributes to telomere length control in hematopoietic cells: based on the data, thymidine alters the physiologic abundance of nucleotides at the telomere to control telomerase activity. Genetic, biochemical, and metabolomic based approaches show how telomerase activity is altered by thymidine metabolism manipulations. Primary human hematopoietic cells are used herein to evaluate how thymidine supplementation alters hematopoietic function and telomere length homeostasis in vitro and in vivo. The experiment helps to define the mechanisms by which nucleotide metabolism regulates telomere length homeostasis in the hematopoietic system. These experiments uncover a novel aspect of telomere length control in humans and lay the groundwork on the use of dNTP metabolism manipulations to therapeutically increase telomere length for the treatment of TBDs and other degenerative diseases.

Orthogonal genome-wide studies associate nucleotide metabolism genes and telomere length. To identify novel pathways regulating telomere length, a genome-wide CRISPR-Cas9 knockout screen was done in human K562 cells by sorting for telomere length using Flow-FISH, a fluorescence based telomere length assay. The screen was robust as indicated by identification of several telomere length associated genes including telomerase reverse transcriptase (TERT) as well as a number of nucleotide metabolism genes. Based on these results, a second round of screening was performed using a custom gRNA library targeting 53 nucleotide metabolism genes. This approach identified the dNTPase SAMHD1 as well as several thymidine metabolism genes as regulators of telomere length (FIG. 1b). As independent support for the validity of these results, two recent genome wide association studies (GWASs) associated several dNTP metabolism genes with telomere length measured in human blood cells including DCK, TK1, TYMP, TYMS, and SAMHD1 (FIG. 1c). Prior to CRISPR screens and these GWASs, none of these genes have been connected to human telomere length. These data provide strong genetic evidence of a novel, unexpected, and unpredictable connection between dNTP metabolism genes and human telomere length homeostasis.

Telomerase is sensitive to changes in nucleotide levels. Telomerase is a reverse transcriptase which synthesizes telomeric TTAGGG repeats using an RNA template encoded by the TERC gene. Studies in yeast and on reconstituted telomerase have indicated that telomerase activity is sensitive to concentrations of dNTPs with dGTP>dTTP>dATP in order of importance. More recent work showed reconstituted telomerase incorporates deoxyribonucleoside diphosphates (dNDPs) as well as dUTP and ribonucleotides which could influence telomere homeostasis. Thus, changes in available nucleotide concentrations influence telomerase activity.

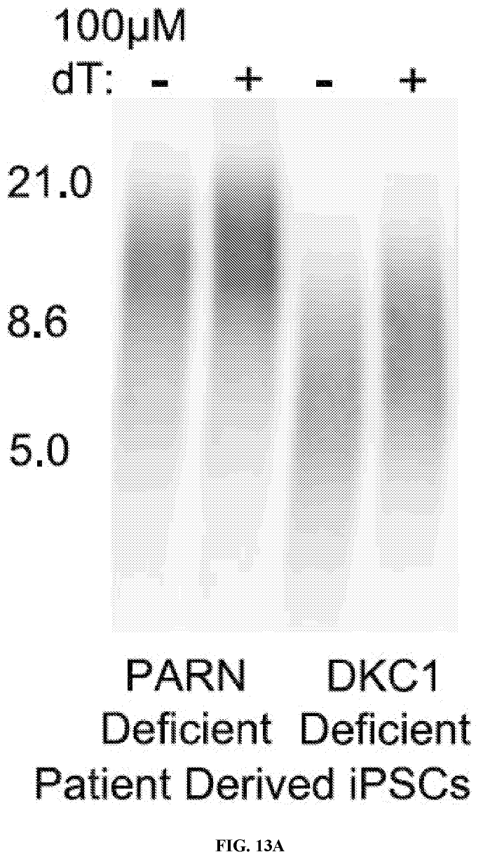

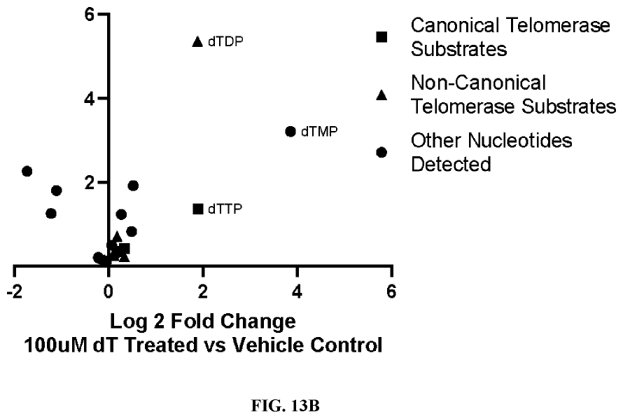

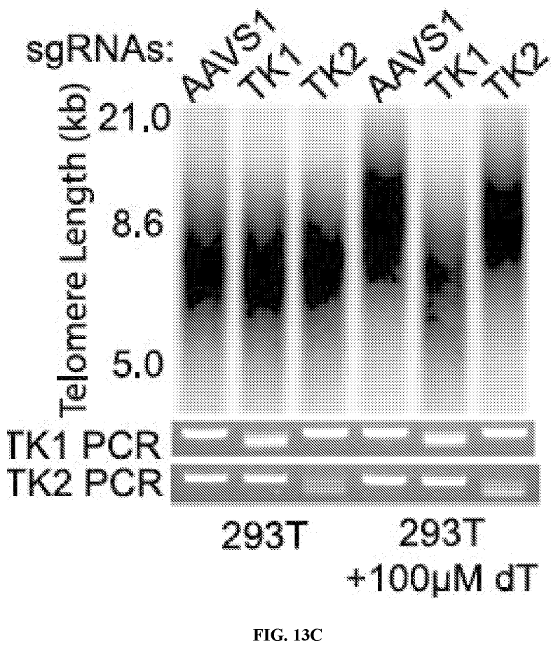

Thymidine metabolism is critical for telomere length control in human cells: to directly investigate how perturbing dNTP metabolism influences telomere length, cell culture media was supplemented with deoxyribonucleosides (dNs) which can be converted to dNTPs by salvage pathway kinases. dN supplementation rapidly and robustly increased telomere length in human cell lines (FIG. 2A). Treatment of telomerase deficient TERC−/− 293T cells with dNs did not alter telomere length (FIG. 2A). These results support a model where dN supplementation impacts telomerase activity to alter telomere length, and argue against other recombination-based telomere elongation mechanisms (known as alternative lengthening of telomeres (ALT)) being at play. Of the canonical dNs, thymidine (dT) was necessary and sufficient for telomere lengthening (FIG. 2B). This result aligns with both screening studies herein and the aforementioned GWASs, which were enriched for hits in dT metabolism genes (FIG. 1A-B). It was further demonstrated that dT treatment increased telomere length in TBD patient-derived induced pluripotent stem cells (iPSCs) with two different disease-causing mutations that cause partial loss of telomerase activity (FIG. 13A). Importantly, the dose of dT used to achieve these effects (100 μM) is lower than the doses typically used to synchronize cells in S phase (1-2 mM). To quantify how dT supplementation alters levels of dTTP as well as other telomerase substrates, liquid chromatography-mass spectrometry (LC-MS) was used to measure polar metabolites (FIG. 13B). dT treatment increased levels of dTTP by four-fold compared to vehicle, while changes in dATP or dGTP levels were not detected. Amongst the known non-canonical telomerase substrates, dT treatment increased dTDP levels fourfold. Collectively, these data show that thymidine metabolism is uniquely important for telomere length maintenance. The data further provide evidence against a nonspecific effect of nucleoside supplementation driving telomere length changes, for example through replication stress or altering cell cycle dynamics.

Thymidine kinase 1 (TK1) is required for telomere lengthening after thymidine treatment. There are separate dTTP pools used for mitochondrial and nuclear DNA synthesis. To study whether dT mediated telomere lengthening occurs through modification of mitochondrial or non-mitochondrial nucleotide pools, this example generated cells knocked out for TK1, which catalyzes the first step in salvage of dTTP in the cytosol for use in nuclear genome synthesis, or TK2, which catalyzes the corresponding step in mitochondrial dTTP production. Knockout of TK1 prevented telomere lengthening following dT treatment, while TK2 knockout did not, indicating that incorporation of supplemented dT into the cytosol and nucleus drives telomere lengthening (FIG. 13C).

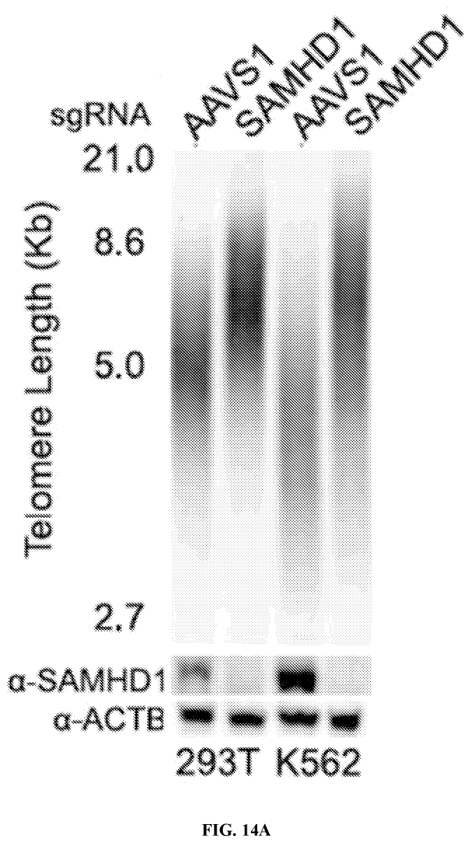

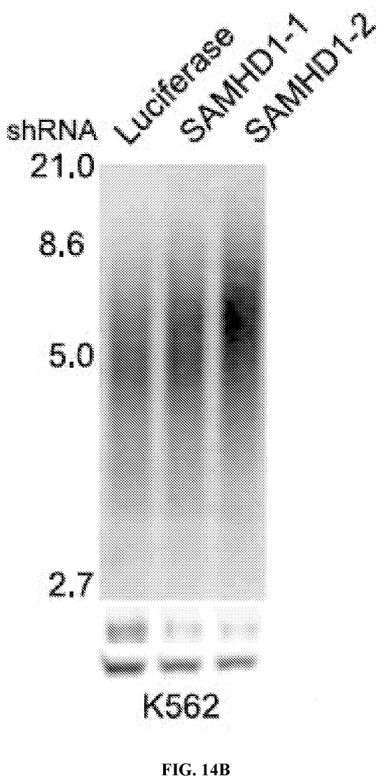

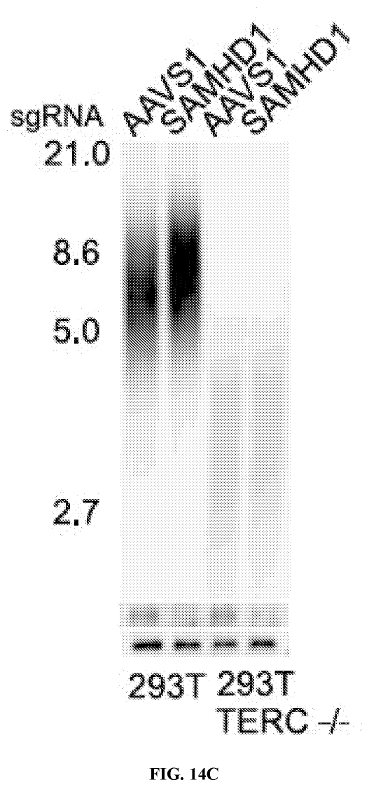

SAMHD1 restricts telomere length in human cells. SAMHD1 is a critical regulator of dNTP pools which degrades dNTPs to dNs. The data and the aforementioned GWASs are the first studies connecting SAMHD1 to telomere length control. A prior study has identified SAMHD1 as being enriched in telomeric replication forks, but did not find telomere length changes upon loss of SAMHD1, likely due to the brief timeframe (5 days) after depletion when telomere length was measured. To validate the screening results, it is shown that SAMHD1 disruption impacts telomere length regulation. Using two approaches, CRISPR/Cas9 gene knockout or shRNA knockdown, it was found that telomeres are elongated in several human cell lines after SAMHD1 disruption, including in three iPSC lines derived from TBD patients (FIG. 14A, 14B, 5H). SAMHD1 knockout in TERC-null 293T cells did not change telomere length (FIG. 14C). These results suggest a change in telomere synthesis by telomerase is driving telomere elongation upon loss of SAMHD1. Collectively these data strongly support the hypothesis that SAMHD1 is a critical negative regulator of telomere length in human cells.

SAMHD1 and thymidine metabolism control hematopoietic cell telomere length by regulating telomere repeat synthesis by telomerase (FIG. 15): While dNTP metabolism has been implicated in yeast telomere biology, prior to the aforementioned GWASs and the data herein, this pathway has not been associated with telomere length control in humans. The mechanisms underlying this connection are described herein, as well as the relevance of dNTP metabolism for hematopoietic telomere length control. In addition, longer telomere length has been associated with several health benefits in Mendelian randomization studies including increased lifespan. Thus, a therapy to increase telomere length has value beyond the treatment of TBDs. Direct oral supplementation of dNs also holds therapeutic value, as they showed promise in a retrospective study in patients with inherited mitochondrial depletion disease due to TK2 deficiency. This study found dT and dC supplementation improved functional outcomes, and survival compared to historical controls, supporting the notion that dNs can be administered orally at doses that alter cellular physiology.

Human telomerase activity is understood to be controlled at several levels, including core telomerase component levels, regulation by cofactor proteins, and the frequency of telomerase-telomere end interactions, yet these insights have yielded limited if any therapeutic interventions. Based on recent human population genetics and the data herein, this example establishes nucleotide metabolism as a critical factor controlling human telomerase activity and telomere length. Nucleotide metabolism manipulation offers a unique and complementary approach to lengthen telomeres in addition to (or in combination with) the other proposed strategies which generally focus on increasing the abundance of telomerase.

SAMHD 1 plays a role in hematopoietic telomere length homeostasis: loss of SAMHD 1 is associated with increased telomere length in human blood cells, an effect which was validated herein in several human cell lines. However, the mechanisms by which this occurs were unknown. Given that: (1) SAMHD1 degrades dNTPs, (2) SAMHD 1 localizes to the telomere, and (3) data from yeast and reconstituted telomerase suggest telomere synthesis rate is controlled by dNTP availability, the example shows that SAMHD1 dNTPase activity in the local environment of the telomere end restricts nucleotides available for telomere synthesis by telomerase. The results herein are expected to (1) investigate the role of SAMHD1 dNTPase activity in telomere length homeostasis, (2) evaluate how SAMHD1 loss impacts telomerase activity in hematopoietic cells, and (3) show how loss of SAMHD1 modulates telomere length using an in vivo model of TBD hematopoiesis.

SAMHD1 dNTPase activity is required for telomere length control. dNTPase activity may contribute to SAMHD1 telomere length restriction. The examples include cloning well-described separation of function SAMHD1 mutations into a doxycycline (dox)-inducible expression construct generated herein which produces physiologic SAMHD1 levels (thus overcoming toxicity from SAMHD1 overexpression (data not shown)). Constructs are created encoding 9 mutations which impair SAMHD1 dNTPase activity using site directed mutagenesis, verified with Sanger sequencing. These lentiviral constructs as well as a dox-inducible wildtype SAMHD1 and EGFP controls are transduced into hematopoietic-derived K562 cells and kidney-derived HEK293T (293T) which have been deleted for endogenous SAMHD1 (FIG. 14A). 48 hours after infection, cells are selected with 2 μg/ml puromycin for 3 days and treated with the appropriate concentration of dox for the remainder of the experiment. SAMHD1 expression is confirmed by western blot for each condition. Telomere length is assessed by terminal restriction fragment Southern blot after 30 days. Changes in telomere length in mutants reveal how dNTPase activity contributes to telomere length restriction by SAMHD1. This experiment is performed in biological triplicate.

SAMHD 1 regulation of telomere homeostasis in hematopoietic cells: loss of SAMHD 1 promotes telomerase processivity in human hematopoietic cells Telomerase activity is understood to be controlled by three factors: (1) the abundance of active telomerase complexes, (2) the frequency of telomerase binding to the telomere end, and (3) the length of telomere added when telomerase binds to the telomere end, termed processivity. The data is expected to show that SAMHD1 loss increases telomerase activity without altering the abundance of telomerase.

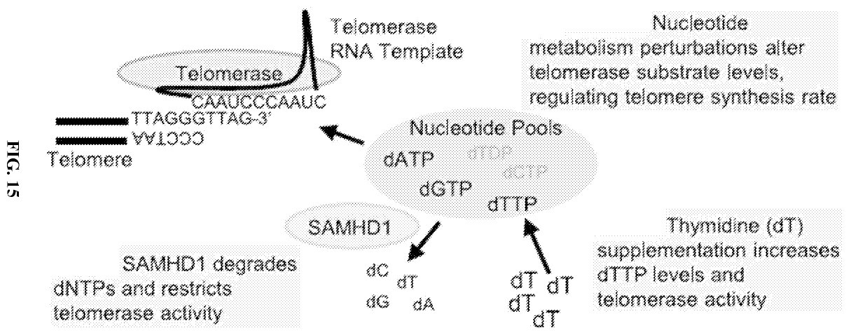

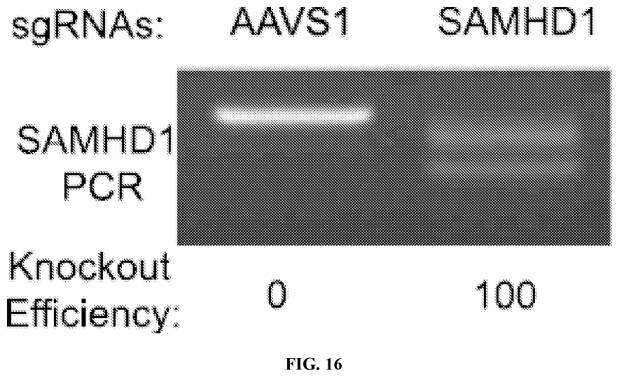

Modeling SAMHD1 loss in human hematopoietic stem and progenitor cells (HSPCs). Human HSPCs have been used to study telomerase biology, and are available from commercial and academic suppliers including the Fred Hutchinson Co-Operative Center for Excellence in Hematology (CCEH). The Fred Hutch CCEH offers information about HSPC donor sex, age, and ethnicity, which are variables associated with telomere length. In this example, HSPCs were selected that address these biologically important variables, and their impact on the phenotype(s) in question were assessed. To model the role of SAMDH1 in telomerase biology in a disease relevant primary tissue, this example is expected to show genetically engineered human HSPCs by using CRISPR/Cas9 to target either SAMHD1, or the AAVS1 control locus (FIG. 16). SAMHD1 loss is expected to increase telomerase activity without altering telomerase abundance, as shown by SAMHD1 and AAVS1 targeted cells by monitoring changes in the abundance of telomerase by measuring levels of TERT by western blot, and of TERC by northern blot. The abundance of functional telomerase complexes in cell extracts is assessed using both direct and PCR based methods. Experiments are performed in technical triplicate and biological duplicate.

SAMHD1 loss changes the frequency and processivity of telomere synthesis by telomerase. SAMHD1 loss could increase telomerase processivity, rather than changing the frequency of telomere synthesis events. To differentiate between these possibilities, a recently described method is applied in which a mutant telomerase RNA template is expressed at a low level such that rare telomeres will be elongated with the alternative template (i.e., TTTGGG vs TTAGGG).

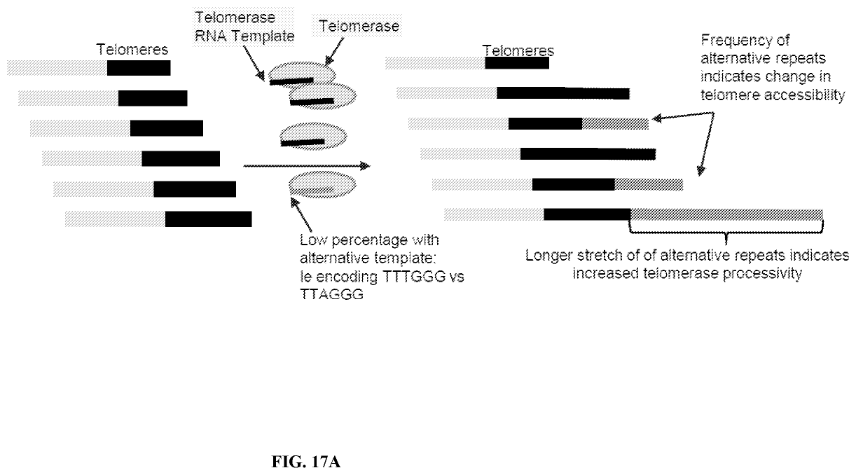

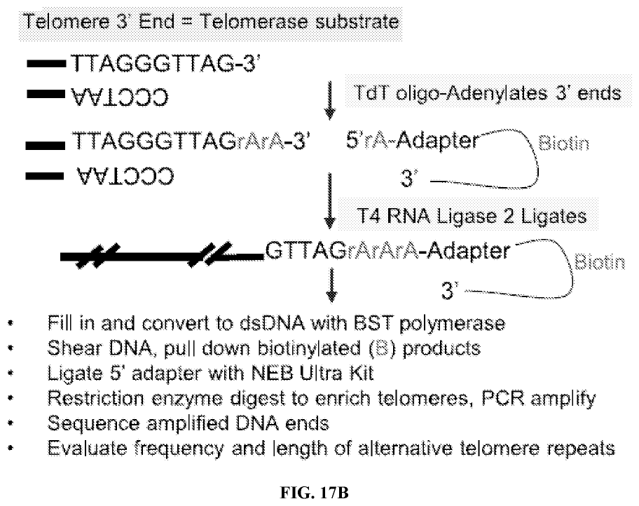

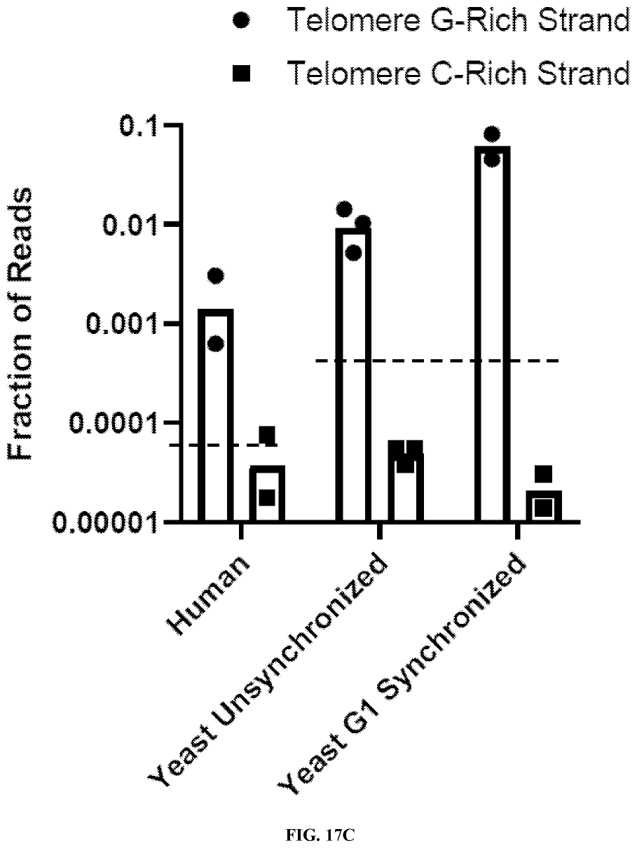

Quantifying the fraction of alternative template positive telomeres measures the frequency of telomerase synthesis events, while the length of alternative repeat stretches quantifies changes in telomerase processivity (FIG. 17A). This method is adapted for a sequencing-based readout to enhance quantification by using TrAEL seq37, which sequences DNA 3′ ends (FIG. 17B). G rich telomere ends are a source of 3′ DNA ends which can be captured by TrAEL seq, or extended by telomerase. To evaluate TrAEL seq telomere end sequencing efficiency, a published dataset from human as well as yeast cells was analyzed and telomere reads were quantified. G rich telomere sequences were overrepresented compared to their genomic abundance, as expected, while C rich strands were not (FIG. 17C). To implement this method, 24 hours after SAMHD1 or AAVS1 editing, primary human HSPCs are transfected with an alternative telomerase template expression plasmid, the cells cultured for another 24 hours, and then a TrAEL seq protocol modified to enrich for telomere repeats is performed (FIG. 17B). Sequencing data are analyzed by adapting an established telomere variant detection method. Sequence analysis pipeline development is performed. SAMHD1 loss is expected to increase telomerase processivity which is detected as longer stretches of alternative template repeats. Because manipulating the telomerase RNA template may change how dNTP substrates impact telomerase activity, this experiment is repeated using four different template variants. Experiments are performed in biological duplicate.

Determine the role of SAMHD1 in telomere biology in an in vivo model of TBD hematopoiesis: loss of SAMHD 1 is expected to promote telomere elongation in TBD-mutant HSPCs in vivo. Telomere length changes can take weeks to become apparent, confounding assessment in short-term HSPC culture experiments. To overcome this challenge, this example uses an in vivo TBD hematopoiesis model. In this experiment, human HSPCs are subject to genome-editing, targeting SAMHD1 or AAVS1, as well as for the TBD-associated gene PARN, as described. 24 hours after editing, cells are transplanted into immunodeficient NBSGW mice, which engraft human cells without radiation or chemotherapy. Six to eight weeks later, this example recovers xenografted human hematopoietic cells by flow cytometry, and evaluates telomere length by flow-FISH, which is able to distinguish mouse versus human cells. To ensure statistical power edited SAMHD1 or AAVS1 cells are each transplanted into 4 mice (2 females and 2 males) providing 95% power to detect a 10% increase in telomere length based on our previous data. This example also evaluates engraftment and multi-lineage differentiation by flow cytometry, and CRISPR-generated indel fractions in the recovered cells compared to those transplanted, which define the impact of SAMHD1 manipulation and telomere length homeostasis on hematopoietic function in vivo. HSPC editing and xenotransplant experiments are performed in biological replicate using HSPCs from one female and one male donor. SAMHD1 loss is expected to increase telomere length in PARN mutant HSPCs and promote hematopoietic output in vivo.

dNTPase activity is required for telomere length restriction, and SAMHD1 loss promotes human hematopoietic cell telomerase activity and telomere lengthening. SAMHD1 loss alters background levels of free DNA 3′ ends, which confounds the telomere end sequencing approach. Treating with E. Coli DNA Ligase to seal nicks prior to 3′ end labeling reduces background non-telomere end derived reads. Another strategy to detect alternative template usage is to perform long read sequencing on purified or PCR amplified telomeres. Long read sequencing has been used to study yeast and human telomeres. It is also possible eight weeks is not sufficient to cause hematopoietic defects in PARN deficient xenografted HSPCs. Strategies to reveal hematopoietic phenotypes in TBD mutant HSPCs include 5-fluorouracil treatment and serially transplanting xenografted cells.

Mechanisms by which thymidine metabolism contributes to telomere length control: supplementing dT rapidly promotes telomere lengthening in human cells (FIG. 2c). However, whether this occurs in hematopoietic cells and how this is regulated mechanistically was unknown. Given that: (1) dT metabolism genes have been associated with telomere length regulation in human blood cells, (2) studies in yeast and using reconstituted telomerase have demonstrated repeat synthesis is sensitive to dTTP concentrations, and (3) dT supplementation increases dTTP levels, the results are expected to show that supplemental dT alters the physiologic abundance of dNTPs at the telomere to promote telomerase activity and telomere lengthening in hematopoietic cells.

This experiment studies how exogenously supplied dT is used by cells, evaluates how dT supplementation impacts HSPC telomerase activity, and studies how supplemental dT impacts telomere lengthening in an in vivo model of TBD hematopoiesis.

Thymidine is incorporated in cells and used by telomerase: Supplemental thymidine is converted to dTTP by cellular kinases which is used by telomerase. This example identifies the components of thymidine metabolism required for telomere lengthening upon dT treatment. The example performs a nucleotide metabolism CRISPR screen using the gRNA library previously utilized (FIG. 1b). 24 hours after gRNA library infection, K562 cells are cultured in media supplemented with 100 μM dT or vehicle control for 10 days followed by flow-FISH sorting. DNA is isolated and subjected to analysis for gRNA representation in sorted long and short telomere populations. The results are expected to show: (1) dTTP salvage genes are required for dT mediated telomere lengthening; (2) de novo dTTP synthesis genes are not required, and (3) loss of dTTP catabolismgenes, for example SAMHD1, promotes dT mediated telomere lengthening. Screening is performed in biological triplicate.

Nucleotide pool changes from dT supplementation impact telomerase activity. The experimental data indicate dT treatment increases dTTP and dTDP levels, both known telomerase substrates. Nucleotide levels vary throughout the cell cycle, and telomerase extends telomeres in S phase. To measure nucleotide levels at the time of telomere synthesis, metabolites are harvested from 100 μM dT treated or control K562 or 293T cells in S phase using serum starvation to synchronize cells. Synchronization was confirmed by DNA staining and flow-cytometry. LC-MS is used to quantify changes in nucleotide levels as above (FIG. 13b).

Nucleotide level changes promote telomerase enzymatic activity. An in vitro telomerase assay is used to manipulate the levels of exogenous nucleotides supplemented from baseline levels based on the significant changes observed following dT treatment. This includes assessment of the canonical (dATP, dGTP, dTTP), and non-canonical (i.e. dTDP) telomerase substrates. Metabolites that are significantly changed are assessed individually and collectively to evaluate which metabolite(s), for example dTTP or dTDP, drive the observed changes. These experiments are performed in dG, dA, or dC treated cells to determine why dT treatment uniquely elongates telomeres. Experiments are performed in biological triplicate.

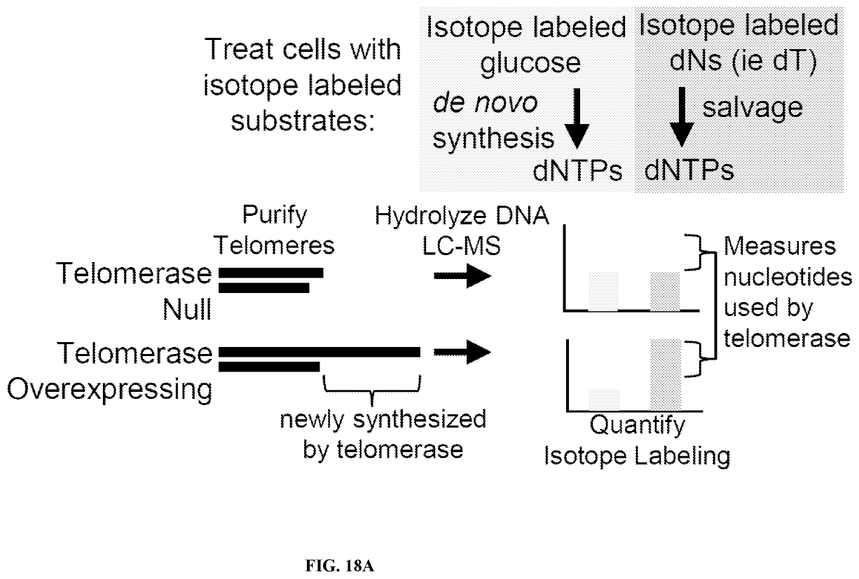

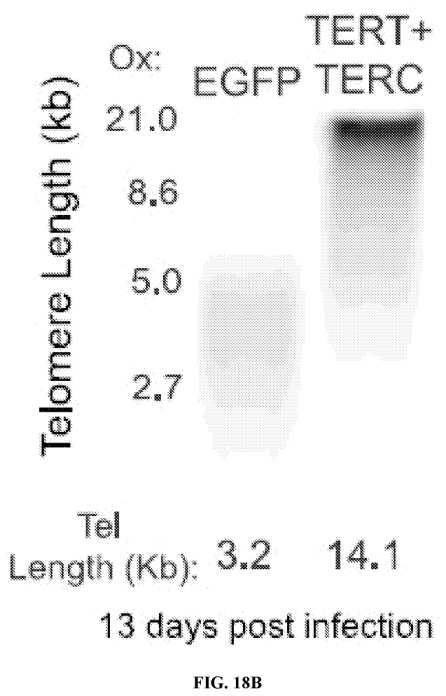

Salvaged thymidine is used for telomere synthesis. There is evidence that salvaged dNTPs and de novo synthesized dNTPs are used in cells in different ways, including being incorporated into DNA at different rates. Supplemental dT may preferentially expand the pool of cellular nucleotides used by telomerase to synthesize telomeres in a way not captured by measuring total free nucleotide pools. Salvaged nucleotides are labeled by treating cells with 5 μM of heavy-isotope labeled [U-13C/15N] dNs, and de novo synthesized nucleotides are labeled using isotope labeled [U-13C] glucose (FIG. 18A), a strategy described previously. 293T cells are treated with these conditions or with the addition of 100 μM labeled dT for 24 hours. Genomic DNA and telomeric DNA is isolated and hydrolyzed to mononucleotides followed by LC-MS. Free nucleotide pools are also evaluated by LC-MS. To purify telomeric DNA, an established strategy is used where non-telomeric DNA is digested with restriction enzymes and intact telomeres are pulled down with a biotinylated telomere complementary probe. To evaluate how telomerase uses de novo synthesized versus salvaged pools, telomere isolation is performed on either telomerase-null 293T cells with short telomeres (FIG. 2A) transfected with either TERT and TERC expression vectors or with EGFP controls 24 hr previously. TERT and TERC overexpression can drive rapid telomere extension by >800 bp per day (FIG. 18B), so after one cell division, newly telomerase-synthesized telomeres make up over 10% of telomeric DNA. Observing a difference in the fraction of de novo or salvaged nucleotides in telomerase overexpressing cell telomeres compared to cells without telomerase would indicate that telomerase uses distinct dNTP pools than those used for genome replication, and reveal a preference for salvaged dTTP explaining why dT treatment promotes robust telomere lengthening. Experiments are performed in biological triplicate.

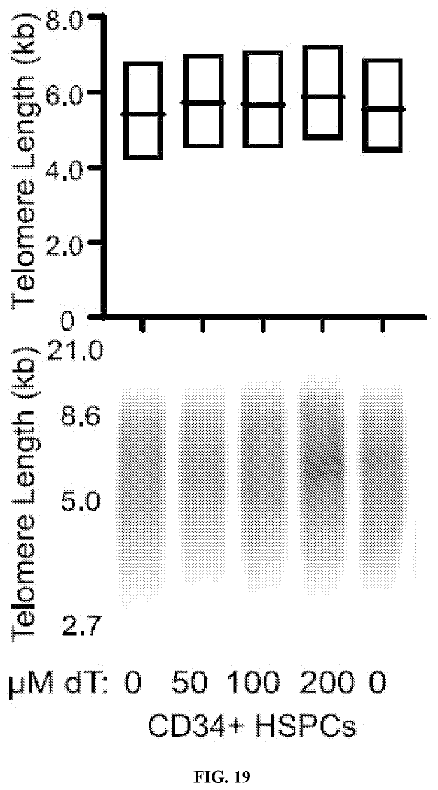

Evaluate how thymidine supplementation alters telomerase activity and telomere length in human hematopoietic cells in vitro and in vivo: dT is expected to promote telomerase processivity and telomere lengthening in human HSPCs. Thymidine is expected to change telomerase activity in HSPCs. To assay changes in telomerase activity from dT treatment, primary HSPCs and K562 cells are treated with 100 μM dT for two days. The data shows that thymidine treatment lengthens telomeres in human HSPCs after just 7 days (FIG. 19), which is expected to occur through increased telomerase processivity. Changes in telomerase levels and activity are assessed in HSPCs and K562 cells as described earlier. For measurement of changes in telomerase processivity and the frequency of telomere addition, cells are treated with 100 μM dT for 48 hours prior to transfection of an alternative template TERC vector. 24 hours after transfection, alternative template utilization is assessed in order to quantify telomerase processivity changes.

As dT treatment halts cell growth at high doses, this example evaluates how dT treatment impacts HSPC cell cycle dynamics using DNA staining and flow-cytometry. In vitro colony forming assays are also used to evaluate how dT treatment alters differentiation into myeloid, lymphoid, and erythroid lineages as previously performed in our lab. Experiments are performed in technical triplicate and biological duplicate.

Thymidine treatment alters telomere length in an in vivo model of TBD hematopoiesis. The xenograft assay is used to evaluate how thymidine supplementation impacts telomere lengthening in an in vivo model of TBD hematopoiesis. The TBD associated PARN gene is disrupted by CRISPR/Cas9 in primary HSPCs, which is then transplanted into two groups of four NBSGW mice. Mice in each group are treated with either 520 mg/kg/day of dT in water by oral gavage or with control water, a dose which increased lifespan in a mouse model of TK2 deficiency. dT supplementation begins 48 hours after transplant and proceeds for eight weeks. Transplanted cells are evaluated for telomere length changes by flow-FISH and for changes in hematopoietic output by flow cytometry as described. This is performed in biological replicate using HSPCs from one female and one male donor.

Without being bound by any theory or speculation, it is believed that the salvage pathway kinases are required for telomere elongation upon dT supplementation, that dT is converted to dTTP and used by telomerase to synthesize telomeres, and that dT supplementation promotes telomerase activity and telomere lengthening in human HSPCs. dT supplementation is expected to disrupt stem cell dynamics by altering dNTP homeostasis and driving replication stress. Furthermore, a dC+dT supplementation strategy drives telomere lengthening (FIG. 2c) and is associated with reduced toxicity compared to dT alone in cell culture.

In brief summary, pyrimidine nucleoside (e.g., thymidine, deoxyuridine, or deoxycytidine, or any combination thereof) supplementation is capable of increasing telomere length in multiple human cell types rapidly. Degree of telomere lengthening resulting from this supplementation is surprising, unexpected, and could not be predicted on the basis of knowledge about thymidine biology ahead of experimentation. The telomere lengthening occurs from pyrimidine nucleoside supplementation in cells from patients with genetic defects in telomere biology. While thymidine is being used in combination with deoxycytidine in clinical trials for a different indication (thymidine kinase 2 deficiency, NCT03701568), nothing in the prior knowledge teaches or suggests using either of these agents, alone or in combination, to prevent or treat telomere biology disorders as shown in this disclosure.

### Additional Discussion of Experimental Data

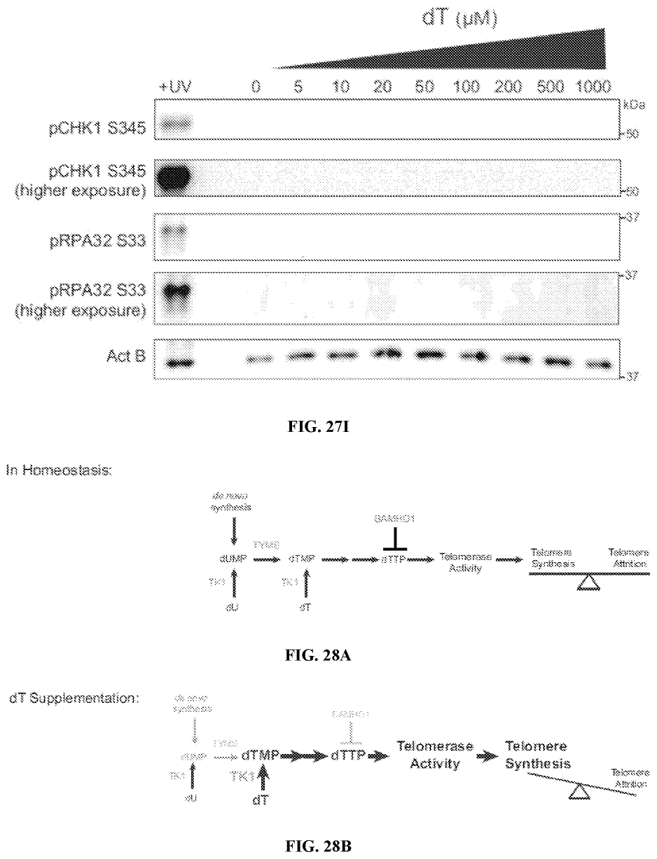

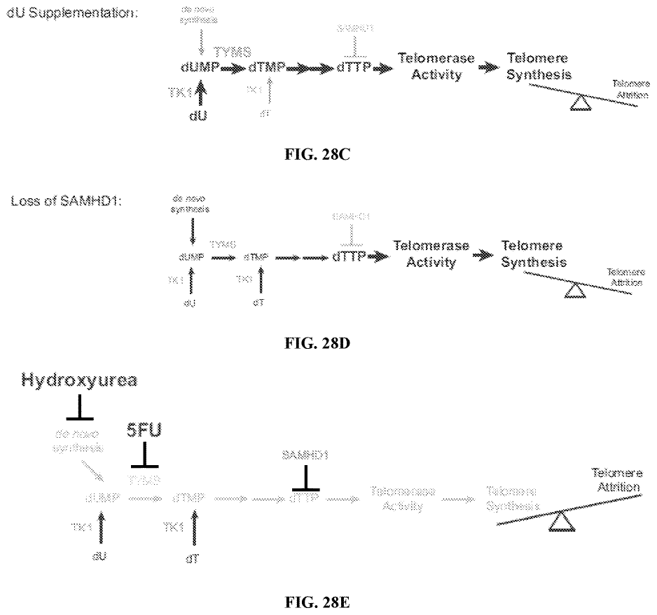

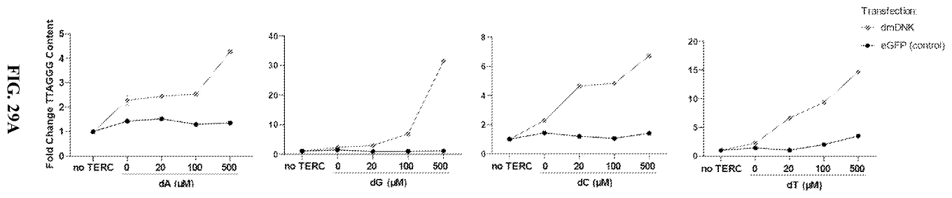

Impaired telomere length maintenance is associated with reduced lifespan and fatal genetic degenerative diseases. Using phenotypic CRISPR/Cas9 screening in intact cells, we identified dT nucleotide metabolism as a critical pathway controlling human telomere length homeostasis. DNA precursor levels are tightly controlled though a balance of de novo synthesis, salvage, and catabolismthe experimental data demonstrates that telomere length is highly sensitive to changes in dT nucleotide metabolism (FIG. 27f and FIG. 28): loss of genes in the dT nucleotide synthesis or salvage pathways reduced telomere length, whereas loss of the dNTP degrading gene SAMHD 1 lengthened telomeres. In addition to genetic perturbations, we find that telomere synthesis is highly sensitive to small molecules targeting dT nucleotide metabolism. Supplementing cells with dT drove robust telomere elongation, whereas treatment with 5FU or hydroxyurea, which limit dTTP production, blocked telomere repeat synthesis by telomerase. Collectively, our work, in line with emerging population and Mendelian genetic data, demonstrates the critical importance of dT nucleotide metabolism in human telomere length control and highlights the additional insights gained from longitudinal functional genetic studies in human cells.

While dTTP is a canonical substrate of human telomerase alongside dATP and dGTP, this striking impact of dT nucleotide metabolism on telomere length in human cells is unexpected given prior studies using reconstituted telomerase and yeast indicating that dGTP is rate-limiting for telomerase activity. Here, using a modified telomerase enzyme that no longer uses dTTP as a substrate, we find evidence for substrate-independent enhancement of telomerase activity by dT nucleotides both in vitro and in living cells. While we cannot exclude a role for secondary effects of dT on dATP and dGTP levels contributing to telomere length changes in cells, a substrate-independent, potentially allosteric effect of dTTP on telomerase activity offers a unifying mechanism to explain our genetic, pharmacological, and biochemical findings, that, coupled with recent human genetic data, firmly establishes a role for dT nucleotide metabolism in telomere length regulation. More specifically, a preponderance of orthogonal evidence, including high-throughput functional genetic screening, 5FU treatment, GWAS, and genetic discovery, collectively implicates TYMS as a critical control point, thus revealing a limiting role for de novo dT nucleotide production in human telomere length regulation.

Prior work connected replication stress signaling with enhanced telomerase recruitment to telomeres and increased telomere length. Experimental data presented herein demonstrates that telomere lengthening from dT can occur at doses that do not result in replication stress or disrupt the cell cycle. Furthermore, 5FU and hydroxyurea, compounds known to cause replication stress, blocked telomere repeat synthesis by telomerase in the experimental system presented herein, rather than extending telomeres. We cannot exclude that replication stress-mediated changes in telomerase recruitment could play a role in telomere elongation at very high dT doses; however, experimental data clearly demonstrates that modulating dT nucleotides at more physiological levels can impact human telomere length by other means, such as activation of telomerase.

While we show that cell cycle inhibition and replication stress cannot explain telomere lengthening from dT, we find that the high doses commonly used to synchronize cells cause significant changes in telomere repeat synthesis. Several investigations of human telomerase biology have used dT or related compounds like bromodeoxyuridine (BrdU) to facilitate measurements such as the timing of telomere synthesis by telomerase during the cell cycle, and the kinetics of repeat addition by telomerase at a given chromosome end. Experimental results presented herein indicates that a more nuanced interpretation of telomere biology effects may be required when using dT and its analogs for synchronization or labeling in human cells.

Defects of nucleotide synthesis are associated with diseases, including mitochondrial genetic disorders and cancer, and manipulation of nucleotide metabolism is widely used in life-saving therapies, including those for cancer, autoimmune, and infectious diseases. Remarkably, supplementation with dT promotes rapid telomere lengthening at low micromolar doses. This effect was evident across various cell lines, including iPSCs derived from patients with TBDs caused by diverse, hypomorphic genetic defects. Based on the findings in vitro and in cultured cells, along with promising clinical trials currently underway using oral dT supplementation to treat a mitochondrial genetic disease, there is a therapeutic window to modulate telomere length via manipulating dT metabolism in patients with a range of genetic degenerative disorders.

dNTP metabolism is commonly considered in relation to DNA replication and repair. Experimental data presented herein uncovers a unique sensitivity of telomerase reverse transcriptase activity to dT nucleotide homeostasis. While dT supplementation alone can inhibit genome replication by DNA polymerases and HIV-1 reverse transcription, dT treatment drove robust increases in telomere synthesis by telomerase. These evidence enhances understanding of how differences in the common pool of cellular nucleotide substrates driven by genetic variation and other factors can have distinct effects on the various DNA synthesis machineries in the cell. Evolutionary pressures on dNTP metabolism have likely faced a tradeoff between telomere length maintenance, nuclear and mitochondrial genomic integrity, and other forces, including the restriction of endogenous or exogenous retro-elements. Telomere length homeostasis offers a new lens to examine the genetic regulation and evolution of DNA precursor metabolism in humans.

Summary of experimental results. Human telomere length is associated with lifespan as well as severe diseases, but the mechanisms underlying telomere maintenance are not fully understood. To identify new pathways regulating telomere length, CRISPR/Cas9 screening was performed in human cells and a critical role for pyrimidine nucleotide metabolism was identified. Specifically, loss of thymidine (dT) nucleotide synthesis genes including TK1, TYMS, and DTYMK led to telomere shortening, while loss of the dNTP degrading gene SAMHD1 drove telomere elongation. Strikingly, supplementing cells with dT nucleoside promoted telomere elongation in a telomerase-dependent manner, at doses that did not inhibit cell growth or induce replication stress. dT treatment increased telomere length in iPSCs derived from patients with dyskeratosis congenita, suggesting that manipulation of dT nucleotide metabolism may be useful to treat telomere biology disorders. In order to investigate the mechanisms of dT-mediated telomere lengthening, we overexpressed TERT and TERC in 293T cells (“super-telomerase” cells), and found that treatment with dT for only 30 hours drove dramatic increases in telomere synthesis, with the maximal rate of telomere elongation increasing from 103 bp/hour 95% CI [34, 172] at baseline, up to 625 bp/hour 95% CI [475, 775] with high-dose dT treatment. In contrast, treatment with 5-fluorouracil (5FU), which blocks de novo dTTP production, ablated telomere lengthening in super-telomerase cells. Bypassing the dTTP production block in 5FU treated cells via dT supplementation fully rescued telomere synthesis, suggesting that the manipulation of dTTP abundance is sufficient to control telomerase activity in cells. Next, because dTTP is used by telomerase in telomere repeat synthesis, it was determined whether the observed effects of dT manipulation on telomeres were dependent on dTTP's role as a telomerase substrate. To address this, TERC was modified to encode 5′-GGAAAG-3′ (“T-free”) repeats rather than wildtype 5′-GGTTAG-3′ repeats. Surprisingly, dT treatment of TERC−/− 293T cells overexpressing T-free TERC plus TERT also enhanced repeat synthesis by telomerase. In contrast, 5FU inhibited T-free telomerase activity, an effect which was rescued by dT supplementation. These data support a model wherein dT nucleotides can promote telomerase activity in a manner distinct from dTTP's utilization as a substrate.

### Experiment 3

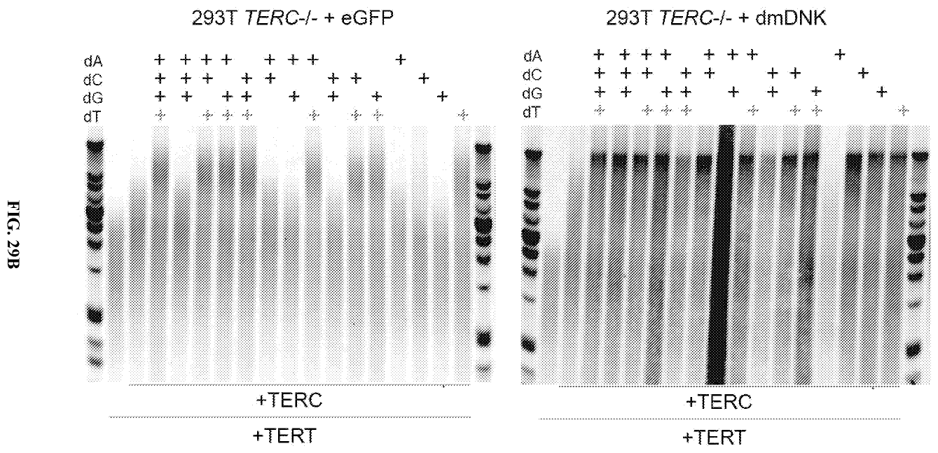

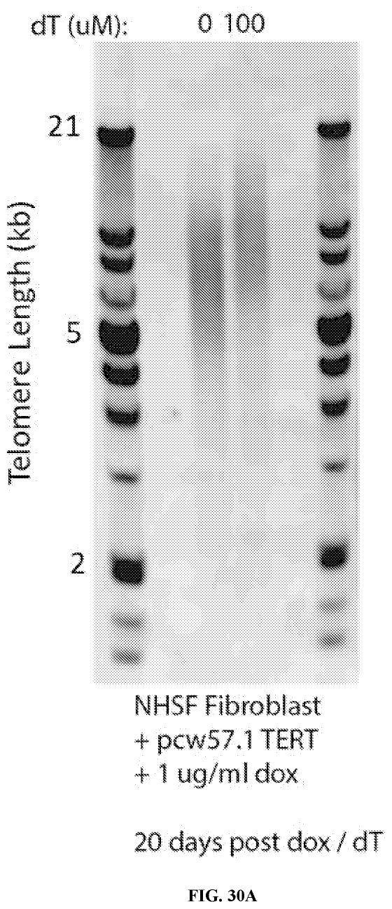

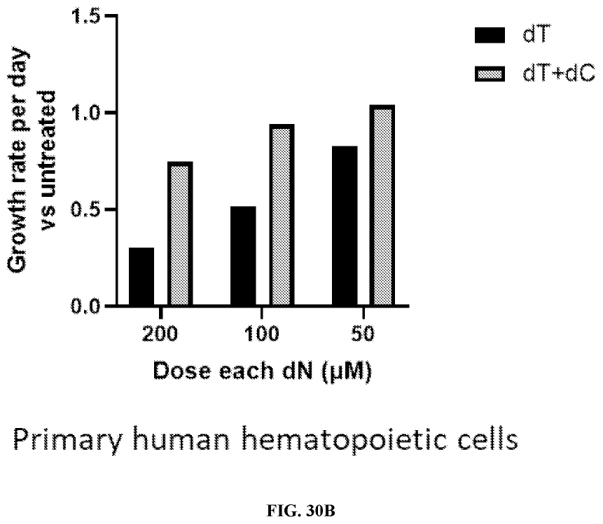

Combining thymidine treatment with expression of a nucleoside kinase to improve efficiency of nucleoside uptake and thereby promote telomere lengthening. combining nucleoside treatment with the expression of TK1, or the deoxynucleoside kinase from Drosophila (dmDNK), either of which can be delivered via mRNA. See FIG. 29A-B. TERC−/− 293T cells stably expressing dmDNK or eGFP (control) were transfected with vectors to express TERT andTERC, then cultured in the indicated doses of nucleoside, followed by analysis of telomere DNA content via blotting and detection with a complementary oligonucleotide probe. Enhanced telomere lengthening from the combination of expressing a nucleoside kinase with dT treatment is a surprising and unexpected finding. Furthermore, these data show that expressing a nucleoside kinase enables telomere lengthening from deoxynucleosides besides dT, which is also a therapeutically useful, surprising and unexpected finding.

### NUMBERED PARAGRAPHS

The invention may be described by reference to the following numbered paragraphs:
Paragraph 1. A method of treating a telomere biology disorder, the method comprising administering to a subject diagnosed with said telomere biology disorder a therapeutically effective amount of a compound of Formula (A):

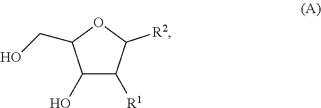

or a pharmaceutically acceptable salt thereof, wherein:
R¹ is selected from H and OH;
R² is selected from any one of the following moieties:

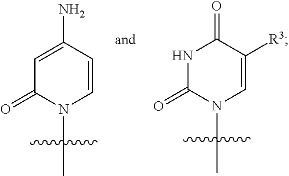

and
R³ is selected from H and CH₃.
Paragraph 2. The method of paragraph 1, wherein the compound has formula:

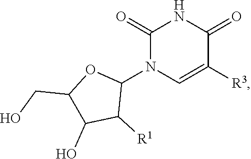

or a pharmaceutically acceptable salt thereof.
Paragraph 3. The method of paragraph 1 or paragraph 2, wherein R³ is H.
Paragraph 4. The method of paragraph 1 or paragraph 2, wherein R³ is CH₃.
Paragraph 5. The method of paragraph 1, wherein the compound has

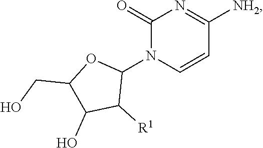

or a pharmaceutically acceptable salt thereof.
Paragraph 6. The method of any one of paragraphs 1-5, wherein R¹ is H.
Paragraph 7. The method of any one of paragraphs 1-5, wherein R¹ is OH.
Paragraph 8. The method of paragraph 1, wherein the compound of Formula (A) has Formula (I):

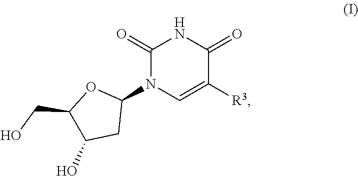

or a pharmaceutically acceptable salt thereof.
Paragraph 9. The method of paragraph 8, wherein the compound of Formula (I) is:

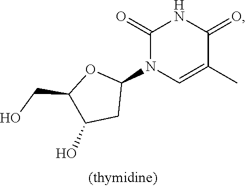 [SMILES: Cc1cn([C@H]2C[C@H](O)[C@@H](CO)O2)c(=O)[nH]c1=O]

or a pharmaceutically acceptable salt thereof.
Paragraph 10. The method of paragraph 8, wherein the compound of Formula (I) is:

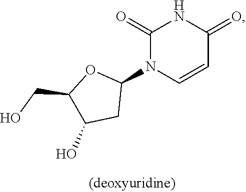 [SMILES: O=c1ccn([C@H]2C[C@H](O)[C@@H](CO)O2)c(=O)[nH]1]

or a pharmaceutically acceptable salt thereof.
Paragraph 11. The method of paragraph 1, wherein the compound of Formula (A) is:

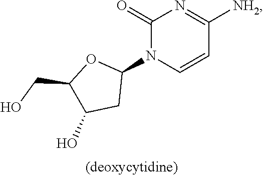 [SMILES: Nc1ccn([C@H]2C[C@H](O)[C@@H](CO)O2)c(=O)n1]

or a pharmaceutically acceptable salt thereof.
Paragraph 12. The method of paragraph 1, wherein the compound of Formula (A) is:

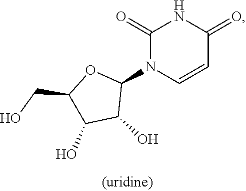 [SMILES: O=c1ccn([C@@H]2O[C@H](CO)[C@@H](O)[C@H]2O)c(=O)[nH]1]

or a pharmaceutically acceptable salt thereof.
Paragraph 13. The method of paragraph 1, wherein the compound of Formula (A) is:

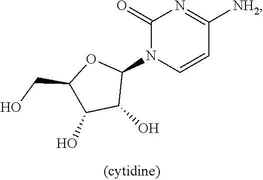 [SMILES: Nc1ccn([C@@H]2O[C@H](CO)[C@@H](O)[C@H]2O)c(=O)n1]

or a pharmaceutically acceptable salt thereof.
Paragraph 14. The method of any one of paragraph 1-13, wherein the telomere biology disorder is selected from dyskeratosis congenita, aplastic anemia, myelodysplastic syndrome, pulmonary fibrosis, idiopathic pulmonary fibrosis, interstitial lung disease, hematological disorder, liver disease, hepatic fibrosis, Hoyeraal-Hreidarsson syndrome, Coats Plus syndrome, and Revesz syndrome.
15. The method of any one of paragraphs 1-13, wherein the telomere biology disorder is selected from dyskeratosis congenita, aplastic anemia, and interstitial lung disease
16. The method of any one of paragraphs 1-13, wherein the telomere biology disorder is dyskeratosis congenita.
17. The method of any one of paragraphs 1-13, wherein the telomere biology disorder is aplastic anemia.
18. The method of any one of paragraphs 1-13, wherein the telomere biology disorder is interstitial lung disease.
Paragraph 19. The method of any one of paragraphs 1-18, comprising administering the compound to the subject orally.
Paragraph 20. The method of paragraph 19, comprising administering the compound in a dosage form selected from a capsule, a tablet, and a sachet.
Paragraph 21. The method of any one of paragraphs 1-18, comprising administering the compound to the subject intravascularly.
Paragraph 22. The method of paragraph 21, comprising administering the compound in a dosage form comprising an injectable or infusible aqueous solution.
Paragraph 23. The method of any one of paragraphs 1-22, the therapeutically effective amount of the compound is from about 50 mg/kg/day to about 500 mg/kg/day.
Paragraph 24. The method of paragraph 23, wherein the therapeutically effective amount of the compound is from about 130 mg/kg/day to about 400 mg/kg/day.
Paragraph 25. The method of paragraph 23, wherein the therapeutically effective amount is about 130 mg/kg/day, about 260 mg/kg/day, or about 400 mg/kg/day.
Paragraph 26. The method of any one of paragraphs 1-25, wherein the compound is administered one a day.
Paragraph 27. The method of any one of paragraphs 1-25, wherein the compound is administered twice a day.
Paragraph 28. The method of any one of paragraphs 1-25, wherein the compound is administered three times a day.
Paragraph 29. The method of any one of paragraphs 1-28, comprising administering at least two compounds of Formula (A), or a pharmaceutically acceptable salt thereof.
Paragraph 30. The method of paragraph 29, comprising co-administering:

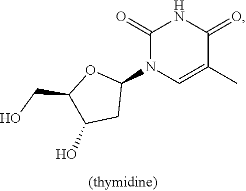 [SMILES: Cc1cn([C@H]2C[C@H](O)[C@@H](CO)O2)c(=O)[nH]c1=O]

or a pharmaceutically acceptable salt thereof, and

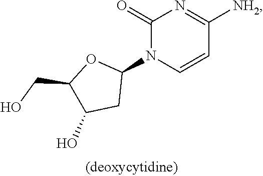 [SMILES: Nc1ccn([C@H]2C[C@H](O)[C@@H](CO)O2)c(=O)n1]

or a pharmaceutically acceptable salt thereof.
Paragraph 31. The method of any one of paragraphs 1-30, wherein the compound is administered in combination with an additional therapeutic agent useful in treating a telomere biology disorder
Paragraph 32. The method of paragraph 31, wherein the additional therapeutic agent is purine nucleoside, or a pharmaceutically acceptable salt thereof.
Paragraph 33. The method of paragraph 32, wherein the purine nucleoside is selected from adenosine (A), deoxyadenosine (dA), guanosine (G), and deoxyguanosine (dG), or a pharmaceutically acceptable salt thereof.
Paragraph 34. The method of paragraph 31, wherein the additional therapeutic agent is an inhibitor of dNTPase SAM domain and HD domain-containing protein 1 (SAMHD1), or a pharmaceutically acceptable salt thereof.
Paragraph 35. The method of paragraph 34, wherein the SAMHD1 inhibitor is selected from miRNA181a/b, an anti-SAMHD1 antibody, a T cell receptor, and a protein having at least 75% identity to VPX or VPR.
Paragraph 36. The method of paragraph 34, wherein the SAMHD1 inhibitor is selected from erythrotyrosine, sennoside A, evans blue, merbromin, phenylmercuric acetate, thiram, bronopol, cephalosporin C, pidolic acid, diphenhydramine, aurothiomalate, rose bengal, chlorambucil, pyrithione zinc, lomofungin, troglitazone, montelukast, pranlukast, L-thyroxine, ergotamine, amrinone, retinoic acid, ethacrynic acid, hexestrol, tolfenamic acid, bexarotene, sulindac, zolmitriptan, nifedipine, tetracycline, nisoldipine, medroxyprogesterone acetate, trifluoperazine, primaquine, adapalene, aprepitant, tolcapone, zafirlukast, delavirdine, topotecan, ceftazidime, zoledronic acid, anethole-trithione, and disulfiram, or a pharmaceutically acceptable salt thereof.
Paragraph 37. The method of paragraph 31, wherein the additional therapeutic agent is an inhibitor of thymidine phosphorylase (TYMP), or a pharmaceutically acceptable salt thereof.
Paragraph 38. The method of paragraph 37, wherein the TYMP inhibitor is selected from tipiracil, 6-aminothymine (6AT), amino-5-chlorouracil (6A5CU), 6-amino-5-bromouracil (6A5BU), 7-deazaxanthine (7DX), 7-(2-aminoethyl)-deazaxanthine, 6-(2-aminoethylamino)-5-chlorouracil (AEAC), 5-chloro-6-(1-imidazolylmethyl)uracil (CIMU), 6-methylenepyridinium, 6-(4-phenylbutylamino)uracil, 5-chloro-6-[1-(2-iminopyrrolidinyl)methyl]uracil hydrochloride (TPI), and N-(2,4-dioxo-1,2,3,4-tetrahydro-thieno[3,2-d]pyrimidin-7-yl)guanidine, or a pharmaceutically acceptable salt thereof.
Paragraph 39. A method of treating a disorder associated with aging, the method comprising administering to a subject diagnosed with said disorder associated with aging a therapeutically effective amount of a compound of Formula (A):

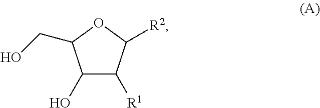

or a pharmaceutically acceptable salt thereof, wherein:
R¹ is selected from H and OH;
R² is selected from any one of the following moieties:

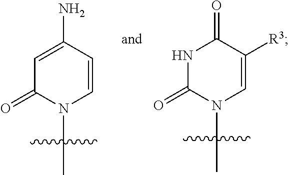

and
R³ is selected from H and CH₃.
Paragraph 40. The method of paragraph 39, wherein the compound has formula:

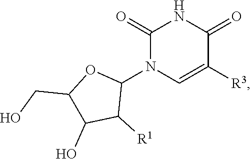

or a pharmaceutically acceptable salt thereof.
Paragraph 41. The method of paragraph 39 or paragraph 40, wherein R³ is H.
Paragraph 42. The method of paragraph 39 or paragraph 40, wherein R³ is CH₃.
Paragraph 43. The method of paragraph 39, wherein the compound has formula:

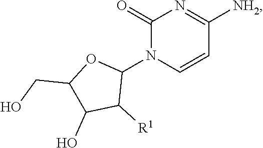

or a pharmaceutically acceptable salt thereof.
Paragraph 44. The method of any one of paragraphs 39-43, wherein R¹ is H.
Paragraph 45. The method of any one of paragraphs 39-43, wherein R¹ is OH.
Paragraph 46. The method of paragraph 39, wherein the compound of Formula (A) has Formula (I):

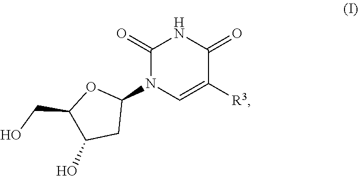

or a pharmaceutically acceptable salt thereof.
Paragraph 47. The method of paragraph 46, wherein the compound of Formula (I) is:

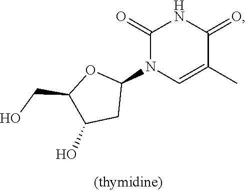 [SMILES: Cc1cn([C@H]2C[C@H](O)[C@@H](CO)O2)c(=O)[nH]c1=O]

or a pharmaceutically acceptable salt thereof.
Paragraph 48. The method of paragraph 46, wherein the compound of Formula (I) is:

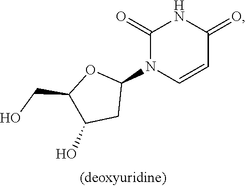 [SMILES: O=c1ccn([C@H]2C[C@H](O)[C@@H](CO)O2)c(=O)[nH]1]

or a pharmaceutically acceptable salt thereof.
Paragraph 49. The method of paragraph 39, wherein the compound of Formula (A) is:

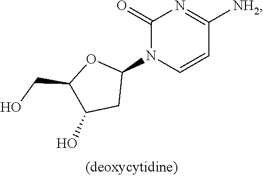 [SMILES: Nc1ccn([C@H]2C[C@H](O)[C@@H](CO)O2)c(=O)n1]

or a pharmaceutically acceptable salt thereof.
Paragraph 50. The method of paragraph 39, wherein the compound of Formula (A) is:

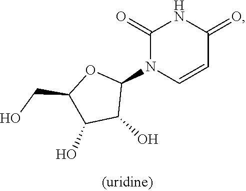 [SMILES: O=c1ccn([C@@H]2O[C@H](CO)[C@@H](O)[C@H]2O)c(=O)[nH]1]

or a pharmaceutically acceptable salt thereof.
Paragraph 51. The method of paragraph 39, wherein the compound of Formula (A) is:

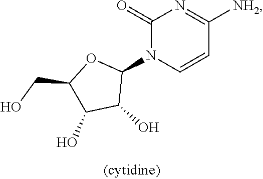 [SMILES: Nc1ccn([C@@H]2O[C@H](CO)[C@@H](O)[C@H]2O)c(=O)n1]

or a pharmaceutically acceptable salt thereof.
Paragraph 52. The method of any one of paragraph 39-51, wherein the disorder associated with aging is selected from inflammatory disease, immune disease, adult disease, infectious disease, cardiovascular disease, dermatological disease, ophthalmic disease, neurological disease, wasting disorder, metabolic disorder, cancer, a pre-cancerous condition, and a disorder of the connective tissue
Paragraph 53. The method of paragraph 52, wherein the disorder associated with aging is selected from age-related anxiety, anemia, anorexia, arteriosclerosis, asthma, balance disorder, Bell's palsy, bone marrow failure, breathlessness, cachexia, chronic infection, cirrhosis, congestive heart failure, deafness, diabetes, emphysema, failure to thrive, flu, frailty, gastrointestinal ulcer, generalized anxiety disorder, gout, hair loss, hearing loss, hepatic insufficiency, high blood pressure, high fat, hip dislocation, hypercholesterolemia, hyperglycemia, hyperhomocysteinemia, hyperlipidemia, immunosenescence, impaired mobility, loss of appetite, loss of bone density, loss of sense of taste, metabolic syndrome, muscle loss, muscle wasting, muscular dystrophy, myocardial infarction, obesity, organ dysfunction, osteoporosis, peripheral artery disease, peripheral vascular disease, pneumonia secondary to impaired immune function, pulmonary disease, pulmonary emphysema, pulmonary fibrosis, reduced fitness, renal disease, renal insufficiency, scoliosis, spinal stenosis, syndrome X, tinnitus, urinary incontinence, vertebral fracture, weight loss, coronary artery disease, diabetes mellitus, type 2 diabetes, osteoarthritis, rheumatoid arthritis, sarcopenia, hypertension, atherosclerosis, ischemia, reperfusion injury, premature death, vascular insufficiency, interstitial lung disease, age-related decline in cognitive function, age-related decline in cardiopulmonary function, age-related decline in muscle strength, age-related decline in vision, and age-related decline in hearing.
Paragraph 54. The method of paragraph 52, wherein the dermatological disease is a senescence-associated dermatological disease selected from rough skin, formation of wrinkles, coloring or spots, abnormal coloration of skin, formation of sagging, easy skin damage, atrophy, diabetic ulcers, and other ulcers.
Paragraph 55. The method of paragraph 52, wherein the ophthalmic disease is senescence-associated ophthalmic disease selected from cataract, corneal abrasion, conjunctivitis, chalazion, glaucoma, macular degeneration, and age-related macular degeneration.
Paragraph 56. The method of paragraph 52, wherein the neurological disease is selected from Alzheimer's disease, hearing loss, dementia, chronic traumatic encephalopathy, brain atrophy, amyotrophic lateral sclerosis, Parkinson's disease, Gillian-Barre syndrome, peripheral neuropathy, Creutzfeldt-Jakob disease, frontotemporal dementia, spinal muscular atrophy, and Friedreich's ataxia, vascular dementia, mild cognitive impairment, severe cognitive impairment, memory loss, pontocerebellar hypoplasia, motor neuron disease, Machado-Joseph disease, spino-cerebellar ataxia, Multiple sclerosis, Huntington's disease, hearing impairment, balance impairment, ataxias, epilepsy, mood disorder, schizophrenia, bipolar disorder, depression, Pick's Disease, stroke, CNS hypoxia, cerebral senility, neural injury, and head trauma.
Paragraph 57. The method of paragraph 52, wherein the cancer is selected from bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.
Paragraph 58. The method of any one of paragraphs 39-57, comprising administering the compound to the subject orally.
Paragraph 59. The method of paragraph 58, comprising administering the compound in a dosage form selected from a capsule, a tablet, and a sachet.
Paragraph 60. The method of any one of paragraphs 39-57, comprising administering the compound to the subject intravascularly.
Paragraph 61. The method of paragraph 60, comprising administering the compound in a dosage form comprising an injectable or infusible aqueous solution.
Paragraph 62. The method of any one of paragraphs 39-61, the therapeutically effective amount of the compound is from about 50 mg/kg/day to about 500 mg/kg/day.
Paragraph 63. The method of paragraph 62, wherein the therapeutically effective amount of the compound is from about 130 mg/kg/day to about 400 mg/kg/day.
Paragraph 64. The method of paragraph 63, wherein the therapeutically effective amount is about 130 mg/kg/day, about 260 mg/kg/day, or about 400 mg/kg/day.
Paragraph 65. The method of any one of paragraphs 39-64, wherein the compound is administered one a day.
Paragraph 66. The method of any one of paragraphs 39-64, wherein the compound is administered twice a day.
Paragraph 67. The method of any one of paragraphs 39-64, wherein the compound is administered three times a day.
Paragraph 68. The method of any one of paragraphs 39-67, comprising administering at least two compounds of Formula (A), or a pharmaceutically acceptable salt thereof.
Paragraph 69. The method of paragraph 68, comprising co-administering:

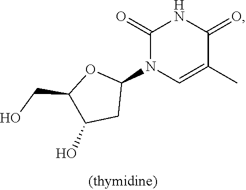 [SMILES: Cc1cn([C@H]2C[C@H](O)[C@@H](CO)O2)c(=O)[nH]c1=O]

or a pharmaceutically acceptable salt thereof, and

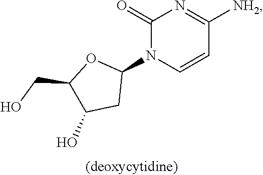 [SMILES: Nc1ccn([C@H]2C[C@H](O)[C@@H](CO)O2)c(=O)n1]

or a pharmaceutically acceptable salt thereof.
Paragraph 70. The method of any one of paragraphs 39-69, wherein the compound is administered in combination with an additional therapeutic agent useful in treating a telomere biology disorder Paragraph 71. The method of paragraph 70, wherein the additional therapeutic agent is purine nucleoside, or a pharmaceutically acceptable salt thereof.
Paragraph 72. The method of paragraph 71, wherein the purine nucleoside is selected from adenosine (A), deoxyadenosine (dA), guanosine (G), and deoxyguanosine (dG), or a pharmaceutically acceptable salt thereof.
Paragraph 73. The method of paragraph 70, wherein the additional therapeutic agent is an inhibitor of dNTPase SAM domain and HD domain-containing protein 1 (SAMHD1), or a pharmaceutically acceptable salt thereof.
Paragraph 74. The method of paragraph 73, wherein the SAMHD1 inhibitor is selected from miRNA181a/b, an anti-SAMHD1 antibody, a T cell receptor, and a protein having at least 75% identity to VPX or VPR.
Paragraph 75. The method of paragraph 73, wherein the SAMHD1 inhibitor is selected from erythrotyrosine, sennoside A, evans blue, merbromin, phenylmercuric acetate, thiram, bronopol, cephalosporin C, pidolic acid, diphenhydramine, aurothiomalate, rose bengal, chlorambucil, pyrithione zinc, lomofungin, troglitazone, montelukast, pranlukast, L-thyroxine, ergotamine, amrinone, retinoic acid, ethacrynic acid, hexestrol, tolfenamic acid, bexarotene, sulindac, zolmitriptan, nifedipine, tetracycline, nisoldipine, medroxyprogesterone acetate, trifluoperazine, primaquine, adapalene, aprepitant, tolcapone, zafirlukast, delavirdine, topotecan, ceftazidime, zoledronic acid, anethole-trithione, and disulfiram, or a pharmaceutically acceptable salt thereof.
Paragraph 76. The method of paragraph 70, wherein the additional therapeutic agent is an inhibitor of thymidine phosphorylase (TYMP), or a pharmaceutically acceptable salt thereof.
Paragraph 77. The method of paragraph 76, wherein the TYMP inhibitor is selected from tipiracil, 6-aminothymine (6AT), amino-5-chlorouracil (6A5CU), 6-amino-5-bromouracil (6A5BU), 7-deazaxanthine (7DX), 7-(2-aminoethyl)-deazaxanthine, 6-(2-aminoethylamino)-5-chlorouracil (AEAC), 5-chloro-6-(1-imidazolylmethyl)uracil (CIMU), 6-methylenepyridinium, 6-(4-phenylbutylamino)uracil, 5-chloro-6-[1-(2-iminopyrrolidinyl)methyl]uracil hydrochloride (TPI), and N-(2,4-dioxo-1,2,3,4-tetrahydro-thieno[3,2-d]pyrimidin-7-yl)guanidine, or a pharmaceutically acceptable salt thereof.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A method of treating a telomere biology disorder, the method comprising administering to a subject diagnosed with said telomere biology disorder a therapeutically effective amount of a compound of Formula (A): 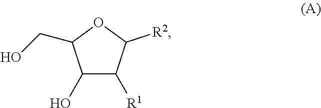
or a pharmaceutically acceptable salt thereof, wherein:
R¹ is selected from H and OH;
R² is selected from any one of the following moieties: 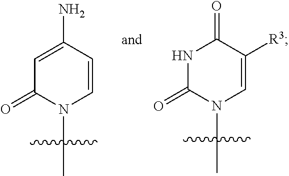
and
R³ is selected from H and CH₃.

2. The method of claim 1, wherein the compound has formula: 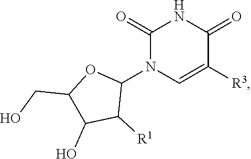
or a pharmaceutically acceptable salt thereof.

3. The method of claim 1, wherein R³ is H.

4. The method of claim 1, wherein R³ is CH₃.

5. The method of claim 1, wherein the compound has formula: 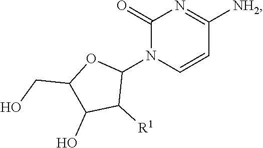
or a pharmaceutically acceptable salt thereof.

6. The method of claim 1, wherein R¹ is H.

7. The method of claim 1, wherein R¹ is OH.

8. The method of claim 1, wherein the compound of Formula (A) has Formula (I): 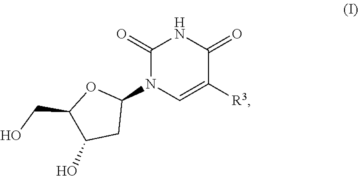
or a pharmaceutically acceptable salt thereof.

9. The method of claim 8, wherein the compound of Formula (I) is: 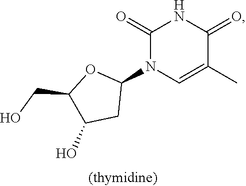 [SMILES: Cc1cn([C@H]2C[C@H](O)[C@@H](CO)O2)c(=O)[nH]c1=O]
or a pharmaceutically acceptable salt thereof.

10. The method of claim 8, wherein the compound of Formula (I) is: 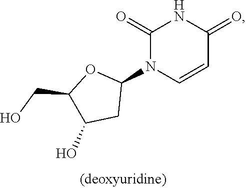 [SMILES: O=c1ccn([C@H]2C[C@H](O)[C@@H](CO)O2)c(=O)[nH]1]
or a pharmaceutically acceptable salt thereof.

11. The method of claim 1, wherein the compound of Formula (A) is: 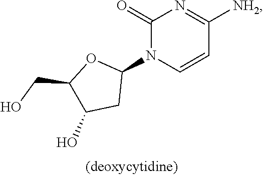 [SMILES: Nc1ccn([C@H]2C[C@H](O)[C@@H](CO)O2)c(=O)n1]
or a pharmaceutically acceptable salt thereof.

12. The method of claim 1, wherein the compound of Formula (A) is: 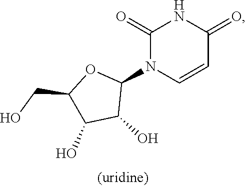 [SMILES: O=c1ccn([C@@H]2O[C@H](CO)[C@@H](O)[C@H]2O)c(=O)[nH]1]
or a pharmaceutically acceptable salt thereof.

13. The method of claim 1, wherein the compound of Formula (A) is: 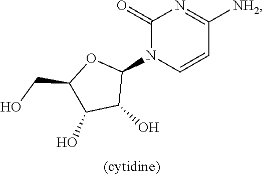 [SMILES: Nc1ccn([C@@H]2O[C@H](CO)[C@@H](O)[C@H]2O)c(=O)n1]
or a pharmaceutically acceptable salt thereof.

14. The method of claim 1, wherein the telomere biology disorder is selected from dyskeratosis congenita, aplastic anemia, myelodysplastic syndrome, pulmonary fibrosis, idiopathic pulmonary fibrosis, interstitial lung disease, hematological disorder, liver disease, hepatic fibrosis, Hoyeraal-Hreidarsson syndrome, Coats Plus syndrome, and Revesz syndrome.

15-18. (canceled)

19. The method of claim 1, comprising administering the compound to the subject orally.

20. (canceled)

21. The method of claim 1, comprising administering the compound to the subject intravascularly.

22-25. (canceled)

26. The method of claim 1, wherein the compound is administered one a day.

27-28. (canceled)

29. The method of claim 1, comprising administering at least two compounds of Formula (A), or a pharmaceutically acceptable salt thereof.

30. The method of claim 29, comprising co-administering: 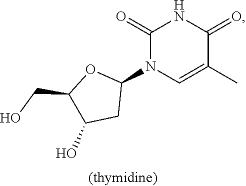 [SMILES: Cc1cn([C@H]2C[C@H](O)[C@@H](CO)O2)c(=O)[nH]c1=O]
or a pharmaceutically acceptable salt thereof, and 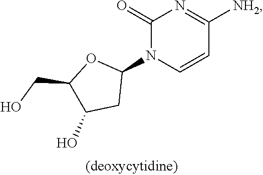 [SMILES: Nc1ccn([C@H]2C[C@H](O)[C@@H](CO)O2)c(=O)n1]
or a pharmaceutically acceptable salt thereof.

31. The method of claim 1, wherein the compound is administered in combination with an additional therapeutic agent useful in treating a telomere biology disorder.

32-38. (canceled)
